(19) United States
(12) Patent Application Publication
(10) Pub. No.: US 2026/0097047 A1
(43) Pub. Date: Apr. 9, 2026

(54) Small molecule inhibitors of the ribosome biogenesis factor NVL2

(71) Applicant: Board of Regents, The University of Texas System, Austin, TX (US)

(72) Inventors: Jef De Brabander, Dallas, TX (US); Deepak Nijhawan, Dallas, TX (US); Ye Tao, Dallas, TX (US); Holly Huizi Guo, Dallas, TX (US)

(73) Assignee: Board of Regents, The University of Texas System, Austin, TX (US)

(21) Appl. No.: 19/408,373

(22) Filed: Dec. 4, 2025

Related U.S. Application Data
(63) Continuation of application No. PCT/US24/32171, filed on Jun. 2, 2024.
(60) Provisional application No. 63/507,089, filed on Jun. 8, 2023.

(51) Int. Cl. A61K 31/554; A61K 31/55; A61P 35/00; C07D 223/18; C07D 401/12; C07D 403/12; C07D 405/12; C07D 417/12; C07D 513/04

(52) U.S. Cl. CPC A61K 31/554; A61K 31/55; A61P 35/00; C07D 223/18; C07D 401/12; C07D 403/12; C07D 405/12; C07D 417/12; C07D 513/04

(57) ABSTRACT

Described are dibenzothiazepinone and dibenzazepinone-based compounds, methods for treating cancers with small molecule inhibitors of the ribosome biogenesis factor NVL2, related pharmaceutical compositions, methods to inhibit ribosome biogenesis or induce p53 or inhibit cancer cells and methods of screening for candidate therapeutics for treating cancer.

### CROSS-REFERENCES TO RELATED APPLICATIONS

This application is a continuation of PCT/US24/32171, filed Jun. 2, 2024, which claims priority to U.S. Provisional Application No. 63/507,089; filed: Jun. 8, 2023, the disclosure of which are hereby incorporated by reference in its entirety for all purposes.

### INTRODUCTION

Ribosomes are large macromolecular ribonucleoprotein (RNP) complexes, composed of a small 40S subunit and a large 60S subunit, responsible for translating protein from messenger RNA. The synthesis of 40S and 60S requires approximately two hundred ribosome biogenesis factors (RBFs). Among these, a large subset is responsible for hierarchical ribosome assembly, including rRNA processing and modifying enzymes, structural proteins that template assembly steps, and diverse ATPases that remodel rRNA or actively remove RBFs at critical junctions to guide ribosome maturation (1-3).

Ribosome abundance and translation activity are tightly coupled to cellular growth rates, and dysregulated ribosome biogenesis can directly signal changes in the cell cycle. The buildup of assembly intermediates that results from a block in ribosome biogenesis triggers a specific cellular checkpoint (4, 5). This process is initiated by 5S RNP modules that fail to be incorporated into pre-60S nucleolar intermediates (6, 7). Unincorporated 5S RNPs adopt a conformation that binds and inhibits MDM2 (8, 9), an E3 ubiquitin ligase that constitutively ubiquitinates p53, leading to its proteasomal degradation (5). Consequently, the inhibition of MDM2 by 5S RNP results in increased p53 protein levels. Transcriptional activation of p53 target genes in turn triggers cell cycle arrest, senescence, or apoptosis (10). Cancer cells have long been known to engage in ribosome biogenesis at significantly enhanced levels to support the protein synthesis required for uncontrolled cell growth. As such, inhibitors of ribosome biogenesis have the potential to block cancer growth and trigger checkpoints that result in p53-dependent cancer cell death (8, 11-13).

Although there are numerous small molecules that block ribosome production, these molecules have alternative cytotoxic targets making it difficult to determine the therapeutic potential of targeting ribosome biogenesis. For example, many cancer chemotherapies that trigger DNA damage, including 5-fluorouracil, doxorubicin, and camptothecin, also have been reported to inhibit ribosome biogenesis (14). Similarly, oxaliplatin uniquely induces a change in nucleolar morphology that coincides with a block in ribosome biogenesis (15-18). Yet, like its related analogs cisplatin and carboplatin, oxaliplatin also leads to DNA adducts that trigger the DNA damage response pathway. Recent studies have given evidence that the ability of oxaliplatin to impede ribosome production might be of cenral importance to its anti-cancer activity (19). Since oxaliplatin also causes DNA damage, however, it is difficult to determine the individual contributions of DNA damage and ribosome biogenesis inhibition to therapeutic benefits and toxic side-effects. Efforts to develop more selective inhibitors of ribosome biogenesis have focused on inhibiting RNA polymerase I, which is responsible for transcribing ribosomal RNA. Unfortunately, the most advanced of these compounds, CX-5461, also induces DNA damage by inhibiting the topoisomerase TOP2B raising the possibility that its anti-cancer activity may be through this latter target (20).

As an alternative to POLI-directed inhibition, the assembly phase of ribosome biogenesis includes many enzymes that could hypothetically be targeted with small molecules. The coupling of ATP hydrolysis to the mechanical removal of specific ribosome biogenesis factors is a critical step in the assembly of the large ribosomal subunit (60S). Ribosome assembly and maturation is mediated by several catalytic AAA+ ATPase enzymes including NVL, MDN1 and SPATA5 (2, 25-28). Small molecules that disrupt AAA+ ATPase function during 60S biogenesis have been characterized for S. cerevisiae Drg1 (2, 29, 30) and S. pombe Mdn1 (31), but no chemical inhibitors have been reported for the homologous mammalian enzymes.

### SUMMARY OF THE INVENTION

The invention provides small molecule inhibitors of the ribosome biogenesis factor NVL2. In one aspect the invention provides a compound of formula (I):

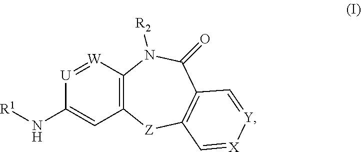

wherein:
R1 is selected from: R3; —C(O)R3; —C(S)R3; —C(O)NHR3; —C(S)NHR3; —C(O)NR5R6; —C(S)NR5R6;
R2 is selected from: C1-C8alkyl; C2-C8alkenyl; C2-C8alkynyl including all isomers thereof and all optionally substituted with one or more of: D, F, OH, C(O)NHR4 and all optionally having one or more methylene units replaced with O, S, NH, NR4, C(O);
R3 is selected from: C1-C7alkyl, C3-C7cycloalkyl optionally bridged with CH₂ or CH₂CH₂, CH₂-linked or CHMe-linked C3-C7cycloalkyl optionally bridged with CH₂ or CH₂CH₂, C2-C7alkenyl, C5-C7cycloalkenyl optionally bridged with CH₂ or CH₂CH₂, CH₂-linked or CHMe-linked C5-C7cycloalkenyl optionally bridged with CH₂ or CH₂CH₂, C2-C7alkynyl, bridged or fused C5-C9cycloalkyl, CH₂-linked or CHMe-linked bridged or fused C5-C9cycloalkyl, C5-C10spiroalkanes, CH₂-linked or CHMe-linked C5-C10spiroalkanes, all including all possible isomers thereof and all optionally substituted with one or more of: D, F, CN, R4, OR4, OH, NHC(O)H, NHC(O)R4, NHSO₂R4, CO₂H, CO₂R4, C(O)NH₂, C(O)NHR4, C(O)NR4R4 and all optionally having one or more methylene units replaced with O, S, SO₂, S(O), NH, NR4, NC(O)H, NC(O)R4, NSO₂R4, C(O), C═NOH, C═NOR4 or C═NR4, phenyl or naphtyl (optionally substituted at one or more of each position with R7), 5- and 6-membered heteroaryls and fused heteroaryls including but not limited to oxazoles, isoxazoles, thiazoles, isothiazoles, furans, thiophenes, pyrazoles, imidazoles, triazoles, tetrazoles, pyridines, pyrimidines, pyrazines, pyridazines, quinolines, isoquinolines, quinazolines, quinoxalines, cinnolines, phtalazines, naphthyridines, pyridopyrazines, pyrazolopyridines, pyridopyrimidines, pyridopyridazines, benzoxazoles, benzoisoxazoles, benzothiazoles, benzoisothiazoles, indoles, indazoles, benzimidazoles, dioxolopyridines, dioxolopyridazines, dioxolopyrimidines, dioxolopyrazines, benzofurans, benzothiophenes, imidazopyridines, benzodioxoles, benzoxazolones, benzoisoxazolones, benzothiazolones, benzoisothiazolones, 1,3-dihydro-2H-benzimidazoles, benzo[d]imidazol-2-ones, benzodioxolones, dioxolopyridines, dioxolopyridazines, dioxolopyrimidines, dioxolopyrazines, coumarins, isocoumarins, and including all possible isomers thereof, and all optionally substituted at one or more of each position with R7;
R4 is selected from: Me; Et; Pr; iPr; cPr; cBu optionally substituted with one or more of: D, F, CN, OH, OCD₃, OCHₙF₍₃₋ₙ₎ (n=0-3);
NR5R6 completes a 4- to 7-membered azacycle or a bridged or fused 6- to 9-membered azacycle or 5- to 9-membered azaspiroalkane including all possible isomers thereof and all optionally substituted with one or more of: D, F, CN, R4, OR4, OH, NHC(O)H, NHC(O)R4, NHSO₂R4, CO₂H, CO₂R4, C(O)NH₂, C(O)NHR4, C(O)NR4R4 and all optionally having one or more methylene units replaced with O, S, SO₂, NH, NR4, NC(O)H, NC(O)R4, NSO₂R4, C(O), C═NOH, C═NR4;
R7 is selected from: D, F, Cl, Br, CN, N₃, OH, OR4, R4, oxetanyl, NHC(O)H, NHC(O)R4, CO₂H, CO₂R4, C(O)NH₂, C(O)NHR4, C(O)NR4R4, SO₂NH₂, SO₂NHR4, SO₂NR4R4, C2-C6alkenyl or C2-C6alkynyl including all isomers thereof and all optionally substituted with one or more of: D, F, CN, OH and all optionally having one or more methylene units replaced with O, S, S(O), SO₂, NH, NR4, NC(O)H, NC(O)R4, NSO₂R4, C(O), C═NOH, C═NR4; aryl or 5- or 6-membered heteroaryl or heterocyclyl all optionally substituted with one or more of: D, F, Cl, CN, R4, OH, OR4);
Z is selected from: S, CH₂, CD₂, CHR4, CDR4, CR4R4, CHOH, CDOH, CHOR4, CDOR4, CR4OH, CR4OR4, CHCN, CDCN, CHF, CDF, CF₂, NH, NR4, NC(O)H, NC(O)R4, NSO₂R4;
X is selected from: CH, CD, CF, CCl, CCN;
Y is selected from: CH, CD, CF, CCl;
U and W are independently selected from: CH, CD, N;
excluding R1=C(O) furan-3-yl, X, Y, U, W=CH, Z=S, R2=(CH₂)₂OMe (MM017), or a salt, hydrate or stereoisomer thereof.

In embodiments the invention provides:

2. The compound of claim 1, further of formula II:

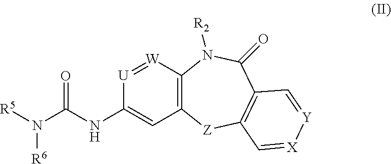

3. The compound of claim 1, further of formula III:

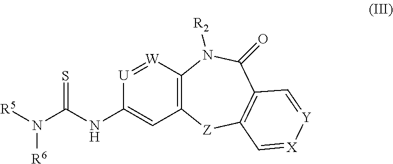

4. The compound of claim 1, further of formula IV:

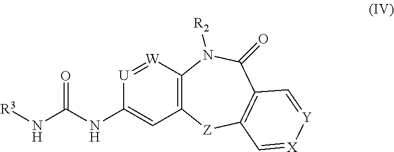

5. The compound of claim 1, further of formula V:

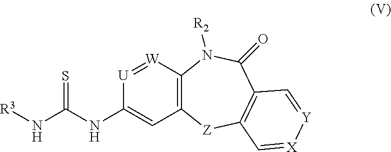

6. The compound of claim 1, further of formula VI:

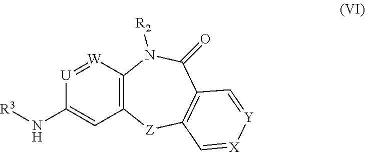

7. The compound of claim 1, further of formula VII:

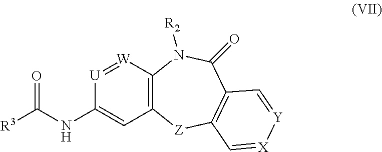

8. The compound of claim 1, further of formula VIII:

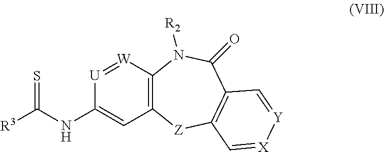

9. A compound of a claim 1-8, wherein Z=S, CH₂, CD₂, CHMe, CHCD₃, CDMe, CDCD₃, CHCN, CDCN, CMeOH, CCD₃OH, preferably S, CH₂, CHMe, CHCD₃.

10. A compound of a claim 1-8, wherein X=CH, CD, CF, CCl, CCN, preferably CH, CF, or CCl.

11. A compound of a claim 1-8, wherein Y=CH, CD, CF, CCl, preferably CH, or CF. 12. A compound of a claim 1-8, wherein W=CH, CD, N.

13. A compound of a claim 1-8, wherein U=CH, CD, N, preferably CH.

14. A compound of a claim 1-8, wherein R2=C2-C7alkyl; C3-C7alkenyl; C3-C7alkynyl including all isomers thereof and all optionally substituted with one or more of: D, F, Cl, and all optionally having one or more methylene units replaced with O.

15. A compound of a claim 1-8, wherein R2=CH₂C≡CH, CH₂C≡CR4, CH₂C≡CCl, CH₂CH═CH₂, CH₂CH═CHR4, (CH₂)₂OR4, (CH₂)₂OCH₂C≡CH, where R2 can contain one or more deuterium.

16. A compound of a claim 1-8, wherein R2=CH₂C≡CH, CH₂C≡CMe, CH₂C≡CCH₂F, CH₂C≡C^{c}Pr, CH₂C≡CCl, CH₂CH═CHMe, CH₂C≡CCF₃ where R2 can contain one or more deuterium.

17. A compound of claim 2-3, wherein:
R2=CH₂C≡CH, CH₂C≡CMe, CH₂C≡CCH₂F, CH₂C≡CCl, CH₂C≡C^{c}Pr, CH₂CH═CHMe, where R2 can contain one or more deuterium;
NR5R6 completes a 4- to 7-membered azacycle or a bridged or fused 6- to 9-membered azacycle or 5- to 9-membered azaspiroalkane including all possible isomers thereof and all optionally substituted with one or more of: D, F, CN, R4, OR4, OH, NHC(O)H, NHC(O)R4, NHSO₂R4, CO₂H, CO₂R4, C(O)NH₂, C(O)NHR4, C(O)NR4R4 and all optionally having one or more methylene units replaced with O, S, SO₂, NH, NR4, NC(O)H, NC(O)R4, NSO₂R4, C(O), C═NOH, C═NR4;
R4=Me; Et; Pr; iPr; cPr; cBu optionally substituted with one or more of: D, F, CN, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CD, CF, CCl;
Y=CH, CD, CF;
W=CH, N; and
U=CH, CD, N preferably CH.

18. A compound of claim 2-3, wherein:
R2=CH₂C≡CH, CH₂C≡CMe, CH₂C≡CCH₂F, CH₂C≡CCl, CH₂C≡C^{c}Pr preferably CH₂C≡CMe, CH₂C≡C^{c}Pr;
NR5R6 completes a 4- to 7-membered azacycle or a 6- to 8-membered azabicycle or a 5- to 7-membered azaspiroalkane, all optionally substituted with one or more of: D, F, CN, R4, OR4, OH and all optionally having one or more methylene units replaced with O, S, SO₂, NH, NR4, C(O), C═NOH, C═NR4;
R4=Me; Et; Pr; iPr; cPr; cBu optionally substituted with one or more of: D, F, CN, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

19. A compound of a claim 4, 5, or 8, wherein:
R2=CH₂C≡CH, CH₂C≡CMe, CH₂C≡CCH₂F, CH₂C≡CCl, CH₂C≡C^{c}Pr, CH₂CH═CHMe, where R2 can contain one or more deuterium;
R3=phenyl (optionally substituted at one or more of each individual position with R7); 5- and 6-membered heteroaryls and fused heteroaryls including but not limited to oxazoles, isoxazoles, thiazoles, isothiazoles, furans, thiophenes, pyrazoles, imidazoles, triazoles, tetrazoles, pyridines, pyrimidines, pyrazines, pyridazines, quinolines, isoquinolines, quinazolines, quinoxalines, cinnolines, phtalazines, naphthyridines, pyridopyrazines, pyrazolopyridines, pyridopyrimidines, pyridopyridazines, benzoxazoles, benzoisoxazoles, benzothiazoles, benzoisothiazoles, indoles, indazoles, benzimidazoles, dioxolopyridines, dioxolopyridazines, dioxolopyrimidines, dioxolopyrazines, benzofurans, benzothiophenes, imidazopyridines, benzodioxoles, benzoxazolones, benzoisoxazolones, benzothiazolones, benzoisothiazolones, 1,3-dihydro-2H-benzimidazoles, benzo[d]imidazol-2-ones, benzodioxolones, dioxolopyridines, dioxolopyridazines, dioxolopyrimidines, dioxolopyrazines, coumarins, isocoumarins, and including all possible isomers thereof, and all optionally substituted at one or more of each individual position with R7;
R7=D, F, Cl, Br, CN, N₃, OH, R4, OR4, C≡CH, NHC(O)H, C(O)NH₂, C(O)NHR4, C(O)NR4R4;
R4=Me, Et, Pr, iPr, cPr, cBu optionally substituted with one or more of: D, F, CN, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

20. A compound of a claim 4, 5, or 8, wherein:
R2=CH₂C≡CH, CH₂C≡CMe, CH₂C≡CCH₂F, CH₂C≡CCl, CH₂C≡C^{c}Pr preferably CH₂C≡CMe, CH₂C≡C^{c}Pr;
R3=a mono-or-disubstituted phenyl ring with substituents at the ortho-position selected from H, D or F and substituents at the meta and para-position selected from H, D, F, Cl, R4, OR4, N₃, C≡CH; a heteroaromatic ring selected from furan, pyridine, pyrimidine, pyrazine, pyridazine, benzo[d]oxazol-5-yl, benzo[d]thiazol-5-yl, 2-oxo-2,3-dihydrobenzo[d]oxazol-5-yl, benzo[d][1,3]dioxol-5-yl and all optionally substituted at one or more of each individual position with H, D, F, Cl, CCH, R4, OR4;
R4=Me; Et; Pr; iPr; cPr; cBu optionally substituted with one or more of: D, F;
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

21. A compound of a claim 4, 5, or 8, wherein:
R2=CH₂C≡CH, CH₂C≡CMe, CH₂C≡CCH₂F, CH₂C≡CCl, CH₂C≡C^{c}Pr, CH₂CH═CHMe, where R2 can contain one or more deuterium;
R3=C1-C7alkyl, C3-C7cycloalkyl optionally bridged with CH₂ or CH₂CH₂, CH₂-linked or CHMe-linked C3-C6cycloalkyl optionally bridged with CH₂ or CH₂CH₂, C2-C7alkenyl, C5-C6cycloalkenyl optionally bridged with CH₂ or CH₂CH₂, CH₂-linked or CHMe-linked C5-C6cycloalkenyl optionally bridged with CH₂ or CH₂CH₂, C2-C7alkynyl, bridged or fused C5-C9cycloalkyl, CH₂-linked or CHMe-linked bridged or fused C5-C9cycloalkyl, C5-C10spiroalkanes, CH₂-linked or CHMe-linked C5-C10spiroalkanes, all including all possible isomers thereof and all optionally substituted with one or more of: D, F, CN, R4, OR4, OH, NHC(O)H, NHC(O)R4, NHSO₂R4, CO₂H, CO₂R4, C(O)NH₂, C(O)NHR4, C(O)NR4R4 and all optionally having one or more methylene units replaced with O, S, SO₂, S(O), NH, NR4, NC(O)H, NC(O)R4, NSO₂R4, C(O), C═NOH, C═NOR4 or C═NR4;
R4=Me, Et, Pr, iPr, cPr, cBu optionally substituted with one or more of: D, F, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

22. A compound of claim 6, wherein:
R2=CH₂C≡CH, CH₂C≡CMe, CH₂C≡CCH₂F, CH₂C≡CCl, CH₂C≡C^{c}Pr, CH₂CH═CHMe, CH₂═CHEt, CH₂CH═CHPr, (CH₂)₂OCH₂CCH where R2 can contain one or more deuterium;
R3=phenyl (optionally substituted at one or more of each individual position with R7); 5- and 6-membered heteroaryls and fused heteroaryls including but not limited to oxazoles, isoxazoles, thiazoles, isothiazoles, furans, thiophenes, pyrazoles, imidazoles, triazoles, tetrazoles, pyridines, pyrimidines, pyrazines, pyridazines, quinolines, isoquinolines, quinazolines, quinoxalines, cinnolines, phtalazines, naphthyridines, pyridopyrazines, pyrazolopyridines, pyridopyrimidines, pyridopyridazines, benzoxazoles, benzoisoxazoles, benzothiazoles, benzoisothiazoles, indoles, indazoles, benzimidazoles, dioxolopyridines, dioxolopyridazines, dioxolopyrimidines, dioxolopyrazines, benzofurans, benzothiophenes, imidazopyridines, benzodioxoles, benzoxazolones, benzoisoxazolones, benzothiazolones, benzoisothiazolones, 1,3-dihydro-2H-benzimidazoles, benzo[d]imidazol-2-ones, benzodioxolones, dioxolopyridines, dioxolopyridazines, dioxolopyrimidines, dioxolopyrazines, coumarins, isocoumarins, and including all possible isomers thereof and all optionally substituted at one or more of each individual position with R7;
R7=D, F, Cl, Br, CN, N₃, OH, R4, OR4, C≡CH, NHC(O)H, C(O)NH₂, C(O)NHR4, C(O)NR4R4;
R4=Me, Et, Pr, iPr, cPr, cBu optionally substituted with one or more of: D, F, CN, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

23. A compound of a claim 6, wherein:
R2=CH₂C≡CH, CH₂C≡CMe, CH₂C≡CCH₂F, CH₂C≡CCl, CH₂C≡C^{c}Pr, CH₂CH═CHMe, CH₂=CHEt, CH₂CH═CHPr;
R3=5- and 6-membered heteroaryls including but not limited to pyrazoles, imidazoles, pyridines, pyrimidines, pyrazines, pyridazines, quinolines, isoquinolines, quinazolines, quinoxalines, cinnolines, phtalazines, and including all possible isomers thereof, and all optionally substituted at one or more of each individual position with D, F, Cl, CN, Me, cPr, CD₃, OCD₃, OH, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

24. A compound of a claim 6, wherein:
R2=CH₂C≡CH, CH₂C≡CMe, CH₂C≡CCH₂F, CH₂C≡CCl, CH₂C≡C^{c}Pr preferably CH₂C≡CMe, CH₂C≡C^{c}Pr;
R3=pyrazoles, pyridines, pyrimidines, pyrazines, pyridazines all optionally substituted at one or more of each individual position with D, F, Cl, CN, Me, cPr, CD₃, OCD₃, OH, OCHₙF₍₃₋ₙ₎ (n=0-3);
Z=S, CH₂, CHMe, CHCN, CMeOH preferably S, CH₂, CHMe;
X=CH, CF, CCl;
Y=CH, CF;
W=CH, N; and
U=CH, N preferably CH.

25. A compound of a claim 6, wherein:
R2=CH₂C≡CH, CH₂C≡CMe, CH₂C≡CCH₂F, CH₂C≡CCl, CH₂C≡C^{c}Pr, where R2 can contain one or more deuterium;
R3=C2-C4alkyl, C2-C4alkenyl, C3-C6cycloalkyl optionally bridged with CH₂ or CH₂CH₂, CH₂-linked or CHMe-linked C3-C6cycloalkyl optionally bridged with CH₂ or CH₂CH₂, C5-C6cycloalkenyl optionally bridged with CH₂ or CH₂CH₂, CH₂-linked or CHMe-linked C5-C6cycloalkenyl optionally bridged with CH₂ or CH₂CH₂, C5-C8spiroalkanes, CH₂-linked or CHMe-linked C5-C8spiroalkanes, and all optionally substituted with one or more of: D, F, CN, OH, R4, OCD₃, OCHₙF₍₃₋ₙ₎ (n=0-3) and all optionally having one or more methylene units replaced with O, S, SO₂, S(O), NH, NR4, NC(O)H, NC(O)R4, NSO₂R4, C(O), C═NOH, C═NOR4 or C═NR4;
R4=Me, Et, Pr, iPr, cPr, cBu optionally substituted with one or more of: D, F, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

26. A compound of a claim 17-18, wherein:
R2=CH₂C≡CMe, CH₂C═CCD₃, CH₂C≡CCH₂F, CH₂C≡C^{c}Pr, CH₂C≡CCl, CH₂C≡CH preferably CH₂C≡CMe, CH₂C≡C^{c}Pr;
NR5R6=N-linked azetidine, pyrrolidine, piperidine, azepine optionally substituted with one or more of: D, F, CN, R4, OR4, OH and optionally bridged with CH₂ or CH₂CH₂ and all optionally having one or more methylene units replaced with O, NR4, N═OH, NOMe, N═OCD₃;
R4=Me, cPr optionally substituted with one or more of: D, F, CN, OH, OCD₃, OCHₙF₍₃₋ₙ₎ (n=0-3);
Z=S, CH₂, CHMe preferably S;
X=CH, CF, CCl;
Y=CH, F preferably CH;
W=CH, N; and
U=CH.

27. A compound of a claim 22-24, wherein:
R2=CH₂C≡CMe, CH₂C═CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=pyridines, pyrimidines, pyrazines, pyridazines all optionally substituted at one or more of each individual position with D, F, Cl, CN, Me, Et, iPr, cPr, OH, OCD₃, OCHₙF₍₃₋ₙ₎ (n=0-3);
Z=S, CH₂, CHMe preferably S;
X=CH, CF, CCl;
Y=CH, CF preferably CH;
W=CH, N; and
U=CH.

28. A compound of a claim 25, wherein:
R2=CH₂C≡CMe, CH₂C═CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=C2-C4alkyl, C2-C4alkenyl, C3-C6cycloalkyl optionally bridged with CH₂ or CH₂CH₂, CH₂-linked or CHMe-linked C3-C6cycloalkyl optionally bridged with CH₂ or CH₂CH₂, C5-C6cycloalkenyl optionally bridged with CH₂ or CH₂CH₂, CH₂-linked or CHMe-linked C5-C6cycloalkenyl optionally bridged with CH₂ or CH₂CH₂, C5-C8spiroalkanes, CH₂-linked or CHMe-linked C5-C8spiroalkanes, all including all possible isomers thereof and all optionally substituted with one or more of: D, F, Me, CD₃, cPr, OH, OCD₃, OCHₙF₍₃₋ₙ₎ (n=0-3), CH₂OH, CHMeOH, CH₂OCD₃, CH₂OCHₙF₍₃₋ₙ₎ (n=0-3), CHMeOCD₃, CHMeOCHₙF₍₃₋ₙ₎ (n=0-3), and all optionally having one or more methylene units replaced with O, S, SO₂NH, NMe, NCD₃, CO, N═OH, NOMe, N═OCD₃;
Z=S, CH₂, CHMe preferably S;
X=CH, CF, CCl;
Y=CH, CF preferably CH;
W=CH, N; and
U=CH.

29. A compound of a claim 20, wherein:
R2=CH₂C≡CMe, CH₂C≡CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=a mono-or-disubstituted phenyl ring with substituents at the ortho-position selected from H, D or F and substituents at the meta and para-position selected from H, D, F, Cl, R4, OR4, N₃, C≡CH; a heteroaromatic ring selected from furan, pyridine, pyrimidine, pyrazine, pyridazine, benzo[d]oxazol-5-yl, benzo[d]thiazol-5-yl, 2-oxo-2,3-dihydrobenzo[d]oxazol-5-yl, benzo[d][1,3]dioxol-5-yl and all optionally substituted at one or more of each individual position with H, D, F, Cl, Me, CD₃, cPr, CCH, OCD₃, OCHₙF₍₃₋ₙ₎ (n=0-3);
Z=S, CH₂, CHMe preferably S;
X=CH, CF, CCl;
Y=CH, CF preferably CH;
W=CH, N; and
U=CH.

30. A compound of claim 21 wherein:
R2=CH₂C≡CMe, CH₂C═CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=C2-C4alkyl, C2-C4alkenyl, C2-C3alkynyl, C3-C6cycloalkyl optionally bridged with CH₂ or CH₂CH₂, CH₂-linked or CHMe-linked C3-C6cycloalkyl optionally bridged with CH₂ or CH₂CH₂, C5-C6cycloalkenyl optionally bridged with CH₂ or CH₂CH₂, CH₂-linked or CHMe-linked C5-C6cycloalkenyl optionally bridged with CH₂ or CH₂CH₂, C5-C8spiroalkanes, CH₂-linked or CHMe-linked C5-C8spiroalkanes, and all including all possible isomers thereof and all optionally substituted with one or more of: D, F, CN, Me, CD₃, cPr, OH, OCD₃, OCHₙF₍₃₋ₙ₎ (n=0-3), CH₂OH, CHMeOH, CH₂OCD₃, CH₂OCHₙF₍₃₋ₙ₎ (n=0-3), CHMeOCD₃, CHMeOCHₙF₍₃₋ₙ₎ (n=0-3), CO₂Me, CO₂CD₃, and all optionally having one or more methylene units replaced with O, S, SO₂, NH, NMe, NCD₃, C(O), N═OH, NOMe, N═OCD₃;
Z=S, CH₂, CHMe preferably S;
X=CH, CF, CCl;
Y=CH, CF preferably CH;
W=CH, N; and
U=CH.

31. A compound of claim 7, wherein:
R2=CH₂C≡CMe, CH₂C≡CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=a mono-or-disubstituted phenyl ring with substituents at the ortho-position selected from H, D or F and substituents at the meta and para-position selected from H, D, F, Cl, R4, OR4, N₃, C≡CH; a heteroaromatic ring selected from furan, pyridine, pyrimidine, pyrazine, pyridazine, benzo[d]oxazol-5-yl, benzo[d]thiazol-5-yl, 2-oxo-2,3-dihydrobenzo[d]oxazol-5-yl, benzo[d][1,3]dioxol-5-yl and all optionally substituted at one or more of each individual position with H, D, F, Cl, Me, CD₃, cPr, CCH, OCD₃, OCHₙF₍₃₋ₙ₎ (n=0-3);
Z=S, CH₂, CHMe preferably S;
X=CH, CF, CCl;
Y=CH, CF preferably CH;
W=CH, N; and
U=CH.

32. A compound of claim 7, wherein:
R2=CH₂C≡CMe, CH₂C≡CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=C2-C4alkyl, C2-C4alkenyl, C2-C3alkynyl, C3-C6cycloalkyl optionally bridged with CH₂ or CH₂CH₂, CH₂-linked or CHMe-linked C3-C6cycloalkyl optionally bridged with CH₂ or CH₂CH₂, C5-C6cycloalkenyl optionally bridged with CH₂ or CH₂CH₂, CH₂-linked or CHMe-linked C5-C6cycloalkenyl optionally bridged with CH₂ or CH₂CH₂, C5-C8spiroalkanes, CH₂-linked or CHMe-linked C5-C8spiroalkanes, and all including all possible isomers thereof and all optionally substituted with one or more of: D, F, CN, Me, CD₃, cPr, OH, OCD₃, OCHₙF₍₃₋ₙ₎ (n=0-3), CH₂OH, CHMeOH, CH₂OCD₃, CH₂OCHₙF₍₃₋ₙ₎ (n=0-3), CHMeOCD₃, CHMeOCHₙF₍₃₋ₙ₎ (n=0-3), CO₂Me, CO₂CD₃, and all optionally having one or more methylene units replaced with O, S, SO₂, NH, NMe, NCD₃, C(O), N═OH, NOMe, N═OCD₃;
Z=S, CH₂, CHMe preferably S;
X=CH, CF, CCl;
Y=CH, CF preferably CH;
W=CH, N; and
U=CH.

33. A compound selected from Tables 1-10

34. A compound selected from Table 7.

35. A compound selected from Tables 1-2.

36. A compound selected from Tables 3-4.

37. A compound selected from Tables 5-6.

38. A compound selected from Table 6.

39. A compound selected from Table 8-10.

40. A compound selected from Table 10.

41. A compound selected from Tables 8-9.

42. A compound selected from Tables 6 and 8.

43. A pharmaceutical composition comprising a compound of claim 1-42, or a pharmaceutically acceptable salt, a hydrate or a stereoisomer thereof, and a pharmaceutically acceptable carrier or excipient, preferably in a pharmaceutically acceptable unit dosage.

44. A method to inhibit ribosome biogenesis, induce p53 or inhibit cancer cells, comprising administering to a person in need thereof a compound of claim 1-42.

45. A method of treating a disease or condition comprising a cancer, tumor or neoplasia, comprising administering to a person in need thereof a compound of a claim 1-42, such as wherein the cancer, tumor or neoplasia comprises breast cancer, lung cancer, colorectal cancer, ovarian cancer, bladder cancer, kidney cancer, esophageal cancer, stomach cancer, cervical cancer, head and neck cancers, liver cancer, prostate cancer, pancreatic cancer, sarcoma, melanoma, leukemia, lymphoma, brain cancer, skin cancer (melanoma), thyroid cancer, testicular cancer, and multiple myeloma.

46. A method of claim 44 or 45, further comprising the antecedent step of detecting or diagnosing a disease or condition indicating the need thereof, and/or the subsequent step of detecting a resultant improvement or delay of progression of the disease or condition.

47. A method of screening for candidate therapeutics for treating cancer, comprising assaying for inhibitors or NVL2.

48. A cell line for evaluating cancer therapeutics comprising and expressing a mutant NVL gene sufficient for MM017 resistance, preferably comprising one or more mutations within the D1 AAA+ ATPase domain, such as NVL^{P307T}, NVL^{R403W} or NVL^{H304R}.

The invention encompasses all combinations of the particular embodiments recited herein, as if each combination had been laboriously recited.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

The term “alkyl” refers to a hydrocarbon group selected from linear and branched saturated hydrocarbon groups of 1-18, or 1-12, or 1-6 carbon atoms. Examples of the alkyl group include methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms; lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C≡C triple bond and of 2-18, or 2-12, or 2-6 carbon atoms. Examples of the alkynyl group include ethynyl, 1-propynyl, 2-propynyl (propargyl), 1-butynyl, 2-butynyl, and 3-butynyl groups.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6 carbon atoms. Even further for example, the cycloalkyl group may be a monocyclic group of 3-12, or 3-8, or 3-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from: 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap with heteroaryl, separately defined below. Hence, if one or more carbocyclic aromatic rings are fused with a heterocyclic aromatic ring, the resulting ring system is heteroaryl, not aryl, as defined herein.

The term “halogen” or “halo” refers to F, Cl, Br or I.

The term “heteroalkyl” refers to alkyl comprising at least one heteroatom.

The term “heteroaryl” refers to a group selected from:
5- to 7-membered aromatic, monocyclic rings comprising 1, 2, 3 or 4 heteroatoms selected from N, O, and S, with the remaining ring atoms being carbon;
8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and
11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocyle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco [3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1, 1-dioxo-1-thiomorpholinyl.

Substituents are selected from: halogen, —R′, —OR′, ═O, ═NR′, ═N—OR′, —NR′R″, —SR′, —SiR′R″R″, —OC(O)R′, —C(O)R′, —CO₂R′, —CONR′R″, —OC(O)NR′R″, —NR″C(O)R′, —NR′—C(O)NR″R″, —NR′—SO₂NR″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN and —NO₂, —N₃, —CH(Ph)₂, perfluoro (C1-C4)alkoxy and perfluoro (C1-C4)alkyl, in a number ranging from zero to three, with those groups having zero, one or two substituents being particularly preferred. R′, R″ and R″ each independently refer to hydrogen, unsubstituted (C1-C8)alkyl and heteroalkyl, unsubstituted aryl, aryl substituted with one to three halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF₃ and —CH₂CF₃), and when the aryl group is 1,2,3,4-tetrahydronaphthalene, it may be substituted with a substituted or unsubstituted (C3-C7)spirocycloalkyl group. The (C3-C7)spirocycloalkyl group may be substituted in the same manner as defined herein for “cycloalkyl”.

Preferred substituents are selected from: halogen, —R′, —OR′, ═O, —NR′R″, —SR′, —SiR′R″R″′, —OC(O)R′, —C(O)R′, —CO₂R′, —CONR′R″, —OC(O)NR′R″, —NR″C(O)R′, —NR″CO₂R′, —NR′—SO₂NR″R″′, —S(O)R′, —SO₂R′, —SO₂NR′R″, —NR″SO₂R, —CN and —NO₂, perfluoro (C1-C4)alkoxy and perfluoro (C1-C4)alkyl, where R′ and R″ are as defined above.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicylclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The compounds may contain an asymmetric center and may thus exist as enantiomers. Where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The compounds of the invention may also contain unnatural proportions of atomic isotopes at one or more of the atoms that constitute such compounds, such as deuterium, e.g. —CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The term “substantially pure” means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds contain olefin double bonds, unless specified otherwise, such double bonds are meant to include both E and Z geometric isomers.

Some of the compounds may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, aluminum, lithium, and ammonium.

In addition, if a compound is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

“Treating,” “treat,” or “treatment” refers to administering at least one compound and/or at least one stereoisomer thereof, and/or at least one pharmaceutically acceptable salt thereof to a subject in recognized need thereof.

An “effective amount” refers to an amount of at least one compound and/or at least one stereoisomer thereof, and/or at least one pharmaceutically acceptable salt thereof effective to “treat” a disease or disorder in a subject, and that will elicit, to some significant extent, the biological or medical response of a tissue, system, animal or human that is being sought, such as when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The term “at least one substituent” includes, for example, from 1 to 4, such as from 1 to 3, further as 1 or 2, substituents. For example, “at least one substituent R” herein includes from 1 to 4, such as from 1 to 3, further as 1 or 2, substituents selected from the list of R as described herein.

The subject compounds and stereoisomers thereof, and pharmaceutically acceptable salts thereof may be employed alone or in combination with at least one other therapeutic agent for treatment. In some embodiments, the compounds, stereoisomers thereof, and pharmaceutically acceptable salts thereof can be used in combination with at least one additional therapeutic agent. The compound and/or one pharmaceutically acceptable salt disclosed herein may be administered with the at least one other therapeutic agent in a single dosage form or as a separate dosage form. When administered as a separate dosage form, the at least one other therapeutic agent may be administered prior to, at the same time as, or following administration of the compound and/or one pharmaceutically acceptable salt disclosed herein.

Also provided is a composition comprising a subject compound and stereoisomers thereof, and pharmaceutically acceptable salts thereof, and at least one pharmaceutically acceptable carrier.

The composition comprising a subject compound and stereoisomers thereof, and pharmaceutically acceptable salts thereof can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The subject compounds and stereoisomers thereof, and pharmaceutically acceptable salts thereof can be administered orally in solid dosage forms, such as capsules, tablets, troches, dragées, granules and powders, or in liquid dosage forms, such as elixirs, syrups, emulsions, dispersions, and suspensions. The subject compounds and stereoisomers thereof, and pharmaceutically acceptable salts thereof disclosed herein can also be administered parenterally, in sterile liquid dosage forms, such as dispersions, suspensions or solutions. Other dosages forms that can also be used to administer the subject compounds and stereoisomers thereof, and pharmaceutically acceptable salts thereof disclosed herein as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules containing the compound and/or the at least one pharmaceutically acceptable salt thereof disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene gycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments. Suitable pharmaceutically acceptable carriers are described in Remington's Pharmaceutical Sciences, A. Osol, and other reference texts in the art.

For administration by inhalation, the subject compounds and stereoisomers thereof, and pharmaceutically acceptable salts thereof may be conveniently delivered in the form of an aerosol spray presentation from pressurized packs or nebulisers. The subject compounds and stereoisomers thereof, and pharmaceutically acceptable salts thereof may also be delivered as powders, which may be formulated and the powder composition may be inhaled with the aid of an insufflation powder inhaler device. One exemplary delivery system for inhalation can be metered dose inhalation (MDI) aerosol, which may be formulated as a suspension or solution of a subject compound and stereoisomers thereof, and pharmaceutically acceptable salts thereof disclosed herein in at least one suitable propellant, selected, for example, from fluorocarbons and hydrocarbons.

For ocular administration, an ophthalmic preparation may be formulated with an appropriate weight percentage of a solution or suspension of the subject compound and stereoisomers thereof, and pharmaceutically acceptable salts thereof in an appropriate ophthalmic vehicle, such that the subject compound and stereoisomers thereof, and at least one pharmaceutically acceptable salts thereof is maintained in contact with the ocular surface for a sufficient time period to allow the compound to penetrate the corneal and internal regions of the eye.

Useful pharmaceutical dosage-forms for administration of the subject compounds and stereoisomers thereof, and pharmaceutically acceptable salts thereof disclosed herein include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, a large number of unit capsules can be prepared by filling standard two-piece hard gelatin capsules each with, for example, 100 milligrams of the subject compound and stereoisomers thereof, and pharmaceutically acceptable salt thereof disclosed herein in powder, 150 milligrams of lactose, 50 milligrams of cellulose, and 6 milligrams magnesium stearate.

In some embodiments, a mixture of the compound, stereoisomers thereof, and pharmaceutically acceptable salts thereof a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, a large number of tablets can be prepared by conventional procedures so that the dosage unit comprises, for example, 100 milligrams of the compound, stereoisomers thereof, and pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, and pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, stereoisomers thereof, and pharmaceutically acceptable salts thereof are administered stepwise or in conjunction with at least one other therapeutic agent. When drugs are administered in physical combination, the dosage form and administration route should be selected depending on the compatibility of the combined drugs. Thus the term coadministration is understood to include the administration of at least two agents concomitantly or sequentially, or alternatively as a fixed dose combination of the at least two active components.

The compounds, stereoisomers thereof, and pharmaceutically acceptable salt thereof disclosed herein can be administered as the sole active ingredient or in combination with at least one second active ingredient.

The subject compounds are incorporated into pharmaceutical compositions or formulations. The compositions will contain pharmaceutically acceptable diluents and/or carriers, i.e. diluents or carriers that are physiologically compatible and substantially free from pathogenic impurities. Suitable excipients or carriers and methods for preparing administrable compositions are known or apparent to those skilled in the art and are described in more detail in such publications as Remington's Pharmaceutical Science, Mack Publishing Co, NJ. The compositions may also be in the form of controlled release or sustained release compositions as known in the art. For many applications the subject compounds are administered for morning/daytime dosing, with off period at night.

The subject compounds may be used per se, or in the form of their pharmaceutically acceptable salts, such as hydrochlorides, hydrobromides, acetates, sulfates, citrates, carbonates, trifluoroacetates and the like. When compounds contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When compounds contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like.

The neutral forms of the compounds may be regenerated by contacting the salt with a base or acid, and isolating the parent compound in the conventional manner. The parent form of the compound differs from the various salt forms in certain physical properties, such as solubility in polar solvents, but otherwise the salts are equivalent to the parent form of the compound for the purposes of this invention.

In addition to salt forms, this invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity.

Certain compounds of the invention can exist in unsolvated forms as well as solvated forms, including hydrated forms. In general, the solvated forms are equivalent to unsolvated forms and are intended to be encompassed within the scope of the present invention. Certain compounds of the invention may exist in multiple crystalline or amorphous forms. In general, all physical forms are equivalent for the uses contemplated by the present invention and are intended to be within the scope of the invention.

Some of the subject compounds possess asymmetric carbon atoms (optical centers) or double bonds; the racemates, diastereomers, geometric isomers and individual isomers are all intended to be encompassed within the scope of the present invention.

The compounds are generally administered in a “therapeutically effective amount”, i.e. the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The contacting is generally effected by administering to the subject an effective amount of one or more compounds having a formula herein, including the various embodiments described above. Generally administration is adjusted to achieve a therapeutic dosage of about 0.1 to 50, preferably 0.5 to 10, more preferably 1 to 10 mg/kg, though optimal dosages are compound specific, and generally empirically determined for each compound.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the mimetic is usually a minor component (from about 0.1 to about 50% by weight or preferably from about 1 to about 40% by weight) with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

### Examples: Development of Dibenzothiazepinones and Dibenzazepinones as Anti-Cancer Agents

As part of a phenotypic screen, we identified a small molecule, a dibenzothiazepinone termed MM017, with toxicity to colorectal cancer cells. Using forward genetics, we showed that mutations in Nuclear Valosin Like 2 (NVL2) lead to 30-fold resistance to MM017. NVL2 is an asymmetric, hexameric AAA-ATPase protein that couples ATP hydrolysis to the removal of WDR74 and its chaperone complex from the nascent 60S ribosome, a required step for 60S biogenesis. Using photo-chemistry and structural biology, we demonstrated that these mutations map to and are important for compound binding. Most of the studies involving NVL2 have been performed in yeast, so we developed a NVL2 degron system to investigate its function in colorectal cancer cells. Degradation of NVL2 led to reduced 60S ribogenesis as measured on polysome profiling consistent with its reported function in yeast. Moreover, MM017 phenocopied the effects of NVL2 degradation consistent with the hypothesis that binding leads to inhibition. Critically, cells harboring binding site mutations showed none of these effects confirming that they were on-target (due to NVL2 binding). We subjected MM017 to a PRISM analysis that involves evaluating potency and efficacy for 902 unique and deeply annotated cell lines. The selectivity of MM017 across these cell lines was most highly correlated (among 1,215 compounds) with oxaliplatin and carmofur. Carmofur is a 5-Flurouracil prodrug that is approved for the treatment of colorectal cancer in Japan and has also been shown to block 60S ribosome biogenesis. These correlations provide independent evidence that oxaliplatin and carmofur are toxic by blocking ribosome biogenesis. In addition, the utility of these drugs in CRC supports the rationale for testing MM017 in models of CRC. From here we developed analogs of MM017 that are optimized for in vivo proof-of-concept studies to assess tolerability, efficacy and pharmacodynamic markers for NVL2-targeting ribosome biogenesis inhibitors. We developed an optimization screening funnel that includes p53/p21 upregulation, known markers of impaired ribosome biogenesis-induced stress, as an on-target pharmacodynamic marker.

Targeting NVL2 provides a translatable strategy for colorectal cancer chemotherapy via a first-in-class alternative mechanism of ribosome biogenesis inhibition compared to clinically used ribotoxic drugs with undefined molecular targets. We disclose (1) design, synthesis, and evaluation of a collection of benzothiazepine-based NVL2 inhibitors to select a candidate suitable for in vivo evaluation, and (2) evaluation of in vivo drug tolerability, pharmacodynamics, and efficacy of optimized NVL inhibitors in a colorectal cancer xenograft model. Innovations include the first and only known small molecule to inhibit NVL2; compound resistant mutations to delineate on and off-target effects in cells; compound resistant xenografts derived from resistant cell lines above to delineate on and off-target pharmacodynamics and efficacy in vivo; and germline compound resistant NVL2^{R403W/+} knock-in mice to delineate on and off target toxicity in mice.

### Results I

We performed a high throughput screen of 99,599 small molecules (UTSW chemical library) for compounds that inhibit the proliferation of HCT116 colorectal cancer cells. A major challenge in phenotypic screening is understanding the mechanism of action often referred to as target ID. To address this challenge, we developed an isogenic, inducible forward genetic system that reveals compound resistant mutations.

As part of this effort, we identified MM017 as a novel small molecule with anti-proliferative activity in HCT116 cells. In total, we isolated 61 HCT116 resistant clones, and 11 of these were subject to whole exome sequencing, which revealed recurrent mutations in Nuclear Valosin Like 2 (NVL2). Follow-up NVL2-focused sequencing studies revealed that all 61 clones harbored mutations in NVL2. NVL2 is a AAA-ATPase protein that in yeast is responsible for removing (or unfolding) the NSA1 module, a set of chaperones bound to a nascent 60S ribosomal subunit in the nucleolus. As a result, NVL2 is essential for the biogenesis of the 60S ribosome. NVL2 consists of an N-terminal domain (NTD) important for localization to the nucleolus, and two tandem ATPase domains. The mutations that lead to resistance cluster in discrete regions of the first ATPase domain implicating this domain in the mechanism of action. We also CRISPR edited this exact mutation into a naïve cell and observed compound resistance.

To test whether NVL2 mutations prevent compound binding, we synthesized MM0514, a crosslinker derivative of MM017. MM0514 contains an aryl azide, and thus is capable of covalently modifying target proteins following exposure to UV light. MM0514 also contains an alkyne group providing a “handle” to purify bound proteins using click chemistry. Critically, MM0514 retains on-target activity to HCT116 cells since NVL2^{R403W} mutant cells are resistant. We crosslinked either parental or NVL2^{R403W} HCT116 cells to MM0514 and analyzed NVL2 binding. In a dose dependent manner, NVL2 bound MM0514 in parental cells but to a lesser degree in NVL2 mutant cells. These observations demonstrate that mutations in NVL2 confer resistance by preventing the direct binding of MM017 derivatives. Taken together, these results confirm that the anti-proliferative impact of MM017 and its derivatives is through binding NVL2. To characterize the binding site, we solved the cryo-EM structure of NVL2 with MM017. MM017 binds to a single site in the asymmetric hexamer near the D subunit. Consistent with our binding data, the mutations map near the binding site, including R403, which is mutated to tryptophan in a resistant clone and predicted to prevent binding of MM017 or related analogs. The cryo-EM structure of NVL2 with MM017 provides further evidence of target identification and also a tool for optimizing a bioavailable compound.

NVL2 in yeast is essential for 60S ribosomal maturation, and therefore, we hypothesized that MM017 might decrease 60S ribosome synthesis. To test this hypothesis, we subjected either parental or NVL mutant cells treated with MM017 for different time points to polysome profiling. Polysome profiling uses a sucrose gradient to fractionate the complete ribosome (80S) from its components, the large (60S) and small (40S) subunit. Parental cells treated with MM017 revealed a time-dependent decrease in the 60S and 80S ribosomal subunits, consistent with reduced 60S biogenesis. Since the 80S is a complex of the 40S and 60S ribosomes, fewer 60S ribosomes are expected to result in increased free 40S subunits. All of these changes are the result of binding NVL2 since they were not present in the NVL2^{R403W} mutant cells. Taken together, these observations indicate that MM017 inhibits NVL2 leading to reduced 60S ribosome.

Inhibition of 60S ribosome biogenesis is known to induce the stability and upregulation of p53, which could serve as a potential pharmacodynamics marker for MM017 in HCT116 cells. Consistently, MM017 upregulates p53 levels in parental HCT116 cells but not NVL2^{R403W} mutant cells. We used PRISM (Broad Institute) to evaluate the selectivity of MM017 across 902 cancer cell lines and then correlated this selectivity to 1,215 clinical and pre-clinical compounds in the PRISM database. The selectivity of carmofur (r=0.53, adjusted p<10⁻³⁰) and oxaliplatin (r=0.5, adjusted p<10⁻³⁰) significantly correlated with MM017 suggesting that all three compounds have a similar mode of action. These observations indicate that oxaliplatin and carmofur, like MM017, target ribosome biogenesis.

### Results II

We describe above a benzothiazepine small molecule that binds the ribosome biogenesis factor NVL2 and kills colorectal cancer cells in a dose-dependent manner that correlates with the upregulation of p53, a downstream effect of ribosome biogenesis inhibition. There are currently no known NVL2 inhibitors and therefore, this novel class of benzothiazepine-based NVL2 inhibitors provides reagents for medicinal chemistry optimization towards in vivo active lead compounds for proof-of-concept evaluation in colorectal cancer xenograft models.

### Medicinal Chemistry Optimization of Dibenzothiazepinone- and Dibenzazepinone-Based NVL2 Inhibitors.

Design and synthesis of benzothiazepine-based NVL inhibitors. We demonstrated that the dibenzothiazepinone scaffold is amenable to medicinal chemistry optimization such that we can synthesize analogs with desirable drug-like properties. We synthesized a large collection of dibenzothiazepinone and dibenzazepinone analogs and revealed its structure activity profile. Analogs were evaluated against the colorectal cancer cell line HCT116, and counter-screened against a compound resistant NVL2^{R403W} clone. Cytotoxicity in wild-type HCT116 was determined. All active analogs displayed on-target cytotoxic activity and were between 10 to 100-fold less potent against an NVL2^{R403W} clone. Initial SAR focused on exploring the replacement of the furan ring of initial dibenzothiazepinone hit MM017. We first explored a wide variety of mono- and di-substituted phenyl rings, including a broad spectrum of functionality (F, Cl, alkyl, alkoxy, fluoroalkoxy, CN, formamide, S(O) Me, carboxyl, . . . ). Ortho-substituents were not allowed, except for fluorine and only 3- or 4-substituted, or 3,4-disubstituted phenyl analogs retained activity similar to the parent. Five-membered heterocycles other than furan significantly reduced potency, whereas benzoxazole, benzothiazole, and 2-oxo-2,3-dihydrobenzoxazoles were equipotent or somewhat more potent than the corresponding phenyl analogs. We also found that several amides derived from non-aromatic acyclic or mono- or spirocyclic carboxylic acids led to active analogs. Because of potential protease mediated cleavage of the amide bond, we investigated replacement of the amide linker (C(O)NH and found that ureas retained similar potency, as did thioamides and thioureas. Reverse amides and sulfonamides on the other hand were inactive. Moving to the side-chain, we found that a longer propargyloxy or hexenyl boosted potency 3 to 10-fold, but at a cost of increased lipophilicity and number of rotatable bonds. A shorter rigid 2-butynyl proved optimal and increased activity >10 to 20-fold.

SAR data correlated with insights gained from co-CryoEM NVL2 structures with several dibenzothiazepinones and dibenzazepinones. This led us to design a series of heteroaromatic analogs where we replaced the amide-linked dibenzothiazepinones or dibenzazepinones with amine-linked dibenzothiazepinones and dibenzazepinones. A variety of aromatic, heteroaromatic, heterocyclic, carbocyclic, and acyclic groups linked to the dibenzothiazepinone or dibenzazepinone core via an amine linkage were found to retain or improve activity.

With input from our current SAR data, we designed and synthesized new analogs based on the generic scaffold. We retain the more potent and less lipophilic alkynyl side-chain while also exploring additional rigidification, metabolic blocks, and truncation. We found that oxidation of the central dibenzothiazepinone sulfur atom (to SO or SO₂), or replacement with an oxygen atom, was not tolerated, but CH₂ substitution reduced activity only ˜2 fold. Based on this finding, we also replaced the central sulfur atom with a CHMe unit and found these to retain similar activity as the sulfur analogs. Replacement with a CHCN or CMe (OH) unit reduced activity somewhat. Therefore, our subsequent designs retain a central sulfur or (optionally substituted) carbon atom. We next explored substituents on the various positions available on the two benzo-rings of the central dibenzothiazepinone core. We found that fluorine, chlorine, and cyano substituents were allowed in the right-hand benzo-ring, retaining full potency if introduced in the two positions not directly adjacent to the benzo-fusion. No such substitutions were allowed in the left-hand benzo-ring. We also sampled analogs where we replaced individual CH-units of the two benzo-rings with nitrogen (pyrido analogs). These were not allowed in the right-hand benzo-ring, whereas they were allowed in the left-hand ring at positions marked U and W in the generic structures of the claims. We further found that replacement of a CH with a nitrogen at position W in the generic structures significantly increased the metabolic stability of the analogs when measured in a microsomal stability assay. All of the central dibenzothiazepinone and dibenzazepinone core scaffolds can be synthesized from readily available starting materials using the same chemistry, herein. We focus on substituents that are small, improve physicochemical properties, and reduce metabolism. The final step of the synthesis involves conjugation of the peripheral units (R3 in the generic structures) with an amino-substituted central core via Pd-catalyzed C—N bond formation (structures of formula VI) or reductive amination, or amide-bond formation with appropriate carboxylic acids (structures of formula VII). Thioamides (structures of formula VIII) are derived from reacting the amino-dibenzothiazepinone core with R³-aldehydes and sulfur powder in aqueous K₂CO₃. Ureas (structures of formula II or IV) and thioureas (structures of formula III or V) are obtained from the same amino-substituted central cores using contemporary methods of urea/thiourea formation using triphosgene or thiophosgene. Preferred substitutions only incorporate druglike substituents or groups with desirable physicochemical properties, and limited predicted metabolic liabilities. Preferred substituents are small, improve physicochemical properties, and reduce metabolism.

Our nextgen analogs retain or improve C Log P and conform to druglike space with zero Lipinski Rule of 5 or Veber rule violations. Introducing sp³-stereocentra could be beneficial for activity and specificity, solubility, and reduced protein binding.

Assessing the ability of NVL2 inhibitors to selectively kill WT versus NVL2^{mut} HCT116, block ribosome biogenesis and induce p53. We evaluate analogs in the colorectal cancer cell line HCT116 and already available compound-resistant clones. One clone has a mutation in the compound binding site (NVL2^{R403W}) and another clone (NVL2^{P307T}) has a mutation distant from the binding site that confers resistance through an unknown effect. Based on our results, analogs that kill HCT116 cells via NVL2 inhibition (on-target) have reduced potency in compound-resistant HCT116 mutant clones. Our analogs have been counter-screened against the NVL2 compound-resistant mutant clones with fold resistance range between 10-100 fold (most analogs 20-50 fold). This counter screen provides a strategy for identifying off-target toxins. Analogs that dose-responsively kill wild-type cancer cells with an IC₅₀<1,000 nM and are at least 20-fold less potent in NVL mutant cell lines are progressed.

Analogs that pass potency and on-target NVL2 inhibition criteria and are metabolically stable (T_{1/2}>30 min in microsomal assays) are further evaluated for their ability to inhibit ribosome biogenesis (polysome profiling, and induction of p53 and/or p21). Antibodies for p53 are used to evaluate p53 as a pharmacodynamic marker for in vivo studies.

Profiling of analogs for in vitro metabolism, plasma stability, solubility, and plasma protein binding. We evaluated the majority of analogs for oxidative metabolism using murine microsomal fractions in the presence of an NADPH regenerating system necessary for phase I metabolism. Half-lives are determined by the method of substrate depletion. Compounds identified as having significant metabolic liabilities (T_{1/2}<30 min) have high in vivo clearance rates and were not pursued further. Methoxy substituents are potentially labile to oxidative metabolism and therefore, introducing fluorine metabolic blocks were explored as well. Because we suspected that some metabolic clearance might be protease mediated, selected analogs were evaluated in microsomal fractions in the absence of an NADPH regenerating system to eliminate phase I oxidative metabolism. We found that some amides still metabolized at an appreciable rate, whereas others were more stable. Unsurprisingly, amines, ureas, and thioureas were stable under these conditions (T_{1/2}>120 min).

Compounds with acceptable metabolic clearance rates are evaluated in additional in vitro assays including aqueous solubility, plasma stability and protein binding using the gold-standard rapid equilibrium dialysis. Current SAR indicates that we can achieve high activity, solubility, and acceptable metabolic stability with more polar compounds (C Log P<4).

Pharmacokinetic studies of NVL2 inhibitors. For pharmacokinetic evaluation we measure the exposure of analogs after iv (3 or 5 mg/kg), ip (10 mg/kg) and oral (20 mg/kg) dosing. Drug levels in plasma and liver are measured (LC-MS/MS) at eight time points (3 mice/time point). Total plasma or tissue exposure (AUC) and half-lives are calculated using the non compartmental analysis tool of WinNonlin. Oral bioavailability is calculated as AUCoral/AUCiv×Doseiv/Doseoral (desired criteria F>30%). The most promising compound are dose escalated for up to 3 dose levels to determine whether increased exposure occurs with increased dose. Whereas initial PK is done in female CDI mice, dose escalation is evaluated in both females and males.

For in vivo studies we prefer orally available compounds, but intraperitoneal (ip) or intravenous (iv) delivery is deemed acceptable for proof-of-concept studies. We select for compounds with a terminal half-life of >2 hours and [Cmax]ᵤ>IC₉₀ (cellular tox in HCT116).

### In Vivo Tolerability, Pharmacodynamics, and Efficacy of NVL2 Inhibitors.

The NVL2 ATPase is required for biogenesis of the 60S ribosome. Preliminary data including cryo-EM structures demonstrates that dibenzothiazepinones and dibenzazepinones bind directly to NVL2 which results in decreases of the 60S and 80S ribosomes, an upregulation of p53, and cell death. The cytotoxic effects of dibenzothiazepinones and dibenzazepinones are consistent with the hypothesis that they selectively target cancers dependent on increased ribosome biogenesis. Oxaliplatin, an effective treatment for colorectal cancer, inhibits ribosome biogenesis through an unknown mechanism. This indicates that dibenzothiazepinones and dibenzazepinones might have efficacy in colorectal cancer. We test this hypothesis by systematically evaluating the antitumor capacity of optimized dibenzothiazepinone and dibenzazepinone analogs in pre-clinical models of colorectal cancer. We also evaluate whether these effects are mediated via inhibition of ribosome biogenesis and upregulation of p53 and determine if there are on-target toxicities resulting from NVL2 inhibition in healthy, non-tumor bearing tissues. We define the tolerability, pharmacodynamic response, and single-agent efficacy against wild-type and compound resistant colorectal cancer xenografts of lead bioavailable NVL2 inhibitors.

Tolerability of dibenzothiazepinone or dibenzazepinone treatment in mice and determine whether toxicities are caused by engaging NVL2. We test the tolerability of chronic, once-daily oral or intravenous treatment (route of administration depending on PK parameters) with doses of dibenzothiazepinone or dibenzazepinone analogs. Three doses are chosen for evaluation in tolerability studies based on the specific PK data and solubility characteristics of each analog. C57Bl/6 mice are randomized to four treatment cohorts per analog (n=5 male and 5 female mice per arm: 1× vehicle, 1× low dose benzothiazepine, 1× intermediate dose benzothiazepine, 1× high dose benzothiazepine). Vehicle and drug treatments are administered once daily via oral gavage (or intravenous tail vain injection) for 14 consecutive days. Primary endpoints for the study are body weight changes and hematologic, renal, and hepatic toxicity. Secondary endpoints are absolute benzothiazepine concentration in plasma. Exploratory endpoints are other standard parameters measured by Complete Blood Counts (CBCs) and serum chemistry analyses. At the end of the 14-day treatment period, we anesthetize mice, collect blood samples via cardiac puncture, and euthanize the animals. Blood samples are evaluated for CBCs and serum chemistry analyses.

Body weight changes provide a metric of gross toxicity. The toxicity threshold for weight changes was selected as a ≥20% decrease relative to vehicle-treated mice. Mice that reach this threshold are humanely euthanized. CBCs are used to assess hematologic toxicity. Low platelet counts (<772K/μL) or hemoglobin levels (<13 g/dL) are used to detect drug-induced thrombocytopenia or anemia. Serum chemistry data are used to assess renal and hepatic toxicity. Elevated creatinine levels (>1.5 mg/dL) serve as a marker of kidney dysfunction. Elevated alanine aminotransferase (ALT) (>346.5 U/L) or aspartate aminotransferase (AST) (>690 U/L) levels indicate liver damage. Threshold values for plasma and serum biomarkers of toxicity were derived by applying Grade 1 adverse event thresholds (Common Terminology Criteria for Adverse Events Version 5.0 published by the NCI) to 95% confidence interval limits for these biomarkers in naïve mice. The MTD is determined for each benzothiazepine analog as the highest dose that can be administered daily for 14 consecutive days without causing average levels of toxicity biomarkers in the cohort to exceed the thresholds defined above. Satellite PK measurements of compound levels in plasma are made to confirm bioavailability.

To interrogate off target versus on target toxicities, we generated knock-in mice harboring a germline compound resistant NVL2^{R403W} mutation. Heterozygous knock-in mice are generated in the C57BI/6 background using CRISPR/Cas9 and can be bred to homozygosity. Next, we treat WT and NVL2^{R403W} mice with vehicle control or three different doses of dibenzothiazepinone or dibenzazepinone analogs (n=5 mice/group/genotype). Mice are age matched and gender matched (1 gender/group unless toxicity differences between male and female mice were observed) and analyzed using the same endpoints described above. Doses are selected based on determination of the MTD and include at least one dose below and one dose above the MTD. For analogs with greater toxicity in WT mice as compared to NVL2^{R403W} mice, that would suggest on-target effects caused by NVL2 engagement by dibenzothiazepinone or dibenzazepinone analogs. To confirm, we compare levels of ribosome biogenesis and p53 induction in compromised tissues or cell types in WT and NVL2^{R403W} mutant mice. For analogs that are toxic in germline NVL mutant mice, we conclude that these effects are caused by off-target effects. In case of on-target mediated toxicities, we then define if there is a therapeutic window available where side-effects can be minimized while retaining antitumor efficacy. In case of off-target effects, we will attempt to determine the offending mechanism and implement appropriate assays in our medicinal chemistry pipeline to identify analogs with reduced off-target toxicity.

Evaluate pharmacodynamic effects of dibenzothiazepinone and dibenzazepinone analogs and identify candidate biomarkers for in vivo target engagement. Our in vitro data demonstrate that dibenzothiazepinones and dibenzazepinones kill cancer cells via direct inhibition of NVL2. We also demonstrated that NVL2 inhibition with dibenzothiazepinones or dibenzazepinones impairs biogenesis of the 60S ribosomal subunit, leading to upregulation of p53. Similar experiments with isogenic dibenzothiazepinone-resistant NVL2^{R403W} knock-in clones do not show inhibition of ribosomal biogenesis, upregulation of p53, or antiproliferative activity when treated with NVL2-targeting dibenzothiazepinones or dibenzazepinones. Therefore, we evaluate p53 upregulation as a viable PD marker for on-target activity. In addition, we assess xenografts for decreases in ribosome biogenesis and activation of molecules downstream of p53, such as p21.

To test whether dibenzothiazepinone or dibenzazepinone analog treatment induces p53 and blocks ribosome biogenesis in vivo, ICR SCID mice are transplanted subcutaneously with 5×10⁶ WT or NVL2^{R403W} HCT116 colorectal cancer cells. Xenografts are formed from the population of HCT116 cells targeted with guides for the NVL2^{R403W} knock-in mutation to eliminate potential artifacts that may arise within a single clone that has incurred off-target mutations. Once tumors reach ˜110 mm³, mice are randomized to vehicle and dibenzothiazepinone or dibenzazepinone treatment arms (n=8 mice per arm; 16 mice total/genotype). NVL inhibitors or vehicle control are administered at the MTD once daily via oral gavage, intraperitoneal, or intravenous tail vain injection for 3 days. On Day 3, we collect blood samples and euthanize mice to harvest liver and xenografts to assess PK (in blood, liver and tumor) and PD markers (in tumor). To nominate candidate biomarkers, tumor tissues collected from control or compound-treated mice are compared by quantitative Western blot analysis of p53, as well as downstream induction of p21 via qPCR.

Determine the efficacy of dibenzothiazepinones and dibenzazepinones in xenograft models of colorectal cancer and validate candidate biomarkers. After evaluating tolerability and PD, we use wild type and NVL2^{R403W} HCT116 xenograft models to assess the antitumor activity of analogs in vivo. We transplant wild type and NVL^{R403W} HCT116 cells subcutaneously and begin treatment. NVL inhibitor analogs are given at the MTD once daily via oral gavage, intraperitoneal, or intravenously via tail vain injection (frequency and route of administration to be determined based on PK). Treatments continue until mice display symptoms of tumor burden that necessitate euthanasia or until 50 days post-randomization, whichever occurs first. We collect remaining tumor tissues as available for PD analysis. The primary endpoint is overall survival. We construct Kaplan Meier curves for each benzothiazepine analog in each xenograft model. Log-rank tests are used to statistically evaluate differences in survival between drug- and vehicle-treated cohorts. In addition, we measure tumor volumes every 3 days and graph tumor growth over time which serve as a complementary endpoint.

Next, we perform a dose response efficacy study by establishing wild-type HCT116 xenografts and administering NVL inhibitor analogs as described above. The top dose is the MTD and two additional lower doses are chosen by referring to PK data (n=8 mice per arm×3 doses and vehicle control; n=32 mice total). Tumor growth over time and survival is quantified as described and remaining tumor tissues are collected as available for PD analysis. The lowest dose with statistically equivalent efficacy to that achieved with the MTD is determined.

Finally, p53 and p21 levels are reassessed in tumor tissues treated at the lowest efficacious dose for 3 days in a separate PD cohort (n=5/arm; vehicle control and 1 dose for 10 mice total). For example, we quantify the difference p53 and p21 levels to determine the magnitude of the change in the biomarker induced by a therapeutic dose of benzothiazepine.

We define the therapeutic window for each dibenzothiazepinone or dibenzazepinone analog by identifying doses that are well tolerated and induce robust tumor PD responses and efficacy in colorectal xenograft models. p53 induction observed in benzothiazepine treated tumors is mitigated in xenografts formed from NVL^{R403W} cancer cells, thereby confirming the mechanism of action in vivo. The levels of p53 induction positively correlate with the degree of dibenzothiazepinone or dibenzazepinone antitumor efficacy in a dose dependent manner.

### Examples: Optimized Dibenzothiazepinone and Dibenzazepinone Chemicals Inhibit NVL-Mediated Ribosome Assembly and Cause p53-Dependent Apoptosis of Cancer Cell Xenografts

In these examples we identify human NVL as the target of a chemical toxin designated, MM017. A cryo-EM structure of NVL bound to MM017 reveals binding sites within the hexameric assembly. Mutations in NVL that cause resistance to MM017 cluster in the binding site and lead to a reduction in compound binding. The NVL-MM017 interaction specifically inhibits the maturation of 60S ribosomal subunits, causing abnormal polysome profiles and accumulation of newly synthesized pre-60S intermediates in the nucleolus. NVL inhibition triggers p53-mediated apoptosis without any evidence of DNA damage. A bioavailable analog of MM017 reduces tumor growth in vivo without overt toxicity.

### Mutations in NVL Confer Resistance to Orphan Cytotoxin MM017.

We recently described an isogenic forward genetic system to rapidly find mutations conferring resistance to compounds with anti-proliferative activity (32). This system uses an engineered colorectal cancer HCT116 cell line (iHCT116) in which the addition of indole acetic acid (IAA) initiates a ubiquitin-mediated degradation of the DNA mismatch repair protein MLH1, thereby providing a mechanism to temporarily increase mutation rates. We applied this strategy to MM017, a dibenzothiazepinone identified in a high-throughput small molecule screen for HCT116 cytotoxicity. MM017 inhibits the growth of iHCT116 cells in a dose-dependent manner, with an IC50 of 0.46 μM. We exposed barcoded iHCT116 cells, that had previously been cultured either with (Mutagenesis-on) or without IAA (Mutagenesis-off), to increasingly lethal doses of MM017. The total number of surviving clones was dramatically higher in the Mutagenesis-on conditions and increased in a dose-dependent manner suggesting a genetic basis for resistance.

We identified 59 clones with unique barcode sequences, which indicates that each clone arose from a unique founder event. The IC50 of MM017 to these clones was between 2.45 to 32.89-fold greater than the parental cell line providing evidence of resistance. The wide degree of resistance suggests that these clones harbored a diverse set of mutations, and therefore, we performed whole exome sequencing on 15 clones spanning the entire range of resistance. From whole exome sequencing, we identified variants that were acquired during IAA-induced mutagenesis with the hypothesis that genes related to resistance will be mutated in multiple clones. Two genes contained acquired variants in at least 3 of the 15 selected clones: TERT, mutated in 3 of 15 clones, and NVL, mutated in all 15 clones. These results suggest that NVL mutations are related to MM017 resistance. Thus, for the remaining clones, we performed PCR based sequencing of NIL exons that were found to be mutated in the exome sequencing data. In sum, 57 out of 59 unique clones harbored NVL mutations. The majority of mutations clustered within the D1 AAA+ ATPase domain and frequently affecting the same codons. To test whether these mutations are sufficient for MM017 resistance, we used CRISPR-Cas9 to knock-in two of the most resistant mutations (NVL^{P307T}, NVL^{R403W}) and a mutation with intermediate resistance (NVL^{H304R}). Transfection of cells with sgRNAs targeting these NVL residues in addition to a mutation-encoding repair template yielded an increased rate of resistance when compared to mock treatments. Following expansion, we confirmed that cells harbored the intended alleles (NVL^{P307T}, NVL^{R403W} or NVL^{H304R}) and were more resistant to MM017 compared to NVL^{WT}, (IC50: NVL^{WT}=0.38 μM; NVL^{R403W}>50 μM; NVL^{P307T}>50 μM; NVL^{H304R}=2.4 μM), but equally sensitive to an unrelated toxin, paclitaxel. Taken together, these observations demonstrate that NVL mutations are specific and sufficient for MM017 resistance.

### MM017 Directly Binds NVL.

To assess whether MM017 binds directly to NVL in cells, we used a MM017 crosslinker (Probe compound (2), Table A) that is UV-activatable and contains an alkyne that can be coupled to biotin for affinity purification. Critically, Probe compound 2 inhibited the proliferation of HCT116 cells (IC50=0.47 μM) with a potency comparable to MM017, and NVL^{P307T} cells were comparatively resistant to both compounds, suggesting that MM017 and MM017-probe function similarly. We purified MM017-probe bound proteins from cells that had been treated with varying doses of MM017-probe either in the presence or absence of UV light. NVL was only detected in conditions that included both the MM017-probe and UV light, indicating that MM017-probe interacts non-covalently with NVL in the absence of UV light. To assess whether the observed binding of MM017-probe to NVL is relevant to its cellular activity, we performed probe-displacement assays using three chemically related derivatives of MM017 (compounds 3-5, Table A) with IC50s of 0.36 μM (3), 2.1 μM (4) and 13 μM (5). Note, this experiment was performed using a cell line in which we knocked-in a FLAG epitope into the C-terminal end of NVL. Co-incubation of increasing doses of compound 3 more effectively displaced MM017-probe compared to compounds 4 and 5, establishing a relationship between NVL binding and cellular activity. We next compared NVL levels following MM017-probe and UV treatment of NVL^{WT} versus NVL^{R403W}, NVL^{P307T}, and NVL^{H304R} cell lines. In comparison to NVL^{WT}, the amount of MM017-probe required to crosslink NVL was substantially higher in the mutant cell lines demonstrating that these mutations affect compound binding. All together these observations establish a relationship between compound binding and cellular activity that is consistent with the hypothesis that NVL is the direct target of MM017.

TABLE A
Structures of MM017, MM927, Probe compound 2, and analogs 3-5.

| | |
|---|---|
| 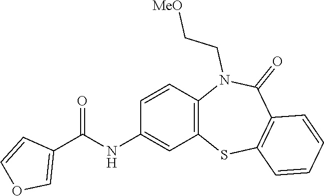 [SMILES: COCCN1C(=O)c2ccccc2Sc2cc(NC(=O)c3ccoc3)ccc21] | MM017 |
| 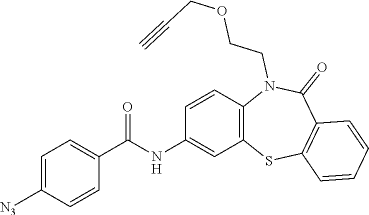 [SMILES: C#CCOCCN1C(=O)c2ccccc2Sc2cc(NC(=O)c3ccc(N=[N+]=[N-])cc3)ccc21] | Probe compound (2) |
| 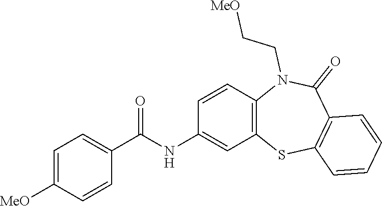 [SMILES: COCCN1C(=O)c2ccccc2Sc2cc(NC(=O)c3ccc(OC)cc3)ccc21] | 3 |
| 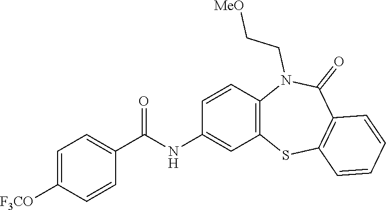 [SMILES: COCCN1C(=O)c2ccccc2Sc2cc(NC(=O)c3ccc(OC(F)(F)F)cc3)ccc21] | 4 |
| 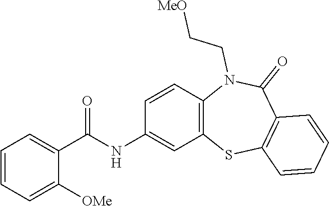 [SMILES: COCCN1C(=O)c2ccccc2Sc2cc(NC(=O)c3ccccc3OC)ccc21] | 5 |
| 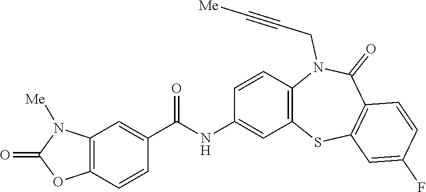 [SMILES: CC#CCN1C(=O)c2ccc(F)cc2Sc2cc(NC(=O)c3ccc4oc(=O)n(C)c4c3)ccc21] | MM927 |

### Cryo-EM Reconstruction of the NVL/MM017 Complex.

To understand the molecular basis of the MM017/NVL interaction, we set out to solve the cryo-EM structure of NVL in complex with MM017. Expressing and purifying the D1/D2 core of wildtype NVL led to low yields and protein instability. Cryo-EM analysis showed that wildtype NVL D1/D2 assembled into long, filament-like spirals rather than the expected hexamers. Double mutations in the Walker B motif of the C. thermophilum NVL homolog Rix7 stabilize the hexameric configuration (33), and recombinant expression and purification of mutated human NVL^{E366Q/E683Q} (NVL^{dEQ}) similarly resulted in stable, monodisperse hexamers, whose stoichiometry was unaffected by the addition of MM017 as determined by mass photometry.

A single particle cryo-EM reconstruction of NVL^{dEQ}+MM017 resulted in a map with an overall resolution of 2.9 Å. Similar to reconstructions of C. thermophilum Rix7, H. sapiens P97 and S. cerevisiae Drg1, the NVL hexamer forms a staircase configuration, with pore loops from five D1 and D2 AAA+ modules engaging an extended peptide within the central channel (2, 27, 34, 35). Following convention, we designate the topmost staircase subunit A and the lowest subunit E. As previously observed, the sixth subunit (F) is in a more dynamic state, transitioning between positions E and A, and is therefore poorly resolved in the cryo-EM map. By analogy to the analysis performed with RIX7 (34) and the bulky nature of sidechain density within the peptide, we hypothesize that the substrate mimic engaged by NVL represents one of the N-terminal 14x-HIS tag on our construct and have therefore modeled it as a poly-histidine chain, even though our tag also includes non-HIS linker residues.

An initial analysis of the cryo-EM maps revealed additional density consistent with MM017 in a single position within subunit E. MM017 is wedged into a mostly hydrophobic pocket in the core domain of the D1 module. The ligand pocket is walled on one side by the central D1 β-sheet (strands 5, 1 and 4) and on the other side by the N-terminal α0 helix and the loop element preceding strand 1. This helix also engages the AAA+ lid-domain of subunit D, which forms the more peripheral boundary of the MM017 binding pocket. There is no ligand binding site in the equivalent regions of NVL subunits A-D because the α0 helix is tightly packed against the central β-sheet of the domain, precluding MM017 binding. To determine if MM017 also engaged the poorly resolved and mobile F subunit, we performed a skip-align classification with a mask around the subunit F D1 module. The resulting reconstruction of this particle subset revealed MM017 density in subunit F in the analogous position to subunit E. Most suppressor mutations cluster around the ligand binding pocket, consistent with these modifications altering the structure or conformation of the compound docking site. MM017 is an elongated molecule with a distinct 105° kink introduced by the central dibenzothiazepinone core. The furan ring of MM017 is exposed to the central solvent channel that separates the D1 and D2 ring stacks and makes hydrophobic contacts with residues M280 and V301 and forms a hydrogen bond with R428. Adjacent to the furan moiety, the planar amide linker forms a hydrogen bond with the backbone carbonyl of amino acid P298. The dibenzothiazepinone ring structure of MM017 is in a hydrophobic environment created by residues M280, M284, P297, L324, R403 and L405. Among these, M284, P297, R403 and L405 are either mutated or immediately adjacent to a mutated residue in our suppressor dataset. In addition, the central carbonyl group is within hydrogen bonding distance of the epsilon-amino group of R403. Finally, the methoxyethyl tail projects out near the loop connecting helix α0 and strand β1.

To gain further insights on how substrate binding may affect the inter-subunit dynamics of NVL, we obtained single particle cryo-EM reconstructions of apo-NVL^{dEQ} to a resolution of 2.6 Å. In the apo structure, the hexameric assembly retains the staircase conformation, but the ligand binding pocket in subunit E is closed and in the same configuration as the equivalent positions within subunits A-D. In the apo structure, the subunit E D1 domain shifts closer to the neighboring AAA+ module in subunit D, closing the bipartite nucleotide binding site at the D/E interface and bringing the arginine finger motif of subunit E into closer proximity to the bound ATP in comparison to the MM017-engaged structure of NVL, suggesting that compound binding allosterically affects the D/E interface within the D1 ring. Consistent with this, one of the most resistant NVL mutations, P307T, maps to the D/E nucleotide interface. We infer that the observed allosteric link between the ligand binding pocket and the nucleotide binding site at the D/E interface hinders the assembly of a functional D1 ring in the presence of MM017.

### MM017-Bound NVL Specifically Blocks 60S Biogenesis in the Nucleolus.

To study the effect of NVL depletion, we engineered a C-terminal FLAG-epitope auxin-inducible degron (AID) tag into NVL in cells that also express the plant E3 ligase TIR1. 5-Phenyl-indole-3-acetic acid (Ph-IAA) treatment caused dose-dependent depletion of NVL-AID but not untagged NVL and in NVL^{AID/AID} cells, reduced viability in a dose-dependent manner. The viability of NVL^{+/+} or NVL^{AID/+} cells was unaffected by Ph-IAA, indicating that although NVL is essential for HCT116 cell proliferation, it is not haploinsufficient.

We next assessed the impact of disrupting NVL on the distribution of ribosomal species using polysome profiling. Addition of Ph-IAA to induce NVL degradation in NVL^{AID/AID} cells led to a time-dependent reduction in free 60S and 80S levels (based on absorbance at 260 nm) and a concomitant increase in 40S levels. Ph-IAA addition also caused half-mer polysomes, which represent unjoined 40S subunits bound at mRNA initiation sites and are characteristic of a 60S biogenesis defect (36). Addition of MM017 to NVL^{WT} cells phenocopied the polysome profiles from Ph-IAA-treated NVL^{AID/AID} cells but caused no changes in polysome profiles from NVL^{P307T} cells. The polysome profiles following NVL disruption in HCT116 cells appear similar to those reported in yeast with RIX7 mutations (37) or in human cells overexpressing catalytic mutants of NVL (38).

The observed reduction in free 60S using polysome profiling could be the consequence of decreased 60S synthesis or accelerated 60S degradation, or a combination of the two. To distinguish between existing and newly synthesized 60S, we engineered HCT116 cells to express a C-terminal SNAP-tag on eL36, a ribosomal protein incorporated into the 60S subunit prior to NVL binding (39) or a control C-terminal SNAP-tag on a component of the small ribosomal subunit, eS17, to monitor 40S biogenesis (40). We first labeled existing eL36 or eS17 in cells with an Oregon green benzylguanine (green). Then, we treated cells for 24 hours with either vehicle or MM017 before adding a 647-SiR benzylguanine to label the pool of proteins synthesized over the 24-hour period and evaluated each polysome profile fraction for either existing or new proteins. Existing eL36-SNAP was present in 60S, 80S, and polysome fractions, indicating that the SNAP-tagged protein can assemble into translating ribosomes. MM017 treatment had no impact on the levels or fractionation pattern of existing eL36-SNAP. By contrast, following MM017 treatment, newly synthesized eL36-SNAP was reduced in the 60S, 80S, and polysome fractions, consistent with the hypothesis that MM017 blocks 60S biogenesis. Upon treatment with vehicle or MM017, existing and newly synthesized eS17-SNAP was detected in the 40S, 80S, and polysome fractions, demonstrating that MM017 has no impact on 40S subunits.

Next, we examined the impact of MM017 on the subcellular localization of eL36-SNAP (60S) or eS17-SNAP (40S). As expected for mature ribosomes, existing eL36-SNAP was nearly completely cytoplasmic, and treatment with MM017 had no impact on its localization. In vehicle treated cells, newly synthesized eL36-SNAP was both nucleolar and cytoplasmic, reflecting its presence in pre-60S and mature ribosomes. By contrast, in cells treated with MM017, new eL36-SNAP localized exclusively to foci within the nucleus. These foci co-localize with the nucleolar protein Fibrillarin, suggesting that the arrested eL36-SNAP intermediates are nucleolar. To test whether the observed block in 60S biogenesis is due to binding to NVL, we engineered cells expressing eL36-SNAP and NVL^{R403W}. Upon treatment of these cells with MM017, newly synthesized eL36-SNAP localized to the nucleolus and cytoplasm, a pattern indistinguishable from the vehicle treated group. These results demonstrate that the arrest of 60S biogenesis is due to inhibition of NVL by MM017. Finally, we asked whether MM017 impacted either degradation or biogenesis of the 40S ribosome by performing analogous experiments using eS17-SNAP cells. MM017 had no impact on the localization of existing or newly synthesized eS17-SNAP. All together, these results demonstrate that MM017 directly inhibits NVL to specifically block nucleolar 60S assembly.

### Inhibition of NVL Leads to p53 Dependent Apoptosis.

To better understand how inhibition of NVL by MM017 leads to cell death, we performed a genome wide pooled CRISPR/Cas9 knockout screen in HCT116 cells using a library of guide RNAs targeting 19,114 genes. Cells were pulsed intermittently with vehicle or MM017 over 21 days, at which point MM017 treated cells had undergone 9.79 fewer population doublings. Massively parallel sequencing of PCR products amplified from genomic DNA was used to determine relative levels of sgRNA sequences to infer the relative impact of each gene on compound sensitivity. The tumor suppressor TP53 (commonly referred to as p53) and one of its effector genes, CDKN1A (also known as p21), were the most enriched genes in this screen, indicating that loss of p53 and, to a lesser degree, p21 protect cells from MM017. To confirm whether p53 and p21 are required for the anti-proliferative activity of MM017, we performed a growth competition experiment between cells expressing ZsGreen with either p53 or p21 knockout (KO) and control cells expressing mCherry. Without treatment, the ratio between green and red cells was unchanged over time. By contrast, MM017 led to a dose-dependent enrichment of either p53 or p21 KO cells. The requirement for these two proteins was specific to MM017 since no competitive advantage was observed in the presence of paclitaxel. Taken together, these results show that MM017 reduces HCT116 cell proliferation in a p53- and p21-dependent manner.

To nominate genetic predictors of MM017 sensitivity across different cancer types, we performed a PRISM screen across 902 cancer cell lines with known genotypes (31). A correlation analysis found that mutations in TP53 most significantly linked with resistance to MM017. These findings are consistent with the isogenic CRISPR screen and indicate that p53 is a critical effector for MM017 in cancer cells from multiple lineages and with differing genotypes.

The essentiality of p53 and p21 to MM017 activity raised the hypothesis that these proteins may be upregulated by inhibition of NVL. MM017 led to an increase in p53 at 6 hours post treatment, and p21 soon thereafter, consistent with its role as a p53 target protein. Importantly, the observed increase in p53 and p21 expression are the result of NVL inhibition because NVL^{P307T} cells showed no changes in the abundance of either protein, while degradation of NVL in NVL^{AID/AID} cells led to a similar increase in p53 levels.

The upregulation of p53 and its effector p21 can arrest cell growth or induce cell death through apoptosis. To distinguish between these two possibilities, we examined cell death by a loss in membrane permeability following a five-day MM017 treatment of HCT116 control and p53 KO cells and found that the number of dead cells was substantially lower in p53 KO cells. The protection from MM017-induced cell death by loss of p53 was specific to MM017, since paclitaxel, an unrelated toxin that stabilizes tubulin polymerization, led to cell death irrespective of p53. Next, we addressed whether cell death was mediated by an apoptotic pathway by assaying for cleaved caspase-3, a bona fide marker of apoptosis. Staurosporine, an unrelated compound that is known to induce apoptosis, led to robust caspase activation in both control and p53 KO cells. By contrast, MM017 induction of cleaved caspase-3 in control cells was substantially reduced in p53 KO cells Taken together, these findings demonstrate that the inhibition of NVL by MM017 leads to p53-dependent apoptosis in HCT116 cells.

p53 upregulation is consistent with the ribosome assembly stress response triggered by 5S RNP mediated inhibition of MDM2 (5). Other inhibitors of ribosome biogenesis, such as oxaliplatin and CX-5461, are also thought to activate the same ribosome assembly stress response, but these compounds are also known to induce DNA damage (15, 20). A biochemical marker for activation of the DNA damage response is phosphorylation of serine 15 of p53, which results in increased p53 protein (41). Thus, we compared the impact of MM017, oxaliplatin, and CX-5461 on both p53 upregulation and p53 serine 15 phosphorylation. All three compounds led to a dose-dependent increase in the levels of p53, but only oxaliplatin and CX-5461 treatment led to a dose-dependent increase in phosphorylated p53. Even at concentrations as high as 10 μM, there was no evidence of serine 15 phosphorylated p53 after MM017 treatment. These observations suggest that p53 upregulation by oxaliplatin and CX-5461 is at least in part triggered by the DNA damage response. Because there was no evidence of serine 15 p53 phosphorylation following treatment with MM017, we conclude that the observed increase in p53 stabilization following treatment with MM017 is independent of the DNA damage response and entirely initiated by the disruption of 60S ribosomal subunit assembly.

A Bioavailable Analog Reduces Tumor Growth in Mouse Xenografts without Overt Toxicity.

Next, we investigated whether inhibitors of NVL could inhibit the growth of cell line derived xenografts tumors in mice, and whether systemic inhibition of NVL leads to specific toxicities. Through a medicinal chemistry campaign we identified analog MM927 (Table A), which inhibited HCT116 proliferation with an IC50 of 79 nM, five times more potent than the parent compound, MM017. Cells harboring NVL^{P307T} mutation are relatively resistant to MM927 (IC50=1.67 μM), suggesting that MM927 functions similarly to MM017. To interpret the toxicity of MM927 in mice, we first asked whether MM927 also inhibits mouse NVL. To test the potency of MM927 against mouse NVL within the same cell type, we ectopically expressed either human or mouse NVL in NVL^{AID/AID} cells. Treatment with Ph-IAA degrades the endogenous protein so that the cells must rely solely on ectopically expressed NVL. In the mock infected cells, degrading NVL^{AID/AID} by the addition of Ph-IAA reduced cell viability as expected, and the viability was completely rescued by expression of either ectopic mouse or human NVL. In these conditions, MM927 inhibited cells expressing ectopic human NVL with an IC50 of 0.051 μM, similar to its potency with endogenous NVL, suggesting that NVL overexpression does not substantially alter sensitivity. MM927 also inhibited mouse NVL with an IC50 of 0.207 μM, suggesting that adequate plasma concentrations would be capable of inhibiting NVL in normal mouse tissue.

In pharmacokinetic studies to evaluate the exposure of MM927, a 10 mg/kg intraperitoneal (IP) injection of MM927 led to peak plasma levels (Cmax) of 2510 ng/ml (5.15 μM) with an elimination half-life of 101.33 minutes. Plasma exposure was dose-dependent up to 35 mg/kg (IP injection) reaching a maximum concentration at 17.86 μM. These levels exceed the anti-proliferative IC99 of MM927 in cells expressing either human or mouse NVL. In a subsequent pharmacodynamic (PD) study, we asked if we could assess on-target NVL inhibition via a subsequent p53 and/or p21 induction in tumor xenografts in mice treated with MM927. We implanted HCT116 NVL^{WT} and NVL^{R403W} cells subcutaneously on either flank of the mice and evaluated the levels of p53 and p21 in either tumor at various time points after IP administration of 35 mg/kg of MM927. The p53 protein levels increased 3 hours after injection and p21 increased soon thereafter. Both p53 and p21 remained elevated at 12 hours and decreased to basal levels by 24 hours, consistent with the pharmacokinetic properties of MM927. Importantly, the levels of p53 and p21 induction in tumor xenografts were comparable to 24 hours of continuous MM017 treatment in cultured cells. Critically, NVL^{R403W} tumors implanted into the same mice on the opposing flank showed no change in p53 and p21, indicating that the observed changes in NVL wild type tumors were the result of NVL inhibition and establishing p53 and p21 as viable PD markers.

Having established the kinetics of MM927 mediated NVL inhibition in xenograft tumors, we administered 35 mg/kg MM927 IP twice a day for 21 days to mice harboring tumors derived from either HCT116 NVL wild type or NVL^{R403W} cells. MM927 treatment significantly reduced the rate of growth of NVL^{WT} tumors but had no significant impact on NVL^{R403W} tumors. The growth of a single NVL^{WT} tumor out of 10 analyzed seemed to initially respond to treatment, but later then progressed suggesting an acquired mechanism of resistance. Nonetheless, endpoint tumor measurements made at either the end of treatment or the time of euthanasia demonstrated that MM927 significantly reduced NVL WT tumor burden but had no effect on NVL^{R403W} tumors. MM927 was detected at micromolar levels in the plasma and tumor of all the treated mice 3 hours after the last treatment dose demonstrating adequate drug exposure over the course of treatment. Importantly, mice treated with MM927 showed no signs of overt toxicity. In addition, there was no significant difference in body weight, levels of red and white blood cells, hemoglobin, platelets, the liver enzyme alanine transaminase, and renal clearance of blood urea nitrogen between vehicle and drug treated groups. Taken together, we concluded that the potent, and bioavailable analog MM927 reduces tumor growth in mouse xenografts by inhibiting NVL without toxicity.

### DISCUSSION

Modern small molecule cancer drugs are ideally optimized to specifically bind a defined protein target to maximize on-target efficacy and minimize off-target toxicity. By contrast, older cancer therapies, which still constitute most small molecule cancer treatments, often influence multiple protein targets leading to poly-pharmacology and target ambiguity (42). Oxaliplatin, for example, is a first line treatment for colorectal cancer, which like its related analogs cisplatin and carboplatin, targets DNA directly leading to DNA adducts that trigger p53 activation through the DNA damage response (15). Oxaliplatin also targets ribosome biogenesis and leads to nucleolar stress, and this activity is through a target other than DNA since neither carboplatin nor cisplatin inhibit ribosome biogenesis (16-18, 43). Recent reports suggest that blocking ribosome production is the on-target activity for oxaliplatin raising the possibility that patients receiving oxaliplatin may have dose limiting toxicities resulting from off-target DNA damaging activity (19). If that is the case, small molecules that selectively target ribosome biogenesis without causing DNA damage may have improved efficacy and reduced toxicity resulting in a wider therapeutic index. Here, we discovered that MM017 and its bioavailable derivative MM927 inhibit NVL to block ribosome assembly, leading to p53 dependent apoptosis. Importantly, we found that MM017 did not trigger the DNA damage response and thus these molecules now provide a tool to specifically target ribosome biogenesis without DNA damage as a confounding factor.

NVL is a member of the type 2 AAA ATPase family of enzymes, which contain two tandem AAA ATPase domains (D1 and D2), that translocate peptide substrates through the central channel of a stacked, double-hexameric ring structure (2, 34, 40). Substrate capture triggers the formation of a staircase-like assembly which is followed by sequential ATP binding and hydrolysis events at AAA+ subunit interfaces in both D1 and D2 domains (27, 44). Nucleotide hydrolysis at the interface between subunit D and E is a key event in the cyclic translocation mechanism of this enzyme family, as it allows the subunit at the “bottom” of the staircase to disengage and transition to the topmost position (45). In a cryo-EM structure with catalytically inactive NVL, we found that MM017 bound to the D1 domain of substrate-bound NVL in two sites at the D/E and E/F interface. This structure indicates that MM017 does not inhibit substrate binding but rather blocks the translocation of the substrate through the central channel, either by inhibiting ATP hydrolysis or by preventing subunit repositioning within the staircase arrangement. This mechanism is distinct from NMS-873 and UPCDC30245, non-competitive p97 inhibitors that bind to all six subunits in a symmetric conformation that promotes substrate disengagement (40, 46).

The discovery of potent, selective, and bioavailable NVL inhibitors indicate that NVL is a ribosome biogenesis enzyme that may be therapeutically targeted with small molecules. The development of NVL inhibitors as cancer drugs will require identifying which tumor types and genotypes are most likely to respond. Myc is a transcription factor that upregulates the transcription of rDNA and the expression of ribosomal proteins that together promote ribosome biogenesis to drive cancer (47). Thus, cancers that depend on the expression of the Myc protein may be more susceptible to NVL inhibition. In addition, in our survey of anti-proliferative activity to more than 900 cancer cell lines, cell lines with p53 mutations were significantly more likely to be resistant to MM017. Consistent with these findings, genome-wide CRISPR screens in a colorectal cancer cell line nominated p53 and its downstream effector p21 as the two most essential genes for MM017 induced cell death. Finally, silencing of p53 in this colorectal cancer cell line protected cells from MM017 induced apoptosis. These findings suggest that cancers expressing wild-type p53 protein will be more likely to respond to NVL inhibition. Although p53 is frequently mutated in cancer, the frequency of mutations varies among different cancer types ranging from more than 90% in non-small cell and small cell lung cancers to less than 20% in leukemias, renal cell cancer, and bone cancers among others (www.cbioportal.com). Our studies also indicate that loss of p53 may be one possible mechanism of resistance, and thus, combination strategies that combine NVL inhibition with drugs that work through alternative pathways may be effective.

### Representative Synthetic Procedures for the Synthesis of Dibenzothiazepinone Analogs.

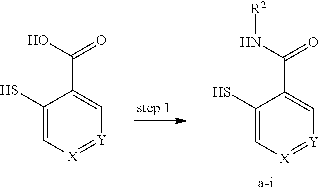

Step 1—General procedure for the synthesis of intermediates a-i: To a stirred suspension of a respective amine (1.0 eq.) in dry DMF (30 mL/mmol) was added a respective carboxylic acid (1.2 eq.), HATU (1.5 eq.) and DIPEA (2.0 eq.). The mixture was stirred at room temperature for 12 hours, after which the reaction mixture was treated with H₂O (60 mL/mmol). The organic layer was separated and dried over Na₂SO₄. The collected filtrate was evaporated in vacuum to give a crude di-sulfide intermediate which was dissolved in MeOH (7 mL/mmol). The reaction mixture was cooled to zero degrees Celcius, followed by a slow addition of NaBH₄ (2.0 eq.). After stirring at rt for 30 minutes, the MeOH was removed under reduced pressure and the residue diluted with 2N aqueous HCl to pH=4. Intermediates a-i were obtained after lyophilization.

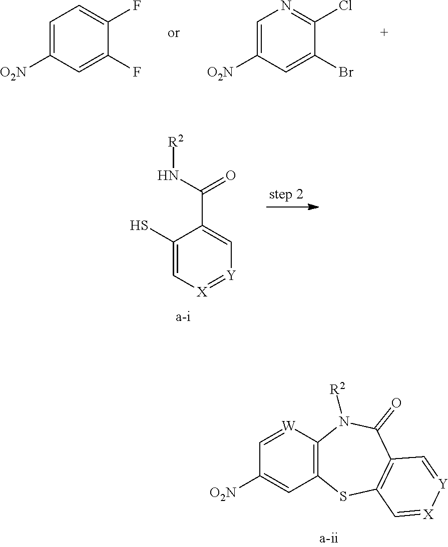

Step 2—General procedure for the synthesis of intermediates a-ii: To a solution compounds a-i (1.0 eq.) in anhydrous DMF (5 mL/mmol) was added 1,2-difluoro-4-nitrobenzene or 3-bromo-2chloro-5-nitropyridine (1.2 eq.) and K₂CO₃ (3.0 eq.). The mixture was stirred at 75° C. for 16 hrs, after which the reaction mixture was partitioned between EtOAc and H₂O. The organic layer was dried over Na₂SO₄, filtered, concentrated and purified by column chromatography silica gel to give intermediates a-ii.

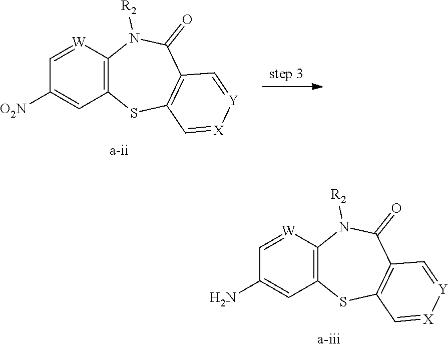

Step 3—General procedure for the synthesis of intermediates a-iii: To a solution of a-ii (1.0 eq.) in MeOH/H₂O (4:1 mL) (10 mL/mmol) was added NH₄Cl (18.0 eq.) and Fe (5.0 eq.). After reflux for 2 hrs, the reaction mixture was filtered over celite, and washed with MeOH. The collected filtrate was evaporated under vacuum to give intermediates a-iii.

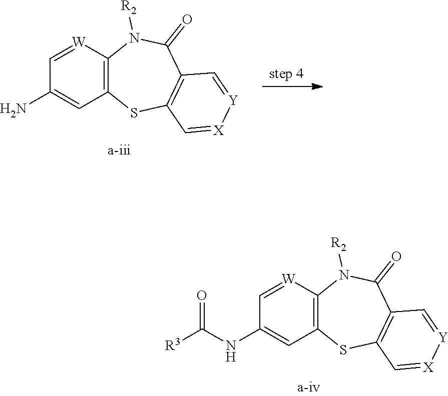

Step 4—General procedure for the synthesis of dibenzothiazepinone analogs a-iv: To a stirred suspension of a-iii (1.0 eq.) in dry DMF (30 mL/mmol) was added the respective carboxylic acid (1.2 eq.), HATU (1.5 eq.) and DIPEA (2.0 eq.). Alternatively, intermediate a-iii (1.0 eq.) and the respective acid chloride (1.2 eq.) were dissolved in dry CH₂Cl₂ (30 mL/mmol). The mixtures were stirred at room temperature for 12 hours, followed by dilution with H₂O. The organic layer was separated, dried over Na₂SO₄, and evaporated in vacuum to give the crude intermediates a-iv which were purified by flash chromatography.

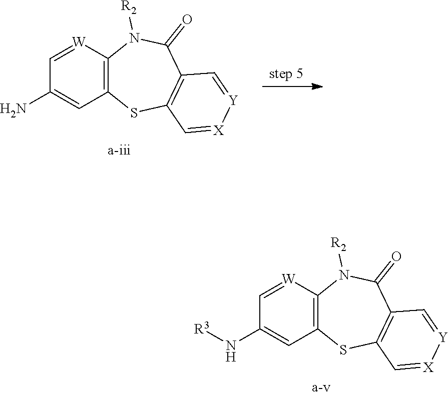

Step 5a—General procedure the synthesis of dibenzothiazepinone analogs a-iv with aromatic or heteroaromatic R³ groups: A solution of intermediates a-iii (1.0 eq.), R³Cl or R³Br (2.0 eq.), Pd₂(dba)₃ (0.2 eq.), DPEPhos (0.4 eq.) and t-BuOK (2.0 eq.) in toluene (20 mL/mmol) was stirred at 100° C. under an Ar atmosphere. For some substrates, a solution of intermediates a-iii (1.0 eq.), R³Cl or R³Br (2.0 eq.), Pd₂ (dba)₃ (0.2 eq.), DPEPhos (0.4 eq.) and Cs₂CO₃ (2.0 eq.) in dioxane (20 mL/mmol) was stirred at 100° C. under an Ar atmosphere. For some other substrates, a solution of intermediates a-iii (1.0 eq.), R³Cl or R³Br (2.0 eq.), Ruphos PdII G1 (0.1 eq.) and t-BuONa (2.0 eq.) in toluene (20 mL/mmol) was stirred at 100° C. under an Ar atmosphere. After completion of the respective reactions as judged by TLC-analysis, the mixture was filtered over celite and the filtrate was concentrated under vacuum. The residue was purified by preparative TLC or HPLC to yield dibenzothiazepinone analogs a-v with aromatic or heteroaromatic R³ groups.

Step 5b—General procedure the synthesis of dibenzothiazepinone analogs a-iv with non-aromatic R³ groups: To a solution of intermediates a-iii (1.0 eq.) and the corresponding non-aromatic ketones (2.0 eq.) in EtOAc/TFA (5:1) (20 mL/mmol) was slowly added NaBH₃CN (2.0 eq.). The resulting mixture was stirred for 2 hrs at 25° C. under an atmosphere of N₂. The reaction mixture was quenched with water and extracted with EtOAc. The organic phase was washed with brine, dried over Na₂SO₄, filtered, and concentrated under vacuum. The residue was purified by preparative HPLC to yield the dibenzothiazepinone analogs a-v with non-aromatic R3 groups.

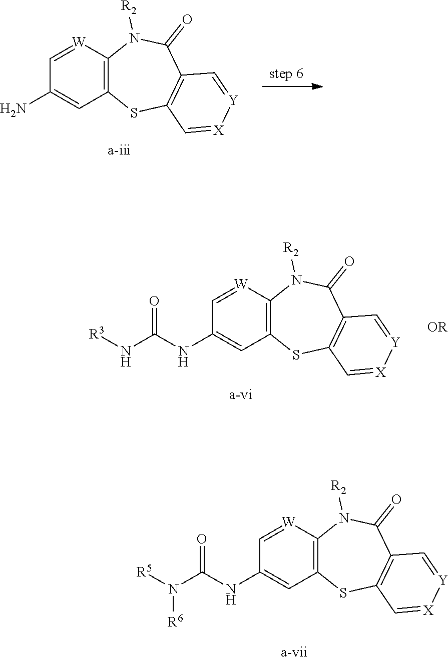

Step 6—General procedure the synthesis of urea-based dibenzothiazepinone analogs a-vi and a-vii: To a solution of a-iii (1.0 eq.) in dry CH₂Cl₂ (15 mL/mmol) was added i-Pr₂NEt (6.0 eq.) and triphosgene (0.4 eq.) at 0° C. under an atmosphere of Ar. After stirring for 3 hrs, the corresponding amines R³NH₂ or R⁵R⁶NH (2.0 eq.) were added and stirring was continued at 25° C. for 6 hrs. The reaction mixture was partitioned between CH₂Cl₂ and H₂O, and the organic layer was dried over anhydrous Na₂SO₄, filtered, concentrated and purified by preparative TLC and HPLC to give urea-based dibenzothiazepinone analogs a-vi or a-vii.

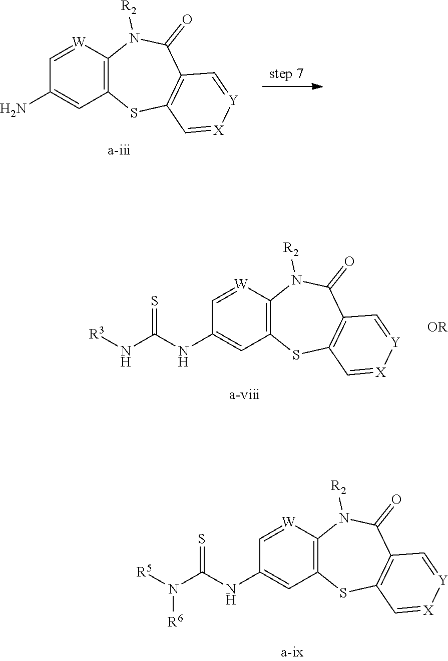

Step 7—General procedure the synthesis of thiourea-based dibenzothiazepinone analogs a-viii and a-ix: The same procedure as for step 6 above was employed with the exception that triphospgene was replaced with thiophosgene.

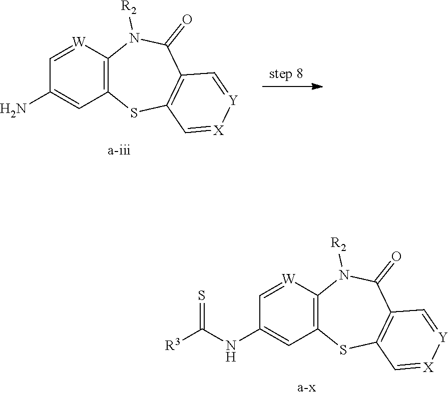

Step 8—General procedure the synthesis of thioamide-based dibenzothiazepinone analogs a-x: Intermediates a-iii (1.0 eq.), the corresponding aldehyde R³CHO (1.5 eq.), sulfur powder (3.0 eq.), K₂CO₃ (2.0 eq.), and water (4 mL/mmol) were placed into a tube sealed with a Teflon septum screw cap. The reaction mixture was stirred at 100° C. for 24 h. Upon completion of the reaction, the mixture was cooled to room temperature and extracted with ethyl acetate. The organic layer was dried over Na₂SO₄, and the solvent was removed under reduced pressure. The residue was purified by silica gel column chromatography to afford the corresponding thioamide-based dibenzothiazepinone analogs a-x.

### Representative Synthetic Procedures for the Synthesis of Dibenzazepinone Analogs.

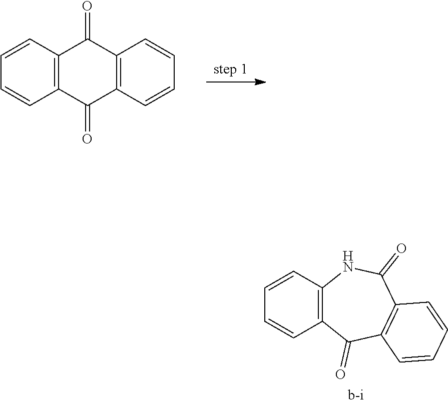 [SMILES: O=C1c2ccccc2C(=O)c2ccccc21 ; O=c1[nH]c2ccccc2c(=O)c2ccccc12]

Step 1—Synthesis of intermediate b-i: A mixture of concentrated sulfuric acid (1.0 mL/mmol) and CH₂Cl₂ (0.4 mL/mmol) was cooled to 0° C., followed by the addition of anthraquinone (1.0 eq.) and sodium azide (1.2 eq., portionwise). The reaction mixture was stirred overnight at room temperature and then poured into ice water. The product was filtered off, washed with water until acid-free and dried under vacuum to give intermediate b-i as white solid.

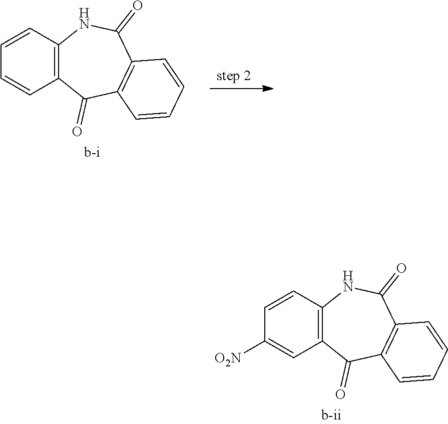 [SMILES: O=c1[nH]c2ccccc2c(=O)c2ccccc12 ; O=c1[nH]c2ccc([N+](=O)[O-])cc2c(=O)c2ccccc12]

Step 2—Synthesis of intermediate b-ii: To a round-bottomed flask was added intermediate b-i (1.0 eq.) and concentrated H₂SO₄ (1.1 mL/mmol). The mixture was stirred until all of the material was dissolved, then cooled to 0° C. and treated with KNO₃ (1.0 eq.) in portions. The reaction mixture was allowed to warm to 25° C., and stirred at that temperature for 1 h. The mixture was basified to pH 9 with 2N aq. NaOH, resulting in a precipitate. The solid was collected by filtration, washed with water and dried to provide the intermediate b-ii as a yellow solid.

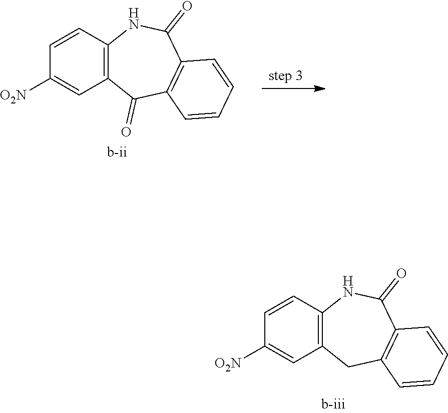 [SMILES: O=c1[nH]c2ccc([N+](=O)[O-])cc2c(=O)c2ccccc12 ; O=C1Nc2ccc([N+](=O)[O-])cc2Cc2ccccc21]

Step 3—Synthesis of intermediate b-iii: A suspension of b-ii (1.0 eq.) and Et₃SiH (10 eq.) in TFA (40 mL/mmol) was for 12 hrs stirring at 25° C., after which the mixture was concentrated under vacuum to yield a residue. The residue was purified by preparative HPLC to give intermediate b-iii as a white solid.

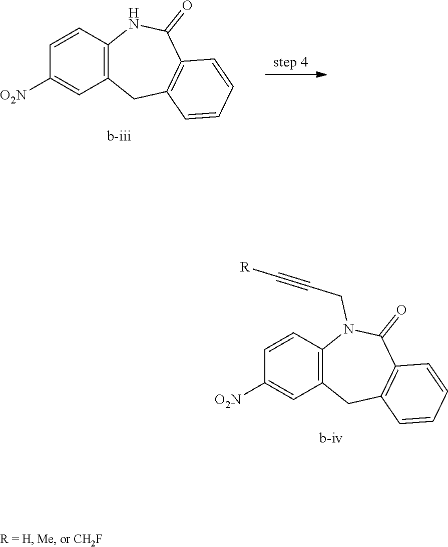

Step 4—General procedure for the synthesis of intermediates b-iv: To the solution of b-iii (1.0 eq.) in DMF (4.0 mL/mmol) was added NaOH (2.0 eq.) and 1-bromo-2-butyne or propargyl bromide or 1-bromo-4-fluoro-2-butyne (1.5 eq.) at room temperature. After stirring for 6 h at 60° C., IN aq. HCl was added to quench the reaction. The organic layer was separated, and the aqueous layer was extracted with CH₂Cl₂. The combined organic layers were washed with brine, dried over Na₂SO₄, and concentrated under vacuum to yield intermediates b-iv after purification by flash chromatography on silica gel.

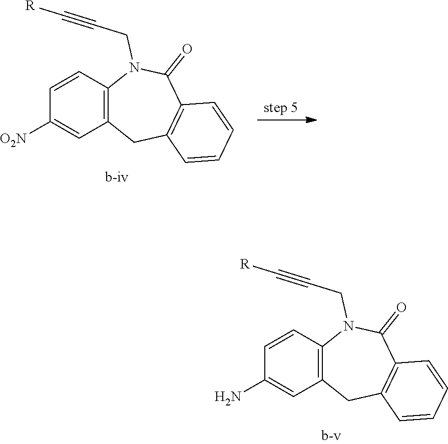

Step 5—General procedure for the synthesis of intermediates b-v: To a solution of intermediates b-iv (1.0 eq.) in MeOH/H₂O (4:1; 10 mL/mmol) was added NH₄Cl (18.0 eq.) and Fe (5.0 eq.). After reflux for 2 hrs, the reaction mixture was filtered over celite and washed with MeOH. The collected filtrate was concentrated under vacuum to yield intermediates b-v.

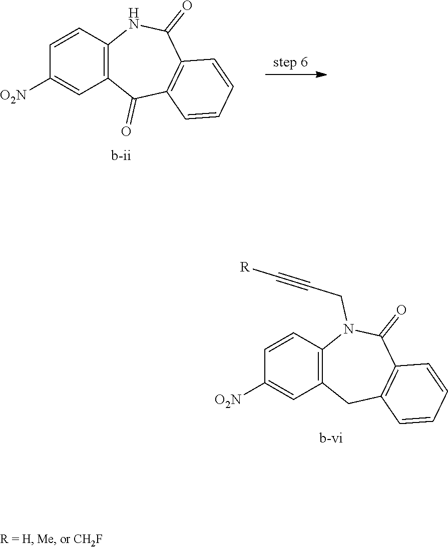

Step 6—General procedure for the synthesis of intermediates b-vi: The synthesis of intermediates b-vi follows the same procedures as for the synthesis of intermediates b-iv above (step 4).

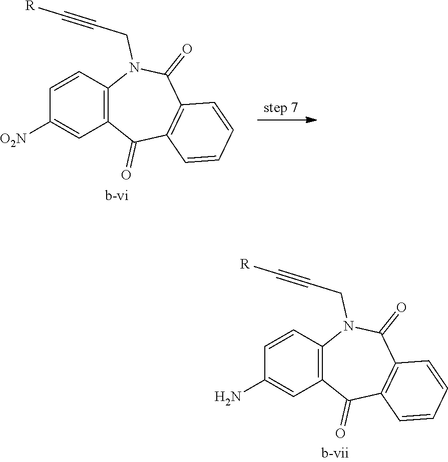

Step 7—General procedure for the synthesis of intermediates b-vii: The synthesis of intermediates b-vii follows the same procedures as for the synthesis of intermediates b-v above (step 5).

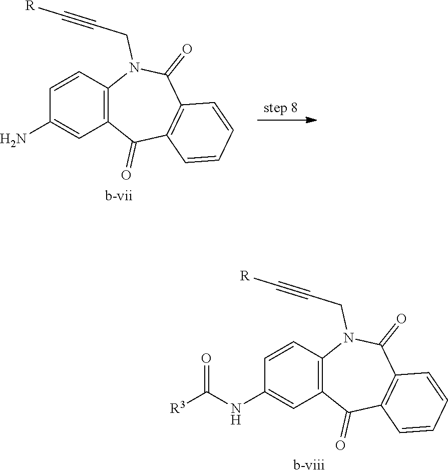

Step 8—General procedure for the synthesis of intermediates b-viii: The synthesis of intermediates b-viii follows the same procedures as for the synthesis of dibenzothiazepinone analogs a-iv described earlier.

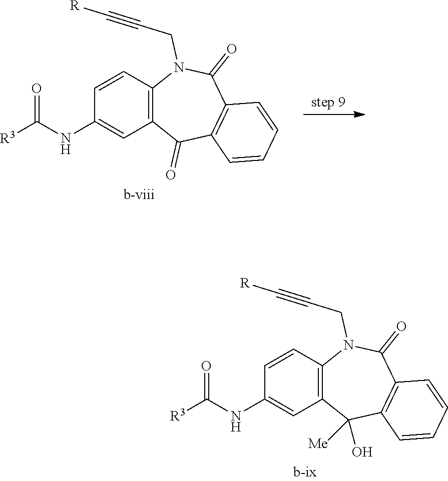

Step 9—General procedure for the synthesis of dibenzazepinone analogs b-ix: To a stirred suspension of b-viii (1.0 eq.) in dry THF (8.0 mL/mmol) was added MeMgBr (2.0 eq.) at 0° C. After stirring for 6 hrs at 25° C., the reaction mixture was treated with aq. NH₄Cl, followed by extraction with ethyl acetate. The combined organic layers were washed with brine, dried over anhydrous Na₂SO₄, filtered and concentrated under vacuum. The residue was purified by flash chromatography on silica gel to afford dibenzapinone analogs b-ix.

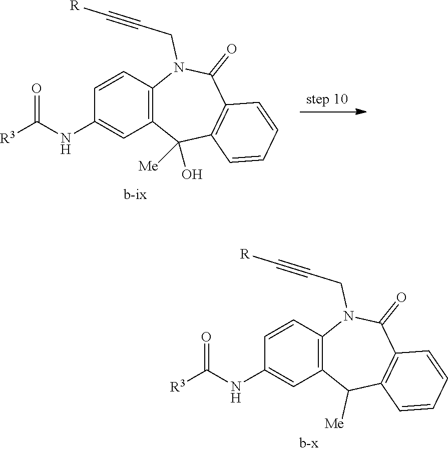

Step 10—General procedure for the synthesis of dibenzazepinone analogs b-x: A suspension of dibenzazepinone analogs b-ix (1.0 eq.) and Et₃SiH (10 eq.) in TFA (40 mL/mmol) was stirred for 12 hrs at 25° C. The mixture was concentrated under vacuum and the residue was purified by preparative HPLC to yield dibenzazepinone analogs b-x.

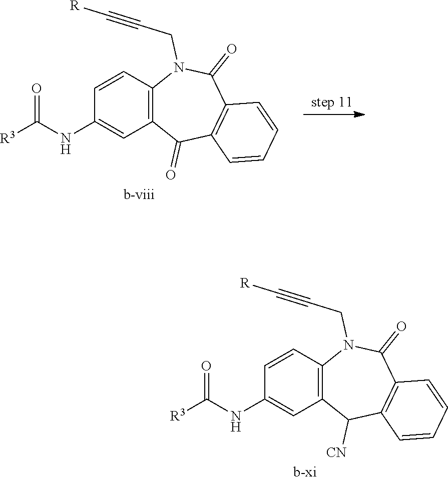

Step 11—General procedure for the synthesis of dibenzazepinone analogs b-xi: To a solution of b-viii (1.0 eq.) in DME (20 mL/mmol) was added TosMIC (1.5 eq.) followed by EtONa (2.0 eq.) at 0° C. under an atmosphere of N₂. The reaction mixture was warmed to 35° C. and stirred for 3 hrs and then quenched with water and extracted with EtOAc. The combined organic layers were washed with brine, dried over Na₂SO₄, filtered, and concentrated under vacuum. The residue was purified by preparative TLC and HPLC to yield dibenzazepinone analogs b-xi.

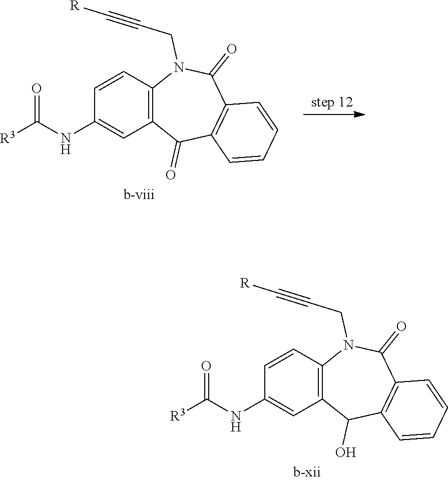

Step 12—General procedure for the synthesis of dibenzazepinone analogs b-xii: To a solution of b-viii (1.0 eq.) in MeOH (10 mL/mmol) at 0° C. was added NaBH₄ (1.5 eq.) portionwise. After 2 h at rt, brine was added, and the organic layer was separated. The aqueous layer was extracted with CH₂Cl₂. The combined organic layers were washed with brine, dried over Na₂SO₄, filtered, and concentrated under vacuum. The residue was purified by flash chromatography on silica gel to yield dibenzazepinone analogs b-xii.

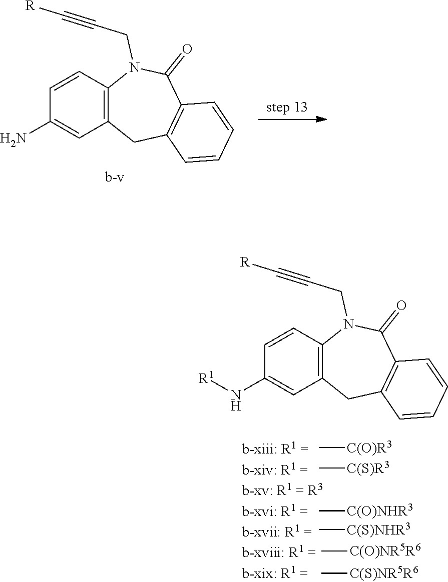

Step 13—General procedure for the synthesis of dibenzazepinone analogs b-xiii-b-xix: The synthesis of dibezazepinone analogs b-xiii-b-xix follows the same procedures as for the corresponding dibenzothiazepinone analogs a-iv-a-x described above.

### Characterization Data for Representative Dibenzothiazepinone and Dibenzazepinone Analogs.

4-Azido-N-(11-oxo-10-(2-(prop-2-yn-1-yloxy)ethyl)-10, 11-dihydro-dibenzo[b,f][1,4]thiazepin-7-yl)benzamide (2). Probe (2) was obtained as a yellow solid (82% yield). ¹H NMR (400 MHz, Chloroform-d) δ 7.92 (d, J=2.5 Hz, 1H), 7.84 (d, J=8.6 Hz, 2H), 7.77 (s, 1H), 7.67 (dd, J=7.6, 1.7 Hz, 1H), 7.60 (dd, J=8.8, 2.5 Hz, 1H), 7.50 (d, J=8.7 Hz, 1H), 7.42 (dd, J=7.5, 1.5 Hz, 1H), 7.35-7.25 (m, 2H), 7.12 (d, J=8.7 Hz, 2H), 4.57 (qd, J=8.9, 8.1, 5.1 Hz, 1H), 4.16 (d, J=2.4 Hz, 2H), 3.97 (dq, J=9.0, 5.0 Hz, 2H), 3.81 (dt, J=9.5, 4.2 Hz, 1H), 2.41 (t, J=2.3 Hz, 1H). ¹³C NMR (101 MHz, Chloroform-d) δ 169.2, 164.8, 144.0, 139.9, 138.9, 138.2, 137.1, 135.9, 131.1, 131.0, 130.8, 130.7, 129.0, 128.7, 126.7, 124.2, 121.4, 119.2, 79.5, 74.7, 67.6, 58.4, 51.6. LC-MS (ESI) calcd for C₂₅H₂₀N₅O₃S [M+H]⁺ 470.1, found 470.1.

N-(10-(2-Methoxyethyl)-11-oxo-10,11-dihydrodibenzo[b,f][1,4]thiazepin-7-yl) acetamide. ¹H NMR (400 MHz, Chloroform-d) δ 8.35 (s, 1H), 7.77 (s, 1H), 7.61 (d, J=7.2 Hz, 1H), 7.45 (d, J 8.7 Hz, 1H), 7.36 (d, J=7.0 Hz, 2H), 7.23 (dd, J=14.0, 7.0 Hz, 2H), 4.53 (dt, J=13.1, 6.2 Hz, 1H), 3.93-3.83 (m, 1H), 3.76 (dt, J=11.5, 5.9 Hz, 1H), 3.60 (dt, J=10.4, 5.4 Hz, 1H), 3.30 (s, 3H), 2.08 (s, 3H). ¹³C NMR (101 MHz, Chloroform-d) δ 169.4, 169.0, 139.3, 139.0, 138.2, 136.9, 136.3, 131.1, 131.0, 130.8, 128.6, 126.6, 123.7, 121.0, 70.0, 58.9, 51.5, 24.3. LC-MS: (ESI) calcd for C₁₈H₁₉N₂O₃S [M+H]⁺ 343.11, found 343.10.

N-(10-(2-Methoxyethyl)-11-oxo-10,11-dihydrodibenzo[b,f][1,4]thiazepin-7-yl) formamide. ¹H NMR (400 MHz, Chloroform-d) δ 8.35 (s, 1H), 7.82 (s, 1H), 7.68 (d, J=7.2 Hz, 1H), 7.40-7.55 (m, 3H), 7.30-7.36 (m, 2H), 7.23 (dd, J=14.0, 7.0 Hz, 2H), 4.53 (dt, J=13.1, 6.2 Hz, 1H), 3.93-3.83 (m, 1H), 3.76 (dt, J=11.5, 5.9 Hz, 1H), 3.60 (dt, J=10.4, 5.4 Hz, 1H), 3.30 (s, 3H). LC-MS: (ESI) calcd for C₁₇H₁₇N₂O₃S [M+H]⁺ 329.09, found 329.10.

4-Fluoro-N-(10-(2-methoxyethyl)-11-oxo-10,11-dihydrodibenzo[b,f][1,4]thiazepin-7-yl)benzamide. ¹H NMR (400 MHz, Chloroform-d) δ 8.31 (s, 1H), 7.92 (d, J=2.5 Hz, 1H), 7.92-7.80 (m, 2H), 7.61 (ddd, J=8.8, 6.1, 2.8 Hz, 2H), 7.46 (d, J=8.8 Hz, 1H), 7.42-7.32 (m, 1H), 7.29-7.19 (m, 2H), 7.10 (t, J=8.6 Hz, 2H), 4.58-4.47 (m, 1H), 3.92 (dt, J=13.8, 5.4 Hz, 1H), 3.84-3.74 (m, 1H), 3.67-3.57 (m, 1H), 3.33 (s, 3H). ¹³C NMR (101 MHz, Chloroform-d) δ 169.3, 166.3, 164.9, 163.8, 139.9, 138.9, 138.2, 137.0, 135.9, 131.1 (d, J=3.4 Hz), 130.8, 130.6 (d, J=3.2 Hz), 129.6, 129.6, 128.6, 126.7, 124.3, 121.5, 116.0, 115.7, 70.1, 58.9, 51.6. LC-MS: (ESI) calcd for C₂₃H₂₀FN₂O₃S [M+H]+ 423.12, found 423.10.

N-(10-(2-Methoxyethyl)-11-oxo-10,11-dihydrodibenzo[b,f][1,4]thiazepin-7-yl)benzamide. ¹H NMR (400 MHz, Chloroform-d) δ 8.27 (s, 1H), 7.94 (d, J=2.5 Hz, 1H), 7.83 (d, J=7.6 Hz, 2H), 7.69-7.58 (m, 2H), 7.52 (t, J=7.6 Hz, 1H), 7.47-7.32 (m, 4H), 7.31-7.19 (m, 2H), 4.53 (dt, J=13.0, 5.9 Hz, 1H), 3.90 (dt, J=13.6, 5.4 Hz, 1H), 3.79 (dt, J=11.5, 5.9 Hz, 1H), 3.62 (dt, J 10.5, 5.5 Hz, 1H), 3.33 (s, 3H). ¹³C NMR (101 MHz, Chloroform-d) δ 169.2, 165.9, 139.9, 139.0, 138.3, 137.0, 136.0, 134.4, 132.1, 131.1, 131.0, 130.7, 129.3, 128.8, 128.6, 127.1, 126.7, 124.2, 121.4, 70.1, 58.9, 51.6. LC-MS: (ESI) calcd for C₂₃H₂₁N₂O₃S [M+H]+ 405.13, found 405.10.

N-(10-(2-Methoxyethyl)-11-oxo-10,11-dihydrodibenzo[b,f][1,4]thiazepin-7-yl)-4-(trifluoromethyl)benzamide. ¹H NMR (400 MHz, Chloroform-d) δ 8.22 (s, 1H), 7.98-7.91 (m, 3H), 7.71 (d, J=8.2 Hz, 2H), 7.68-7.59 (m, 2H), 7.51 (d, J=8.8 Hz, 1H), 7.40 (dd, J=7.0, 2.0 Hz, 1H), 7.32-7.21 (m, 2H), 4.59-4.48 (m, 1H), 3.94 (dt, J=13.8, 5.4 Hz, 1H), 3.86-3.76 (m, 1H), 3.64 (dt, J=10.5, 5.4 Hz, 1H), 3.34 (s, 3H). ¹³C NMR (101 MHz, Chloroform-d) δ 169.2, 164.5, 140.4, 138.8, 138.2, 137.7, 137.2, 135.5, 133.9, 133.6, 131.1, 131.0, 130.8, 128.7, 127.6, 126.8, 125.9 (t, J=3.8 Hz), 124.3, 121.4, 70.2, 58.9, 51.7. LC-MS: (ESI) calcd for C₂₄H₂₀F₃N₂O₃S [M+H]⁺ 473.11, found 473.10.

N-(10-(2-Methoxyethyl)-11-oxo-10,11-dihydrodibenzo[b,f][1,4]thiazepin-7-yl)cyclohexanecarboxamide. ¹H NMR (400 MHz, Chloroform-d) δ 7.86 (d, J=2.4 Hz, 1H), 7.64 (dd, J=7.0, 2.1 Hz, 1H), 7.58 (s, 1H), 7.47 (dd, J=8.7, 2.5 Hz, 1H), 7.45-7.35 (m, 2H), 7.32-7.20 (m, 2H), 4.60-4.49 (m, 1H), 3.89 (dt, J=13.7, 5.5 Hz, 1H), 3.79 (dt, J=11.9, 6.1 Hz, 1H), 3.62 (dt, J=10.5, 5.6 Hz, 1H), 3.32 (s, 3H), 2.19 (tt, J=11.6, 3.3 Hz, 1H), 1.92-1.75 (m, 4H), 1.68 (d, J=6.3 Hz, 1H), 1.50 (q, J=11.6 Hz, 2H), 1.32-1.14 (m, 3H). ¹³C NMR (101 MHz, Chloroform-d) δ 174.7, 169.2, 139.4, 139.0, 138.3, 137.0, 136.2, 131.1, 130.7, 128.6, 126.6, 123.7, 120.8, 70.1, 58.9, 51.5, 46.3, 29.6, 25.6. LC-MS: (ESI) calcd for C₂₃H₂₇N₂O₃S [M+H]⁺ 411.18, found 411.10.

7-((4-Fluorobenzyl)amino)-10-(2-methoxyethyl)dibenzo[b,f][1,4]thiazepin-11 (10H)-one. ¹H NMR (400 MHz, Chloroform-d) δ 7.67 (dd, J=7.4, 1.8 Hz, 1H), 7.39 (dd, J=7.4, 1.6 Hz, 1H), 7.34-7.20 (m, 5H), 7.02 (t, J=8.6 Hz, 2H), 6.81 (d, J=2.8 Hz, 1H), 6.51 (dd, J=8.7, 2.8 Hz, 1H), 4.54 (dt, J=15.2, 7.4 Hz, 1H), 4.24 (s, 2H), 3.88-3.73 (m, 2H), 3.66-3.56 (m, 1H), 3.34 (s, 3H). ¹³C NMR (101 MHz, Chloroform-d) δ 169.3, 163.4, 161.0, 145.9, 139.2, 138.7, 137.5, 134.3, 133.8, 131.1, 130.9, 130.4, 129.1, 129.0, 128.4, 127.1, 115.9, 115.7, 115.5, 113.7, 70.0, 58.8, 51.3, 47.6. LC-MS: (ESI) calcd for C₂₃H₂₂FN₂O₂S [M+H]⁺ 409.14, found 409.10.

N-(10-(2-Methoxyethyl)-11-oxo-10,11-dihydrodibenzo[b,f][1,4]thiazepin-7-yl) nicotinamide. ¹H NMR (400 MHz, Chloroform-d) δ 9.07 (d, J=2.9 Hz, 1H), 8.73 (dd, J=4.9, 1.7 Hz, 1H), 8.62 (s, 1H), 8.17 (dt, J=8.1, 2.0 Hz, 1H), 7.94 (d, J=2.5 Hz, 1H), 7.67-7.56 (m, 2H), 7.48 (d, J=8.8 Hz, 1H), 7.42-7.32 (m, 2H), 7.31-7.19 (m, 2H), 4.57-4.46 (m, 1H), 3.93 (dt, J=13.8, 5.4 Hz, 1H), 3.85-3.75 (m, 1H), 3.71-3.58 (m, 1H), 3.33 (s, 3H). ¹³C NMR (101 MHz, Chloroform-d) & 169.3, 164.1, 152.6, 148.1, 140.3, 138.8, 138.2, 137.1, 135.6, 135.4, 131.0, 130.8, 130.3, 128.7, 126.8, 124.4, 123.7, 121.6, 70.1, 58.9, 51.7. LC-MS: (ESI) calcd for C₂₂H₂₀N₃O₃S [M+H]⁺ 406.12, found 406.00.

N-(10-(2-Methoxyethyl)-11-oxo-10,11-dihydrodibenzo[b,f][1,4]thiazepin-7-yl) isonicotinamide. ¹H NMR (400 MHz, Chloroform-d) δ 8.72 (s, 3H), 7.95 (d, J=2.4 Hz, 1H), 7.71-7.65 (m, 2H), 7.66-7.58 (m, 2H), 7.50 (d, J=8.8 Hz, 1H), 7.42-7.33 (m, 1H), 7.28-7.19 (m, 2H), 4.57-4.46 (m, 1H), 3.93 (dt, J=13.8, 5.3 Hz, 1H), 3.86-3.74 (m, 1H), 3.62 (dt, J 10.4, 5.4 Hz, 1H), 3.33 (s, 3H). ¹³C NMR (101 MHz, Chloroform-d) & 169.3, 164.0, 150.7, 141.6, 140.4, 138.8, 138.1, 137.1, 135.5, 131.1, 131.0, 130.9, 128.7, 126.8, 124.4, 121.6, 121.0, 70.1, 58.9, 51.7. LC-MS: (ESI) calcd for C₂₂H₂₀N₃O₃S [M+H]⁺ 406.12, found 406.00.

N-(10-(2-Methoxyethyl)-11-oxo-10,11-dihydrodibenzo[b,f][1,4]thiazepin-7-yl)-4-methylbenzamide. ¹H NMR (400 MHz, Chloroform-d) δ 8.17 (s, 1H), 7.93 (d, J=2.5 Hz, 1H), 7.73 (d, J=8.2 Hz, 2H), 7.68-7.55 (m, 2H), 7.44 (d, J=8.8 Hz, 1H), 7.38 (dd, J=7.2, 1.8 Hz, 1H), 7.31-7.19 (m, 4H), 4.60-4.49 (m, 1H), 3.96-3.85 (m, 1H), 3.84-3.74 (m, 1H), 3.67-3.57 (m, 1H), 3.33 (s, 3H), 2.40 (s, 3H). ¹³C NMR (101 MHz, Chloroform-d) δ 169.2, 165.8, 142.7, 139.8, 138.9, 138.3, 137.0, 136.1, 131.6, 131.1, 131.0, 130.7, 129.5, 128.6, 127.1, 126.7, 124.1, 121.3, 70.1, 58.9, 51.5, 21.5. LC-MS: (ESI) calcd for C₂₄H₂₃N₂O₃S [M+H]⁺ 419.14, found 419.10.

4-Azido-N-(10-(2-methoxyethyl)-11-oxo-10,11-dihydrodibenzo[b,f][1,4]thiazepin-7-yl)benzamide. ¹H NMR (400 MHz, Chloroform-d) δ 8.37 (s, 1H), 7.94 (d, J=2.5 Hz, 1H), 7.84 (d, J 8.6 Hz, 2H), 7.61 (dd, J=8.7, 2.4 Hz, 2H), 7.46 (d, J=8.8 Hz, 1H), 7.41-7.32 (m, 1H), 7.29-7.18 (m, 2H), 7.03 (d, J=8.6 Hz, 2H), 4.59-4.48 (m, 1H), 3.92 (dt, J=13.8, 5.4 Hz, 1H), 3.84-3.74 (m, 1H), 3.67-3.57 (m, 1H), 3.33 (s, 3H). ¹³C NMR (101 MHz, Chloroform-d) δ 169.3, 164.9, 144.0, 139.9, 138.9, 138.2, 137.0, 136.0, 131.1, 131.0, 130.8, 129.0, 128.6, 126.7, 124.2, 121.4, 119.1, 70.1, 58.9, 51.6. LC-MS: (ESI) calcd for C₂₃H₂₀N₅O₃S [M+H]⁺ 446.13, found 446.10.

N-(10-(2-Methoxyethyl)-11-oxo-10,11-dihydrodibenzo[b,f][1,4]thiazepin-7-yl) oxazole-4-carboxamide. ¹H NMR (400 MHz, Chloroform-d) δ 8.71 (s, 1H), 8.32 (d, J=1.0 Hz, 1H), 7.95 (d, J 2.5 Hz, 1H), 7.92 (d, J=1.0 Hz, 1H), 7.75-7.62 (m, 2H), 7.50 (d, J=8.8 Hz, 1H), 7.42 (dd, J 7.5, 1.5 Hz, 1H), 7.35-7.23 (m, 2H), 4.60-4.49 (m, 1H), 3.94 (dt, J=13.8, 5.5 Hz, 1H), 3.87-3.77 (m, 1H), 3.73-3.60 (m, 1H), 3.34 (s, 3H). ¹³C NMR (101 MHz, Chloroform-d) δ 169.0, 158.1, 150.6, 142.2, 140.2, 138.8, 138.4, 137.2, 135.8, 135.1, 131.2, 131.0, 130.7, 128.7, 126.8, 123.7, 120.9, 70.2, 58.9, 51.6. LC-MS: (ESI) calcd for C₂₀H₁₈N₃O₄S [M+H]⁺ 396.10, found 396.00.

4-Azido-N-(10-(2-methoxyethyl)-11-oxo-10,11-dihydrodibenzo[b,f][1,4]thiazepin-7-yl)-2-(prop-2-yn-1-yloxy)benzamide. ¹H NMR (400 MHz, Chloroform-d) δ 9.65 (s, 1H), 8.28 (d, J=8.5 Hz, 1H), 7.97 (d, J=2.5 Hz, 1H), 7.71-7.59 (m, 2H), 7.53-7.39 (m, 2H), 7.35-7.22 (m, 2H), 6.89 (dd, J=8.6, 2.0 Hz, 1H), 6.63 (d, J=2.0 Hz, 1H), 4.89 (d, J=2.4 Hz, 2H), 4.62-4.50 (m, 1H), 3.93 (dt, J=13.7, 5.5 Hz, 1H), 3.87-3.77 (m, 1H), 3.69-3.58 (m, 1H), 3.35 (s, 3H), 2.75 (t, J 2.4 Hz, 1H). ¹³C NMR (101 MHz, Chloroform-d) δ 169.1, 162.1, 156.4, 145.4, 139.8, 138.9, 138.4, 137.0, 136.2, 134.4, 131.2, 131.0, 130.7, 128.6, 126.7, 124.1, 121.3, 118.5, 112.8, 104.0, 77.8, 76.5, 70.2, 58.9, 57.6, 51.5. LC-MS: (ESI) calcd for C₂₆H₂₂N₅O₄S [M+H]⁺ 500.14, found 500.10.

2-Azido-N-(10-(2-methoxyethyl)-11-oxo-10,11-dihydrodibenzo[b,f][1,4]thiazepin-7-yl)benzamide. ¹H NMR (400 MHz, Chloroform-d) δ 7.67 (d, J=2.5 Hz, 1H), 7.39 (d, J=2.5 Hz, 1H), 7.31-7.21 (m, 7H), 6.87 (d, J=2.0 Hz, 1H), 6.64-6.55 (m, 1H), 4.56 (ddd, J=13.4, 6.7, 2.9 Hz, 1H), 3.79 (tdd, J=12.6, 6.0, 2.7 Hz, 2H), 3.61 (s, 1H), 3.34 (s, 3H). LC-MS: (ESI) calcd for C₂₃H₂₀N₅O₃S [M+H]⁺ 446.13, found 446.20.

3-Azido-N-(10-(2-methoxyethyl)-11-oxo-10,11-dihydrodibenzo[b,f][1,4]thiazepin-7-yl)benzamide. ¹H NMR (400 MHz, Chloroform-d) § 8.21 (s, 1H), 7.92 (d, J=2.5 Hz, 1H), 7.67-7.58 (m, 2H), 7.56 (dt, J=7.8, 1.4 Hz, 1H), 7.51 (t, J=2.0 Hz, 1H), 7.50-7.41 (m, 2H), 7.41-7.36 (m, 1H), 7.30-7.22 (m, 2H), 7.18 (dd, J=7.5, 2.8 Hz, 1H), 4.59-4.48 (m, 1H), 3.93 (dt, J 13.8, 5.4 Hz, 1H), 3.86-3.76 (m, 1H), 3.68-3.59 (m, 1H), 3.34 (s, 3H). ¹³C NMR (101 MHz, Chloroform-d) δ 169.2, 164.9, 141.1, 140.2, 138.8, 138.2, 137.1, 136.3, 135.7, 131.1, 131.0, 130.8, 130.2, 128.6, 126.8, 124.2, 123.2, 122.4, 121.4, 118.1, 70.2, 58.9, 51.6. LC-MS: (ESI) calcd for C₂₃H₂₀N₅O₃S [M+H]⁺ 446.13, found 446.00.

4-Azido-N-(11-oxo-10-(2-(prop-2-yn-1-yloxy)ethyl)-10,11-dihydrodibenzo[b,f][1,4]thiazepin-7-yl)benzamide. ¹H NMR (400 MHz, Chloroform-d) δ 7.92 (d, J 2.5 Hz, 1H), 7.84 (d, J=8.6 Hz, 2H), 7.77 (s, 1H), 7.67 (dd, J=7.6, 1.7 Hz, 1H), 7.60 (dd, J 8.8, 2.5 Hz, 1H), 7.50 (d, J=8.7 Hz, 1H), 7.42 (dd, J=7.5, 1.5 Hz, 1H), 7.35-7.25 (m, 2H), 7.12 (d, J=8.7 Hz, 2H), 4.57 (qd, J=8.9, 8.1, 5.1 Hz, 1H), 4.16 (d, J=2.4 Hz, 2H), 3.97 (dq, J=9.0, 5.0 Hz, 2H), 3.81 (dt, J=9.5, 4.2 Hz, 1H), 2.41 (t, J=2.3 Hz, 1H). ¹³C NMR (101 MHz, Chloroform-d) δ 169.2, 164.8, 144.0, 139.9, 138.9, 138.2, 137.1, 135.9, 131.1, 131.0, 130.8, 130.7, 129.0, 128.7, 126.7, 124.2, 121.4, 119.2, 79.5, 74.7, 67.6, 58.4, 51.6. LC-MS: (ESI) calcd for C₂₅H₂₀N₅O₃S [M+H]⁺ 470.13, found 470.10.

1-(10-(2-Methoxyethyl)-11-oxo-10,11-dihydrodibenzo[b,f][1,4]thiazepin-7-yl)-3-phenylurea. ¹H NMR (400 MHz, Chloroform-d) δ 7.87 (s, 1H), 7.76 (s, 1H), 7.63 (d, J=5.2 Hz, 1H), 7.48 (d, J=2.3 Hz, 1H), 7.37 (dt, J=7.5, 3.7 Hz, 1H), 7.33-7.17 (m, 6H), 7.01 (t, J=7.2 Hz, 1H), 4.63-4.52 (m, 1H), 3.86 (dt, J=13.6, 4.8 Hz, 1H), 3.78-3.68 (m, 1H), 3.59 (dt, J 10.3, 5.3 Hz, 1H), 3.28 (s, 3H). ¹³C NMR (101 MHz, Chloroform-d) δ 170.0, 153.3, 139.1, 138.2, 138.0, 137.7, 137.3, 136.9, 131.2, 131.0, 130.8, 129.1, 128.8, 126.6, 123.7, 123.1, 120.5, 120.2, 69.9, 58.9, 51.6. LC-MS: (ESI) calcd for C₂₃H₂₂N₃O₃S [M+H]⁺ 420.14, found 420.10.

N-(10-(2-Methoxyethyl)-11-oxo-10,11-dihydrodibenzo[b,f][1,4]thiazepin-7-yl)cyclobutanecarboxamide. ¹H NMR (400 MHz, Chloroform-d) δ 7.83 (d, J=2.5 Hz, 1H), 7.65 (dd, J=7.5, 1.7 Hz, 1H), 7.49 (dd, J=8.7, 2.5 Hz, 1H), 7.45-7.36 (m, 3H), 7.33-7.20 (m, 2H), 4.60-4.49 (m, 1H), 3.95-3.84 (m, 1H), 3.79 (dd, J=16.0, 6.0 Hz, 1H), 3.67-3.57 (m, 1H), 3.33 (s, 3H), 3.17-3.04 (m, 1H), 2.42-2.27 (m, 2H), 2.24-2.11 (m, 2H), 2.06-1.85 (m, 2H). 13C NMR (101 MHz, Chloroform-d) δ 173.4, 169.2, 139.5, 138.9, 138.3, 137.0, 136.1, 131.1, 131.0, 130.7, 128.6, 126.6, 123.5, 120.7, 70.1, 58.9, 51.5, 40.7, 25.2, 18.1. LC-MS: (ESI) calcd for C₂₁H₂₃N₂O₃S [M+H]⁺ 383.15, found 383.10.

N-(10-(2-Methoxyethyl)-11-oxo-10,11-dihydrodibenzo[b,f][1,4]thiazepin-7-yl)tetrahydro-2H-pyran-4-carboxamide. ¹H NMR (400 MHz, Chloroform-d) δ 7.86 (d, J=2.4 Hz, 1H), 7.65 (dd, J 7.4, 1.9 Hz, 1H), 7.60 (s, 1H), 7.51-7.35 (m, 3H), 7.33-7.21 (m, 2H), 4.59-4.48 (m, 1H), 4.07-3.98 (m, 2H), 3.91 (dt, J=13.7, 5.5 Hz, 1H), 3.84-3.74 (m, 1H), 3.67-3.57 (m, 1H), 3.40 (td, J=11.6, 2.3 Hz, 2H), 3.33 (s, 3H), 2.45 (tt, J=11.3, 4.0 Hz, 1H), 1.86 (td, J=11.4, 3.4 Hz, 2H), 1.77 (d, J=13.0 Hz, 2H). ¹³C NMR (101 MHz, Chloroform-d) δ 172.7, 169.2, 139.7, 138.9, 138.3, 137.0, 135.8, 131.1, 131.0, 130.7, 128.6, 126.7, 123.7, 120.9, 70.1, 67.1, 58.9, 51.6, 43.1, 29.7. LC-MS: (ESI) calcd for C₂₂H₂₅N₂O₄S [M+H]⁺ 413.16, found 413.10.

N-(10-(2-Methoxyethyl)-11-oxo-10,11-dihydrodibenzo[b,f][1,4]thiazepin-7-yl)cycloheptanecarboxamide. ¹H NMR (400 MHz, Chloroform-d) δ 7.84 (d, J=2.2 Hz, 1H), 7.65 (dd, J=7.5, 1.8 Hz, 1H), 7.52-7.36 (m, 4H), 7.33-7.20 (m, 2H), 4.60-4.49 (m, 1H), 3.95-3.84 (m, 1H), 3.84-3.74 (m, 1H), 3.68-3.57 (m, 1H), 3.33 (s, 3H), 2.35 (td, J=9.7, 4.1 Hz, 1H), 1.92 (ddd, J=14.1, 7.2, 4.2 Hz, 2H), 1.84-1.66 (m, 4H), 1.63-1.53 (m, 4H), 1.53-1.38 (m, 2H). ¹³C NMR (101 MHz, Chloroform-d) δ 175.7, 169.1, 139.5, 138.9, 138.3, 137.0, 136.1, 131.1, 131.0, 130.7, 128.6, 126.6, 123.6, 120.7, 70.1, 58.9, 51.5, 48.2, 31.5, 28.2, 26.5. LC-MS: (ESI) calcd for C₂₄H₂₉N₂O₃S [M+H]⁺ 426.20, found 426.20.

4-Chloro-N-(10-(2-methoxyethyl)-11-oxo-10,11-dihydrodibenzo[b,f][1,4]thiazepin-7-yl)benzamide. ¹H NMR (400 MHz, Chloroform-d) δ 8.05 (s, 1H), 7.92 (d, J=2.5 Hz, 1H), 7.77 (d, J 8.5 Hz, 2H), 7.69-7.57 (m, 2H), 7.49 (d, J=8.8 Hz, 1H), 7.45-7.36 (m, 3H), 7.33-7.21 (m, 2H), 4.59-4.48 (m, 1H), 3.98-3.88 (m, 1H), 3.86-3.76 (m, 1H), 3.73-3.59 (m, 1H), 3.34 (s, 3H). ¹³C NMR (101 MHz, Chloroform-d) δ 169.1, 164.7, 140.2, 138.8, 138.5, 138.3, 137.1, 135.6, 132.8, 131.1, 131.0, 130.8, 129.1, 128.7, 128.5, 126.8, 124.2, 121.3, 70.2, 58.9, 51.6. LC-MS: (ESI) calcd for C₂₃H₂₀ClN₂O₃S [M+H]⁺ 439.09, found 439.1.

3-Chloro-N-(10-(2-methoxyethyl)-11-oxo-10,11-dihydrodibenzo[b,f][1,4]thiazepin-7-yl)benzamide. ¹H NMR (400 MHz, Chloroform-d) δ 8.19 (s, 1H), 7.92 (d, J=2.5 Hz, 1H), 7.82 (s, 1H), 7.70 (d, J=7.8 Hz, 1H), 7.67-7.54 (m, 2H), 7.48 (d, J=8.8 Hz, 2H), 7.44-7.33 (m, 2H), 7.32-7.20 (m, 2H), 4.58-4.48 (m, 1H), 3.93 (dt, J=13.8, 5.4 Hz, 1H), 3.86-3.76 (m, 1H), 3.64 (dt, J=10.5, 5.5 Hz, 1H), 3.34 (s, 3H). ¹³C NMR (101 MHz, Chloroform-d) δ 169.2, 164.5, 140.2, 138.8, 138.2, 137.1, 136.2, 135.6, 135.0, 132.1, 131.1, 131.0, 130.8, 130.1, 128.6, 127.4, 126.8, 125.2, 124.2, 121.4, 70.2, 58.9, 51.6. LC-MS: (ESI) calcd for C₂₃H₂₀ClN₂O₃S [M+H]⁺ 439.09, found 439.00.

4-Methoxy-N-(10-(2-methoxyethyl)-11-oxo-10,11-dihydrodibenzo[b,f][1,4]thiazepin-7-yl)benzamide. ¹H NMR (400 MHz, Chloroform-d) δ 7.92 (d, J=2.5 Hz, 1H), 7.87 (s, 1H), 7.81 (d, J 8.8 Hz, 2H), 7.67 (dd, J=7.3, 2.0 Hz, 1H), 7.60 (dd, J=8.8, 2.5 Hz, 1H), 7.47 (d, J=8.8 Hz, 1H), 7.41 (dd, J=7.3, 1.7 Hz, 1H), 7.34-7.20 (m, 2H), 6.96 (d, J=8.8 Hz, 2H), 4.61-4.50 (m, 1H), 3.98-3.88 (m, 1H), 3.87 (s, 3H), 3.85-3.74 (m, 1H), 3.65 (dd, J=10.0, 5.6 Hz, 1H), 3.34 (s, 3H). ¹³C NMR (101 MHz, Chloroform-d) δ 169.1, 165.2, 162.7, 139.8, 138.9, 138.4, 137.1, 136.0, 131.1, 131.0, 130.7, 129.0, 128.6, 126.7, 126.5, 124.0, 121.2, 114.1, 70.2, 58.9, 55.5, 51.5. LC-MS: (ESI) calcd for C₂₄H₂₃N₂O₄S [M+H]⁺ 435.14, found 435.30.

4-Cyano-N-(10-(2-methoxyethyl)-11-oxo-10,11-dihydrodibenzo[b,f][1,4]thiazepin-7-yl)benzamide. ¹H NMR (400 MHz, Chloroform-d) δ 8.05 (s, 1H), 7.98-7.91 (m, 3H), 7.81-7.73 (m, 2H), 7.69-7.58 (m, 2H), 7.53 (d, J=8.7 Hz, 1H), 7.41 (dd, J=7.2, 1.7 Hz, 1H), 7.34-7.23 (m, 2H), 4.53 (ddd, J=13.8, 6.7, 5.3 Hz, 1H), 3.95 (dt, J=13.8, 5.4 Hz, 1H), 3.82 (ddd, J=10.1, 6.6, 5.1 Hz, 1H), 3.65 (dt, J=10.5, 5.4 Hz, 1H), 3.35 (s, 3H). ¹³C NMR (101 MHz, Chloroform-d) δ 169.1, 163.9, 140.7, 138.7, 138.3, 138.2, 137.2, 135.2, 132.7, 131.2, 131.0, 130.8, 128.7, 127.8, 126.9, 124.3, 121.4, 117.7, 115.7, 70.2, 58.9, 51.7, 29.7. LC-MS: (ESI) calcd for C₂₄H₂₀N₃O₃S [M+H]⁺ 430.12, found 430.10.

7-(Benzylamino)-10-(2-methoxyethyl)dibenzo[b,f][1,4]thiazepin-11 (10H)-one. ¹H NMR (400 MHz, Chloroform-d) δ 7.67 (dd, J=7.5, 1.8 Hz, 1H), 7.39 (dd, J=7.3, 1.6 Hz, 1H), 7.37-7.27 (m, 4H), 7.26-7.21 (m, 2H), 6.82 (d, J=2.8 Hz, 1H), 6.52 (dd, J=8.7, 2.8 Hz, 1H), 4.60-4.48 (m, 1H), 4.27 (s, 2H), 4.13 (s, OH), 3.87-3.73 (m, 2H), 3.66-3.55 (m, 1H), 3.34 (s, 3H). 13C NMR (101 MHz, Chloroform-d) δ 169.3, 146.1, 139.2, 138.7, 138.6, 137.4, 133.6, 131.1, 130.9, 130.3, 128.7, 128.4, 127.5, 127.0, 115.8, 113.6, 70.0, 58.8, 51.2, 48.2. LC-MS: (ESI) calcd for C₂₃H₂₃N₂O₂S [M+H]⁺ 391.15, found 391.10.

N-(11-Oxo-10-(2-(prop-2-yn-1-yloxy)ethyl)-10,11-dihydrodibenzo[b,f][1,4]thiazepin-7-yl) furan-3-carboxamide. ¹H NMR (400 MHz, Chloroform-d) δ 8.10 (s, 1H), 8.06 (s, 1H), 7.87 (d, J 2.5 Hz, 1H), 7.64 (dd, J=7.5, 1.8 Hz, 1H), 7.58 (dd, J=8.8, 2.5 Hz, 1H), 7.48-7.41 (m, 2H), 7.41-7.33 (m, 1H), 7.31-7.17 (m, 3H), 6.75 (d, J=1.1 Hz, 1H), 4.63-4.51 (m, 1H), 4.15 (d, J 2.4 Hz, 2H), 4.02-3.88 (m, 2H), 3.85-3.72 (m, 1H), 2.41 (t, J=2.3 Hz, 1H). ¹³C NMR (101 MHz, Chloroform-d) δ 169.3, 160.9, 145.5, 144.7, 139.7, 138.9, 138.2, 137.0, 135.8, 131.1, 131.0, 130.9, 130.7, 130.5, 128.6, 128.4, 127.1, 126.6, 124.2, 122.7, 121.4, 118.4, 116.0, 108.5, 79.5, 74.7, 67.5, 58.4, 51.5. LC-MS: (ESI) calcd for C₂₃H₁₉N₂O₄S [M+H]⁺ 419.11, found 419.30.

4-Ethyl-N-(10-(2-methoxyethyl)-11-oxo-10,11-dihydrodibenzo[b,f][1,4]thiazepin-7-yl)benzamide. ¹H NMR (400 MHz, Chloroform-d) δ 7.96 (s, 1H), 7.93 (d, J=2.5 Hz, 1H), 7.76 (d, J 8.3 Hz, 2H), 7.66 (dd, J=7.4, 1.8 Hz, 1H), 7.60 (dd, J=8.8, 2.5 Hz, 1H), 7.47 (d, J=8.8 Hz, 1H), 7.44-7.37 (m, 1H), 7.33-7.21 (m, 4H), 4.60-4.49 (m, 1H), 3.92 (dt, J=13.8, 5.5 Hz, 1H), 3.86-3.76 (m, 1H), 3.71-3.59 (m, 1H), 3.34 (s, 3H), 2.71 (q, J=7.6 Hz, 2H), 1.26 (t, J=7.6 Hz, 3H). ¹³C NMR (101 MHz, Chloroform-d) δ 169.1, 165.7, 149.0, 139.9, 138.9, 138.3, 137.1, 136.0, 131.8, 131.1, 131.0, 130.7, 128.6, 128.3, 127.2, 126.7, 124.0, 121.2, 70.2, 58.9, 51.5, 28.8, 15.3. LC-MS: (ESI) calcd for C₂₅H₂₅N₂O₃S [M+H]⁺ 433.16, found 433.30.

4-Ethynyl-N-(10-(2-methoxyethyl)-11-oxo-10,11-dihydrodibenzo[b,f][1,4]thiazepin-7-yl)benzamide. ¹H NMR (400 MHz, Chloroform-d) δ 8.21 (s, 1H), 7.93 (d, J=2.5 Hz, 1H), 7.79 (d, J 8.4 Hz, 2H), 7.67-7.58 (m, 2H), 7.55 (d, J=8.4 Hz, 2H), 7.47 (d, J=8.8 Hz, 1H), 7.42-7.34 (m, 1H), 7.32-7.21 (m, 2H), 4.59-4.48 (m, 1H), 3.98-3.87 (m, 1H), 3.80 (dd, J=15.3, 6.6 Hz, 1H), 3.68-3.58 (m, 1H), 3.34 (s, 3H), 3.24 (s, 1H). ¹³C NMR (101 MHz, Chloroform-d) δ 169.2, 165.0, 140.1, 138.8, 138.3, 137.1, 135.8, 134.3, 132.5, 131.1, 131.0, 130.7, 128.6, 127.1, 126.7, 126.0, 124.2, 121.4, 82.6, 80.1, 70.1, 58.9, 51.6. LC-MS: (ESI) calcd for C₂₅H₂₁N₂O₃S [M+H]⁺ 429.13, found 429.10.

N-(10-(2-Methoxyethyl)-11-oxo-10,11-dihydrodibenzo[b,f][1,4]thiazepin-7-yl)-2-naphthamide. ¹H NMR (400 MHz, Chloroform-d) δ 8.42 (s, 1H), 8.34 (s, 1H), 7.99 (d, J=2.5 Hz, 1H), 7.89 (d, J=7.1 Hz, 4H), 7.72-7.63 (m, 2H), 7.61-7.50 (m, 2H), 7.47 (d, J=8.8 Hz, 1H), 7.38 (dd, J=7.0, 2.1 Hz, 1H), 7.31-7.17 (m, 2H), 4.60-4.49 (m, 1H), 3.98-3.87 (m, 1H), 3.86-3.75 (m, 1H), 3.68-3.57 (m, 1H), 3.34 (s, 3H). ¹³C NMR (101 MHz, Chloroform-d) δ 169.2, 166.0, 139.9, 138.9, 138.3, 137.0, 136.1, 134.9, 132.5, 131.7, 131.1, 131.0, 130.7, 129.0, 128.8, 128.6, 128.1, 127.8, 127.7, 127.0, 126.7, 124.1, 123.5, 121.3, 70.1, 58.9, 51.6. LC-MS: (ESI) calcd for C₂₇H₂₃N₂O₃S [M+H]⁺ 455.14, found 455.20.

N-(10-(2-Methoxyethyl)-11-oxo-10,11-dihydrodibenzo[b,f][1,4]thiazepin-7-yl)-[1,1′-biphenyl]-4-carboxamide. ¹H NMR (400 MHz, Chloroform-d) δ 8.09 (s, 1H), 7.97 (d, J=2.5 Hz, 1H), 7.91 (d, J=8.3 Hz, 2H), 7.73-7.58 (m, 6H), 7.53-7.45 (m, 3H), 7.44-7.37 (m, 2H), 7.34-7.21 (m, 2H), 4.61-4.50 (m, 1H), 3.93 (dt, J=13.7, 5.4 Hz, 1H), 3.87-3.77 (m, 1H), 3.65 (dt, J 10.5, 5.5 Hz, 1H), 3.34 (s, 3H). ¹³C NMR (101 MHz, Chloroform-d) δ 169.1, 165.5, 145.0, 140.0, 139.7, 138.9, 138.3, 137.1, 135.9, 133.0, 131.2, 131.0, 130.7, 129.0, 128.6, 128.2, 127.6, 127.5, 127.2, 126.8, 124.1, 121.3, 70.2, 58.9, 51.6. LC-MS: (ESI) calcd for C₂₉H₂₅N₂O₃S [M+H]⁺ 481.16, found 481.10.

4-Methoxy-N-(11-oxo-10-(2-(prop-2-yn-1-yloxy)ethyl)-10,11-dihydrodibenzo[b,f][1,4]thiazepin-7-yl)benzamide. ¹H NMR (400 MHz, Chloroform-d) δ 7.97 (s, 1H), 7.92 (d, J=2.5 Hz, 1H), 7.81 (d, J=8.8 Hz, 2H), 7.65 (dd, J=7.4, 1.8 Hz, 1H), 7.59 (dd, J 8.8, 2.6 Hz, 1H), 7.46 (d, J=8.8 Hz, 1H), 7.40 (dd, J=7.0, 1.9 Hz, 1H), 7.33-7.19 (m, 2H), 6.95 (d, J=8.8 Hz, 2H), 4.57 (ddd, J=15.6, 8.7, 5.2 Hz, 1H), 4.15 (d, J=2.5 Hz, 2H), 4.01-3.90 (m, 1H), 3.86 (s, 3H), 3.79 (dt, J=9.5, 4.8 Hz, 1H), 2.41 (t, J=2.4 Hz, 1H). ¹³C NMR (101 MHz, Chloroform-d) δ 169.2, 165.2, 162.7, 139.7, 138.9, 138.3, 137.1, 136.1, 131.1, 131.0, 130.7, 129.0, 128.6, 126.7, 126.5, 124.0, 121.2, 114.0, 79.5, 74.7, 67.6, 58.4, 55.5, 51.5. LC-MS: (ESI) calcd for C₂₆H₂₃N₂O₄S [M+H]⁺ 459.14, found 459.10.

N-(10-(2-Methoxyethyl)-11-oxo-10,11-dihydrodibenzo[b,f][1,4]thiazepin-7-yl)cyclopropanecarboxamide. ¹H NMR (400 MHz, Chloroform-d) δ 7.82 (s, 1H), 7.75 (s, 1H), 7.65 (dd, J=7.5, 1.7 Hz, 1H), 7.48-7.35 (m, 3H), 7.32-7.20 (m, 2H), 4.60-4.49 (m, 1H), 3.90 (dt, J=13.7, 5.5 Hz, 1H), 3.84-3.74 (m, 1H), 3.68-3.57 (m, 1H), 3.33 (s, 3H), 1.48 (tt, J=8.1, 4.4 Hz, 1H), 1.10-1.01 (m, 2H), 0.89-0.78 (m, 2H). ¹³C NMR (101 MHz, Chloroform-d) δ 172.1, 169.2, 139.4, 138.9, 138.3, 137.0, 136.1, 131.1, 131.0, 130.7, 128.6, 126.6, 123.5, 120.7, 70.1, 58.9, 51.5, 29.7, 15.6, 8.2. LC-MS: (ESI) calcd for C₂₀H₂₁N₂O₃S [M+H]⁺ 369.13, found 369.10.

N-(10-(2-Methoxyethyl)-11-oxo-10,11-dihydrodibenzo[b,f][1,4]thiazepin-7-yl)-3-oxocyclobutane-1-carboxamide. ¹H NMR (400 MHz, Chloroform-d) δ 8.14 (s, 1H), 7.85 (d, J=2.5 Hz, 1H), 7.69-7.60 (m, 1H), 7.52 (dd, J=8.8, 2.5 Hz, 1H), 7.45 (d, J=8.8 Hz, 1H), 7.40 (dd, J 7.2, 1.8 Hz, 1H), 7.33-7.21 (m, 2H), 4.60-4.49 (m, 1H), 3.92 (dt, J=13.8, 5.3 Hz, 1H), 3.84-3.74 (m, 1H), 3.68-3.58 (m, 1H), 3.59-3.45 (m, 2H), 3.32 (s, 3H), 3.27-3.10 (m, 3H). 13C NMR (101 MHz, Chloroform-d) δ 203.3, 171.6, 169.3, 139.8, 138.9, 138.2, 137.1, 135.8, 131.1, 131.0, 130.8, 128.7, 126.7, 123.8, 120.9, 70.1, 58.9, 51.7, 38.7, 29.5. LC-MS: (ESI) calcd for C₂₁H₂₁N₂O₄S [M+H]⁺ 397.12, found 397.10.

N-(10-(2-Methoxyethyl)-11-oxo-10,11-dihydrodibenzo[b,f][1,4]thiazepin-7-yl)-4-(trifluoromethoxy)benzamide. ¹H NMR (400 MHz, Chloroform-d) δ 8.41 (s, 1H), 7.94 (d, J=2.5 Hz, 1H), 7.89 (d, J=8.8 Hz, 2H), 7.66-7.58 (m, 2H), 7.47 (d, J=8.8 Hz, 1H), 7.43-7.33 (m, 1H), 7.24 (q, J=2.9, 2.2 Hz, 4H), 4.58-4.47 (m, 1H), 3.92 (dt, J=13.8, 5.4 Hz, 1H), 3.84-3.74 (m, 1H), 3.62 (dt, J=10.4, 5.4 Hz, 1H), 3.33 (s, 3H). ¹³C NMR (101 MHz, Chloroform-d) δ 169.3, 164.6, 151.8, 140.1, 138.8, 138.2, 137.1, 135.8, 132.8, 131.1 (d, J=3.3 Hz), 130.8, 129.1, 128.6, 126.7, 124.3, 121.5, 120.7, 70.1, 58.9, 51.7. LC-MS: (ESI) calcd for C₂₄H₂₀F₃N₂O₄S [M+H]⁺ 489.11, found 489.10.

3,4-Dichloro-N-(10-(2-methoxyethyl)-11-oxo-10,11-dihydrodibenzo[b,f][1,4]thiazepin-7-yl)benzamide. ¹H NMR (400 MHz, Chloroform-d) δ 8.06 (s, 1H), 7.92 (dd, J=10.4, 2.3 Hz, 2H), 7.70-7.58 (m, 3H), 7.52 (dd, J=12.3, 8.5 Hz, 2H), 7.40 (dd, J=7.1, 1.8 Hz, 1H), 7.31-7.27 (m, 2H), 4.59-4.48 (m, 1H), 3.95 (dt, J=13.8, 5.4 Hz, 1H), 3.87-3.77 (m, 1H), 3.65 (dt, J=10.5, 5.5 Hz, 1H), 3.35 (s, 3H). ¹³C NMR (101 MHz, Chloroform-d) δ 169.2, 163.6, 140.4, 138.8, 138.2, 137.2, 136.7, 135.4, 133.4, 131.1, 131.0, 130.9, 130.8, 129.3, 128.7, 126.9, 126.2, 124.2, 121.4, 70.2, 58.9, 51.7. LC-MS: (ESI) calcd for C₂₃H₁₉C12N₂O₃S [M+H]⁺ 473.05, found 473.00.

N-(10-(2-Methoxyethyl)-11-oxo-10,11-dihydrodibenzo[b,f][1,4]thiazepin-7-yl)spiro[2.2]pentane-1-carboxamide. ¹H NMR (400 MHz, Chloroform-d) δ 7.83 (s, 1H), 7.65 (dd, J 6.6, 2.0 Hz, 2H), 7.48-7.42 (m, 1H), 7.41-7.35 (m, 2H), 7.32-7.20 (m, 2H), 4.60-4.49 (m, 1H), 3.94-3.84 (m, 1H), 3.84-3.74 (m, 1H), 3.66-3.57 (m, 1H), 3.32 (s, 3H), 1.91 (dd, J=7.6, 4.1 Hz, 1H), 1.56-1.49 (m, 1H), 1.38 (dd, J=7.6, 4.0 Hz, 1H), 1.09-1.00 (m, 1H), 0.92 (d, J 4.1 Hz, 3H). ¹³C NMR (101 MHz, Chloroform-d) δ 171.4, 169.2, 139.3, 138.9, 138.3, 137.0, 131.1, 131.1, 131.0, 130.7, 128.6, 126.6, 123.4, 120.6, 70.1, 58.8, 51.5, 23.1, 18.3, 15.0, 6.7, 5.1. LC-MS: (ESI) calcd for C₂₂H₂₃N₂O₃S [M+H]⁺ 395.15, found 395.00.

N-(10-(2-Methoxyethyl)-11-oxo-10,11-dihydrodibenzo[b,f][1,4]thiazepin-7-yl)spiro[3.3]heptane-2-carboxamide. ¹H NMR (400 MHz, Chloroform-d) δ 7.83 (d, J=2.4 Hz, 1H), 7.64 (dd, J=7.0, 2.2 Hz, 1H), 7.53-7.43 (m, 2H), 7.43-7.33 (m, 2H), 7.32-7.20 (m, 2H), 4.59-4.48 (m, 1H), 3.89 (dt, J=13.7, 5.5 Hz, 1H), 3.84-3.74 (m, 1H), 3.66-3.57 (m, 1H), 3.32 (s, 3H), 2.93 (p, J=8.5 Hz, 1H), 2.33-2.23 (m, 2H), 2.18 (t, J=10.3 Hz, 2H), 2.02 (t, J=7.2 Hz, 2H), 1.92 (t, J=7.2 Hz, 2H), 1.80 (q, J=8.5 Hz, 2H). ¹³C NMR (101 MHz, Chloroform-d) δ 173.5, 169.2, 139.4, 138.9, 138.3, 137.0, 136.2, 131.1, 131.0, 130.7, 128.6, 126.6, 123.5, 120.7, 70.1, 65.9, 58.9, 51.5, 39.8, 37.8, 35.5, 35.2, 34.3, 16.2. LC-MS: (ESI) calcd for C₂₄H₂₇N₂O₃S [M+H]⁺ 423.18, found 423.10.

N-(10-Hexyl-11-oxo-10, 11-dihydrodibenzo[b,f][1,4]thiazepin-7-yl) furan-3-carboxamide. ¹H NMR (400 MHz, Chloroform-d) δ 8.24 (s, 1H), 8.07 (s, 1H), 7.83 (d, J=2.5 Hz, 1H), 7.67-7.58 (m, 2H), 7.44 (t, J=1.8 Hz, 1H), 7.33 (dd, J=7.4, 1.6 Hz, 1H), 7.27 (d, J=5.3 Hz, 1H), 7.22 (td, J=7.2, 1.8 Hz, 2H), 6.77 (d, J=1.1 Hz, 1H), 4.69 (dd, J=14.5, 6.8 Hz, 1H), 3.59-3.47 (m, 1H), 1.61 (p, J=7.4 Hz, 2H), 1.43-1.12 (m, 6H), 0.90-0.79 (m, 3H). ¹³C NMR (101 MHz, Chloroform-d) δ 169.0, 161.0, 151.9, 150.5, 145.5, 145.0, 144.0, 139.0, 138.9, 138.2, 137.4, 135.8, 131.2, 131.0, 130.6, 129.6, 128.6, 126.1, 124.2, 122.7, 121.3, 121.0, 51.0, 31.4, 28.0, 26.6, 22.5, 14.0. LC-MS: (ESI) calcd for C₂₄H₂₅N₂O₃S [M+H]⁺ 421.16, found 421.10.

10-(2-Methoxyethyl)-7-((1-phenylethyl)amino) dibenzo[b,f][1,4]thiazepin-11 (10H)-one. ¹H NMR (400 MHz, Chloroform-d) δ 7.68-7.61 (m, 1H), 7.42-7.30 (m, 4H), 7.28 (ddd, J=5.2, 3.5, 1.7 Hz, 2H), 7.23 (dt, J=7.4, 1.8 Hz, 2H), 7.15 (d, J=8.7 Hz, 1H), 6.72 (dd, J=20.6, 2.8 Hz, 1H), 6.37 (ddd, J=12.8, 8.7, 2.8 Hz, 1H), 4.54-4.46 (m, 1H), 4.45-4.35 (m, 1H), 3.84-3.70 (m, 2H), 3.62-3.52 (m, 1H), 3.31 (d, J=1.8 Hz, 3H), 1.49 (d, J=6.7 Hz, 3H). ¹³C NMR (101 MHz, Chloroform-d) δ 169.3, 145.3, 145.2, 144.4, 139.2, 138.7, 137.2, 133.3, 131.1 (d, J=4.0 Hz), 130.9 (d, J=3.2 Hz), 130.3 (d, J=1.4 Hz), 128.8, 128.4 (d, J=1.5 Hz), 127.2, 126.9 (d, J 3.5 Hz), 125.8 (d, J=3.7 Hz), 116.7, 116.3, 113.9, 113.6, 70.0, 58.8, 53.6, 51.2, 29.7, 24.7. LC-MS: (ESI) calcd for C₂₄H₂₅N₂O₂S [M+H]⁺ 405.17, found 405.10.

N-(6-(2-Methoxyethyl)-5-oxo-5,6-dihydrobenzo[b]pyrido[3,2-f][1,4]thiazepin-9-yl)benzamide. ¹H NMR (400 MHz, Methanol-d₄) δ 8.30 (dd, J=4.9, 1.8 Hz, 1H), 8.25 (s, 1H), 7.81 (dd, J=7.6, 1.8 Hz, 1H), 7.78-7.71 (m, 1H), 7.63 (s, 1H), 7.48 (d, J=6.0 Hz, 2H), 7.16 (dd, J 7.6, 4.8 Hz, 1H), 6.95 (s, 1H), 3.58 (dp, J=8.5, 4.3, 3.6 Hz, 4H), 3.40 (s, 3H). ¹³C NMR (101 MHz, Methanol-d₄) δ 168.0, 167.5, 161.9, 157.1, 150.2, 150.0, 138.0, 137.0, 135.5, 135.3, 134.6, 131.8, 129.7, 128.3, 127.3, 119.7, 119.2, 116.1, 107.8, 107.6, 70.4, 57.6, 39.4. LC-MS: (ESI) calcd for C₂₂H₂₀N₃O₃S [M+Na]⁺ 428.10, found 428.00.

N-(10-(2-Methoxyethyl)-11-oxo-10,11-dihydrodibenzo[b,f][1,4]thiazepin-7-yl) azetidine-1-carboxamide. ¹H NMR (400 MHz, Methanol-d₄) δ 7.78 (d, J=2.4 Hz, 1H), 7.63-7.55 (m, 1H), 7.52-7.37 (m, 3H), 7.40-7.29 (m, 2H), 4.55 (ddd, J=14.0, 6.8, 5.3 Hz, 1H), 4.06 (t, J=7.6 Hz, 4H), 3.90 (dt, J=14.0, 5.3 Hz, 1H), 3.75-3.65 (m, 1H), 3.60 (dt, J=10.5, 5.4 Hz, 1H), 3.29 (s, 3H), 2.34-2.22 (m, 2H). ¹³C NMR (101 MHz, Methanol-d₄) δ 170.0, 157.3, 139.1, 138.1, 138.0, 137.5, 136.6, 130.7, 130.3, 128.4, 126.2, 122.8, 120.4, 69.6, 57.6, 50.7, 49.2. LC-MS: (ESI) calcd for C₂₀H₂₂N₃O₃S [M+H]⁺ 384.14, found 384.10.

Phenyl (10-(2-methoxyethyl)-11-oxo-10,11-dihydrodibenzo[b,f][1,4]thiazepin-7-yl) carbamate. ¹H NMR (400 MHz, Chloroform-d) δ 7.73 (s, 1H), 7.68 (dd, J=7.5, 1.7 Hz, 1H), 7.45-7.33 (m, 6H), 7.32-7.26 (m, 2H), 7.25-7.20 (m, 1H), 7.14 (d, J=7.4 Hz, 2H), 4.61-4.50 (m, 1H), 3.91 (dt, J=13.7, 5.5 Hz, 1H), 3.86-3.76 (m, 1H), 3.70-3.60 (m, 1H), 3.33 (s, 3H). ¹³C NMR (101 MHz, Chloroform-d) δ 169.2, 151.6, 150.4, 139.4, 138.9, 138.3, 137.2, 135.5, 131.1, 131.0, 130.7, 129.4, 128.6, 126.8, 125.9, 122.6, 121.6, 119.8, 70.2, 58.9, 51.6. LC-MS: (ESI) calcd for C₂₃H₂₁N₂O₄S [M+H]⁺ 421.12, found 421.10.

3-Methoxy-N-(10-(2-methoxyethyl)-11-oxo-10,11-dihydrodibenzo[b,f][1,4]thiazepin-7-yl) azetidine-1-carboxamide. ¹H NMR (400 MHz, Methanol-d₄) δ 7.78 (d, J=2.4 Hz, 1H), 7.63-7.56 (m, 1H), 7.53-7.39 (m, 3H), 7.39-7.26 (m, 2H), 4.55 (ddd, J=14.0, 6.8, 5.3 Hz, 1H), 4.21 (d, J=6.5 Hz, 3H), 3.96-3.81 (m, 3H), 3.75-3.65 (m, 1H), 3.65-3.55 (m, 1H), 3.30 (d, J=2.0 Hz, 6H). ¹³C NMR (101 MHz, Methanol-d4) δ 170.0, 157.4, 139.1, 138.1, 137.9, 137.6, 136.7, 130.7, 130.4, 128.4, 126.3, 122.9, 120.4, 69.6, 68.6, 57.6, 56.2, 55.0, 50.8. LC-MS: (ESI) calcd for C₂₁H₂₄N₃O₄S [M+H]⁺ 414.15, found 414.10.

1-(11-Oxo-10-(2-(prop-2-yn-1-yloxy)ethyl)-10,11-dihydrodibenzo[b,f][1,4]thiazepin-7-yl)-3-phenylurea. ¹H NMR (400 MHz, Chloroform-d) δ 7.84 (s, 1H), 7.71 (s, 1H), 7.65-7.59 (m, 1H), 7.47 (d, J=2.4 Hz, 1H), 7.43-7.35 (m, 1H), 7.38-7.17 (m, 8H), 7.06-6.97 (m, 1H), 4.64-4.53 (m, 1H), 4.10 (d, J=1.5 Hz, 2H), 3.96-3.83 (m, 2H), 3.81-3.71 (m, 1H), 2.37 (t, J=2.4 Hz, 1H). ¹³C NMR (101 MHz, Chloroform-d) δ 170.1, 153.2, 139.1, 138.1, 138.0, 137.6, 137.2, 137.0, 131.2, 131.0, 130.8, 129.1, 128.8, 126.5, 123.7, 123.1, 120.5, 120.3, 79.3, 74.9, 67.2, 58.4, 51.5. LC-MS: (ESI) calcd for C₂₅H₂₂N₃O₃S [M+H]⁺ 444.14, found 444.10.

4-(tert-butyl)-N-(11-Oxo-10-(2-(prop-2-yn-1-yloxy)ethyl)-10,11-dihydrodibenzo[b,f][1,4]thiazepin-7-yl)benzamide. ¹H NMR (400 MHz, Chloroform-d) δ 7.94 (d, J=2.5 Hz, 1H), 7.89 (s, 1H), 7.77 (d, J=8.5 Hz, 2H), 7.67 (dd, J=7.0, 2.2 Hz, 1H), 7.60 (dd, J 8.8, 2.5 Hz, 1H), 7.48 (dd, J=8.6, 6.4 Hz, 3H), 7.41 (dd, J=7.3, 1.5 Hz, 1H), 7.29 (dd, J=7.4, 1.9 Hz, 2H), 4.64-4.52 (m, 1H), 4.16 (d, J=2.4 Hz, 2H), 4.02-3.89 (m, 2H), 3.81 (dq, J=10.0, 5.3, 4.8 Hz, 1H), 2.41 (t, J=2.4 Hz, 1H), 1.35 (s, 9H). ¹³C NMR (101 MHz, Chloroform-d) δ 169.1, 165.6, 155.9, 139.9, 138.9, 138.3, 137.1, 136.0, 131.5, 131.2, 131.1, 130.7, 128.6, 126.9, 126.7, 125.8, 124.0, 121.2, 79.5, 74.6, 67.6, 58.4, 51.5, 35.0, 31.1. LC-MS: (ESI) calcd for C₂₉H₂₉N₂O₃S [M+H]⁺ 485.19, found 485.10.

4-Methoxy-3-methyl-N-(11-oxo-10-(2-(prop-2-yn-1-yloxy)ethyl)-10,11-dihydrodibenzo[b,f][1,4]thiazepin-7-yl)benzamide. ¹H NMR (400 MHz, Chloroform-d) δ 7.91 (d, J 2.5 Hz, 1H), 7.84 (s, 1H), 7.73-7.57 (m, 4H), 7.46 (d, J=8.8 Hz, 1H), 7.43-7.36 (m, 1H), 7.33-7.20 (m, 2H), 6.90-6.83 (m, 1H), 4.64-4.52 (m, 1H), 4.16 (d, J=2.5 Hz, 2H), 3.96 (h, J 5.0 Hz, 2H), 3.89 (s, 3H), 3.87-3.73 (m, 1H), 2.41 (t, J=2.4 Hz, 1H), 2.26 (s, 3H). ¹³C NMR (101 MHz, Chloroform-d) δ 169.1, 165.4, 160.9, 144.6, 139.7, 138.9, 138.3, 137.1, 136.1, 131.2, 131.1, 131.0, 130.9, 130.7, 130.4, 129.5, 128.6, 128.4, 127.2, 127.1, 126.7, 126.6, 126.0, 123.9, 121.1, 118.4, 116.0, 109.6, 79.5, 74.5, 67.5, 58.4, 55.5, 51.5, 51.2, 29.7, 16.3. LC-MS: (ESI) calcd for C₂₇H₂₅N₂O₄S [M+H]⁺ 473.16, found 473.20.

1-Cyclohexyl-3-(11-oxo-10-(2-(prop-2-yn-1-yloxy)ethyl)-10,11-dihydrodibenzo[b,f][1,4]thiazepin-7-yl) urea. ¹H NMR (400 MHz, Chloroform-d) δ 7.66-7.59 (m, 1H), 7.55 (d, J=2.5 Hz, 1H), 7.42-7.35 (m, 1H), 7.32 (d, J=8.8 Hz, 1H), 7.24 (dt, J=8.6, 2.8 Hz, 3H), 7.09 (s, 1H), 5.10 (d, J=7.9 Hz, 1H), 4.56 (ddd, J=16.3, 9.0, 5.3 Hz, 1H), 4.13 (dd, J 2.4, 1.2 Hz, 2H), 3.96-3.85 (m, 2H), 3.82-3.70 (m, 1H), 3.60 (tq, J=11.3, 3.8 Hz, 1H), 2.39 (t, J=2.4 Hz, 1H), 1.94-1.86 (m, 2H), 1.70-1.65 (m, 1H), 1.56 (dt, J=8.0, 3.9 Hz, 1H), 1.41-1.21 (m, 3H), 1.21-0.96 (m, 3H). ¹³C NMR (101 MHz, Chloroform-d) δ 169.6, 154.6, 139.1, 138.2, 137.7, 137.6, 137.0, 131.1, 131.0, 130.8, 128.6, 126.6, 122.9, 120.2, 79.4, 74.7, 67.4, 58.4, 51.4, 48.9, 33.6, 25.5, 24.8. LC-MS: (ESI) calcd for C₂₅H₂₈N₃O₃S [M+H]⁺ 450.19, found 450.10.

N-(11-Oxo-10-(2-(prop-2-yn-1-yloxy)ethyl)-10,11-dihydrodibenzo[b,f][1,4]thiazepin-7-yl) azetidine-1-carboxamide. ¹H NMR (400 MHz, Chloroform-d) δ 7.64 (d, J=9.2 Hz, 2H), 7.37 (d, J=9.1 Hz, 3H), 7.32-7.19 (m, 2H), 6.16 (s, 1H), 4.63-4.50 (m, 1H), 4.14 (d, J=2.4 Hz, 2H), 4.05 (t, J=7.6 Hz, 4H), 3.98-3.86 (m, 2H), 3.82-3.71 (m, 1H), 2.40 (t, J=2.3 Hz, 1H), 2.28 (p, J=7.5 Hz, 2H). ¹³C NMR (101 MHz, Chloroform-d) δ 169.2, 155.8, 139.0, 138.4, 138.3, 136.9, 136.8, 131.1, 131.0, 130.6, 128.5, 126.6, 122.8, 120.1, 79.5, 74.6, 67.5, 58.3, 51.3, 49.2, 15.2. LC-MS: (ESI) calcd for C₂₂H₂₂N₃O₃S [M+H]⁺ 408.14, found 408.10.

N-(10-(2-Methoxyethyl)-11-oxo-10,11-dihydrodibenzo[b,f][1,4]thiazepin-7-yl) pyrrolidine-1-carboxamide. ¹H NMR (400 MHz, Chloroform-d) δ 7.70 (d, J=2.2 Hz, 1H), 7.65 (dd, J=7.4, 1.8 Hz, 2H), 7.44-7.32 (m, 3H), 7.32-7.19 (m, 2H), 6.37 (s, 1H), 4.61-4.50 (m, 1H), 3.87 (dt, J 13.5, 5.6 Hz, 1H), 3.78 (dt, J=10.0, 6.2 Hz, 1H), 3.66-3.56 (m, 1H), 3.46-3.35 (m, 4H), 3.32 (s, 3H), 2.00-1.88 (m, 4H). ¹³C NMR (101 MHz, Chloroform-d) δ 169.2, 153.6, 139.1, 138.4, 138.2, 137.3, 136.8, 131.1, 131.0, 130.5, 128.5, 126.4, 123.2, 120.5, 70.0, 58.8, 51.3, 48.0, 45.8, 25.6. LC-MS: (ESI) calcd for C₂₁H₂₄N₃O₃S [M+H]⁺ 398.16, found 398.10.

N-(10-(2-Methoxyethyl)-11-oxo-10,11-dihydrodibenzo[b,f][1,4]thiazepin-7-yl) piperidine-1-carboxamide. ¹H NMR (400 MHz, Chloroform-d) δ 7.64 (dt, J=5.1, 2.0 Hz, 2H), 7.43-7.33 (m, 2H), 7.31 (d, J=2.5 Hz, 1H), 7.24 (dd, J=7.4, 2.0 Hz, 2H), 6.55 (s, 1H), 4.61-4.50 (m, 1H), 3.87 (dt, J=13.5, 5.5 Hz, 1H), 3.82-3.73 (m, 1H), 3.65-3.56 (m, 1H), 3.41 (t, J=5.3 Hz, 4H), 3.32 (s, 3H), 1.69-1.55 (m, 6H). ¹³C NMR (101 MHz, Chloroform-d) δ 169.2, 154.5, 139.1, 138.4, 138.3, 137.4, 136.8, 131.1, 131.0, 130.6, 128.5, 126.4, 123.5, 120.8, 70.0, 58.8, 51.3, 45.3, 25.7, 24.3. LC-MS: (ESI) calcd for C₂₂H₂₆N₃O₃S [M+H]⁺ 412.17, found 412.10.

N-(10-(2-Methoxyethyl)-11-oxo-10,11-dihydrodibenzo[b,f][1,4]thiazepin-7-yl) azepane-1-carboxamide. ¹H NMR (400 MHz, Chloroform-d) δ 7.62 (dd, J=7.3, 1.9 Hz, 1H), 7.56 (d, J=2.5 Hz, 1H), 7.44-7.29 (m, 2H), 7.29-7.18 (m, 3H), 5.29 (t, J=5.7 Hz, 1H), 4.60-4.49 (m, 1H), 3.86 (dt, J=13.7, 5.3 Hz, 1H), 3.80-3.70 (m, 1H), 3.65-3.55 (m, 1H), 3.31 (s, 3H), 3.18 (p, J 7.2 Hz, 3H), 1.66 (s, 1H), 1.50-1.37 (m, 3H), 0.86 (q, J=6.8, 5.2 Hz, 4H). ¹³C NMR (101 MHz, Chloroform-d) δ 169.6, 155.5, 139.1, 138.2, 137.8, 137.6, 137.0, 131.1, 130.9, 130.8, 128.6, 126.6, 122.9, 120.3, 70.0, 58.9, 51.4, 40.3, 31.5, 30.0, 26.6, 22.6, 14.0. LC-MS: (ESI) calcd for C₂₃H₂₈N₃O₃S [M+H]⁺ 426.19, found 426.10.

(S)-3-Fluoro-N-(10-(2-methoxyethyl)-11-oxo-10,11-dihydrodibenzo[b,f][1,4]thiazepin-7-yl) pyrrolidine-1-carboxamide. ¹H NMR (400 MHz, Chloroform-d) δ 7.70-7.63 (m, 2H), 7.46-7.36 (m, 3H), 7.32-7.25 (m, 3H), 6.14 (s, 1H), 5.36 (d, J=3.9 Hz, 0.5H), 5.23 (t, J=3.6 Hz, 0.5H), 4.55 (dt, J=13.1, 6.2 Hz, 1H), 3.93-3.76 (m, 3H), 3.74-3.47 (m, 5H), 3.34 (s, 3H). LC-MS: (ESI) calcd for C₂₁H₂₃FN₃O₃S [M+H]⁺ 416.14, found 415.10.

3-Cyano-N-(10-(2-methoxyethyl)-11-oxo-10,11-dihydrodibenzo[b,f][1,4]thiazepin-7-yl) azetidine-1-carboxamide. ¹H NMR (400 MHz, Chloroform-d) δ 7.74-7.60 (m, 2H), 7.47-7.32 (m, 3H), 7.31-7.21 (m, 2H), 6.51 (s, 1H), 4.53 (dt, J=12.2, 6.4 Hz, 1H), 4.42-4.21 (m, 1H), 3.89 (dt, J=13.7, 5.4 Hz, 1H), 3.83-3.73 (m, 1H), 3.62 (dt, J=10.5, 5.5 Hz, 1H), 3.53-3.41 (m, 1H), 3.33 (s, 3H). ¹³C NMR (101 MHz, Chloroform-d) δ 169.2, 153.6, 139.1, 138.4, 138.2, 137.3, 136.8, 131.1, 131.0, 130.5, 128.5, 126.4, 123.2, 120.5, 70.0, 58.8, 51.3, 48.0, 45.8, 25.6. LC-MS: (ESI) calcd for C₂₁H₂₁N₄O₃S [M+H]⁺ 409.14, found 409.30.

N-(10-(2-Methoxyethyl)-11-oxo-10,11-dihydrodibenzo[b,f][1,4]thiazepin-7-yl)-2-oxa-6-azaspiro[3.3]heptane-6-carboxamide. ¹H NMR (400 MHz, Chloroform-d) δ 7.69-7.61 (m, 2H), 7.43-7.32 (m, 3H), 7.32-7.20 (m, 2H), 6.13 (s, 1H), 4.79 (s, 4H), 4.54 (dd, J=12.9, 6.9 Hz, 1H), 4.18 (s, 4H), 3.89 (dt, J=13.7, 5.5 Hz, 1H), 3.84-3.74 (m, 1H), 3.67-3.57 (m, 1H), 3.33 (s, 3H). ¹³C NMR (101 MHz, Chloroform-d) δ 169.1, 155.6, 138.8, 138.4, 136.4, 131.1, 131.0, 130.6, 128.6, 126.7, 123.0, 120.3, 80.8, 70.1, 58.8, 51.5, 37.7, 29.8. LC-MS: (ESI) calcd for C₂₂H₂₄N₃O₄S [M+H]⁺ 426.15, found 426.30.

2-Methoxy-N-(10-(2-methoxyethyl)-11-oxo-10,11-dihydrodibenzo[b,f][1,4]thiazepin-7-yl)benzamide. ¹H NMR (400 MHz, Chloroform-d) δ 8.05 (s, 1H), 7.93 (d, J=2.5 Hz, 1H), 7.68-7.57 (m, 2H), 7.47 (d, J=8.8 Hz, 1H), 7.42-7.37 (m, 2H), 7.37-7.33 (m, 2H), 7.31-7.22 (m, 2H), 7.12-7.03 (m, 1H), 4.54 (ddd, J=13.8, 6.6, 5.5 Hz, 1H), 3.92 (dt, J=13.8, 5.5 Hz, 1H), 3.85 (s, 3H), 3.83-3.77 (m, 1H), 3.68-3.59 (m, 1H), 3.34 (s, 3H). ¹³C NMR (101 MHz, Chloroform-d) δ 169.1, 165.6, 160.0, 140.0, 138.9, 138.3, 137.1, 135.9, 135.8, 131.1, 131.0, 130.7, 129.8, 128.6, 126.7, 124.1, 121.2, 118.7, 118.2, 112.6, 70.2, 58.9, 55.5, 51.6. LC-MS: (ESI) calcd for C₂₄H₂₃N₂O₄S [M+H]⁺ 435.14, found 435.30.

N-(10-(2-Methoxyethyl)-11-oxo-10,11-dihydrodibenzo[b,f][1,4]thiazepin-7-yl)cyclopropanecarboxamide. ¹H NMR (400 MHz, Chloroform-d) δ 8.04 (s, 1H), 7.93 (d, J=2.5 Hz, 1H), 7.71-7.57 (m, 2H), 7.47 (d, J=8.7 Hz, 1H), 7.41 (s, 2H), 7.35 (d, J=5.3 Hz, 2H), 7.27 (pd, J=7.4, 1.7 Hz, 2H), 7.12-7.03 (m, 1H), 4.60-4.49 (m, 1H), 3.92 (dt, J=13.8, 5.5 Hz, 1H), 3.85 (s, 3H), 3.81 (dd, J=5.9, 4.1 Hz, 1H), 3.69-3.59 (m, 1H), 3.34 (s, 3H). ¹³C NMR (101 MHz, Chloroform-d) & 169.1, 165.6, 160.0, 140.0, 138.9, 138.3, 137.1, 135.9, 135.8, 131.1, 131.0, 130.7, 129.8, 128.6, 126.7, 124.1, 121.2, 118.7, 118.2, 112.6, 70.2, 58.9, 55.5, 51.6. LC-MS: (ESI) calcd for C₂₄H₂₃N₂O₄S [M+H]⁺ 435.14, found 435.30.

3-Bromo-4-methoxy-N-(10-(2-methoxyethyl)-11-oxo-10,11-dihydrodibenzo[b,f][1,4]thiazepin-7-yl)benzamide. ¹H NMR (400 MHz, Chloroform-d) δ 8.17 (s, 1H), 8.04 (d, J=2.3 Hz, 1H), 7.91 (d, J=2.5 Hz, 1H), 7.81 (dd, J=8.6, 2.3 Hz, 1H), 7.67-7.57 (m, 2H), 7.46 (d, J=8.8 Hz, 1H), 7.38 (dd, J=6.7, 2.3 Hz, 1H), 7.33-7.19 (m, 2H), 6.91 (d, J 8.7 Hz, 1H), 4.59-4.48 (m, 1H), 3.95 (s, 3H), 3.91 (m, 1H), 3.86-3.76 (m, 1H), 3.68-3.59 (m, 1H), 3.34 (s, 3H). ¹³C NMR (101 MHz, Chloroform-d) δ 169.2, 164.1, 158.8, 139.9, 138.8, 138.3, 137.0, 135.9, 132.3, 131.1, 131.0, 130.7, 128.6, 128.2, 127.8, 126.7, 124.2, 121.4, 111.9, 111.4, 70.1, 58.9, 56.5, 51.6. LC-MS: (ESI) calcd for C₂₄H₂₂BrN₂O₄S [M+H]⁺ 513.05, found 513.30.

3-Fluoro-4-methoxy-N-(10-(2-methoxyethyl)-11-oxo-10,11-dihydrodibenzo[b,f][1,4]thiazepin-7-yl)benzamide. ¹H NMR ¹H NMR (400 MHz, Chloroform-d) δ 8.13 (s, 1H), 7.92 (d, J=2.5 Hz, 1H), 7.68-7.56 (m, 4H), 7.47 (d, J=8.8 Hz, 1H), 7.39 (dd, J 7.3, 1.6 Hz, 1H), 7.32-7.20 (m, 2H), 6.99 (t, J=8.2 Hz, 1H), 4.60-4.49 (m, 1H), 3.95 (s, 3H), 3.95-3.87 (m, 1H), 3.86-3.76 (m, 1H), 3.69-3.59 (m, 1H), 3.34 (s, 3H). ¹³C NMR (101 MHz, Chloroform-d) δ 169.2, 164.3, 153.2, 150.7, 140.0, 138.9, 138.3, 137.0, 135.9, 131.1, 131.0, 130.7, 128.6, 126.7, 124.2, 123.8, 121.3, 115.4, 115.2, 112.8, 70.1, 58.9, 56.3, 51.6. LC-MS: (ESI) calcd for C₂₄H₂₂FN₂O₄S [M+H]⁺ 453.13, found 453.30.

3,4-Dichloro-N-(11-oxo-10-(2-(prop-2-yn-1-yloxy)ethyl)-10,11-dihydrodibenzo[b,f][1,4]thiazepin-7-yl)benzamide. ¹H NMR (400 MHz, Chloroform-d) δ 8.30 (s, 1H), 7.92 (dd, J=4.9, 2.3 Hz, 2H), 7.72-7.57 (m, 3H), 7.50 (dd, J=10.2, 8.6 Hz, 2H), 7.46-7.34 (m, 1H), 7.31-7.20 (m, 2H), 4.62-4.49 (m, 1H), 4.16 (d, J=2.4 Hz, 2H), 4.03-3.91 (m, 2H), 3.85-3.75 (m, 1H), 2.41 (t, J=2.4 Hz, 1H). ¹³C NMR (101 MHz, Chloroform-d) δ 169.3, 163.7, 140.2, 138.8, 138.1, 137.1, 136.6, 135.5, 134.2, 133.3, 131.0, 130.8, 129.3, 128.7, 126.8, 126.3, 124.3, 121.5, 79.4, 74.7, 67.6, 58.4, 51.6. LC-MS: (ESI) calcd for C₂₅H₁₉Cl₂N₂O₃S [M+H]⁺ 497.05, found 497.10.

N-(10-(2-Methoxyethyl)-11-oxo-10,11-dihydrodibenzo[b,f][1,4]thiazepin-7-yl)-1H-benzo[d][1,2,3]triazole-5-carboxamide. ¹H NMR (400 MHz, Methanol-d₄) δ 8.53 (s, 1H), 8.11 (d, J 2.5 Hz, 1H), 8.04 (dd, J=8.7, 1.5 Hz, 1H), 7.93 (d, J=8.7 Hz, 1H), 7.74 (dd, J=8.8, 2.5 Hz, 1H), 7.66-7.56 (m, 2H), 7.59-7.41 (m, 1H), 7.43-7.28 (m, 2H), 4.59 (ddd, J=14.1, 6.8, 5.1 Hz, 1H), 4.02-3.91 (m, 1H), 3.78-3.69 (m, 1H), 3.65 (dt, J=10.5, 5.4 Hz, 1H), 3.32 (s, 3H). 13C NMR (101 MHz, Methanol-d₄) δ 169.9, 166.7, 139.3, 139.0, 138.1, 137.0, 136.8, 130.8, 130.7, 130.4, 128.5, 126.5, 124.3, 121.8, 69.7, 57.6, 50.9. LC-MS: (ESI) calcd for C₂₃H₂₀N₅O₃S [M+H]⁺ 446.13, found 446.30.

N-(10-(2-Methoxyethyl)-11-oxo-10,11-dihydrodibenzo[b,f][1,4]thiazepin-7-yl)-2-methylfuran-3-carboxamide. ¹H NMR (400 MHz, Chloroform-d) δ 7.88 (d, J=2.5 Hz, 1H), 7.65 (dd, J=7.5, 1.8 Hz, 1H), 7.59 (s, 1H), 7.52 (dd, J=8.8, 2.5 Hz, 1H), 7.44 (d, J=8.7 Hz, 1H), 7.39 (dd, J=7.0, 2.0 Hz, 1H), 7.34-7.24 (m, 2H), 6.53 (d, J=2.1 Hz, 1H), 3.91 (dt, J=13.7, 5.5 Hz, 1H), 3.85-3.75 (m, 1H), 3.69-3.58 (m, 1H), 3.33 (s, 3H), 2.62 (s, 3H). ¹³C NMR (101 MHz, Chloroform-d) δ 169.1, 162.1, 158.5, 140.7, 139.8, 138.9, 138.3, 137.0, 135.8, 131.1, 131.0, 130.7, 128.6, 126.6, 124.0, 121.2, 115.4, 108.0, 70.1, 58.9, 51.5, 13.7. LC-MS: (ESI) calcd for C₂₂H₂₁N₂O₄S [M+H]⁺ 409.12, found 409.20.

N-(10-(2-Methoxyethyl)-11-oxo-10,11-dihydrodibenzo[b,f][1,4]thiazepin-7-yl)-2,5-dimethylfuran-3-carboxamide. ¹H NMR (400 MHz, Chloroform-d) δ 7.88 (d, J=2.5 Hz, 1H), 7.67 (dd, J=7.6, 1.7 Hz, 1H), 7.50 (dd, J=8.8, 2.5 Hz, 1H), 7.45 (d, J=8.7 Hz, 1H), 7.41 (dd, J=7.1, 1.8 Hz, 1H), 7.34 (s, 1H), 7.33-7.27 (m, 2H), 6.07 (s, 1H), 4.61-4.50 (m, 1H), 3.91 (dt, J=13.7, 5.5 Hz, 1H), 3.86-3.75 (m, 1H), 3.68-3.58 (m, 1H), 3.34 (s, 3H), 2.57 (s, 3H), 2.27 (s, 3H). 13C NMR (101 MHz, Chloroform-d) δ 169.1, 162.2, 156.8, 150.5, 139.7, 138.9, 138.4, 137.1, 135.8, 131.2, 131.0, 130.7, 128.6, 126.7, 123.8, 121.0, 115.8, 103.6, 70.1, 58.9, 51.5, 13.6, 13.3. LC-MS: (ESI) calcd for C₂₃H₂₃N₂O₄S [M+H]⁺ 423.14, found 423.20.

N-(10-(2-Methoxyethyl)-11-oxo-10,11-dihydrodibenzo[b,f][1,4]thiazepin-7-yl)benzo[d]oxazole-5-carboxamide. ¹H NMR (400 MHz, Chloroform-d) δ 8.35 (s, 1H), 8.29 (d, J 1.7 Hz, 1H), 8.19 (s, 1H), 8.03-7.94 (m, 3H), 7.65 (ddd, J=7.8, 5.3, 2.5 Hz, 3H), 7.50 (d, J=8.8 Hz, 1H), 7.41 (dd, J=7.4, 1.6 Hz, 1H), 7.28 (qd, J=7.1, 1.7 Hz, 2H), 4.61-4.50 (m, 1H), 3.94 (dt, J=13.8, 5.4 Hz, 1H), 3.87-3.77 (m, 1H), 3.70-3.60 (m, 1H), 3.35 (s, 3H). ¹³C NMR (101 MHz, Chloroform-d) δ 169.1, 165.3, 153.9, 152.2, 140.3, 140.1, 138.9, 138.3, 137.1, 135.9, 131.7, 131.1, 131.0, 130.7, 128.6, 126.8, 125.5, 124.2, 121.4, 119.7, 111.5, 70.2, 58.9, 51.6. LC-MS: (ESI) calcd for C₂₄H₂₀N₃O₄S [M+H]⁺ 446.12, found 446.00.

N-(10-(2-Methoxyethyl)-11-oxo-10,11-dihydrodibenzo[b,f][1,4]thiazepin-7-yl)-4-propoxybenzamide. ¹H NMR (400 MHz, Chloroform-d) δ 8.00 (s, 1H), 7.92 (d, J=2.5 Hz, 1H), 7.80 (d, J=8.8 Hz, 2H), 7.65 (dd, J=7.4, 1.8 Hz, 1H), 7.59 (dd, J=8.8, 2.5 Hz, 1H), 7.45 (d, J 8.8 Hz, 1H), 7.39 (dd, J=7.4, 1.6 Hz, 1H), 7.34-7.12 (m, 2H), 6.93 (d, J=8.8 Hz, 2H), 4.61-4.50 (m, 1H), 3.97 (t, J=6.6 Hz, 2H), 3.91 (dd, J=13.8, 5.5 Hz, 1H), 3.85-3.75 (m, 1H), 3.68-3.58 (m, 1H), 3.33 (s, 3H), 1.83 (q, J=7.6, 7.2 Hz, 2H), 1.05 (t, J=7.4 Hz, 3H). ¹³C NMR (101 MHz, Chloroform-d) δ 169.2, 165.3, 162.3, 139.7, 138.9, 138.3, 137.0, 136.1, 131.1, 131.0, 129.0, 128.6, 126.7, 126.2, 124.0, 121.2, 114.5, 70.1, 69.8, 58.9, 51.5, 22.5, 10.5. LC-MS: (ESI) calcd for C₂₆H₂₇N₂O₄S [M+H]⁺ 463.17, found 463.30.

N-(10-(2-Methoxyethyl)-11-oxo-10,11-dihydrodibenzo[b,f][1,4]thiazepin-7-yl)cyclopropanecarboxamide. ¹H NMR (400 MHz, Chloroform-d) δ 7.93 (s, 1H), 7.90 (d, J=2.5 Hz, 1H), 7.65 (dd, J=7.1, 2.1 Hz, 1H), 7.57 (dd, J=8.8, 2.5 Hz, 1H), 7.46 (d, J=8.7 Hz, 1H), 7.38 (ddd, J=13.8, 7.5, 1.8 Hz, 2H), 7.32 (d, J=1.8 Hz, 1H), 7.30-7.22 (m, 2H), 6.84 (d, J=8.1 Hz, 1H), 6.05 (s, 2H), 4.60-4.49 (m, 1H), 3.97-3.87 (m, 1H), 3.87-3.76 (m, 1H), 3.63 (dt, J 10.4, 5.4 Hz, 1H), 3.34 (s, 3H). ¹³C NMR (101 MHz, Chloroform-d) δ 169.1, 165.0, 150.9, 148.3, 139.9, 138.9, 138.3, 137.1, 135.9, 131.1, 131.0, 130.7, 128.6, 128.5, 126.7, 124.0, 121.8, 121.2, 108.2, 107.7, 101.9, 70.1, 58.9, 51.6. LC-MS: (ESI) calcd for C₂₄H₂₁N₂O₅S [M+H]⁺ 449.12, found 449.20.

2-(7-(Furan-3-carboxamido)-11-oxodibenzo[b,f][1,4]thiazepin-10(11H)-yl)ethyl pent-4-enoate. ¹H NMR (400 MHz, Chloroform-d) δ 8.07-8.02 (m, 1H), 7.85 (d, J=2.5 Hz, 1H), 7.72 (s, 1H), 7.69-7.61 (m, 2H), 7.48 (t, J=1.7 Hz, 1H), 7.40 (dd, J=7.0, 1.9 Hz, 1H), 7.36-7.22 (m, 3H), 6.72 (d, J=1.1 Hz, 1H), 5.04-4.93 (m, 2H), 4.91-4.84 (m, 1H), 4.36 (td, J=5.9, 1.9 Hz, 2H), 3.87 (dt, J=14.1, 6.1 Hz, 1H), 2.39-2.18 (m, 4H). ¹³C NMR (101 MHz, Chloroform-d) δ 172.8, 169.1, 160.7, 145.4, 144.2, 139.4, 138.8, 137.9, 137.5, 136.6, 135.8, 131.3, 131.1, 130.9, 128.8, 126.3, 124.1, 122.6, 121.3, 115.5, 108.3, 61.9, 49.8, 33.4, 28.6. LC-MS: (ESI) calcd for C₂₅H₂₃N₂O₅S [M+H]⁺ 463.13, found 463.20.

4-Methoxy-N-(11-oxo-10-(2-propoxyethyl)-10,11-dihydrodibenzo[b,f][1,4]thiazepin-7-yl)benzamide. ¹H NMR (400 MHz, Chloroform-d) δ 7.89 (d, J=2.5 Hz, 1H), 7.85-7.76 (m, 2H), 7.72 (s, 1H), 7.67 (dd, J=7.5, 1.7 Hz, 1H), 7.61 (dd, J=8.8, 2.5 Hz, 1H), 7.54 (d, J=8.8 Hz, 1H), 7.42 (dd, J=7.5, 1.5 Hz, 1H), 7.35-7.27 (m, 2H), 7.01-6.93 (m, 2H), 4.51 (dt, J=13.6, 5.8 Hz, 1H), 4.02-3.92 (m, 1H), 3.92-3.83 (m, 4H), 3.73-3.63 (m, 1H), 3.45-3.38 (m, 2H), 1.55 (q, J=7.1 Hz, 2H), 0.87 (t, J=7.4 Hz, 3H). LC-MS: (ESI) calcd for C₂₆H₂₇N₂O₄S [M+H]⁺ 463.17, found 463.30.

(E)-N-(10-(Hex-2-en-1-yl)-11-oxo-10,11-dihydrodibenzo[b,f][1,4]thiazepin-7-yl) furan-3-carboxamide. ¹H NMR (400 MHz, Chloroform-d) δ 8.06 (s, 1H), 7.98 (s, 1H), 7.80 (d, J=2.5 Hz, 1H), 7.66 (dd, J=7.4, 1.8 Hz, 1H), 7.60 (dd, J=8.7, 2.5 Hz, 1H), 7.45 (t, J=1.7 Hz, 1H), 7.36 (td, J=5.8, 5.0, 3.1 Hz, 2H), 7.26-7.19 (m, 2H), 6.75 (s, 1H), 5.64 (q, J=5.2 Hz, 2H), 4.77 (dd, J 14.9, 4.3 Hz, 1H), 4.47 (dd, J=14.7, 4.6 Hz, 1H), 2.03-1.93 (m, 2H), 1.34 (h, J=6.9 Hz, 2H), 0.83 (t, J=7.4 Hz, 3H). ¹³C NMR (101 MHz, Chloroform-d) δ 168.9, 160.8, 145.4, 144.1, 139.5, 138.8, 138.2, 136.7, 135.7, 134.5, 131.3, 131.0, 130.7, 130.6, 128.6, 126.2, 125.9, 124.9, 124.2, 124.0, 122.7, 121.3, 121.1, 108.4, 53.4, 34.3, 22.2, 13.6. LC-MS: (ESI) calcd for C₂₄H₂₃N₂O₃S [M+H]⁺ 419.15, found 419.30.

N-(10-(2-Methoxyethyl)-11-oxo-10,11-dihydrodibenzo[b,f][1,4]thiazepin-7-yl)-2-methylfuran-3-carboxamide. ¹H NMR (400 MHz, Chloroform-d) δ 7.88 (d, J=2.5 Hz, 1H), 7.65 (dd, J=7.5, 1.8 Hz, 1H), 7.59 (s, 1H), 7.52 (dd, J=8.8, 2.5 Hz, 1H), 7.44 (d, J=8.7 Hz, 1H), 7.39 (dd, J=7.0, 2.0 Hz, 1H), 7.34-7.24 (m, 2H), 6.53 (d, J=2.1 Hz, 1H), 3.91 (dt, J=13.7, 5.5 Hz, 1H), 3.85-3.75 (m, 1H), 3.69-3.58 (m, 1H), 3.33 (s, 3H), 2.62 (s, 3H). ¹³C NMR (101 MHz, Chloroform-d) & 169.1, 162.1, 158.5, 140.7, 139.8, 138.9, 138.3, 137.0, 135.8, 131.1, 131.0, 130.7, 128.6, 126.6, 124.0, 121.2, 115.4, 108.0, 70.1, 58.9, 51.5, 13.7. LC-MS: (ESI) calcd for C₂₂H₂₁N₂O₄S [M+H]⁺ 409.12, found 409.20.

N-(10-(2-Methoxypropyl)-11-oxo-10,11-dihydrodibenzo[b,f][1,4]thiazepin-7-yl) furan-3-carboxamide (report as racemic rotamers). ¹H NMR (400 MHz, Chloroform-d) δ 8.02 (dt, J=2.6, 1.2 Hz, 1H), 7.87 (dd, J=11.1, 2.4 Hz, 1H), 7.68 (ddd, J=7.3, 3.3, 1.8 Hz, 1H), 7.62-7.52 (m, 2H), 7.49 (q, J=1.3, 0.8 Hz, 1H), 7.45-7.38 (m, 2H), 7.36-7.27 (m, 2H), 6.72-6.66 (m, 1H), 4.76 (dd, J=13.5, 4.5 Hz, 0.5H), 4.40 (dd, J=13.6, 6.5 Hz, 0.5H), 3.90 (h, J=6.2 Hz, 0.5H), 3.70 (ddd, J=23.8, 12.6, 5.7 Hz, 1H), 3.49 (dd, J=13.5, 7.4 Hz, 0.5H), 3.38 (s, 1.5H), 3.23 (s, 1.5H), 1.22 (d, J=6.2 Hz, 1.5H), 1.18 (d, J=6.2 Hz, 1.5H). LC-MS: (ESI) calcd for C₂₂H₂₁N₂O₄S [M+H]⁺ 409.12, found 409.30.

N-(11-Oxo-10-(2-(pent-4-en-1-yloxy)ethyl)-10,11-dihydrodibenzo[b,f][1,4]thiazepin-7-yl) furan-3-carboxamide. ¹H NMR (600 MHz, Chloroform-d) δ 8.04 (s, 1H), 7.84 (d, J=2.5 Hz, 1H), 7.71 (s, 1H), 7.65 (dd, J=7.4, 1.9 Hz, 1H), 7.59 (dd, J=8.7, 2.5 Hz, 1H), 7.50 (d, J=8.8 Hz, 1H), 7.47 (t, J=1.7 Hz, 1H), 7.39 (dd, J=7.4, 1.6 Hz, 1H), 7.33-7.21 (m, 2H), 6.71 (dd, J 1.9, 0.9 Hz, 1H), 5.82-5.73 (m, 1H), 5.00-4.86 (m, 2H), 4.57-4.49 (m, 1H), 3.95 (dt, J=13.8, 5.5 Hz, 1H), 3.85 (ddd, J=10.1, 6.6, 5.3 Hz, 1H), 3.71-3.63 (m, 1H), 3.48-3.39 (m, 2H), 2.08-2.01 (m, 2H), 1.61 (dt, J=13.7, 6.7 Hz, 2H). ¹³C NMR (151 MHz, Chloroform-d) δ 169.1, 160.7, 145.4, 144.2, 140.1, 138.8, 138.3, 138.3, 137.0, 135.5, 131.1, 131.0, 130.7, 128.6, 126.8, 124.0, 122.7, 121.2, 114.7, 108.3, 70.6, 68.3, 51.8, 30.3, 28.9. LC-MS: (ESI) calcd for C₂₅H₂₅N₂O₄S [M+H]⁺ 449.15, found 449.10.

3,4-Difluoro-N-(10-(2-methoxyethyl)-11-oxo-10, 11-dihydrodibenzo[b,f][1,4]thiazepin-7-yl)benzamide. ¹H NMR (600 MHz, Chloroform-d) δ 8.24 (s, 1H), 7.91 (d, J=2.5 Hz, 1H), 7.76-7.69 (m, 1H), 7.66-7.56 (m, 3H), 7.49 (d, J=8.7 Hz, 1H), 7.39 (dd, J=6.9, 2.2 Hz, 1H), 7.30-7.19 (m, 3H), 4.53 (ddd, J=13.9, 6.7, 5.3 Hz, 1H), 3.94 (dt, J=13.9, 5.4 Hz, 1H), 3.81 (ddd, J 10.1, 6.7, 5.2 Hz, 1H), 3.67-3.60 (m, 1H), 3.34 (s, 3H). ¹³C NMR (151 MHz, Chloroform-d) δ 169.2, 163.6, 153.7, 151.9, 151.1, 149.5, 140.2, 138.8, 138.2, 137.1, 135.6, 131.4, 131.0 (d, J=6.9 Hz), 130.8, 128.7, 126.8, 124.3, 123.7 (d, J=3.6 Hz), 121.5, 117.8, 117.6, 117.1, 117.0, 70.1, 58.9, 51.7. LC-MS: (ESI) calcd for C₂₃H₁₉F₂N₂O₃S [M+H]⁺ 441.11, found 441.20.

4-Chloro-3-fluoro-N-(10-(2-methoxyethyl)-11-oxo-10,11-dihydrodibenzo[b,f][1,4]thiazepin-7-yl)benzamide. ¹H NMR (600 MHz, Chloroform-d) δ 8.19 (s, 1H), 7.93 (dd, J=6.8, 2.3 Hz, 1H), 7.90 (d, J=2.5 Hz, 1H), 7.74 (ddd, J=8.6, 4.4, 2.3 Hz, 1H), 7.65-7.56 (m, 2H), 7.49 (d, J=8.7 Hz, 1H), 7.39 (dd, J=6.9, 2.2 Hz, 1H), 7.28-7.23 (m, 2H), 7.21 (t, J=8.5 Hz, 1H), 4.53 (ddd, J=13.9, 6.7, 5.4 Hz, 1H), 3.94 (dt, J=13.9, 5.4 Hz, 1H), 3.81 (ddd, J=10.1, 6.7, 5.2 Hz, 1H), 3.69-3.61 (m, 1H), 3.34 (s, 3H). ¹³C NMR (151 MHz, Chloroform-d) δ 169.2, 163.6, 161.2, 159.5, 140.3, 138.8, 138.2, 137.1, 135.5, 131.6 (d, J=3.6 Hz), 131.1, 131.0, 130.8, 130.1, 128.7, 127.4 (d, J=8.2 Hz), 126.8, 124.3, 121.4, 117.1, 116.9, 70.1, 58.9, 51.7. LC-MS: (ESI) calcd for C₂₃H₁₉FClN₂O₃S [M+H]⁺ 457.08, found 457.10.

N-(10-(2-Cyanoethyl)-11-oxo-10,11-dihydrodibenzo[b,f][1,4]thiazepin-7-yl) furan-3-carboxamide. ¹H NMR (400 MHz, Chloroform-d) δ 8.10-8.01 (m, 1H), 7.85 (d, J=2.5 Hz, 1H), 7.72-7.63 (m, 2H), 7.50 (dd, J=4.1, 2.3 Hz, 2H), 7.42 (td, J=7.3, 1.7 Hz, 1H), 7.35-7.28 (m, 3H), 7.10 (d, J=8.6 Hz, 1H), 5.01-4.78 (m, 1H), 3.91-3.70 (m, 1H), 2.96-2.86 (m, 2H) . . . . LC-MS: (ESI) calcd for C₂₁H₁₆N₃O₃S [M+H]⁺ 390.08, found 390.20.

4-Chloro-3-methoxy-N-(10-(2-methoxyethyl)-11-oxo-10,11-dihydrodibenzo[b,f][1,4]thiazepin-7-yl)benzamide. ¹H NMR (600 MHz, Chloroform-d) δ 8.36 (s, 1H), 7.93 (d, J=2.5 Hz, 1H), 7.89 (d, J=2.3 Hz, 1H), 7.77 (dd, J=8.6, 2.3 Hz, 1H), 7.66-7.57 (m, 2H), 7.46 (d, J=8.7 Hz, 1H), 7.38 (dd, J=7.4, 1.5 Hz, 1H), 7.26-7.21 (m, 2H), 6.94 (d, J 8.7 Hz, 1H), 4.58-4.50 (m, 1H), 3.95 (s, 3H), 3.92 (dt, J=13.8, 5.5 Hz, 1H), 3.84-3.77 (m, 1H), 3.67-3.60 (m, 1H), 3.34 (s, 3H). ¹³C NMR (151 MHz, Chloroform-d) δ 169.2, 157.9, 139.8, 138.9, 138.2, 137.0, 136.0, 131.1, 131.0, 130.7, 129.3, 128.6, 127.5, 127.4, 126.7, 124.2, 122.8, 121.4, 111.5, 70.1, 58.9, 56.4, 51.6. LC-MS: (ESI) calcd for C₂₄H₂₂ClN₂O₄S [M+H]: 469.10, found 469.30.

4-Cyano-3-methoxy-N-(10-(2-methoxyethyl)-11-oxo-10,11-dihydrodibenzo[b,f][1,4]thiazepin-7-yl)benzamide. ¹H NMR (600 MHz, Chloroform-d) δ 8.66 (s, 1H), 8.17-8.11 (m, 2H), 7.97 (d, J=2.5 Hz, 1H), 7.60 (dd, J=8.8, 2.5 Hz, 2H), 7.47 (d, J=8.8 Hz, 1H), 7.39-7.33 (m, 1H), 7.27-7.19 (m, 2H), 7.03 (d, J=8.9 Hz, 1H), 4.57-4.50 (m, 1H), 4.00 (s, 3H), 3.93 (dt, J=13.9, 5.4 Hz, 1H), 3.80 (dt, J=10.5, 6.0 Hz, 1H), 3.63 (dt, J=10.5, 5.4 Hz, 1H), 3.33 (s, 3H). ¹³C NMR (151 MHz, Chloroform-d) δ 169.3, 163.5, 139.9, 138.8, 138.1, 137.0, 135.9, 134.5, 132.9, 131.0, 131.0, 130.8, 128.6, 127.2, 126.7, 124.4, 121.6, 115.8, 111.5, 101.7, 70.1, 58.9, 56.6, 51.6. LC-MS: (ESI) calcd for C₂₅H₂₂N₃O₄S [M+H]⁺ 460.13, found 460.30.

3,5-Difluoro-4-methoxy-N-(10-(2-methoxyethyl)-11-oxo-10,11-dihydrodibenzo[b,f][1,4]thiazepin-7-yl)benzamide. ¹H NMR (600 MHz, Chloroform-d) δ 8.25 (s, 1H), 7.90 (d, J=2.5 Hz, 1H), 7.62 (dd, J=6.7, 2.5 Hz, 1H), 7.58 (dd, J=8.8, 2.6 Hz, 1H), 7.48 (d, J=8.7 Hz, 1H), 7.44 (d, J=8.1 Hz, 2H), 7.38 (dd, J=7.3, 1.8 Hz, 1H), 7.26-7.19 (m, 2H), 4.52 (ddd, J=13.9, 6.7, 5.3 Hz, 1H), 4.09 (s, 3H), 3.94 (dt, J=13.9, 5.4 Hz, 1H), 3.84-3.78 (m, 1H), 3.64 (dt, J=10.5, 5.5 Hz, 1H), 3.34 (s, 3H). ¹³C NMR (151 MHz, Chloroform-d) δ 169.3, 163.2, 155.8, 154.1, 140.2, 138.8, 138.1, 137.1, 135.5, 131.0, 130.8, 128.6, 126.8, 124.3, 121.5, 111.7 (d, J=6.1 Hz), 111.6 (d, J=6.0 Hz), 70.1, 58.9, 51.7. LC-MS: (ESI) calcd for C₂₄H₂₁F₂N₂O₄S [M+H]⁺ 471.12, found 471.30.

4-formamido-N-(10-(2-methoxyethyl)-11-oxo-10,11-dihydrodibenzo[b,f][1,4]thiazepin-7-yl)benzamide. ¹H NMR (600 MHz, Chloroform-d) δ 8.44 (d, J=1.6 Hz, 1H), 7.93 (dd, J=4.8, 2.6 Hz, 1H), 7.89-7.79 (m, 2H), 7.71-7.65 (m, 2H), 7.61 (ddd, J=7.3, 5.0, 2.5 Hz, 1H), 7.51 (dd, J=9.0, 5.7 Hz, 1H), 7.26 (s, 3H), 7.19-7.14 (m, 1H), 4.56 (ddd, J=13.6, 6.6, 5.3 Hz, 1H), 3.94 (dtd, J=13.9, 5.5, 3.5 Hz, 1H), 3.83 (tdd, J=6.7, 5.1, 3.2 Hz, 1H), 3.66 (dtd, J=10.4, 5.5, 3.0 Hz, 1H), 3.35 (s, 3H). LC-MS: (ESI) calcd for C₂₄H₂₂N₃O₄S [M+H]⁺ 448.13, found 448.30.

N-(10-(2-(Cyanomethoxy)ethyl)-11-oxo-10,11-dihydrodibenzo[b,f][1,4]thiazepin-7-yl) furan-3-carboxamide. ¹H NMR (600 MHz, Chloroform-d) δ 8.05 (d, J=9.3 Hz, 1H), 8.02 (s, 1H), 7.72 (d, J=8.7 Hz, 1H), 7.55 (s, 1H), 7.47 (s, 1H), 7.43 (s, 1H), 7.37-7.30 (m, 1H), 7.21-7.14 (m, 1H), 6.82 (d, J=8.3 Hz, 1H), 6.78 (d, J=7.6 Hz, 1H), 6.70 (s, 1H), 5.02 (s, 2H), 3.72 (t, J 5.3 Hz, 2H), 3.32 (t, J=5.3 Hz, 2H). ¹³C NMR (151 MHz, Chloroform-d) δ 167.8, 164.5, 145.1, 144.0, 142.6, 133.8, 132.0, 130.6, 126.2, 125.9, 124.8, 124.1, 114.5, 113.5, 111.3, 108.2, 60.9, 48.9, 45.8. LC-MS: (ESI) calcd for C₂₂H₁₇N₃O₄SNa [M+Na]⁺ 442.08, found 442.10.

N-(10-(2-Hydroxyethyl)-11-oxo-10,11-dihydrodibenzo[b,f][1,4]thiazepin-7-yl) furan-3-carboxamide. ¹H NMR (400 MHz, Chloroform-d) δ 8.50 (d, J=2.6 Hz, 1H), 8.19 (dd, J=8.9, 2.6 Hz, 1H), 7.76-7.69 (m, 1H), 7.62 (d, J=8.9 Hz, 1H), 7.54-7.46 (m, 1H), 7.43-7.32 (m, 2H), 4.65-4.55 (m, 1H), 4.16-3.74 (m, 3H). LC-MS: (ESI) calcd for C₂₀H₁₇N₂O₄S [M+Na]⁺ 381.08, found 381.10.

N-(10-(1-Methoxypropan-2-yl)-11-oxo-10,11-dihydrodibenzo[b,f][1,4]thiazepin-7-yl) furan-3-carboxamide. ¹H NMR (400 MHz, Chloroform-d) δ 8.02 (dt, J=2.6, 1.2 Hz, 1H), 7.87 (dd, J=11.1, 2.4 Hz, 1H), 7.68 (ddd, J=7.3, 3.3, 1.8 Hz, 1H), 7.62-7.52 (m, 2H), 7.49 (q, J=1.3, 0.8 Hz, 1H), 7.45-7.38 (m, 2H), 7.36-7.27 (m, 2H), 6.72-6.66 (m, 1H), 4.76 (dd, J=13.5, 4.5 Hz, 0.5H), 4.40 (dd, J=13.6, 6.5 Hz, 0.5H), 3.90 (h, J=6.2 Hz, 0.5H), 3.70 (ddd, J=23.8, 12.6, 5.7 Hz, 1H), 3.49 (dd, J=13.5, 7.4 Hz, 0.5H), 3.38 (s, 1.5H), 3.23 (s, 1.5H), 1.22 (d, J=6.2 Hz, 1.5H), 1.18 (d, J=6.2 Hz, 1.5H). LC-MS: (ESI) calcd for C₂₂H₂₁N₂O₄S [M+H]⁺ 409.12, found 409.20.

N-(10-Allyl-11-oxo-10,11-dihydrodibenzo[b,f][1,4]thiazepin-7-yl) furan-3-carboxamide. 1H NMR (600 MHz, Chloroform-d) δ 8.08-8.03 (m, 2H), 7.81 (d, J=2.5 Hz, 1H), 7.65 (dd, J=7.6, 1.7 Hz, 1H), 7.59 (dd, J=8.8, 2.5 Hz, 1H), 7.45 (t, J=1.8 Hz, 1H), 7.40-7.31 (m, 2H), 7.29-7.21 (m, 2H), 6.75 (s, 1H), 6.05-5.95 (m, 1H), 5.30 (dd, J=17.2, 1.5 Hz, 1H), 5.20 (dd, J=10.3, 1.4 Hz, 1H), 4.80 (dd, J=15.6, 5.4 Hz, 1H), 4.56 (dd, J=15.6, 5.8 Hz, 1H). ¹³C NMR (151 MHz, Chloroform-d) δ 168.9, 160.9, 145.5, 144.1, 139.4, 138.7, 138.0, 136.5, 135.8, 133.2, 131.3, 131.0, 130.9, 128.7, 125.6, 124.2, 122.7, 121.3, 117.5, 108.4, 54.0. LC-MS: (ESI) calcd for C₂₁H₁₇N₂O₃S [M+H]⁺ 377.10, found 377.10.

(E)-N-(10-(5-Hydroxypent-2-en-1-yl)-11-oxo-10,11-dihydrodibenzo[b,f][1,4]thiazepin-7-yl) furan-3-carboxamide. ¹H NMR (400 MHz, Chloroform-d) δ 9.73 (t, J=1.7 Hz, 1H), 8.06-8.01 (m, 1H), 7.84 (d, J=2.6 Hz, 1H), 7.69 (dd, J=7.5, 1.8 Hz, 1H), 7.66-7.57 (m, 1H), 7.53-7.47 (m, 2H), 7.46-7.38 (m, 2H), 7.36-7.28 (m, 1H), 6.69 (dd, J=2.0, 0.9 Hz, 1H). 5.83-5.65 (m, 1H), 5.58-5.47 (m, 1H), 4.85 (d, J=5.2 Hz, 1H), 4.48 (dd, J=15.1, 6.0 Hz, 1H), 2.42-2.34 (m, 4H). LC-MS: (ESI) calcd for C₂₃H₂₁N₂O₄S [M+H]⁺ 421.11, found 421.30.

(E)-6-(7-(Furan-3-carboxamido)-11-oxodibenzo[b,f][1,4]thiazepin-10(11H)-yl) hex-4-enoic acid. ¹H NMR (400 MHz, Methanol-d₄) δ 8.23 (dd, J=1.6, 0.9 Hz, 1H), 8.04 (d, J=2.5 Hz, 1H), 7.73-7.60 (m, 3H), 7.58-7.45 (m, 2H), 7.45-7.34 (m, 2H), 6.94 (dd, J=2.0, 0.9 Hz, 1H), 5.83-5.65 (m, 1H), 5.58-5.47 (m, 1H), 4.85 (d, J=5.2 Hz, 1H), 4.48 (dd, J=15.1, 6.0 Hz, 1H), 2.42-2.34 (m, 4H). LC-MS: (ESI) calcd for C₂₄H₂₁N₂O₅S [M+H]: 449.11, found 449.30.

4-Methoxy-N-(11-oxo-10-(2-(prop-2-yn-1-yloxy)ethyl)-10,11-dihydrodibenzo[b,f][1,4]oxazepin-7-yl)benzamide. ¹H NMR (600 MHz, Chloroform-d) δ 7.91 (s, 1H), 7.90-7.84 (m, 2H), 7.78 (dd, J=7.9, 1.7 Hz, 1H), 7.74 (s, 1H), 7.21 (ddd, J=8.7, 7.1, 1.8 Hz, 1H), 7.11 (d, J=1.3 Hz, 2H), 7.02-6.96 (m, 2H), 6.88 (td, J=8.0, 7.5, 1.1 Hz, 1H), 6.48 (d, J 8.5 Hz, 1H), 4.63 (d, J=2.4 Hz, 2H), 4.61-4.56 (m, 2H), 3.95-3.90 (m, 2H), 3.88 (s, 3H), 2.47 (t, J=2.4 Hz, 1H). ¹³C NMR (151 MHz, Chloroform-d) & 172.0, 165.3, 162.7, 153.3, 146.4, 137.2, 133.6, 133.4, 131.9, 129.0, 127.8, 126.7, 120.5, 120.0, 119.7, 114.1, 113.8, 107.1, 77.9, 75.8, 65.5, 56.3, 55.4, 54.5. LC-MS: (ESI) calcd for C₂₆H₂₃N₂O₅ [M+H]⁺ 443.16, found 443.20.

N-(5-(2-Methoxyethyl)-6-oxo-6,11-dihydro-5H-dibenzo[b,e]azepin-2-yl) furan-3-carboxamide. ¹H NMR (600 MHz, Chloroform-d) δ 8.03 (s, 1H), 7.76 (dd, J=7.7, 1.5 Hz, 1H), 7.65 (d, J=2.1 Hz, 1H), 7.59 (s, 1H), 7.48 (t, J=1.8 Hz, 1H), 7.35-7.28 (m, 3H), 7.23 (t, J=6.9 Hz, 1H), 7.13 (d, J=7.0 Hz, 1H), 6.71 (s, 1H), 4.72 (ddd, J=14.1, 7.0, 4.8 Hz, 1H), 4.23 (d, J 13.1 Hz, 1H), 3.94-3.87 (m, 1H), 3.68 (qt, J=10.3, 5.3 Hz, 2H), 3.49 (d, J=13.2 Hz, 1H), 3.31 (s, 3H). ¹³C NMR (151 MHz, Chloroform-d) δ 168.9, 160.6, 145.2, 144.1, 142.5, 139.7, 136.4, 135.4, 132.9, 131.5, 130.6, 126.9, 126.0, 124.6, 122.8, 118.8, 118.7, 108.2, 69.9, 58.6, 49.7, 38.7. LC-MS: (ESI) calcd for C₂₂H₂₁N₂O₄ [M+H]⁺ 377.15, found 377.20.

N-(11-Oxo-10-(2-(prop-2-yn-1-yloxy)ethyl)-10,11-dihydrodibenzo[b,f][1,4]thiazepin-7-yl)benzo[d]thiazole-5-carboxamide. ¹H NMR (400 MHz, Chloroform-d) δ 9.08 (s, 1H), 8.58 (s, 1H), 8.51 (s, 1H), 7.98 (dd, J=17.5, 7.0 Hz, 3H), 7.65 (d, J=7.7 Hz, 2H), 7.48 (d, J=8.7 Hz, 1H), 7.38 (d, J=8.7 Hz, 1H), 7.26 (d, J=6.1 Hz, 2H), 4.62-4.51 (m, 1H), 4.16 (s, 2H), 4.03-3.92 (m, 2H), 3.81 (q, J=6.5, 5.0 Hz, 1H), 2.42 (d, J=2.3 Hz, 1H). ¹³C NMR (151 MHz, Chloroform-d) & 169.2, 165.44, 155.8, 153.0, 140.0, 138.9, 138.2, 137.4, 137.1, 136.0, 133.0, 131.1, 131.0, 130.8, 128.6, 126.7, 124.6, 124.2, 122.5, 122.1, 121.4, 79.5, 74.7, 67.6, 58.4, 51.6, 38.6. LC-MS: (ESI) calcd for C₂₆H₂₀N₃O₃S₂ [M+H]⁺ 486.10, found 486.10.

N-(10-(2-Methoxyethyl)-11-oxo-10,11-dihydrodibenzo[b,f][1,4]thiazepin-7-yl) isoxazole-4-carboxamide. ¹H NMR (400 MHz, Chloroform-d) δ 9.07 (s, 1H) 7.73 (d, J=10 Hz, 1H), 7.78 (d, J 2.0 Hz, 1H), 7.58-7.60 (m, 2H), 7.48 (d, J=8.8 Hz, 1H), 7.33-7.35 (m, 1H), 7.19-7.26 (m, 2H), 4.52-4.57 (m, 1H), 3.92-3.96 (m, 1H), 3.75-3.78 (m, 1H), 3.60-3.65 (m, 1H), 3.34 (s, 3H). 13C NMR (151 MHz, Chloroform-d) δ 169.6, 159.4, 158.0, 148.0, 139.9, 138.8, 138.0, 137.1, 135.4, 131.1, 130.9, 128.7, 126.8, 124.3, 121.5, 118.0, 70.1, 58.9, 51.8. LC-MS: (ESI) calcd for C₂₀H₁₈N₃O₄S [M+H]⁺ 396.10, found 396.10.

N-(10-(2-Methoxyethyl)-11-oxo-10,11-dihydrodibenzo[b,f][1,4]thiazepin-7-yl) thiazole-4-carboxamide. ¹H NMR (400 MHz, Chloroform-d) δ 9.25 (s, 1H) 8.80 (d, J=1.6 Hz, 1H), 8.28 (d, J=2.0 Hz, 1H), 8.00 (d, J=2.4 Hz, 1H), 7.67-7.70 (m, 2H), 7.50 (d, J=8.8 Hz, 1H), 7.42-7.44 (m, 1H), 7.27-7.31 (m, 2H), 4.54-4.57 (m, 1H), 3.92-3.96 (m, 1H), 3.81-3.84 (m, 1H), 3.64-3.66 (m, 1H), 3.35 (s, 3H). ¹³C NMR (151 MHz, Chloroform-d) δ 169.1, 158.7, 152.9, 150.7, 140.0, 138.8, 138.4, 137.2, 135.5, 131.2, 131.0, 130.7, 128.6, 126.8, 124.4, 123.6, 120.7, 70.2, 58.9, 51.6. LC-MS: (ESI) calcd for C₂₀H₁₈N₃O₃S₂ [M+H]⁺ 412.08, found 412.00.

N-(10-(2-Methoxyethyl)-11-oxo-10,11-dihydrodibenzo[b,f][1,4]thiazepin-7-yl)benzo[d]thiazole-5-carboxamide. ¹H NMR (400 MHz, Chloroform-d) δ 9.11 (s, 1H), 8.58 (s, 1H), 8.26 (s, 1H), 8.06 (d, J=8.4 Hz, 1H), 7.98 (d, J=9.2 Hz, 2H), 7.63-7.67 (m, 2H), 7.51 (d, J 4.8 Hz, 1H), 7.40-7.42 (m, 1H), 7.26-7.29 (m, 2H), 4.52-4.57 (m, 1H), 3.92-3.96 (m, 1H), 3.80-3.85 (m, 1H), 3.64-3.67 (m, 1H), 3.35 (s, 3H). ¹³C NMR (151 MHz, Chloroform-d) δ 169.1, 165.3, 155.9, 153.0, 140.2, 138.8, 138.3, 137.2, 135.7, 133.0, 131.1, 131.0, 130.7, 128.7, 126.8, 124.5, 124.1, 122.6, 121.9, 121.3, 70.2, 58.9, 51.6. LC-MS: (ESI) calcd for C₂₄H₂₀N₃O₃S₂ [M+H]⁺ 462.10, found 462.10.

N-(10-(2-Methoxyethyl)-11-oxo-10,11-dihydrodibenzo[b,f][1,4]thiazepin-7-yl) imidazo[1,2-a]pyridine-6-carboxamide. ¹H NMR (400 MHz, Chloroform-d): δ 8.82 (s, 1H), 8.23 (s, 1H), 7.94 (s, 1H), 7.73 (s, 1H), 7.61-7.68 (m, 4H), 7.48-7.53 (m, 2H), 7.46-7.47 (d, J=1.6 Hz, 1H), 7.23-7.40 (m, 2H), 4.50-4.57 (m, 1H), 3.92-3.98 (m, 1H), 3.80-3.85 (m, 1H), 3.35-3.68 (m, 1H), 3.25 (s, 3H). ¹³C NMR (151 MHz, Chloroform-d) & 169.2, 163.2, 145.0, 140.3, 138.7, 138.2, 137.1, 135.7, 134.6, 131.1, 131.0, 130.8, 128.7, 128.4, 126.8, 124.4, 122.4, 121.6, 120.7, 117.0, 113.9, 70.2, 58.9, 51.7. LC-MS: (ESI) calcd for C₂₄H₂₁N₄O₃S [M+H]⁺ 445.14, found 445.10.

N-(10-(2-Methoxyethyl)-11-oxo-10,11-dihydrodibenzo[b,f][1,4]thiazepin-7-yl) imidazo[1,2-a]pyridine-6-carboxamide. ¹H NMR (400 MHz, Chloroform-d): δ 9.62 (s, 1H), 8.34 (s, 1H), 8.18-8.20 (d, J=8.0 Hz, 1H), 8.11 (s, 1H), 7.73-7.74 (d, J=4.0 Hz, 1H), 7.65-7.72 (m, 3H), 7.49-7.52 (d, J=12.0 Hz, 1H), 7.41-7.42 (m, 1H), 7.25-7.40 (m, 4H), 4.53-4.57 (m, 1H), 3.93-3.97 (m, 1H), 3.82-3.84 (m, 1H), 3.64-3.67 (m, 1H), 3.35 (s, 3H). ¹³C NMR (151 MHz, Chloroform-d) δ 169.1, 164.3, 143.8, 140.1, 138.9, 138.3, 137.1, 136.4, 135.0, 131.3, 131.2, 131.0, 130.7, 128.6, 126.7, 126.0, 124.2, 121.3, 116.4, 113.7, 111.8, 70.2, 58.9, 51.6. LC-MS: (ESI) calcd for C₂₄H₂₁N₄O₃S [M+H]⁺ 445.14, found 445.10.

N-(10-(2-Methoxyethyl)-11-oxo-10,11-dihydrodibenzo[b,f][1,4]thiazepin-7-yl)pyrimidine-2-carboxamide. ¹H NMR (400 MHz, Chloroform-d) δ 9.95 (s, 1H), 8.94 (d, J=4.8 Hz, 2H), 7.98 (d, J=2.4 Hz, 1H), 7.85 (dd, J=2.8, 2.4 Hz, 1H), 7.69 (dd, J=2.0, 1.4 Hz, 1H), 7.50-7.55 (m, 2H), 7.44 (dd, J=1.2, 1.6 Hz, 1H), 7.26-7.33 (m, 2H), 4.54-4.57 (m, 1H), 3.94-3.97 (m, 1H), 3.81-3.84 (m, 1H), 3.64-3.67 (m, 1H), 3.35 (s, 3H). ¹³C NMR (151 MHz, Chloroform-d) δ 169.0, 159.7, 157.7, 157.2, 140.4, 138.8, 138.4, 137.2, 135.1, 131.2, 131.0, 130.7, 128.7, 127.0, 123.7, 122.9, 120.9, 70.2, 58.9, 51.6. LC-MS: (ESI) calcd for C₂₁H₁₉N₄O₃S [M+H]⁺ 407.12, found 407.10.

N-(10-(2-Methoxyethyl)-11-oxo-10,11-dihydrodibenzo[b,f][1,4]thiazepin-7-yl)pyrimidine-4-carboxamide. ¹H NMR (400 MHz, Chloroform-d) δ 9.87 (s, 1H), 9.30 (d, J=0.8 Hz, 1H), 9.05 (d, J=3.2 Hz, 1H), 8.20 (dd, J=1.2, 1.2 Hz, 1H), 8.05 (d, J=2.8 Hz, 1H), 7.75 (d, J=2.4 Hz, 1H), 7.73 (d, J=2.4 Hz, 1H), 7.70 (d, J=2.0 Hz, 1H), 7.55 (d, J=8.8 Hz, 1H), 7.26-7.45 (m, 2H), 4.53-4.56 (m, 1H), 3.94-3.98 (m, 1H), 3.82-3.85 (m, 1H), 3.65-3.69 (m, 1H), 3.35 (s, 3H). ¹³C NMR (151 MHz, Chloroform-d) & 169.0, 160.3, 159.8, 157.7, 155.7, 140.7, 138.7, 138.3, 137.3, 134.7, 131.2, 131.0, 130.8, 128.7, 127.0, 123.8, 120.9, 118.6, 70.2, 58.9, 51.7. LC-MS: (ESI) calcd for C₂₁H₁₉N₄O₃S [M+H]⁺ 407.12, found 407.10.

N-(10-(2-Methoxyethyl)-11-oxo-10,11-dihydrodibenzo[b,f][1,4]thiazepin-7-yl) pyrazine-2-carboxamide. ¹H NMR (400 MHz, Chloroform-d) δ 9.67 (s, 1H), 9.50 (s, 1H), 8.82 (d, J=2.0 Hz, 1H), 8.59 (s, 1H), 8.05 (d, J=2.4 Hz, 1H), 7.67-7.75 (m, 2H), 7.54 (d, J=8.8 Hz, 1H), 7.44 (dd, J 1.2, 1.2 Hz, 1H), 7.27-7.34 (m, 2H), 4.52-4.58 (m, 1H), 3.93-3.99 (m, 1H), 3.81-3.86 (m, 1H), 3.64-3.69 (m, 1H), 3.35 (s, 3H). ¹³C NMR (151 MHz, Chloroform-d) δ 169.1, 160.7, 147.8, 144.7, 143.9, 142.4, 140.4, 138.8, 138.3, 137.3, 135.0, 131.2, 131.0, 130.8, 128.7, 126.9, 123.7, 120.8, 70.2, 58.9, 51.7. LC-MS: (ESI) calcd for C₂₁H₁₉N₄O₃S [M+H]⁺ 407.12, found 407.10.

3-Fluoro-4-formamido-N-(10-(2-methoxyethyl)-11-oxo-10,11-dihydrodibenzo[b,f][1,4]thiazepin-7-yl)benzamide. ¹H NMR (400 MHz, Chloroform-d) δ 8.47-8.52 (m, 2H), 8.13 (s, 1H), 7.92 (d, J=2.8 Hz, 1H), 7.58-7.75 (m, 5H), 7.49 (d, J=8.8 Hz, 1H), 7.40 (dd, J=1.2, 1.4 Hz, 1H), 7.26-7.31 (m, 2H), 4.53-4.56 (m, 1H), 3.92-3.95 (m, 1H), 3.80-3.88 (m, 1H), 3.64-3.66 (m, 1H), 3.35 (s, 3H). ¹³C NMR (151 MHz, Chloroform-d) δ 169.1, 158.9, 152.6, 151.0, 140.1, 138.8, 138.2, 137.0, 131.1 (d, J=15.2 Hz), 130.8, 128.7, 126.8, 124.2, 123.0, 121.4, 114.8 (d, J=21.0 Hz), 70.2, 58.9, 51.6. LC-MS: (ESI) calcd for C₂₄H₂₁FN₃O₄S [M+H]⁺ 466.13, found 466.10.

3-Fluoro-N-(10-(2-methoxyethyl)-11-oxo-10,11-dihydrodibenzo[b,f][1,4]thiazepin-7-yl)-4-(trifluoromethoxy)benzamide. ¹H NMR (400 MHz, Chloroform-d): δ7.91 (d, J=2.0 Hz, 1H), 7.87 (br, 1H), 7.72-7.75 (m, 1H), 7.62-7.68 (m, 2H), 7.57-7.60 (m, 1H), 7.51-7.54 (m, 1H), 7.41-7.44 (m, 2H), 7.26-7.32 (m, 2H), 4.49-4.56 (m, 1H), 3.92-3.98 (m, 1H), 3.80-3.86 (m, 1H), 3.62-3.68 (m, 1H), 3.35 (s, 3H). ¹³C NMR (151 MHz, Chloroform-d) δ 169.1, 163.3, 155.2, 153.5, 140.6, 138.7, 138.2, 137.2, 135.2, 134.7, 131.1, 131.0, 130.8, 128.7, 126.9, 124.3, 123.7, 123.2, 121.4, 116.9, 116.7, 70.2, 58.9, 51.7. LC-MS: (ESI) calcd for C₂₄H₁₉F₄N₂O₄S [M−H]⁺ 507.10, found 505.20.

N-(4-Fluoro-10-(2-methoxyethyl)-11-oxo-10,11-dihydrodibenzo[b,f][1,4]thiazepin-7-yl) furan-3-carboxamide. ¹H NMR (400 MHz, MeOD): δ 8.21 (s, 1H), 8.03 (d, J=2.4 Hz, 1H), 7.75 (dd, J=2.4, 2.4 Hz, 1H), 7.59-7.61 (m, 2H), 7.35-7.43 (m, 2H), 7.20-7.24 (m, 1H), 6.91 (d, J=1.2 Hz, 1H), 4.54-4.60 (m, 1H), 3.92-3.97 (m, 1H), 3.69-3.72 (m, 1H), 3.63-3.67 (m, 1H), 3.33 (s, 3H). ¹³C NMR (151 MHz, Chloroform-d) δ 160.7, 158.5, 145.4, 144.3, 140.5, 140.3, 136.0, 135.6, 129.9 (d, J=8.1 Hz), 127.0, 126.5, 124.4, 122.6, 121.7, 117.5, 117.4, 108.2, 70.1, 58.9, 51.6. LC-MS: (ESI) calcd for C₂₁H₁₈FN₂O₄S [M+H]⁺ 413.10, found 413.10.

N-(10-(2-Methoxyethyl)-11-oxo-10,11-dihydrodibenzo[b,f][1,4]thiazepin-7-yl) isoxazole-5-carboxamide. ¹H NMR (400 MHz, Chloroform-d): δ 8.39 (s, 1H), 8.28 (m, 1H), 7.95 (s, 1H), 7.68 (d, J=7.6 Hz, 1H), 7.62 (d, J=8.8 Hz, 1H), 7.54 (d, J=8.8 Hz, 1H), 7.43 (d, J=7.2 Hz, 1H), 7.28-7.33 (m, 2H), 7.04 (s, 1H), 4.48-4.55 (m, 1H), 3.93-3.99 (m, 1H), 3.81-3.86 (m, 1H), 3.63-3.68 (m, 1H), 3.35 (s, 3H). ¹³C NMR (151 MHz, Chloroform-d) δ 169.0, 162.3, 153.3, 151.4, 141.1, 138.6, 138.3, 137.3, 134.2, 131.2, 131.0, 130.8, 128.7, 127.0, 124.1, 121.2, 107.5, 70.2, 58.9, 51.8. LC-MS: (ESI) calcd for C₂₀H₁₈N₃O₄S [M−H]⁺ 394.08, found 394.20.

N-(10-(2-Methoxyethyl)-11-oxo-10,11-dihydrodibenzo[b,f][1,4]thiazepin-7-yl)pyrimidine-5-carboxamide. ¹H NMR (400 MHz, Chloroform-d) δ 9.36 (s, 1H), 9.19 (s, 2H), 8.39 (s, 1H), 7.90-7.91 (d, J=4.0 Hz, 1H), 7.60-7.62 (m, 2H), 7.52-7.54 (d, J=8.0 Hz, 1H), 7.38-7.41 (m, 1H), 7.25-7.27 (m, 2H), 4.47-4.54 (m, 1H), 3.93-3.99 (m, 1H), 3.78-3.84 (m, 1H), 3.62-3.67 (m, 1H), 3.34 (s, 3H). ¹³C NMR (151 MHz, Chloroform-d) δ 169.5, 162.1, 160.7, 155.9, 140.3, 138.8, 138.0, 137.1, 135.3, 131.1, 130.9 (d, J=5.4 Hz), 128.7, 128.1, 126.8, 124.5, 121.7, 70.1, 58.9, 51.8. LC-MS: (ESI) calcd for C₂₁H₁₉N₄O₃S [M+H]⁺ 407.12, found 407.10.

3-Chloro-4-formamido-N-(10-(2-methoxyethyl)-11-oxo-10,11-dihydrodibenzo[b,f][1,4]thiazepin-7-yl)benzamide. ¹H NMR (400 MHz, Chloroform-d): δ 8.58 (s, 1H), 7.88-7.96 (m, 4H), 7.61-7.76 (m, 3H), 7.42-7.43 (d, J=4.0 Hz, 1H), 7.40-7.41 (d, J=4.0 Hz, 1H), 7.25-7.30 (m, 2H), 4.50-4.57 (m, 1H), 3.85-3.97 (m, 1H), 3.79-3.84 (m, 1H), 3.62-3.67 (m, 1H), 3.34 (s, 3H). ¹³C NMR (151 MHz, Chloroform-d) δ 169.2, 163.7, 159.0, 140.2, 138.8, 138.2, 137.1, 136.7, 135.7, 131.1, 131.0, 130.8 (d, J=11.3 Hz), 128.8, 128.7, 126.8, 126.2, 124.2, 122.9, 121.4, 121.2, 70.2, 58.9, 51.6. LC-MS: (ESI) calcd for C₂₄H₂₁ClN₃O₄S [M+H]⁺ 482.10, found 482.10.

4-(Difluoromethoxy)-3-fluoro-N-(10-(2-methoxyethyl)-11-oxo-10,11-dihydrodibenzo[b,f][1,4]thiazepin-7-yl)benzamide. ¹H NMR (400 MHz, Chloroform-d): δ7.91-7.92 (d, J=4.0 Hz, 2H), 7.60-7.72 (m, 4H), 7.50-7.52 (d, J=8.0 Hz, 1H), 7.26-7.42 (m, 3H), 6.45-6.81 (m, 1H), 4.50-4.57 (m, 1H), 3.85-3.97 (m, 1H), 3.79-3.84 (m, 1H), 3.62-3.67 (m, 1H), 3.34 (s, 3H). ¹³C NMR (151 MHz, Chloroform-d) δ 169.2, 163.6, 154.6, 152.9, 140.3, 138.8, 138.2, 137.1, 135.5, 133.0 (d, J=5.9 Hz), 131.0 (d, J=4.1 Hz), 130.8, 128.7, 126.8, 124.3, 123.4 (d, J=3.7 Hz), 122.6, 121.5, 117.0, 116.6, 116.5, 115.2, 113.4, 70.1, 58.9, 51.7, 29.7. LC-MS: (ESI) calcd for C₂₄H₂₀F₃N₂O₄S [M+H]⁺ 489.11, found 489.10.

3-Fluoro-N-(10-(2-methoxyethyl)-11-oxo-10,11-dihydrodibenzo[b,f][1,4]thiazepin-7-yl)-4-(methylsulfonyl)benzamide. 1HNMR (400 MHz, Chloroform-d): δ9.04 (s, 1H), 7.99 (d, J=2.4 Hz, 1H), 7.87 (d, J=6.8 Hz, 1H), 7.71-7.74 (m, 2H), 7.65 (dd, J=2.0, 2.4 Hz, 1H), 7.57-7.59 (m, 1H), 7.50 (d, J=4.8 Hz, 1H), 7.37-7.39 (m, 1H), 7.22-7.25 (m, 2H), 4.53-4.55 (m, 1H), 3.91-3.96 (m, 1H), 3.76-3.80 (m, 1H), 3.62-3.66 (m, 1H), 3.35 (s, 3H), 325 (s, 3H). ¹³C NMR (151 MHz, Chloroform-d) & 169.4, 163.1, 160.1, 158.4, 142.5, 140.3, 138.8, 138.1, 137.1, 135.5, 131.1, 131.0, 130.9, 130.6, 130.5, 130.1, 128.7, 126.8, 124.4, 123.3, 121.6, 116.8, 116.7, 70.1, 58.9, 51.7, 43.9. LC-MS: (ESI) calcd for C₂₄H₂₂FN₂O₅S₂ [M+H]⁺ 501.10, found 501.10.

3-Fluoro-N-(10-(2-methoxyethyl)-11-oxo-10,11-dihydrodibenzo[b,f][1,4]thiazepin-7-yl)-4-(methylsulfinyl)benzamide. ¹H NMR (400 MHz, Chloroform-d): δ8.91 (s, 1H), 8.00 (d, J=2.4 Hz, 1H), 7.75-7.82 (m, 2H), 7.62-7.69 (m, 3H), 7.49-7.51 (m, 1H), 7.38-7.41 (m, 1H), 7.27-7.30 (m, 1H), 7.22-7.25 (m, 1H), 4.50-4.57 (m, 1H), 3.91-3.97 (m, 1H), 3.78-3.84 (m, 1H), 3.62-3.67 (m, 1H), 3.34 (s, 3H), 2.87 (s, 3H). ¹³C NMR (151 MHz, Chloroform-d) δ 169.2, 163.8, 158.2, 156.5, 140.2, 139.9 (d, J=6.5 Hz), 138.8, 138.2, 137.0, 136.1, 136.0, 135.8, 131.1 (d, J=7.5 Hz), 130.8, 128.7, 126.8, 125.9, 124.3, 123.7, 121.5, 115.7, 115.5, 70.1, 58.9, 51.7, 41.9. LC-MS: (ESI) calcd for C₂₄H₂₂FN₂O₄S₂ [M+H]⁺ 485.10, found 485.10.

N-(2-Fluoro-10-(2-methoxyethyl)-11-oxo-10,11-dihydrodibenzo[b,f][1,4]thiazepin-7-yl) furan-3-carboxamide. ¹H NMR (400 MHz, Chloroform-d): δ 8.04 (s, 1H), 7.98 (s, 1H), 7.88 (d, J=2.4 Hz, 1H), 7.55-7.58 (m, 1H), 7.43-7.46 (m, 2H), 7.31-7.35 (m, 2H), 6.90-6.95 (m, 1H), 6.73 (s, 1H), 4.47-4.54 (m, 1H), 3.88-3.95 (m, 1H), 3.74-3.80 (m, 1H), 3.58-3.64 (m, 1H), 3.32 (s, 3H). ¹³C NMR (151 MHz, Chloroform-d) δ 168.0, 163.4, 161.8, 160.8, 145.4, 144.2, 140.1, 139.6, 137.0, 135.8, 134.3 (d, J=3.4 Hz), 132.9 (d, J=7.8 Hz), 126.9, 124.0, 122.6, 121.4, 118.2, 108.4, 70.0, 58.9, 51.5. LC-MS: (ESI) calcd for C₂₁H₁₈FN₂O₄S [M+H]⁺ 413.10, found 413.10.

N-(3-Fluoro-10-(2-methoxyethyl)-11-oxo-10,11-dihydrodibenzo[b,f][1,4]thiazepin-7-yl) furan-3-carboxamide. ¹H NMR (400 MHz, Chloroform-d): δ 8.04 (s, 1H), 7.87 (s, 1H), 7.86 (s, 1H), 7.59-7.74 (m, 2H), 7.49-7.48 (m, 2H), 7.11 (dd, J=2.4, 2.4 Hz, 1H), 6.96-7.00 (m, H), 6.72 (d, J=0.8 Hz, 1H) 4.53-4.56 (m, 1H), 3.98-4.04 (m, 1H), 3.77-3.80 (m, 1H), 3.62-3.66 (m, 1H), 3.33 (s, 3H). ¹³C NMR (151 MHz, Chloroform-d) δ 168.3, 163.8, 162.1, 160.8, 145.4 (d, J=6.6 Hz), 144.2 (d, J=4.4 Hz), 140.9 (d, J=8.3 Hz), 139.8, 136.2, 135.8, 134.4, 126.8, 124.1, 122.6, 118.0, 117.9, 116.1, 115.9, 108.3, 108.3, 70.1, 58.9, 51.6. LC-MS: (ESI) calcd for C₂₁H₁₈FN₂O₄S [M+H]⁺ 413.10, found 413.10.

(E)-10-(Hex-2-en-1-yl)-7-(pyridin-2-ylamino)dibenzo[b,f][1,4]thiazepin-11 (10H)-one. 1H NMR (400 MHz, Chloroform-d): δ 8.21 (s, 1H), 7.71 (d, J=7.2 Hz, 1H), 7.61 (s, 1H), 7.53 (t, J=7.2 Hz, 1H), 7.41 (d, J=7.2 Hz, 1H), 7.26-7.33 (m, 3H), 6.73-6.80 (m, 3H), 5.60-5.72 (m, 2H), 4.78 (d, J=13.6 Hz, 1H), 4.44 (d, J=13.6 Hz, 1H), 1.98-2.02 (m, 2H), 1.31-1.40 (m, 2H), 0.84 (t, J=7.2 Hz, 3H). ¹³C NMR (151 MHz, Chloroform-d) δ 168.7, 154.9, 147.9, 138.9, 138.5, 138.4, 138.0, 137.7, 137.0, 131.0, 130.6, 128.6, 126.2, 122.9 (d, J=16.8 Hz), 120.2, 115.7 (d, J=12.4 Hz), 109.4 (d, J 11.4 Hz), 53.3, 34.3, 22.3, 13.6. LC-MS: (ESI) calcd for C₂₄H₂₄N₃OS [M+H]⁺ 402.17, found 402.20.

4-Methoxy-N-(10-(2-methoxyethyl)-11-oxo-10,11-dihydrodibenzo[b,f][1,4]thiazepin-7-yl)-2-methylbenzamide. ¹H NMR (400 MHz, Chloroform-d) δ 7.90 (d, J=2.4 Hz, 1H), 7.66 (dd, J 2.0, 1.2 Hz, 1H), 7.53-7.56 (m, 2H), 7.46 (d, J=8.8 Hz, 1H), 7.40-7.43 (m, 2H), 7.27-7.32 (m, 2H), 6.73-6.77 (m, 2H), 4.52-4.58 (m, 1H), 3.87-3.94 (m, 1H), 3.78-3.83 (m, 4H), 3.61-3.66 (m, 1H), 3.34 (s, 3H), 2.48 (s, 3H). ¹³C NMR (151 MHz, Chloroform-d) δ 169.07, 167.66, 161.17, 139.82, 139.46, 138.87, 138.31, 137.14, 136.07, 131.06, 130.69, 128.62, 128.52, 128.03, 126.73, 123.64, 120.83, 116.91 (d, J=13.7 Hz), 111.04 (d, J=13.5 Hz), 70.15, 58.89, 55.31, 20.41. LC-MS: (ESI) calcd for C₂₅H₂₅N₂O₄S [M+H]⁺ 449.16, found 449.20.

2-Fluoro-4-methoxy-N-(10-(2-methoxyethyl)-11-oxo-10,11-dihydrodibenzo[b,f][1,4]thiazepin-7-yl)benzamide. ¹H NMR (400 MHz, Chloroform-d) δ 8.37 (d, J=16.8 Hz, 1H), 8.10 (t, J=9.2, Hz, 1H), 7.94 (d, J=2.4 Hz, 1H), 7.67 (dd, J=2.0, 1.6 Hz, 1H), 7.61 (dd, J=2.4, 2.4 Hz, 1H), 7.48 (d, J=8.8 Hz, 1H), 7.43 (dd, J=1.2, 1.6 Hz, 1H), 7.25-7.33 (m, 2H), 6.84 (dd, J=2.4, 2.4 Hz, 1H), 6.67 (dd, J=0.64, 0.2 Hz, 1H), 4.53-4.59 (m, 1H), 3.90-3.96 (m, 1H), 3.80-3.87 (m, 4H), 3.61-3.67 (m, 1H), 3.34 (s, 3H). ¹³C NMR (151 MHz, Chloroform-d) δ 169.05, 164.14, 162.30, 161.22, 160.67, 140.03, 138.85, 138.36, 137.06, 135.72, 133.49, 131.04, 130.69, 128.63, 126.72, 124.28, 121.54, 113.00 (d, J=11.4 Hz), 111.15 (d, J=11.5 Hz), 101.69, 70.15, 58.86, 55.89, 51.26. LC-MS: (ESI) calcd for C₂₄H₂₂FN₂O₄S [M+H]⁺ 453.13, found 453.10.

3-((10-(2-Methoxyethyl)-11-oxo-10,11-dihydrodibenzo[b,f][1,4]thiazepin-7-yl) carbamoyl)benzoic acid. ¹H NMR (400 MHz, DMSO-d6) δ 10.58 (s, 1H), 8.52 (s, 1H), 8.13-8.17 (m, 3H), 7.79 (dd, J=2.4, 2.4 Hz, 1H), 7.64-7.69 (m, 2H), 7.59-7.61 (m, 1H), 7.53 (dd, J 2.4, 4.0 Hz, 1H), 7.39-7.41 (m, 2H), 4.51-4.57 (m, 1H), 3.84-3.89 (m, 1H), 3.56-3.61 (m, 1H), 3.50-3.57 (m, 1H), 3.21 (s, 3H). ¹³C NMR (151 MHz, DMSO-d6) δ 167.1, 166.2, 164.3, 137.9, 137.5, 136.3, 135.1, 134.2, 131.8, 131.4, 130.6, 130.5-130.2 (m), 128.3 (d, J=9.1 Hz), 127.8, 126.1, 123.1, 120.9, 68.7, 57.5, 49.3. LC-MS: (ESI) calcd for C₂₄H₂₁N₂O₅S [M+H]⁺ 449.12, found 449.10.

N-(10-(2-Methoxyethyl)-11-oxo-10,11-dihydrodibenzo[b,f][1,4]thiazepin-7-yl) isophthalamide. ¹H NMR (400 MHz, Methanol-d4) δ 8.43 (s, 1H), 8.06-8.09 (m, 3H), 7.71-7.73 (m, H), 7.58-7.62 (m, 3H), 7.48 (dd, J=2.0, 4.0 Hz, 1H), 7.36 (d, J=4.0, Hz, 2H), 4.56-4.61 (m, 1H), 3.94-3.98 (m, 1H), 3.71-3.75 (m, 1H), 3.63-3.66 (m, 1H), 3.31 (s, 3H). ¹³C NMR (151 MHz, Methanol-d4) δ 169.9, 166.6, 139.3, 139.0, 138.1, 136.9, 136.8, 135.0, 134.2, 130.8, 130.8, 130.6, 130.5, 130.4, 128.6, 128.5, 126.6, 126.5, 124.2, 121.7, 69.7, 57.7, 50.9. LC-MS: (ESI) calcd for C₂₄H₂₂N₃O₄S [M+H]⁺ 448.14, found 448.10.

N1-(10-(2-Methoxyethyl)-11-oxo-10,11-dihydrodibenzo[b,f][1,4]thiazepin-7-yl)-N4-methylterephthalamide. ¹H NMR (400 MHz, Chloroform-d) δ 9.02 (s, 1H), 8.17 (s, 1H), 7.99 (d, J=2.4, Hz, 1H), 7.92 (d, J=7.6, Hz, 1H), 7.78 (d, J=2.0, Hz, 1H), 7.62-7.66 (m, 2H), 7.36-7.45 (m, 3H), 7.23-7.26 (m, 2H), 6.80 (d, J=4.4, Hz, 1H), 4.50-4.55 (m, 1H), 3.87-3.93 (m, 1H), 3.76-3.81 (m, 1H), 3.62-3.66 (m, 1H), 3.33 (s, 3H), 2.92 (d, J=4.8, Hz, 3H). ¹³C NMR (151 MHz, Chloroform-d) & 169.3, 167.9, 165.4, 139.9, 138.9, 138.1, 137.0, 136.0, 134.5, 131.1 (d, J=5.1 Hz), 130.8, 130.7, 130.1, 129.2, 128.7, 126.7, 125.4, 124.2, 121.4, 70.1, 58.9, 51.6, 27.0. LC-MS: (ESI) calcd for C₂₅H₂₄N₃O₄S [M+H]⁺ 462.15, found 462.20.

4-((10-(2-Methoxyethyl)-11-oxo-10,11-dihydrodibenzo[b,f][1,4]thiazepin-7-yl) carbamoyl)benzoic acid. ¹H NMR (400 MHz, DMSO-d6) δ10.55 (s, 1H), 8.17 (d, J=2.4, Hz, 1H), 8.06 (d, J=7.6, Hz, 2H), 8.01 (d, J=8.8, Hz, 2H), 7.77 (dd, J=2.4, 2.4 Hz, 2H), 7.65 (d, J 8.8, Hz, 2H), 7.59-7.61 (m, 1H), 7.52-7.54 (m, 1H), 7.39-7.41 (m, 2H), 4.52-4.54 (m, 1H), 3.84-3.87 (m, 1H), 3.50-3.54 (m, 1H), 3.56-3.60 (m, 1H), 3.21 (s, 3H). ¹³C NMR (151 MHz, DMSO-d6) δ 167.1, 166.3, 164.5, 137.9, 137.5, 137.2, 136.3, 135.1, 130.3, 128.7, 128.3, 127.2, 126.1, 123.0, 120.9, 68.7, 57.5, 49.3. LC-MS: (ESI) calcd for C₂₄H₂₀N₂O₅S [M+H]⁺ 449.12, found 449.10.

N-(10-(2-Methoxyethyl)-11-oxo-10,11-dihydrodibenzo[b,f][1,4]thiazepin-7-yl) terephthalamide. ¹H NMR (400 MHz, DMSO-d6) δ10.51 (s, 1H), 8.16 (d, J=16.4, Hz, 2H), 8.00 (s, 4H), 7.79 (d, J=8.8, Hz, 1H), 7.51-7.66 (m, 4H), 7.41 (d, J=3.6, Hz, 2H), 4.53-4.56 (m, 1H), 3.83-3.88 (m, 1H), 3 . . . 50-3.60 (m, 2H), 3.21 (s, 3H). ¹³C NMR (151 MHz, DMSO-d6) δ 167.1, 166.5, 164.4, 137.8, 137.5, 136.4, 136.3, 136.1, 135.1, 130.3 (d, J=6.6 Hz), 128.3, 127.0, 126.9, 126.1, 123.0, 120.9, 68.7, 57.5, 49.3. LC-MS: (ESI) calcd for C₂₄H₂₂N₃O₄S [M+H]⁺ 448.14, found 448.20.

N1-(10-(2-Methoxyethyl)-11-oxo-10,11-dihydrodibenzo[b,f][1,4]thiazepin-7-yl)-N4-methylterephthalamide. ¹H NMR (400 MHz, DMSO-d6) δ10.5 (s, 1H), 8.61 (d, J=4.8, Hz, 1H), 8.17 (d, J=2.4, Hz, 1H), 7.97 (dd, J=8.4, 8.4 Hz, 4H), 7.97 (dd, J=2.0, 2.0 Hz, 1H), 7.59-7.66 (m, 2H), 7.52-7.54 (m, 1H), 7.40 (t, J=4.4, Hz, 2H), 4.52-4.56 (m, 1H), 3.84-3.87 (m, 1H), 3 . . . 50-3.60 (m, 2H), 3.21 (s, 3H), 2.82 (t, J=4.4, Hz, 3H). ¹³C NMR (151 MHz, DMSO-d6) δ 167.1, 165.2, 164.4, 137.9, 137.5, 136.6, 136.3, 136.0, 135.1, 130.4, 128.3, 127.1, 126.5, 126.1, 120.9, 68.7, 57.5, 49.3, 25.7. LC-MS: (ESI) calcd for C₂₅H₂₄N₃O₄S [M+H]⁺ 462.15, found 462.20.

N1-(10-(2-Methoxyethyl)-11-oxo-10,11-dihydrodibenzo[b,f][1,4]thiazepin-7-yl)-N4,N4-dimethylterephthalamide. ¹H NMR (400 MHz, Chloroform-d) δ 9.04 (s, 1H), 8.08 (d, J=2.4, Hz, 1H), 7.77-7.82 (m, 3H), 7.68 (dd, J=1.6, 1.6 Hz, 4H), 7.49 (d, J=8.8, Hz, 1H), 7.42 (dd, J=1.2, 1.6 Hz, 1H), 7.25-7.31 (m, 4H), 4.54-4.59 (m, 1H), 3.90-3.95 (m, 1H), 3.79-3.85 (m, 1H), 3.64-3.68 (m, 1H), 3.35 (s, 3H), 3.16 (s, 3H), 2.94 (s, 3H). ¹³C NMR (151 MHz, Chloroform-d) δ 171.0, 169.1, 165.6, 139.7, 139.0, 138.6, 138.4, 136.8, 136.5, 135.9, 131.2, 131.0, 130.7, 128.6, 127.7, 126.8, 126.5, 124.3, 121.4, 70.1, 58.9, 51.5, 39.5, 35.6. LC-MS: (ESI) calcd for C₂₆H₂₆N₃O₄S [M+H]⁺ 476.17, found 476.20.

3-(1-Cyanoethyl)-N-(10-(2-methoxyethyl)-11-oxo-10,11-dihydrodibenzo[b,f][1,4]thiazepin-7-yl)benzamide. ¹H NMR (400 MHz, Chloroform-d): δ 8.00 (s, 1H), 7.93 (s, 1H), 7.84 (s, 1H), 7.76-7.78 (d, J=8.0 Hz, 1H), 7.61-7.67 (m, 2H), 7.50-7.51 (d, J 4.0 Hz, 1H), 7.42-7.48 (m, 2H), 7.39-7.40 (d, J=4.0 Hz, 1H), 7.26-7.31 (m, 2H), 4.52-4.56 (m, 1H), 3.91-3.98 (m, 2H), 3.71-3.83 (m, 1H), 3.62-3.68 (m, 1H), 3.34 (s, 3H), 1.67-1.68 (d, J=4.0 Hz, 3H). ¹³C NMR (151 MHz, Chloroform-d) δ 169.1, 165.1, 140.2, 138.8, 138.2, 138.0, 137.1, 135.6, 135.5, 131.1, 131.0, 130.8, 130.3, 129.7, 128.7, 126.8, 126.6, 125.8, 124.2, 121.4, 121.1, 70.13, 58.9, 51.6, 31.2, 21.3. LC-MS: (ESI) calcd for C₂₆H₂₄N₃O₃S [M+H]⁺ 458.16, found 458.20.

3-(Cyanomethyl)-N-(10-(2-methoxyethyl)-11-oxo-10,11-dihydrodibenzo[b,f][1,4]thiazepin-7-yl)benzamide. ¹H NMR (400 MHz, Chloroform-d): δ 7.92 (s, 2H), 7.77-7.92 (m, 2H), 7.61-7.67 (m, 2H), 7.49-7.53 (m, 3H), 7.40-7.43 (m, 1H), 7.26-7.30 (m, 2H), 4.51-4.57 (m, 1H), 3.91-3.97 (m, 1H), 3.81-3.85 (m, 1H), 3.79 (s, 2H), 3.62-3.67 (m, 1H), 3.32 (s, 3H). ¹³C NMR (151 MHz, Chloroform-d) δ 169.1, 165.0, 140.2, 138.8, 138.2, 137.1, 135.6, 135.5, 131.5, 131.1, 131.0, 130.9, 130.8, 129.7, 128.7, 126.9, 126.8, 126.7, 124.2, 121.4, 117.3, 70.1, 58.9, 51.6, 23.5. LC-MS: (ESI) calcd for C₂₅H₂₂N₃O₃S [M+H]⁺ 444.14, found 444.10.

tert-butyl-4-(Aminomethyl)-N-(10-(2-methoxyethyl)-11-oxo-10,11-dihydrodibenzo[b,f][1,4]thiazepin-7-yl)benzamide hydrochloride. ¹H NMR (400 MHz, d4-MeOD) δ8.50 (br, 1H), 8.08 (d, J=2.4 Hz, 1H), 8.01 (d, J=8.0 Hz, 2H), 7.71-7.74 (m, 1H), 7.59-7.63 (m, 4H), 7.48-7.51 (m, 1H), 7.34-7.41 (m, 2H), 4.55-4.62 (m, 1H), 4.20 (s, 2H), 3.93-4.00 (m, 1H), 3.71-3.76 (m, 1H), 3.62-3.68 (m, 1H), 3.32 (s, 3H). ¹³C NMR (151 MHz, Methanol-d4) δ 169.9, 166.5, 139.4, 138.9, 138.1, 137.3, 136.8 (d, J=2.8 Hz), 135.1, 130.8, 130.8, 130.4, 128.7, 128.5, 128.1, 126.5, 124.3, 121.8, 69.7, 57.6, 50.9, 42.5. LC-MS: (ESI) calcd for C₂₄H₂₄N₃O₃S [M+H]⁺ 434.16, found 434.20.

(E)-4-((10-(2-Methoxyethyl)-11-oxo-10,11-dihydrodibenzo[b,f][1,4]thiazepin-7-yl)amino)-4-oxobut-2-enoic acid. ¹H NMR (400 MHz, Methanol-d4) δ 8.03 (d, J=2.0, Hz, 1H), 7.56-7.62 (m, 3H), 7.47 (s, 1H), 7.35-7.37 (m, 2H), 7.10 (d, J=15.6, Hz, 1H), 6.81 (d, J=15.6, Hz, 1H), 4.53-4.60 (m, 1H), 3.90-3.97 (m, 1H), 3.68-3.72 (m, 1H), 3.62-3.65 (m, 1H), 3.61 (s, 3H). ¹³C NMR (151 MHz, Methanol-d4) δ 169.9, 162.9, 139.4, 138.8, 138.0, 137.0, 136.6, 136.0, 131.7, 130.9, 130.8, 130.4, 128.6, 126.6, 123.3, 120.9, 69.7, 57.6, 50.9. LC-MS: (ESI) calcd for C₂₀H₁₉N₂O₅S [M+H]⁺ 399.10, found 399.10.

2-Cyano-N-(10-(2-methoxyethyl)-11-oxo-10,11-dihydrodibenzo[b,f][1,4]thiazepin-7-yl) acetamide. ¹H NMR (400 MHz, Chloroform-d): δ 8.19 (s, 1H), 7.75-7.77 (d, J=8.0 Hz, 1H), 7.63-7.64 (d, J=4.0 Hz, 1H), 7.41-7.42 (d, J=4.0 Hz, 2H), 7.29-7.30 (d, J=4.0 Hz, 1H), 7.25-7.29 (m, 2H), 4.48-4.54 (m, 1H), 3.90-3.96 (m, 1H), 3.76-3.81 (m, 1H), 3.53-3.66 (m, 1H), 3.33 (s, 3H). ¹³C NMR (151 MHz, Chloroform-d) δ 169.5, 159.5, 140.3, 138.7, 138.0, 137.2, 134.9, 131.2, 131.0, 131.0, 128.8, 126.8, 124.2, 121.4, 114.3, 70.0, 58.9, 51.7, 26.8. LC-MS: (ESI) calcd for C₁₉H₁₈N₃O₃S [M+H]⁺ 468.11, found 368.10.

2-((10-(2-Methoxyethyl)-11-oxo-10,11-dihydrodibenzo[b,f][1,4]thiazepin-7-yl)amino)-2-oxoacetic acid. ¹H NMR (400 MHz, Chloroform-d) δ 8.96 (s, 1H), 7.91 (s, 1H), 7.67-7.69 (m, 1H), 7.54-7.56 (m, 2H), 7.42-7.44 (d, J=8.0 Hz, 1H), 7.29-7.34 (m, 2H), 4.48-4.54 (m, 1H), 3.94-3.99 (m, 1H), 3.82-3.87 (m, 1H), 3.66-3.71 (m, 1H), 3.35 (s, 3H). ¹³C NMR (151 MHz, Methanol-d4) δ 169.8, 139.9, 138.8, 138.0, 136.9, 135.6, 130.9, 130.8, 130.4, 128.6, 126.6, 123.9, 121.4, 69.7, 57.6, 50.9. LC-MS: (ESI) calcd for C₁₈H₁₇N₂O₅S [M+H]⁺ 373.09, found 373.10.

(E)-10-(Hex-2-en-1-yl)-7-(isoquinolin-1-ylamino)dibenzo[b,f][1,4]thiazepin-11 (10H)-one. ¹H NMR (400 MHz, Chloroform-d): δ7.86 (d, J=4.0 Hz, 2H), 7.80 (t, J=8.4 Hz, 1H), 7.72 (d, J=7.6 Hz, 1H), 7.67 (t, J=6.8 Hz, 1H), 7.42-7.44 (m, 2H), 7.28-7.38 (m, 5H), 7.21-7.24 (m, 1H), 5.63-5.75 (m, 2H), 4.72-4.76 (m, 1H), 4.52-4.57 (m, 1H), 2.01-2.09 (m, 2H), 1.35-1.44 (m, 2H), 0.88 (t, J=7.6 Hz, 3H). ¹³C NMR (151 MHz, Chloroform-d) δ 168.8, 162.5, 162.2, 151.9, 142.3, 138.9, 138.0, 137.9, 137.4, 135.6, 134.9, 134.6, 131.4, 131.1, 131.1, 128.9, 128.5, 128.1, 128.0, 127.1, 127.0, 126.6, 124.8, 124.2, 118.7, 116.8, 114.8, 53.7, 34.3, 22.2, 13.6. LC-MS: (ESI) calcd for C₂₈H₂₆N₃OS [M+H]⁺ 452.18, found 452.30.

2-Cyano-4-methoxy-N-(10-(2-methoxyethyl)-11-oxo-10,11-dihydrodibenzo[b,f][1,4]thiazepin-7-yl)benzamide. ¹H NMR (400 MHz, Chloroform-d): δ 10.22 (s, 1H), 7.92 (s, 1H), 7.74-7.80 (m, 3H), 7.62-7.64 (m, 1H), 7.51-7.54 (m, 2H), 7.41-7.43 (m, 2H), 7.26-7.29 (m, 1H), 4.52-4.57 (m, 1H), 3.89-3.95 (m, 1H), 3.92 (s, 3H), 3.60-3.64 (m, 1H), 3.54-3.58 (m, 1H), 3.21 (s, 3H). ¹³C NMR (151 MHz, Chloroform-d) & 168.9, 166.4, 164.7, 156.6, 144.6, 138.2, 137.6, 132.1, 131.3, 131.1, 129.0, 127.7, 126.6, 125.7, 125.3, 120.0, 118.7, 106.4, 106.2, 70.2, 59.0, 56.1, 52.3. LC-MS: (ESI) calcd for C₂₅H₂₂N₃O₄S [M+H]⁺ 460.14, found 460.20.

N1-(10-(2-Methoxyethyl)-11-oxo-10,11-dihydrodibenzo[b,f][1,4]thiazepin-7-yl)-N4,N4-dimethylterephthalamide. ¹H NMR (400 MHz, Chloroform-d) δ9.24 (s, 1H), 8.07 (d, J=2.4, Hz, 1H), 7.82 (d, J=7.6, Hz, 1H), 7.79 (s, 1H), 7.71 (dd, J=2.4, 2.4 Hz, 1H), 7.66 (dd, J=1.6, 1.6 Hz, 1H), 7.45 (dd, J=6.4, 7.2 Hz, 1H), 7.34-7.38 (m, 3H), 7.25-7.29 (m, 2H), 4.53-4.58 (m, 1H), 3.90-3.94 (m, 1H), 3.78-3.84 (m, 1H), 3.63-3.67 (m, 1H), 3.35 (s, 3H), 3.13 (s, 3H), 2.96 (s, 3H). ¹³C NMR (151 MHz, Chloroform-d) δ 171.2, 169.2, 165.4, 139.8, 139.0, 138.3, 137.0, 136.3, 135.6, 135.1, 131.1, 131.1, 130.7, 129.9, 129.2, 129.0, 128.6, 126.6, 125.7, 124.1, 121.3, 70.1, 58.9, 51.5, 39.7, 35.6. LC-MS: (ESI) calcd for C₂₆H₂₆N₃O₄S [M+H]⁺ 476.17, found 476.20.

N-(10-(2-Methoxyethyl)-11-oxo-10,11-dihydrodibenzo[b,f][1,4]thiazepin-7-yl)-4-sulfamoylbenzamide. 1HNMR (400 MHz, DMSO-d6) δ 10.55 (s, 1H), 8.16 (d, J=2.4 Hz, 1H), 8.08 (d, J=8.4, Hz, 2H), 7.96 (d, J=8.1 Hz, 2H), 7.76 (d, J=2.4, Hz, 1H), 7.66 (d, J=8.8, Hz, 1H), 7.59 (d, J=4.8 Hz, 1H), 7.52 (t, J=4.0, Hz, 3H), 7.39-7.42 (m, 2H), 4.53-4.56 (m, 1H), 3.83-3.89 (m, 1H), 3.49-3.61 (m, 2H), 3.21 (s, 3H). ¹³C NMR (151 MHz, DMSO-d6) δ 167.1, 164.1, 146.1, 138.0, 137.5, 136.8, 136.1, 135.1, 130.4, 130.3, 128.3, 127.8, 126.1, 125.1, 123.0, 120.9, 68.7, 57.5, 49.3. LC-MS: (ESI) calcd for C₂₃H₂₂N₃O₅S₂ [M+H]⁺ 484.10, found 484.10.

4-(Difluoromethoxy)-N-(10-(2-methoxyethyl)-11-oxo-10,11-dihydrodibenzo[b,f][1,4]thiazepin-7-yl)benzamide. ¹H NMR (400 MHz, Chloroform-d): δ 8.02 (s, 1H), 7.87 (s, 1H), 7.65-7.66 (d, J=4.0 Hz, 2H), 7.60-7.61 (d, J=4.0 Hz, 1H), 7.58-7.59 (d, J 4.0 Hz, 1H), 7.47-7.48 (d, J=4.0 Hz, 1H), 7.41-7.42 (d, J=4.0 Hz, 1H), 7.20-7.30 (m, 2H), 7.18-7.20 (d, J=8.0 Hz, 1H), 6.40-6.77 (m, 1H), 4.50-4.57 (m, 1H), 3.91-3.96 (m, 1H), 3.82-3.90 (m, 1H), 3.62-3.80 (m, 1H), 3.34 (s, 3H). ¹³C NMR (151 MHz, Chloroform-d) δ 169.2, 164.7, 153.9, 140.0, 138.8, 138.2, 137.1, 135.8, 131.3, 131.1, 131.0, 130.8, 129.1, 128.6, 126.8, 124.2, 121.4, 119.2, 117.1, 115.3, 113.6, 70.1, 58.9, 51.6. LC-MS: (ESI) calcd for C₂₄H₂₁F₂N₂O₄S [M+H]⁺ 471.12, found 471.10.

4-(Formamidomethyl)-N-(10-(2-methoxyethyl)-11-oxo-10,11-dihydrodibenzo[b,f][1,4]thiazepin-7-yl)benzamide. ¹H NMR (600 MHz, Chloroform-d) δ 8.32 (s, 1H), 8.00 (s, 1H), 7.95 (d, J=2.5 Hz, 1H), 7.80 (d, J=8.3 Hz, 2H), 7.67 (dd, J=7.6, 1.6 Hz, 1H), 7.62 (dt, J=8.7, 2.3 Hz, 1H), 7.50 (d, J=8.7 Hz, 1H), 7.41 (dd, J=7.6, 1.4 Hz, 1H), 7.38 (d, J 8.1 Hz, 2H), 7.29 (ddd, J=15.1, 7.5, 5.9 Hz, 2H), 4.58-4.48 (m, 3H), 3.93 (dt, J=13.8, 5.5 Hz, 1H), 3.86-3.79 (m, 1H), 3.68-3.61 (m, 1H), 3.35 (s, 3H). ¹³C NMR (151 MHz, Chloroform-d) δ 169.1, 161.1, 161.0, 142.0, 140.1, 138.8, 138.3, 135.7, 133.8, 131.2, 131.0, 130.7, 128.7, 128.0, 127.5, 126.8, 124.1, 121.2, 70.2, 58.9, 51.6, 41.7. LC-MS: (ESI) calcd for C₂₅H₂₄N₃O₄S [M+H]⁺ 462.15, found 462.10.

4-(Acetamidomethyl)-N-(10-(2-methoxyethyl)-11-oxo-10,11-dihydrodibenzo[b,f][1,4]thiazepin-7-yl)benzamide. ¹H NMR (400 MHz, Chloroform-d) δ8.69 (s, 1H), 7.99 (s, 1H), 7.74 (d, J=8.0 Hz, 2H), 7.62-7.67 (m, 2H), 7.44 (d, J=8.8 Hz, 1H), 7.37 (d, J=7.2 Hz, 1H), 7.21-7.26 (m, 4H), 6.39 (br, 1H), 4.50-4.56 (m, 1H), 4.40 (d, J=4.2 Hz, 2H), 3.86-3.93 (m, 1H), 3.76-3.81 (m, 1H), 3.59-3.65 (m, 1H), 3.32 (s, 3H), 2.03 (s, 3H). ¹³C NMR (151 MHz, Chloroform-d) δ 170.3, 169.1, 165.6, 142.6, 140.0, 138.8, 138.3, 137.0, 136.0, 133.6, 131.2, 131.0, 130.7, 128.6, 127.8, 127.5, 126.7, 124.1, 121.3, 70.1, 58.9, 51.5, 43.2, 29.7, 23.3. LC-MS: (ESI) calcd for C₂₆H₂₆N₃O₄S [M+H]⁺ 476.17, found 476.20.

6-Ethynyl-N-(10-(2-methoxyethyl)-11-oxo-10,11-dihydrodibenzo[b,f][1,4]thiazepin-7-yl) nicotinamide. ¹H NMR (400 MHz, DMSO-d6) δ 9.05 (s, 1H), 8.28-8.30 (d, J=8.0 Hz, 2H), 8.14 (s, 1H), 7.73-7.76 (m, 2H), 7.60-7.61 (d, J=4.0 Hz, 1H), 7.58-7.59 (m, 1H). 7.51-7.53 (m, 1H), 7.39-7.41 (m, 1H), 4.57 (s, 1H), 4.50-4.55 (m, 1H), 3.82-3.88 (m, 1H), 3.50-3.52 (m, 1H), 3.53-3.61 (m, 1H), 3.20 (s, 3H). ¹³C NMR (151 MHz, DMSO-d6) δ 167.1, 162.8, 148.6, 143.5, 138.1, 137.5, 136.0, 135.5, 135.1, 130.4, 130.3, 129.0, 128.3, 126.4, 126.2, 123.0, 120.9, 82.0, 68.7, 57.5, 49.3. LC-MS: (ESI) calcd for C₂₄H₂₀N₃O₃S [M+H]: 430.12, found 430.10.

2-Ethynyl-N-(10-(2-methoxyethyl)-11-oxo-10,11-dihydrodibenzo[b,f][1,4]thiazepin-7-yl) isonicotinamide. ¹H NMR (400 MHz, Chloroform-d) δ 8.68-8.69 (d, J=4.0 Hz, 1H), 8.52 (s, 1H), 7.92-7.93 (d, J=4.0 Hz, 1H), 7.85 (s, 1H), 7.60-7.65 (m, 3H), 7.50-7.52 (d, J=8.0 Hz, 1H), 7.37-7.39 (m, 1H), 7.25-7.26 (m, 1H), 4.47-4.54 (m, 1H), 3.91-3.97 (m, 1H), 3.66-3.83 (m, 1H), 3.61-3.65 (m, 1H), 3.33 (s, 3H), 3.18 (s, 1H). ¹³C NMR (151 MHz, Chloroform-d) δ 169.3, 163.2, 150.8, 143.3, 142.1, 140.5, 138.7, 138.1, 137.1, 135.3, 131.1, 131.0, 130.9, 128.7, 126.8, 124.8, 124.5, 121.6, 120.8, 82.0, 78.8, 70.1, 58.9, 51.8. LC-MS: (ESI) calcd for C₂₄H₂₀N₃O₃S [M+H]⁺ 430.12, found 430.10.

6-Methoxy-N-(10-(2-methoxyethyl)-11-oxo-10,11-dihydrodibenzo[b,f][1,4]thiazepin-7-yl) nicotinamide. 1HNMR (400 MHz, Chloroform-d) δ 8.67 (d, J=2.4 Hz, 1H), 8.19 (s, 1H), 8.06 (dd, J=2.4, 2.0 Hz, 1H), 7.92 (d, J=2.4 Hz, 1H), 7.62-7.64 (m, 1H), 7.58 (d, J=2.4, 2.4 Hz, 1H), 7.47 (d, J=8.8, Hz, 1H), 7.37-7.39 (m, 1H), 7.23-7.27 (m, 2H), 6.79 (d, J=4.4 Hz, 1H), 4.51-4.57 (m, 1H), 4.00 (s, 3H), 3.89-3.96 (m, 1H), 3.77-3.83 (m, H), 3.61-3.66 (m, H), 3.33 (s, 3H). 13C NMR (151 MHz, Chloroform-d) δ 169.3, 166.2, 164.0, 146.6, 139.9, 138.8, 138.2, 138.1, 137.0, 135.8, 131.0, 130.8, 128.6, 126.7, 124.3, 123.6, 121.4, 111.0, 70.1, 58.9, 54.2, 51.6. LC-MS: (ESI) calcd for C₂₃H₂₂N₃O₄S [M+H]⁺ 436.14, found 436.10.

2-Methoxy-N-(10-(2-methoxyethyl)-11-oxo-10,11-dihydrodibenzo[b,f][1,4]thiazepin-7-yl) isonicotinamide. ¹H NMR (400 MHz, Chloroform-d): δ 8.27-8.28 (d, J=4.0 Hz, 1H), 8.16 (s, 1H), 7.92-7.93 (d, J=4.0 Hz, 1H), 7.60-7.64 (m, 2H), 7.48-7.50 (d, J=8.0 Hz, 1H), 7.38-7.39 (d, J 4.0 Hz, 1H), 7.22-7.28 (m, 3H), 7.10 (s, 1H), 4.50-4.53 (m, 1H), 3.97 (s, 3H), 3.91-3.95 (m, 1H), 3.79-3.82 (m, 1H), 3.62-3.65 (s, 3H), 3.34 (s, 3H). ¹³C NMR (151 MHz, Chloroform-d) δ 169.2, 164.8, 163.9, 148.1, 144.4, 140.4, 138.7, 138.2, 137.1, 135.3, 131.1, 131.0, 130.8, 128.7, 126.8, 124.3, 121.4, 113.9, 108.8, 70.2, 58.9, 53.9, 51.7. LC-MS: (ESI) calcd for C₂₃H₂₂N₃O₄S [M+H]⁺ 436.14, found 436.20.

4-Fluoro-3-methoxy-N-(10-(2-methoxyethyl)-11-oxo-10,11-dihydrodibenzo[b,f][1,4]thiazepin-7-yl)benzamide. ¹H NMR (400 MHz, Chloroform-d): δ 7.96 (s, 1H), 7.90-7.91 (d, J=4.0 Hz, 1H), 7.48-7.66 (m, 4H), 7.39-7.40 (d, J=4.0 Hz, 1H), 7.25-7.38 (m, 3H), 7.10-7.14 (m, 1H), 4.52-4.55 (m, 1H), 3.90-3.96 (m, 4H), 3.80-3.84 (m, 1H), 3.63-3.67 (m, 1H), 3.34 (s, 3H). ¹³C NMR (151 MHz, Chloroform-d) δ 169.2, 164.9, 155.6, 153.9, 148.2, 140.0, 138.8, 138.2, 137.1, 135.8, 131.1, 131.0, 130.8, 128.6, 126.8, 124.2, 121.3, 119.2, 116.1, 115.9, 113.1 (d, J=2.9 Hz), 70.1, 58.9, 56.3, 51.6. LC-MS: (ESI) calcd for C₂₄H₂₂FN₂O₄S [M+H]⁺ 453.13, found 453.20.

N-(10-(2-Methoxyethyl)-11-oxo-10,11-dihydrodibenzo[b,f][1,4]thiazepin-7-yl)benzo[d]oxazole-6-carboxamide. ¹H NMR (400 MHz, Chloroform-d) δ 8.24 (s, 1H), 8.15 (d, J 0.8 Hz, 1H), 7.95 (t, J=4.4 Hz, 2H), 7.82-7.89 (m, 2H), 7.61-7.68 (m, 2H), 7.52 (d, J=8.8, Hz, 1H), 7.42 (dd, J=1.6, 1.6 Hz, 1H), 7.26-7.32 (m, 2H), 4.51-4.56 (m, 1H), 3.92-3.97 (m, 1H), 3.80-3.86 (m, 1H), 3.64-3.68 (m, 1H), 3.35 (s, 3H). ¹³C NMR (151 MHz, Chloroform-d) & 169.2, 165.1, 154.7, 149.9, 143.1, 140.1, 138.8, 138.2, 137.1, 135.8, 132.3, 131.1, 131.0, 130.8, 128.6, 126.8, 124.2, 123.5, 121.4, 120.8, 110.9, 70.1, 58.9, 51.6. LC-MS: (ESI) calcd for C₂₄H₂₀N₃O₄S [M+H]⁺ 446.12, found 446.10.

N-(10-(2-Methoxyethyl)-11-oxo-10,11-dihydrodibenzo[b,f][1,4]thiazepin-7-yl)-2-methylbenzo[d]oxazole-5-carboxamide. ¹H NMR (400 MHz, Chloroform-d) δ 8.11 (s, 1H), 7.94-7.95 (d, J=4.0 Hz, 2H), 7.84-7.86 (d, J=8.0 Hz, 1H), 7.60-7.68 (m, 2H), 7.43-7.56 (m, 2H), 7.41-7.42 (d, J=4.0 Hz, 1H), 7.26-7.30 (m, 2H), 4.52-4.58 (m, 1H), 3.85-3.97 (m, 1H), 3.79-3.81 (m, 1H), 3.63-3.67 (m, 1H), 3.35 (s, 3H), 2.68 (s, 3H). ¹³C NMR (151 MHz, Chloroform-d) δ 169.1, 165.6, 165.5, 153.2, 141.8, 140.0, 138.8, 138.3, 137.1, 135.9, 131.1, 131.1, 131.0, 130.7, 128.6, 126.8, 124.4, 124.1, 121.3, 118.4, 110.6, 70.1, 58.9, 51.6, 14.6. LC-MS: (ESI) calcd for C₂₅H₂₂N₃O₄S [M+H]⁺ 460.14, found 460.20.

1-(Benzo[d]oxazol-5-yl)-3-(10-(2-methoxyethyl)-11-oxo-10,11-dihydrodibenzo[b,f][1,4]thiazepin-7-yl) urea. ¹H NMR (400 MHz, MeOH-d4) δ 8.03 (s, 1H), 7.83 (d, J=20.5, Hz, 2H), 7.72 (s, 1H), 7.63 (d, J=9.2, Hz, 1H), 7.51 (d, J=2.0, Hz, 1H), 7.36-7.40 (m, 2H), 7.26-7.30 (m, 3H), 7.22 (t, J=3.6, Hz, 2H), 4.56-4.61 (m, 1H), 3.84-3.88 (m, 1H), 3.71-3.76 (m, 1H), 3.59-3.63 (m, 1H), 3.29 (s, 3H). ¹³C NMR (151 MHz, Chloroform-d) δ 169.9, 153.4, 146.6, 140.5, 139.1, 138.0, 137.9, 137.1, 137.0, 135.2, 131.2, 130.9, 130.8, 128.7, 126.6, 123.1, 120.5, 119.5, 112.5, 111.0, 69.9, 58.9, 51.5. LC-MS: (ESI) calcd for C₂₄H₂₁N₄₀₄S [M+H]⁺ 461.13, found 461.10.

(E)-10-(Hex-2-en-1-yl)-7-((4-methylpyridin-2-yl)amino)dibenzo[b,f][1,4]thiazepin-11 (10H)-one. ¹H NMR (400 MHz, Chloroform-d): δ 7.71-7.76 (m, 2H), 7.54-7.55 (d, J=4.0 Hz, 1H), 7.41-7.46 (m 2H), 7.31-7.37 (m, 2H), 7.20-7.26 (m, 2H), 6.71-6.72 (d, J=4.0 Hz, 1H), 5.64-5.68 (m, 2H), 4.77-4.82 (m, 1H), 4.47-4.52 (m, 1H), 2.38 (s, 3H), 1.99-2.03 (m, 2H), 1.33-1.40 (m, 2H), 0.83-0.86 (t, J=8.0 Hz, 3H). ¹³C NMR (151 MHz, Chloroform-d) δ 168.5, 157.4, 152.6, 141.9, 138.2, 138.0, 137.8, 136.5, 134.7, 134.1, 131.6, 131.1, 131.0, 128.9, 126.8, 125.0, 124.8, 115.6, 110.0, 53.3, 34.3, 22.5, 22.2, 13.6. LC-MS: (ESI) calcd for C₂₅H₂₆N₃OS [M+H]⁺ 416.18, found 416.30.

(E)-2-((10-(Hex-2-en-1-yl)-11-oxo-10,11-dihydrodibenzo[b,f][1,4]thiazepin-7-yl)amino) isonicotinonitrile. ¹H NMR (400 MHz, Chloroform-d): δ 8.32-8.33 (d, J=4.0 Hz, 1H), 7.72-7.73 (d, J=4.0 Hz, 1H), 7.26-7.43 (m, 5H), 6.92-6.93 (d, J=4.0 Hz, 1H), 6.87 (s, 1H), 5.61-5.71 (m, 2H), 4.77-4.82 (m, 1H), 4.46-4.50 (m, 1H), 2.38 (s, 3H), 1.99-2.03 (m, 2H), 1.31-1.40 (m, 2H), 0.82-0.86 (t, J=8.0 Hz, 3H). ¹³C NMR (151 MHz, Chloroform-d) δ 168.8, 155.6, 149.6, 138.8, 138.7, 138.3, 137.2, 137.1, 134.5, 131.4, 131.1, 130.8, 128.7, 126.2, 125.0, 124.0, 121.6, 121.0, 116.8, 115.9, 111.3, 53.4, 34.3, 22.2, 13.6. LC-MS: (ESI) calcd for C₂₅H₂₃N₄OS [M+H]⁺ 427.16, found 427.20.

N-(6-(2-Methoxyethyl)-5-oxo-5,6-dihydrobenzo[b]pyrido[4,3-f][1,4]thiazepin-9-yl) furan-3-carboxamide. ¹H NMR (400 MHz, Chloroform-d): δ 8.69 (s, 1H), 8.56 (s, 1H), 8.06 (s, 1H), 7.90 (s, 1H), 7.83 (d, J=2.4 Hz, 1H), 7.71-7.74 (m, 1H), 7.65 (d, J=4.4 Hz, 1H), 7.47-7.52 (m, 2H), 6.74 (d, J=0.8 Hz, 1H), 4.50-4.56 (m, 1H), 3.91-3.98 (m, 1H), 3.76-3.81 (m, 1H), 3.62-3.67 (m, 1H), 3.33 (s, 3H). ¹³C NMR (151 MHz, Chloroform-d) δ 166.5, 160.9, 149.1, 147.9, 147.1, 145.6, 144.3, 138.9, 136.3, 135.7, 127.4, 125.3, 124.3, 122.5, 121.9, 108.3, 69.8, 58.9, 51.8. LC-MS: (ESI) calcd for C₂₀H₁₈N₃O₄S [M+H]⁺ 396.10, found 396.30.

N-(10-(2-Methoxyethyl)-11-oxo-10,11-dihydrobenzo[b]pyrido[3,4-f][1,4]thiazepin-7-yl) furan-3-carboxamide. ¹H NMR (400 MHz, Methanol-d4): δ 8.77 (s, 1H), 8.49-8.51 (d, J=8.0 Hz, 1H), 8.21 (s, 1H), 8.10-8.11 (d, J=4.0 Hz, 1H), 7.71-7.74 (m, 1H), 7.61-7.66 (m, 3H), 6.91 (m, 1H), 4.62-4.68 (m, 1H), 3.92-3.98 (m, 1H), 3.62-3.71 (m, 2H), 3.30 (s, 3H). ¹³C NMR (151 MHz, Methanol-d₄) δ 166.7, 162.1, 151.6, 149.7, 149.0, 145.9, 144.1, 138.4, 137.2, 134.1, 127.0, 125.6, 124.4, 122.5, 122.1, 108.4, 69.6, 57.6, 50.6. LC-MS: (ESI) calcd for C₂₀H₁₈N₃O₄S [M+H]⁺ 396.10, found 396.20.

(E)-10-(Hex-2-en-1-yl)-7-(pyrazin-2-ylamino)dibenzo[b,f][1,4]thiazepin-11 (10H)-one. 1H NMR (400 MHz, Chloroform-d): δ8.18 (s, 1H), 8.13-8.14 (d, J=4.0 Hz, 1H), 8.02 (s, 1H), 7.70-7.75 (m, 3H), 7.41-7.44 (m, 2H), 7.25-7.37 (m, 3H), 6.58 (s, 1H), 5.64-5.67 (m, 2H), 4.77-4.82 (m, 1H), 4.44-4.48 (m, 1H), 1.97-2.02 (m, 2H), 1.32-1.38 (m, 2H), 0.82-0.85 (t, J=12.0 Hz, 3H). 13C NMR (151 MHz, Chloroform-d) δ 168.8, 151.5, 141.7, 138.8, 138.4, 138.2, 137.4, 137.1, 135.4, 134.4, 133.8, 131.4, 131.0, 130.7, 128.6, 126.1, 125.1, 122.9, 120.1, 53.3, 34.3, 22.2, 13.6. LC-MS: (ESI) calcd for C₂₃H₂₃N₄OS [M+H]⁺ 403.16, found 403.10.

(E)-10-(Hex-2-en-1-yl)-7-(pyrimidin-2-ylamino)dibenzo[b,f][1,4]thiazepin-11 (10H)-one. ¹H NMR (400 MHz, Chloroform-d): δ 9.43 (s, 1H), 8.47 (d, J=3.2 Hz, 2H), 7.97 (d, J=2.4 Hz, 1H), 7.71 (dd, J=2.0, 1.6 Hz, 1H), 7.55 (dd, J=2.4, 2.4 Hz, 1H), 7.44 (dd, J=1.2, 1.2 Hz, 1H), 7.38 (d, J=8.8 Hz, 1H), 7.25-7.33 (m, 2H), 6.85 (t, J=4.8 Hz, 1H), 5.64-5.67 (m, 1H), 4.75-4.80 (m, 1H), 4.46-4.50 (m, 1H), 1.97-2.02 (m, 2H), 1.35 (q, J=7.2 Hz, 2H), 0.84 (t, J=7.6 Hz, 3H). ¹³C NMR (151 MHz, Chloroform-d) δ 168.7, 157.5, 157.2, 139.1, 138.7, 138.4, 136.8, 136.0, 134.4, 131.4, 131.0, 130.7, 128.6, 125.9, 125.1, 123.7, 121.0, 112.2, 53.3, 34.3, 22.2, 13.6. LC-MS: (ESI) calcd for C₂₃H₂₃N₄OS [M+H]⁺ 403.16, found 403.10.

N-(10-(6-(Methylamino)-6-oxohexyl)-11-oxo-10,11-dihydrodibenzo[b,f][1,4]thiazepin-7-yl) furan-3-carboxamide. ¹H NMR (400 MHz, Chloroform-d): δ8.75 (s, 1H), 8.13 (s, 1H), 7.81 (d, J 2.4, Hz, 1H), 7.61-7.62 (m, 2H), 7.44 (t, J=1.6, Hz, 1H), 7.31-7.33 (m, 1H), 7.21-7.28 (m, 3H), 6.84 (d, J=1.2, Hz, 1H), 6.38 (d, J=4.4, Hz, 1H), 4.69-7.76 (m, 1H), 3.47-3.54 (m, 1H), 2.71 (d, J 4.8, Hz, 3H), 2.15 (t, J=7.2, Hz, 2H), 1.50-1.64 (m, 4H), 1.34-1.48 (m, 2H). ¹³C NMR (151 MHz, Chloroform-d) δ 174.9, 169.5, 161.4, 145.8, 144.0, 138.8, 138.4, 138.0, 137.2, 136.2, 131.1, 131.0, 130.9, 128.7, 126.1, 124.6, 122.6, 121.8, 108.7, 50.4, 36.1, 27.5, 26.5, 26.2, 25.2. LC-MS: (ESI) calcd for C₂₅H₂₆N₃O₄S [M+H]⁺ 464.17, found 464.20.

(E)-10-(Hex-2-en-1-yl)-7-((3-methylpyridin-2-yl)amino)dibenzo[b,f][1,4]thiazepin-11 (10H)-one. ¹H NMR (400 MHz, Chloroform-d): δ 7.93-7.94 (d, J=4.0 Hz, 1H), 7.79-7.81 (d, J 8.0 Hz, 1H), 7.69-7.72 (m 1H), 7.33-7.39 (m, 4H), 7.33-7.34 (d, J=4.0 Hz, 1H), 7.04-7.32 (m, 2H), 5.62-5.66 (m, 2H), 4.76-4.81 (m, 1H), 4.45-4.50 (m, 1H), 1.97-2.01 (m, 2H), 1.93 (s, 3H), 1.32-1.38 (m, 2H), 0.83-0.86 (t, J=8.0 Hz, 3H). ¹³C NMR (151 MHz, Chloroform-d) δ 168.6, 163.8, 163.5, 151.1, 146.4, 140.7, 138.2, 138.1, 137.0, 136.2, 136.1, 134.6, 131.5, 131.0, 130.9, 128.9, 126.4, 125.9, 125.6, 125.0, 123.1, 116.3, 53.3, 34.3, 22.2, 19.5, 13.6. LC-MS: (ESI) calcd for C₂₅H₂₆N₃OS [M+H]⁺ 416.18, found 416.30.

(E)-2-((10-(Hex-2-en-1-yl)-11-oxo-10,11-dihydrodibenzo[b,f][1,4]thiazepin-7-yl)amino) nicotinonitrile. ¹H NMR (400 MHz, Chloroform-d): δ 8.39-8.41 (m, 1H), 7.88-7.89 (d, J=4.0 Hz, 1H), 7.79-7.81 (m, 1H), 7.69-7.72 (m, 1H), 7.53-7.56 (m, 1H), 7.31-7.44 (m, 2H), 7.26-7.30 (m, 2H), 5.65-5.68 (m, 2H), 4.75-4.79 (m, 1H), 4.50-4.51 (m, 1H), 1.99-2.01 (m, 2H), 1.33-1.39 (m, 2H), 0.83-0.87 (t, J=8.0 Hz, 3H). ¹³C NMR (151 MHz, Chloroform-d) δ 168.6, 155.4, 152.3, 141.7, 139.1, 138.7, 138.4, 136.8, 136.3, 134.3, 131.4, 131.0, 130.7, 128.6, 125.9, 125.1, 124.2, 121.3, 116.0, 114.7, 93.6, 53.3, 34.3, 22.2, 13.6. LC-MS: (ESI) calcd for C₂₅H₂₃N₄OS [M+H]⁺ 427.16, found 427.20.

N-(3-Fluoro-11-oxo-10-(2-(prop-2-yn-1-yloxy)ethyl)-10,11-dihydrodibenzo[b,f][1,4]thiazepin-7-yl)benzo[d]thiazole-5-carboxamide. ¹H NMR (400 MHz, Chloroform-d): δ 9.11 (s, 1H), 8.57 (s, 1H), 8.15 (s, 1H), 8.07 (d, J=8.4 Hz, 1H), 7.97-8.01 (m, 2H), 7.65-7.69 (m, 2H), 7.51 (d, J=8.8 Hz, 1H), 7.13-7.16 (m, 1H), 6.97-7.02 (m, 1H), 4.55-4.61 (m, 1H), 4.16 (d, J=2.4 Hz, 2H), 3.93-4.00 (m, 2H), 3.78-3.84 (m, 1H), 2.42 (t, J=2.0 Hz, 1H). ¹³C NMR (151 MHz, Chloroform-d) δ 168.3, 165.3, 163.8, 162.1, 155.9, 140.9 (d, J=8.2 Hz), 140.1, 137.6, 136.4, 136.0, 134.4 (d, J=3.5 Hz), 133.3 (d, J=9.2 Hz), 132.9, 126.9, 124.5, 124.2, 122.6, 121.9, 121.5, 118.0, 117.9, 115.9, 79.4, 74.7, 67.6, 58.4, 51.5. LC-MS: (ESI) calcd for C₂₆H₁₉FN₃O₃S₂ [M+H]⁺ 504.09, found 504.10.

N-(10-(2-Methoxyethyl)-11-oxo-10,11-dihydrodibenzo[b,f][1,4]thiazepin-7-yl)-2-(4-methoxyphenyl) acetamide. ¹H NMR (400 MHz, Chloroform-d): δ 7.63-7.65 (m, 1H), 7.36-7.41 (m, 3H), 7.15-7.28 (m, 5H), 6.92 (d, J=8.0 Hz, 2H), 4.48-4.54 (m, 1H), 3.84-3.90 (m, 1H), 3.82 (s, 3H), 3.74-3.78 (m, 1H), 3.64 (s, 2H), 3.57-3.62 (m, 1H), 3.31 (s, 3H). ¹³C NMR (151 MHz, Chloroform-d) δ 169.7, 169.1, 159.2, 139.8, 138.8, 138.3, 136.9, 135.6, 131.1, 131.0, 130.7, 130.6, 128.6, 126.6, 125.9, 123.5, 120.8, 114.7, 70.1, 58.8, 55.3, 51.5, 43.8. LC-MS: (ESI) calcd for C₂₅H₂₅N₂O₄S [M+H]⁺ 449.16, found 449.10.

N-(10-(2-Methoxyethyl)-11-oxo-10,11-dihydrodibenzo[b,f][1,4]thiazepin-7-yl)benzothioamide. ¹H NMR (600 MHz, Chloroform-d) δ 8.97 (s, 1H), 8.05 (s, 1H), 7.91 (s, 1H), 7.80 (s, 1H), 7.68 (dd, J=7.6, 1.7 Hz, 1H), 7.60 (d, J=8.7 Hz, 1H), 7.51 (t, J=7.3 Hz, 1H), 7.42 (d, J=8.2 Hz, 2H), 7.36-7.27 (m, 3H), 4.49 (dt, J=13.3, 6.0 Hz, 1H), 4.01 (dt, J=13.9, 5.3 Hz, 1H), 3.86 (ddd, J=10.2, 6.8, 5.0 Hz, 1H), 3.67 (dt, J=10.4, 5.4 Hz, 1H), 3.36 (s, 3H). LC-MS: (ESI) calcd for C₂₃H₂₁N₂O₂S₂ [M+H]⁺ 421.10, found 421.10.

N-(10-(But-2-yn-1-yl)-11-oxo-10,11-dihydrodibenzo[b,f][1,4]thiazepin-7-yl)benzo[d]thiazole-5-carboxamide. ¹H NMR (400 MHz, Chloroform-d) δ 9.08 (s, 1H), 8.59 (d, J=1.7 Hz, 1H), 8.46 (s, 1H), 8.06-7.94 (m, 3H), 7.77-7.65 (m, 3H), 7.43-7.34 (m, 1H), 7.32-7.22 (m, 2H), 4.94 (dd, J=17.0, 2.4 Hz, 1H), 4.44 (dd, J=17.0, 2.4 Hz, 1H), 1.85 (t, J=2.4 Hz, 3H). ¹³C NMR (151 MHz, Chloroform-d) δ 168.5, 165.4, 155.8, 153.0, 139.9, 138.5, 137.5, 137.5, 136.1, 135.8, 131.8, 131.1, 131.1, 128.6, 125.1, 124.6, 124.1, 122.5, 122.0, 121.2, 80.2, 74.7, 41.2, 3.9. LC-MS: (ESI) calcd for C₂₅H₁₈N₃₀₂S₂ [M+H]⁺ 456.09, found 456.10.

N-(10-(But-2-yn-1-yl)-11-oxo-10,11-dihydrodibenzo[b,f][1,4]thiazepin-7-yl)-3-methoxybenzamide. ¹H NMR (400 MHz, Chloroform-d) δ 8.12 (s, 1H), 7.95 (d, J=2.4 Hz, 1H), 7.78-7.61 (m, 3H), 7.45-7.33 (m, 4H), 7.32-7.27 (m, 2H), 7.07 (dt, J=7.4, 2.6 Hz, 1H), 4.93 (dd, J=17.0, 2.4 Hz, 1H), 4.43 (dd, J=17.0, 2.4 Hz, 1H), 3.85 (s, 3H), 1.85 (s, 3H). ¹³C NMR (151 MHz, Chloroform-d) δ 168.5, 165.7, 160.0, 144.8, 139.7, 138.6, 137.6, 136.1, 135.9, 135.8, 131.8, 131.7, 131.1, 131.1, 131.0, 130.8, 129.8, 128.6, 128.4, 125.6, 125.1, 124.1, 121.2, 118.8, 118.3, 118.2, 115.8, 112.5, 80.2, 74.7, 55.5, 41.2, 3.8. LC-MS: (ESI) calcd for C₂₅H₂₁N₂O₃S [M+H]⁺ 429.13, found 429.20.

(E)-10-(Hex-2-en-1-yl)-7-((3-methoxypyridin-2-yl)amino)dibenzo[b,f][1,4]thiazepin-11 (10H)-one. ¹H NMR (400 MHz, Chloroform-d): δ 7.91-7.92 (d, J=4.0 Hz, 1H), 7.82-7.84 (m, 1H), 7.68-7.72 (m, 2H), 7.40-7.42 (m, 1H), 7.24-7.33 (m, 2H), 6.96-7.01 (m, 1H), 6.72-6.75 (m, 1H), 5.65-5.67 (m, 2H), 4.75-4.78 (m, 1H), 4.46-4.47 (m, 1H), 3.89 (s, 3H), 1.98-2.00 (m, 2H), 1.33-1.38 (m, 2H), 0.83-0.86 (t, J=8.0 Hz, 3H). ¹³C NMR (151 MHz, Chloroform-d) δ 168.8, 145.8, 142.3, 139.1, 138.6, 138.5, 138.3, 136.8, 136.6, 134.1, 131.3, 131.0, 130.4, 128.4, 125.9, 125.3, 121.9, 119.2, 114.9, 114.7, 55.4, 53.3, 34.3, 22.3, 13.6. LC-MS: (ESI) calcd for C₂₅H₂₆N₃₀₂S [M+H]⁺ 432.18, found 432.30.

(E)-10-(Hex-2-en-1-yl)-7-((3-(trifluoromethyl)pyridin-2-yl)amino)dibenzo[b,f][1,4]thiazepin-11 (10H)-one. ¹H NMR (400 MHz, Chloroform-d): δ 8.36 (d, J=4.0 Hz, 1H), 7.76-7.82 (m, 2H), 7.71 (d, J=4.0 Hz, 1H), 7.51-7.54 (m, 1H), 7.30-7.43 (m, 2H), 7.26-7.29 (m, 2H), 6.85-6.88 (m, 1H), 6.69 (s, 1H), 5.66-5.68 (m, 2H), 4.73-4.77 (m, 1H), 4.47-4.51 (m, 1H), 1.98-2.02 (m, 2H), 1.34-1.39 (m, 2H), 0.85 (t, J=8.0 Hz, 3H). ¹³C NMR (151 MHz, Chloroform-d) & 168.7, 151.5, 151.3, 138.8, 138.7, 138.5, 137.0, 136.6, 135.7 (q, J=5.2 Hz), 134.2, 131.4, 131.0, 130.6, 128.6, 125.8, 125.2, 124.3, 121.6, 114.6, 53.3, 34.3, 22.3, 13.6. LC-MS: (ESI) calcd for C₂₅H₂₃F₃N₃OS [M+H]⁺ 470.15, found 470.20.

(E)-7-((3-Fluoropyridin-2-yl)amino)-10-(hex-2-en-1-yl)dibenzo[b,f][1,4]thiazepin-11 (10H)-one. ¹H NMR (400 MHz, Chloroform-d): δ 8.02-8.03 (d, J=4.0 Hz, 1H), 7.89-7.90 (d, J=4.0 Hz, 1H), 7.63-7.71 (m, 1H), 7.61-7.62 (m, 1H), 7.41-7.43 (m, 1H), 7.25-7.35 (m, 4H), 6.74-6.78 (m, 1H), 6.60-6.61 (m, 1H), 5.65-5.67 (m, 1H), 4.76-4.79 (m, 1H), 4.47-4.48 (m, 1H), 1.98-2.01 (m, 2H), 1.33-1.38 (m, 2H), 0.82-0.86 (t, J=8.0 Hz, 3H). ¹³C NMR (151 MHz, Chloroform-d) δ 168.8, 147.8, 146.1, 144.7 (d, J=9.2 Hz), 142.6 (d, J=6.1 Hz), 138.9, 138.5, 137.6, 136.7, 134.2, 131.4, 131.0, 130.5, 128.5, 125.9, 125.2, 122.4, 121.3, 121.2, 119.6, 115.2, 53.3, 34.3, 22.2, 13.6. LC-MS: (ESI) calcd for C₂₄H₂₃FN₃OS [M+H]⁺ 420.16, found 420.10.

N-(10-Allyl-3-fluoro-11-oxo-10,11-dihydrodibenzo[b,f][1,4]thiazepin-7-yl)benzo[d]thiazole-5-carboxamide. ¹H NMR (400 MHz, Chloroform-d): δ 9.08 (s, 1H), 8.57 (s, 1H), 8.49 (s, 1H), 8.02 (d, J=8.4 Hz, 1H), 7.95-7.99 (m, 2H), 7.66-7.70 (m, 2H), 7.39 (d, J=8.4 Hz, 1H), 7.09-7.12 (m, 1H), 6.94-6.99 (m, 1H), 5.95-6.04 (m, 1H), 5.29 (d, J=17.2 Hz, 1H), 5.20 (d, J=10.4 Hz, 1H), 4.81-4.86 (m, 1H), 4.51-4.56 (m, 1H). ¹³C NMR (151 MHz, Chloroform-d) δ 167.9, 165.4, 163.9, 162.2, 155.8, 153.1, 140.7 (d, J=8.2 Hz), 139.7, 137.6, 136.0, 135.9, 134.3 (d, J=3.3 Hz), 133.6 (d, J=8.9 Hz), 133.2, 132.9, 125.9, 124.5, 124.2, 122.6, 122.0, 121.4, 118.0, 117.9, 117.7, 116.1, 116.0, 53.9. LC-MS: (ESI) calcd for C₂₄H₁₇FN₃O₂S₂ [M+H]⁺ 462.08, found 462.10.

N-(10-(2-Methoxyethyl)-11-oxo-10,11-dihydrodibenzo[b,f][1,4]thiazepin-7-yl)-2-(3-methoxyphenyl) acetamide. ¹H NMR (400 MHz, Chloroform-d): δ 7.64-7.65 (m, 1H), 7.36-7.41 (m, 3H), 7.24-7.33 (m, 3H), 7.17 (s, 1H), 6.83-6.89 (m, 2H), 6.80 (s, 1H), 4.48-4.54 (m, 1H), 3.84-3.90 (m, 1H), 3.82 (s, 3H), 3.74-3.78 (m, 1H), 3.68 (s, 2H), 3.57-3.63 (m, 1H), 3.31 (s, 3H). ¹³C NMR (151 MHz, Chloroform-d) δ 169.0, 160.2, 139.9, 138.8, 138.3, 136.9, 135.5, 135.4, 131.1, 131.0, 130.7, 130.4, 128.6, 126.7, 123.6, 121.6, 120.9, 115.2, 113.2, 70.1, 58.8, 55.3, 51.5, 44.8. LC-MS: (ESI) calcd for C₂₅H₂₅N₂O₄S [M+H]⁺ 449.16, found 449.10.

N-(10-(But-2-yn-1-yl)-3-fluoro-11-oxo-10,11-dihydrodibenzo[b,f][1,4]thiazepin-7-yl)-6-methoxynicotinamide. ¹H NMR (400 MHz, Chloroform-d): δ 8.68 (s, 1H), 8.12 (s, 1H), 8.05-8.08 (m, 1H), 7.94 (d, J=2.0 Hz, 1H), 7.64-7.75 (m, 3H), 7.10-7.13 (m, 1H), 6.96-7.00 (m, 1H), 6.80 (d, J 8.4 Hz, 1H), 4.89-4.93 (m, 1H), 4.40-4.44 (m, 1H), 4.00 (s, 3H), 1.85 (s, 3H). ¹³C NMR (151 MHz, Chloroform-d) δ 167.5, 166.5, 164.1, 163.9, 162.4, 146.5, 140.6 (d, J=11.0 Hz), 139.9, 137.8, 135.9, 135.0, 134.1 (d, J=9.2 Hz), 133.7 (d, J=15.9 Hz), 125.3, 124.1, 123.4, 121.4, 118.1, 117.9, 116.1, 116.0, 111.3, 80.3, 74.6, 54.1, 41.2, 3.8. LC-MS: (ESI) calcd for C₂₄H₁₉FN₃O₃S [M+H]⁺ 448.12, found 448.10.

N-(10-(But-2-yn-1-yl)-3-fluoro-11-oxo-10,11-dihydrodibenzo[b,f][1,4]thiazepin-7-yl)-4-(trifluoromethoxy)benzamide. ¹H NMR (400 MHz, Chloroform-d): δ 7.83-7.94 (m, 3H), 7.79 (s, 1H), 7.69-7.78 (m, 2H), 7.62-7.65 (m, 1H), 7.33 (d, J=8.0 Hz, 2H), 7.14-7.26 (m, 1H), 7.00-7.04 (m, 1H), 4.89-4.94 (m, 1H), 4.41-4.46 (m, 1H), 1.85 (s, 3H). ¹³C NMR (151 MHz, Chloroform-d) δ 167.7, 164.5, 164.1, 162.4, 151.9, 140.6 (d, J=8.3 Hz), 139.9, 136.1, 135.0, 134.0 (d, J=9.1 Hz), 133.7 (d, J=3.3 Hz), 132.7, 129.1, 125.2, 124.2, 121.6, 120.8, 118.1, 118.0, 116.1, 116.0, 80.4, 74.6, 41.3, 3.8. LC-MS: (ESI) calcd for C₂₅H₁₇F₄N₂O₃S [M+H]⁺ 501.09, found 501.10.

N-(10-(But-2-yn-1-yl)-3-fluoro-11-oxo-10,11-dihydrodibenzo[b,f][1,4]thiazepin-7-yl)-4-(difluoromethoxy)benzamide. ¹H NMR (400 MHz, Chloroform-d): δ7.94 (s, 1H), 7.79-7.89 (m, 3H), 7.72-7.77 (m, 2H), 7.63-7.66 (m, 1H), 7.21-7.26 (m, 2H), 7.13-7.16 (m, 1H), 6.99-7.03 (m, 1H), 6.41-6.78 (m, 1H), 4.89-4.94 (m, 1H), 4.41-4.46 (m, 1H), 1.85 (s, 3H). ¹³C NMR (151 MHz, Chloroform-d) δ 167.6, 164.7, 164.0, 162.4, 153.9, 140.6 (d, J=8.2 Hz), 139.8, 136.1, 135.0, 134.0 (d, J=8.9 Hz), 133.7 (d, J=3.4 Hz), 131.2, 129.1, 125.2, 124.2, 121.5, 119.3, 118.1, 117.9, 117.0, 116.1, 115.9, 115.3, 113.6, 80.4, 74.6, 41.2, 3.8. 13C NMR (151 MHz, DMSO-d6) δ 162.9, 162.6, 161.0, 148.6, 143.5, 139.6 (d, J=8.6 Hz), 137.5, 136.4, 135.5, 133.6, 133.3 (d, J=9.8 Hz), 128.9, 126.5, 125.1, 123.2, 121.1, 117.3 (d, J=22.8 Hz), 115.8 (d, J=21.5 Hz), 82.1, 79.3, 74.3, 2.6. LC-MS: (ESI) calcd for C₂₅H₁₈F₃N₂O₃S [M+H]⁺ 483.10, found 483.00.

N-(10-(But-2-yn-1-yl)-3-fluoro-11-oxo-10,11-dihydrodibenzo[b,f][1,4]thiazepin-7-yl)benzo[d]thiazole-5-carboxamide. ¹H NMR (400 MHz, Chloroform-d): δ 9.11 (s, 1H), 8.58 (d, J=1.2 Hz, 1H), 8.21 (s, 1H), 8.05-8.08 (m, 1H), 7.98-8.01 (m, 2H), 7.74-7.79 (m, 2H), 7.68-7.71 (m, 1H), 7.13-7.16 (m, 1H), 6.98-7.03 (m, 1H), 4.90-4.95 (m, 1H), 4.42-4.47 (m, 1H), 1.86 (t, J=2.0 Hz, 3H). ¹³C NMR (151 MHz, Chloroform-d) δ 167.6, 165.3, 164.0, 162.4, 155.9, 153.1, 140.6 (d, J 8.4 Hz), 139.9, 137.6, 136.1, 135.1, 134.0 (d, J=9.2 Hz), 133.76 (d, J=3.4 Hz), 132.9, 125.3, 124.5, 124.1, 122.6, 121.9, 121.4, 118.1, 117.9, 116.1, 115.9, 80.3, 74.6, 41.2, 3.8. LC-MS: (ESI) calcd for C₂₅H₁₇FN₃O₂S₂ [M+H]⁺ 474.08, found 474.10.

N-(10-(But-2-yn-1-yl)-3-fluoro-11-oxo-10,11-dihydrodibenzo[b,f][1,4]thiazepin-7-yl)-3-(1-cyanoethyl)benzamide. ¹H NMR (400 MHz, Chloroform-d): δ 8.08 (s, 1H), 7.95 (d, J=2.4 Hz, 1H), 7.86 (s, 1H), 7.81 (s, 1H), 7.00-7.79 (m, 3H), 7.68 (d, J=2.4 Hz, 1H), 7.50-7.58 (m, 1H), 7.12-7.15 (m, 1H), 6.97-7.02 (m, 1H), 4 . . . 89-4.94 (m, 1H), 4.41-4.46 (m, 1H), 3.96-4.02 (m, 1H), 1.86 (t, J=2.0 Hz, 3H), 1.68 (t, J=7.2 Hz, 3H). ¹³C NMR (151 MHz, Chloroform-d) δ 167.6, 165.1, 164.0, 162.3, 140.6 (d, J=8.4 Hz), 139.9, 138.0, 136.0, 135.4, 134.9, 134.0 (d, J=9.2 Hz), 133.7 (d, J=3.3 Hz), 130.4, 129.8, 126.7, 125.8, 125.2, 124.3, 121.6, 121.1, 118.1, 117.9, 116.1, 115.9, 80.4, 74.6, 41.2, 31.2, 21.3, 3.8. LC-MS: (ESI) calcd for C₂₇H₂₁FN₃O₂S [M+H]: 470.14, found 470.10.

10-(But-2-yn-1-yl)-3-fluoro-7-((3-methylpyrazin-2-yl)amino)dibenzo[b,f][1,4]thiazepin-11 (10H)-one. ¹H NMR (400 MHz, Chloroform-d): δ 8.03 (d, J=2.4, Hz, 1H), 7.96 (d, J=2.4, Hz, 1H), 7.86 (d, J=2.4, Hz, 1H), 7.77-7.80 (m, 1H), 7.71 (d, J=8.8, Hz, 1H), 7.61-7.64 (m, 1H), 7.14-7.17 (m, 1H), 6.99-7.04 (m, 1H), 6.64 (s, 1H), 4.90-4.95 (m, 1H), 4.37-4.43 (m, 1H), 2.54 (s, 3H), 1.86 (t, J=2.4 Hz, 3H). ¹³C NMR (151 MHz, Chloroform-d) δ 167.6, 164.0, 162.3, 149.7, 141.0, 140.7, 139.3, 138.2, 137.9, 134.9, 134.0 (d, J=9.4 Hz), 125.2, 123.2, 120.9, 118.0, 117.9, 116.0, 115.9, 80.1, 74.8, 41.2, 20.4, 3.9. LC-MS: (ESI) calcd for C₂₂H₁₈FN₄OS [M+H]⁺ 405.12, found 405.20.

10-(But-2-yn-1-yl)-3-fluoro-7-((3-methoxypyrazin-2-yl)amino)dibenzo[b,f][1,4]thiazepin-11 (10H)-one. ¹H NMR (400 MHz, Chloroform-d): δ 8.02 (s, 1H), 7.77-7.80 (m, 1H), 7.67-7.74 (m, 3H), 7.53 (d, J=2.8, Hz, 1H), 7.15-7.18 (m, 1H), 7.11 (s, 1H), 6.99-7.04 (m, 1H), 4.89-4.94 (m, 1H), 4.39-4.44 (m, 1H), 4.05 (s, 3H), 1.85 (d, J=2.4 Hz, 3H). ¹³C NMR (151 MHz, Chloroform-d) δ 167.6, 164.0, 162.3, 148.4, 141.6, 140.8 (d, J=8.3 Hz), 137.8, 137.7, 134.9, 134.0 (d, J=9.2 Hz), 133.1, 129.9, 129.0, 128.4, 125.2, 122.3, 119.9, 118.0, 117.9, 116.0, 115.8, 80.0, 74.8, 53.9, 41.2, 3.8. LC-MS: (ESI) calcd for C₂₂H₁₈FN₄O₂S [M+H]⁺ 421.12, found 421.10.

N-(10-(But-2-yn-1-yl)-3-fluoro-11-oxo-10,11-dihydrodibenzo[b,f][1,4]thiazepin-7-yl)benzo[d]oxazole-5-carboxamide. ¹H NMR (400 MHz, Chloroform-d): δ 8.29 (s, 1H), 8.21 (s, 1H), 7.95-7.99 (m, 3H), 7.65-7.80 (m, 4H), 7.15-7.17 (m, 1H), 6.99-7.04 (m, 1H), 4.90-4.95 (m, 1H), 4.43-4.48 (m, 1H), 1.86 (t, J=4.4 Hz, 3H). ¹³C NMR (151 MHz, Chloroform-d) δ 167.6, 165.3, 164.0, 162.4, 154.0, 152.2, 140.6 (d, J=8.6 Hz), 140.3, 139.9, 136.1, 135.0, 134.0 (d, J 9.1 Hz), 133.8 (d, J=3.4 Hz), 131.6, 125.5, 125.3, 124.2, 121.5, 119.6, 118.1, 117.9, 116.1, 115.9, 111.6, 80.4, 74.6, 41.2, 3.8. LC-MS: (ESI) calcd for C₂₅H₁₇FN₃O₃S [M+H]⁺ 458.10, found 458.10.

N-(10-(3-Cyclopropylprop-2-yn-1-yl)-11-oxo-10,11-dihydrodibenzo[b,f][1,4]thiazepin-7-yl)benzo[d]thiazole-5-carboxamide. ¹H NMR (600 MHz, Chloroform-d) δ 9.13 (s, 1H), 8.62 (d, J 1.7 Hz, 1H), 8.21 (d, J=2.6 Hz, 1H), 8.07 (d, J=8.4 Hz, 1H), 8.03-7.98 (m, 2H), 7.78-7.72 (m, 1H), 7.72-7.66 (m, 2H), 7.46-7.40 (m, 1H), 7.30 (td, J=7.1, 1.8 Hz, 2H), 4.89 (dd, J 17.2, 1.9 Hz, 1H), 4.54 (dd, J=17.2, 2.0 Hz, 1H), 1.25 (td, J=5.1, 2.5 Hz, 1H), 0.78-0.72 (m, 2H), 0.71-0.61 (m, 2H). ¹³C NMR (151 MHz, Chloroform-d) & 168.4, 165.2, 156.3, 156.0, 153.0, 151.9, 139.8, 138.6, 137.6, 136.2, 135.9, 133.0, 131.8, 131.1, 131.1, 128.7, 125.4, 124.6, 124.0, 122.6, 121.9, 121.0, 87.9, 70.7, 41.0, 8.2, 8.1. LC-MS: (ESI) calcd for C₂₇H₂₀N₃O₂S₂ [M+H]⁺ 482.10, found 482.10.

N-(10-(But-2-yn-1-yl)-3-fluoro-11-oxo-10,11-dihydrodibenzo[b,f][1,4]thiazepin-7-yl)-6-ethynylnicotinamide. ¹H NMR (400 MHz, DMSO-d6): δ 10.67 (s, 1H), 9.07 (s, 1H), 8.29-8.32 (m, 1H), 8.18 (s, 1H), 7.69-7.81 (m, 4H), 7.46-7.48 (m, 1H), 7.27-7.31 (m, 1H), 4.83-4.88 (m, 1H), 4.66-4.71 (m, 1H), 4.58 (s, 1H), 1.77 (s, 3H). ¹³C NMR (151 MHz, DMSO-d6) δ 165.7, 162.9, 162.6, 161.0, 148.6, 143.5, 139.6 (d, J=8.6 Hz), 137.5, 136.4, 135.5, 133.6, 133.3 (d, J=9.8 Hz), 128.9, 126.5, 125.1, 123.2, 121.1, 117.3, 117.2, 115.8, 115.7, 82.1, 79.3, 74.3, 2.6. LC-MS: (ESI) calcd for C₂H₁₇FN₃O₂S [M+H]⁺ 442.10, found 442.10.

1-(Benzo[d]thiazol-5-yl)-3-(10-(but-2-yn-1-yl)-3-fluoro-11-oxo-10,11-dihydrodibenzo[b,f][1,4]thiazepin-7-yl) urea. ¹H NMR (400 MHz, DMSO-d6): δ 9.35 (s, 1H), 9.26 (s, 2H), 8.33 (s, 1H), 8.04 (d, J=8.8 Hz, 1H), 7.95-7.96 (m, 1H), 7.70-7.72 (m, 1H), 7.41-7.50 (m, 3H), 7.26-7.30 (m, 1H), 4.78-4.83 (m, 1H), 4.64-4.69 (m, 1H), 1.77 (t, J=2.4 Hz, 3H). ¹³C NMR (151 MHz, DMSO-d6) δ 165.8, 162.6, 160.9, 156.2, 153.2, 152.0, 139.7 (d, J=8.4 Hz), 137.6 (d, J 7.2 Hz), 135.6, 133.7, 133.4 (d, J=3.1 Hz), 133.2 (d, J=9.4 Hz), 126.1, 125.1, 121.7, 120.9, 119.2, 117.2 (d, J=23.0 Hz), 117.1, 115.7, 115.6, 111.2, 79.1, 74.4, 2.6. LC-MS: (ESI) calcd for C₂₅H₁₈FN₄O₂S₂ [M+H]⁺ 489.09, found 489.00.

10-(But-2-yn-1-yl)-3-fluoro-7-((3-methylpyridin-2-yl)amino)dibenzo[b,f][1,4]thiazepin-11 (10H)-one. ¹H NMR (400 MHz, Chloroform-d): δ 7.98 (d, J=4.0 Hz, 1H), 7.69-7.81 (m, 3H), 7.33 (s, 1H), 7.18-7.21 (m, 1H), 7.12-7.15 (m, 1H), 7.02-7.06 (m, 2H), 4.88-4.93 (m, 1H), 4.41-4.46 (m, 1H), 2.01 (s, 3H), 1.84 (t, J=4.4 Hz, 3H). ¹³C NMR (151 MHz, Chloroform-d) δ 167.4, 164.2, 162.5, 151.1, 146.4, 140.7, 140.1 (d, J=8.4 Hz), 136.5, 136.3, 135.2, 134.2 (d, J=9.3 Hz), 133.6 (d, J=3.5 Hz), 125.9, 125.7, 125.6, 123.5, 118.1, 117.9, 116.4, 116.3, 116.2, 80.5, 74.5, 41.2, 19.5, 3.8. LC-MS: (ESI) calcd for C₂₃H₁₉FN₃OS [M+H]⁺ 404.13, found 404.20.

2-((10-(But-2-yn-1-yl)-3-fluoro-11-oxo-10,11-dihydrodibenzo[b,f][1,4]thiazepin-7-yl)amino) nicotinonitrile. ¹H NMR (400 MHz, Chloroform-d): δ 7.94 (d, J=4.0 Hz, 1H), 7.81-7.83 (m, 1H), 7.77-7.80 (m, 2H), 7.72-7.74 (m, 1H), 7.57-7.60 (m, 1H), 7.15-7.18 (m, 1H), 6.99-7.05 (m, 2H), 6.85-6.88 (m, 1H), 4.90-4.95 (m, 1H), 4.39-4.44 (m, 1H), 1.86 (t, J=4.4 Hz, 3H). ¹³C NMR (151 MHz, Chloroform-d) δ 167.5, 164.0, 162.3, 155.4, 152.3, 141.8, 140.6 (d, J=8.2 Hz), 139.1, 136.7, 134.8, 134.1 (d, J=9.1 Hz), 133.9 (d, J=3.5 Hz), 125.1, 124.1, 121.7, 118.1, 117.9, 116.0 (d, J=7.1 Hz), 115.9, 114.9, 93.7, 80.2, 74.7, 41.2, 3.9. LC-MS: (ESI) calcd for C₂₃H₁₆FN₄OS [M+H]⁺ 415.11, found 415.10.

10-(But-2-yn-1-yl)-3-fluoro-7-((3-fluoropyridin-2-yl)amino)dibenzo[b,f][1,4]thiazepin-11 (10H)-one. ¹H NMR (400 MHz, Chloroform-d): δ 8.04 (d, J=3.2, Hz, 1H), 7.97 (d, J=3.2, Hz, 1H), 7.77-7.81 (m, 1H), 7.64-7.71 (m, 2H), 7.26-7.33 (m, 1H), 6.99-7.04 (m, 1H), 6.76-6.80 (m, 1H), 6.64 (s, 1H), 4.89-4.93 (m, 1H), 4.40-4.44 (m, 1H), 1.86 (t, J=2.4 Hz, 3H). ¹³C NMR (151 MHz, Chloroform-d) δ 167.7, 164.0, 162.3, 147.8, 146.1, 144.6 (d, J=8.5 Hz), 142.6 (d, J=6.2 Hz), 140.9 (d, J=8.3 Hz), 138.0, 137.6, 134.9, 134.0 (d, J=9.3 Hz), 125.2, 122.3, 121.4, 121.3, 120.0, 118.0, 117.9, 115.9, 115.8, 115.4 (d, J=2.1 Hz), 80.0, 74.8, 41.2, 3.9. LC-MS: (ESI) calcd for C₂₂H₁₆F₂N₃OS [M+H]⁺ 408.10, found 408.10.

10-(But-2-yn-1-yl)-3-fluoro-7-((3-methoxypyridin-2-yl)amino)dibenzo[b,f][1,4]thiazepin-11 (10H)-one. ¹H NMR (400 MHz, Chloroform-d): δ 7.99 (d, J=2.8 Hz, 1H), 7.83-7.85 (m, 1H), 7.71-7.80 (m, 2H), 7.65 (d, J=8.8 Hz, 1H), 7.15-7.17 (m, 3H), 6.74-6.77 (m, 1H), 4.87-4.92 (m, 1H), 4.39-4.44 (m, 1H), 3.90 (s, 3H), 1.85 (t, J=4.8 Hz, 3H). ¹³C NMR (151 MHz, Chloroform-d) δ 167.8, 163.9, 162.2, 145.7, 142.3, 141.1 (d, J=8.2 Hz), 138.9, 138.3, 136.8, 134.7, 134.1 (d, J 3.5 Hz), 133.9 (d, J=9.1 Hz), 125.1, 121.9, 119.6, 118.0, 117.9, 115.8, 115.7, 115.0, 114.9, 79.9, 74.9, 55.5, 41.2, 3.9. LC-MS: (ESI) calcd for C₂₃H₁₉FN₃O₂S [M+H]⁺ 420.12, found 420.20.

10-(But-2-yn-1-yl)-7-((3-cyclopropylpyridin-2-yl)amino)-3-fluorodibenzo[b,f][1,4]thiazepin-11 (10H)-one. ¹H NMR (400 MHz, Methanol-d4): δ 7.92 (d, J=8.4, Hz, 1H), 7.85 (d, J=7.2, Hz, 1H), 7.80 (d, J=2.4, Hz, 1H), 7.30-7.36 (m, 1H), 7.67 (d, J=5.6, Hz, 1H), 7.53-7.56 (m, 1H), 7.31-7.33 (m, 1H), 7.17-7.22 (m, 1H), 7.02 (d, J=6.4, Hz, 1H), 4.72 (d, J=2.4, Hz, 1H), 4.68 (d, J=2.4, Hz, 1H), 1.87-1.90 (m, 1H), 1.81 (t, J=2.4, Hz, 3H), 1.13-1.17 (m, 2H), 0.78-0.83 (m, 2H). ¹³C NMR (151 MHz, Methanol-d4) δ 167.5, 163.9, 139.8, 134.7, 133.5, 133.1, 126.0, 117.5, 117.3, 115.7, 115.6, 114.2, 39.9, 9.7, 5.2. LC-MS: (ESI) calcd for C₂₅H₂₁FN₃OS [M+H]⁺ 430.14, found 430.30.

3-Cyano-N-(10-(2-methoxyethyl)-11-oxo-10,11-dihydrodibenzo[b,f][1,4]thiazepin-7-yl)benzamide. ¹H NMR (400 MHz, Chloroform-d): δ 8.15 (s, 1H), 8.07-8.11 (m, 2H), 7.93 (s 1H), 7.84 (d, J=8.0 Hz, 1H), 7.59-7.66 (m, 3H), 7.53 (d, J=8.8 Hz, 1H), 7.40-7.42 (m, 1H), 7.26-7.30 (m, 2H), 4.49-4.56 (m, 1H), 3.92-3.98 (m, 1H), 3.80-3.85 (m, 1H), 3.62-3.68 (m, 1H), 3.35 (s, 3H). ¹³C NMR (151 MHz, Chloroform-d) δ 169.2, 163.6, 140.4, 138.7, 138.2, 137.1, 135.7, 135.4, 135.1, 131.7, 131.0, 130.9, 130.8, 129.8, 128.7, 126.8, 124.4, 121.5, 118.0, 113.0, 70.1, 58.9, 51.7. LC-MS: (ESI) calcd for C₂₄H₂₀N₃O₃S [M+H]⁺ 430.12, found 430.10.

3-Fluoro-N-(10-(2-methoxyethyl)-11-oxo-10,11-dihydrodibenzo[b,f][1,4]thiazepin-7-yl)benzamide. ¹H NMR (400 MHz, Chloroform-d): δ 7.98 (s, 1H), 7.92 (d, J=2.4 Hz, 1H), 7.66 (d, J=2.0 Hz, 1H), 7.51-7.65 (m, 3H), 7.40-7.49 (m, 3H), 7.23-7.31 (m, 3H), 4.50-4.57 (m, 1H), 3.91-3.97 (m, 1H), 3.79-3.84 (m, 1H), 3.62-0.367 (m, 1H), 3.34 (s, 3H). ¹³C NMR (151 MHz, Chloroform-d) δ 169.1, 164.4, 163.6, 162.0, 140.3, 138.8, 138.2, 137.1, 136.6, 135.5, 131.1 (d, J 12.4 Hz), 130.8, 130.6 (d, J=7.9 Hz), 128.7, 126.8, 124.2, 122.5 (d, J=3.0 Hz), 121.3, 119.3, 119.1, 114.7, 114.5, 70.2, 58.9, 51.6. LC-MS: (ESI) calcd for C₂₃H₂₀FN₂O₃S [M+H]⁺ 423.12, found 423.10.

N-(10-(2-Methoxyethyl)-11-oxo-10,11-dihydrodibenzo[b,f][1,4]thiazepin-7-yl)-3-(trifluoromethoxy)benzamide. ¹H NMR (400 MHz, Chloroform-d): δ 8.19 (s, 1H), 7.92 (d, J=2.4 Hz, 1H), 7.72-7.76 (m, 2H), 7.50-7.60 (m, 2H), 7.48-7.52 (m, 2H), 7.38-7.41 (m, 2H), 7.24-7.28 (m, 2H), 4.49-4.56 (m, 1H), 3.90-3.96 (m, 1H), 3.78-3.83 (m, 1H), 3.61-366 (m, 1H), 3.34 (s, 3H). ¹³C NMR (151 MHz, Chloroform-d) δ 169.2, 164.2, 149.5, 140.3, 138.8, 138.2, 137.1, 136.5, 135.5, 131.1, 131.0, 130.8, 130.4, 128.7, 126.8, 125.1, 124.4, 124.3, 121.4, 120.1, 70.2, 58.9, 51.7. LC-MS: (ESI) calcd for C₂₄H₂₀F₃N₂O₄S [M+H]: 489.11, found 489.10.

3-(Difluoromethoxy)-N-(10-(2-methoxyethyl)-11-oxo-10,11-dihydrodibenzo[b,f][1,4]thiazepin-7-yl)benzamide. ¹H NMR (400 MHz, Chloroform-d): δ 7.91 (d, J 2.4 Hz, 1H), 7.86 (s, 1H), 7.59-7.68 (m, 4H), 7.47-7.52 (m, 2H), 7.40-7.43 (m, 1H), 7.26-7.33 (m, 3H), 6.39-6.76 (m, 1H), 4.50-4.57 (m, 1H), 3.91-3.96 (m, 1H), 3.79-3.85 (m, 1H), 3.63-3.67 (m, 1H), 3.35 (s, 3H). ¹³C NMR (151 MHz, Chloroform-d) δ 169.1, 164.6, 151.3 (t, J=2.8 Hz), 140.3, 138.8, 138.2, 137.1, 136.4, 135.5, 131.1, 131.0, 130.8, 130.3, 128.7, 126.8, 124.2, 123.7, 123.3, 121.4, 118.6, 117.3, 115.5, 113.8, 70.2, 58.9, 51.6. LC-MS: (ESI) calcd for C₂₄H₂₁F₂N₂O₄S [M+H]⁺ 471.12, found 471.10.

N-(10-(But-2-yn-1-yl)-3-fluoro-11-oxo-10,11-dihydrodibenzo[b,f][1,4]thiazepin-7-yl)-3-methyl-2-oxo-2,3-dihydrobenzo[d]oxazole-5-carboxamide. ¹H NMR (400 MHz, Chloroform-d): δ 7.93 (d, J=2.8 Hz, 1H), 7.80-7.88 (m, 1H), 7.68-7.74 (m, 2H), 7.66-7.67 (m, 1H), 7.56-7.58 (m, 2H), 7.26-7.29 (m, 1H), 7.14-7.16 (m, 1H), 7.00-7.05 (m, 1H), 4.90-4.95 (m, 1H), 4.42-4.47 (m, 1H), 3.47 (s, 3H), 1.86 (t, J=4.4 Hz, 3H). ¹³C NMR (151 MHz, Chloroform-d) δ 167.6, 164.8, 164.0, 162.3, 154.4, 145.2, 140.6 (d, J=8.2 Hz), 139.9, 136.1, 134.9, 134.0 (d, J=9.1 Hz), 133.7 (d, J=3.3 Hz), 132.5, 130.7, 125.3, 124.2, 121.5, 121.3, 118.0, 117.9, 116.1, 116.0, 109.7, 108.1, 80.4, 74.6, 41.3, 28.4, 3.8. LC-MS: (ESI) calcd for C₂₆H₁₉FN₃O₄S [M+H]⁺ 488.11, found 488.10.

1-(Benzo[d]oxazol-5-yl)-3-(10-(but-2-yn-1-yl)-3-fluoro-11-oxo-10,11-dihydrodibenzo[b,f][1,4]thiazepin-7-yl) urea. ¹H NMR (400 MHz, Methanol-d4): δ 8.29 (s, 1H), 8.03 (d, J=2.4, Hz, 1H), 7.80 (d, J=2.0, Hz, 1H), 7.67-7.71 (m, 1H), 7.61-7.63 (m, 1H), 7.39-7.42 (m, 1H), 7.32 (d, J=2.0, Hz, 1H), 7.11-7.16 (m, 2H), 6.81 (t, J=8.8, Hz, 1H), 4.71-4.76 (m, 1H), 4.63-4.67 (m, 1H), 1.79 (s, 3H). ¹³C NMR (151 MHz, Methanol-d4) δ 167.9, 163.8, 162.1, 160.2, 153.7, 143.0, 138.1, 134.8, 133.6, 132.9 (d, J=9.3 Hz), 130.0, 125.3, 124.9, 121.8, 119.6, 117.3, 116.9, 115.4 (d, J=21.9 Hz), 114.4, 113.7, 79.3, 73.5, 39.8. 1.4. LC-MS: (ESI) calcd for C₂₅H₁₈FN₄O₃S [M+H]⁺ 473.11, found 473.10.

(E)-3-((10-(Hex-2-en-1-yl)-11-oxo-10,11-dihydrodibenzo[b,f][1,4]thiazepin-7-yl)amino) pyrazine-2-carbonitrile. ¹H NMR (400 MHz, Chloroform-d): δ 8.33 (d, J=2.4 Hz, 1H), 8.12 (d, J=2.4 Hz, 1H), 7.88 (d, J=2.4 Hz, 1H), 7.70-7.73 (m, 1H), 7.50-7.53 (m, 1H), 7.40-7.44 (m, 2H), 7.26-7.34 (m, 2H), 7.15 (s, 1H), 5.61-5.70 (m, 2H), 4.76-4.81 (m, 1H), 4.47-4.51 (m, 1H), 2.00-2.03 (m, 2H), 1.31-1.39 (m, 2H), 1.85 (d, J=7.2 Hz, 3H). ¹³C NMR (151 MHz, Chloroform-d) & 168.6, 153.0, 145.4, 139.9, 138.5, 138.3, 137.0, 136.0, 135.1, 134.5, 131.5, 131.0, 130.8, 128.7, 126.0, 125.1, 124.5, 121.5, 115.9, 114.6, 53.3, 34.3, 22.2, 13.6. LC-MS: (ESI) calcd for C₂₄H₂₂N₅OS [M+H]⁺ 428.16, found 428.10.

10-(But-2-yn-1-yl)-7-((3-(trifluoromethyl)pyridin-2-yl)amino)dibenzo[b,f][1,4]thiazepin-11 (10H)-one. ¹H NMR (400 MHz, Chloroform-d): δ 8.37 (d, J=4.8 Hz, 1H), 7.76-7.82 (m, 3H), 7.70 (d, J=4.8 Hz, 1H), 7.56-7.59 (m, 1H), 7.42-7.44 (m, 1H), 7.26-7.34 (m, 2H), 6.86-6.89 (m, 1H), 6.72 (s, 1H), 4.93-7.98 (m, 1H), 4.39-4.44 (m, 1H), 1.87 (d, J=2.0 Hz, 3H). ¹³C NMR (151 MHz, Chloroform-d) δ 168.5, 151.5, 151.3, 138.8, 138.6, 137.8, 137.2, 135.7 (q, J=5.2 Hz), 135.6, 131.9, 131.1, 131.0, 128.6, 125.0, 124.3, 121.8, 114.6, 79.9, 74.9, 41.2, 3.9. LC-MS: (ESI) calcd for C₂₃H₁₇F₃N₃OS [M+H]⁺ 440.11, found 440.10.

Methyl 2-((10-(but-2-yn-1-yl)-11-oxo-10,11-dihydrodibenzo[b,f][1,4]thiazepin-7-yl)amino) nicotinate. ¹H NMR (400 MHz, Chloroform-d): δ 10.23 (s, 1H), 8.38-8.40 (m, 1H), 8.23-8.25 (m, 1H), 7.98-7.99 (m, 1H), 7.66-7.78 (m, 4H), 7.43 (d, J=8.8 Hz, 1H), 7.26-7.33 (m, 2H), 6.75-6.78 (m, 1H), 4.92-4.97 (m, 1H), 4.43-4.44 (m, 1H), 3.93 (s, 3H), 1.86 (t, J=4.4 Hz, 3H). ¹³C NMR (151 MHz, Chloroform-d) δ 168.6, 167.9, 155.6, 153.1, 140.3, 138.8, 138.1, 137.9 (d, J=2.0 Hz), 135.4, 131.8, 131.1, 130.9, 128.5, 124.9, 124.1, 121.5, 114.0, 107.4, 79.8, 75.0, 52.4, 41.2, 3.9. LC-MS: (ESI) calcd for C₂₄H₂₀N₃O₃S [M+H]⁺ 430.12, found 430.10.

4-Azido-N-(11-oxo-10-(prop-2-yn-1-yl)-10,11-dihydrodibenzo[b,f][1,4]thiazepin-7-yl)benzamide. ¹H NMR (400 MHz, Chloroform-d) δ 8.02 (s, 1H), 7.95 (d, J=2.2 Hz, 1H), 7.85 (d, J 8.6 Hz, 2H), 7.82-7.71 (m, 1H), 7.71-7.60 (m, 2H), 7.41 (dt, J=7.8, 3.0 Hz, 1H), 7.35-7.25 (m, 2H), 7.09 (d, J=8.6 Hz, 2H), 4.94 (dd, J=17.3, 2.5 Hz, 1H), 4.55 (dd, J=17.3, 2.5 Hz, 1H), 2.36 (t, J=2.4 Hz, 1H). ¹³C NMR (101 MHz, Chloroform-d) δ 168.5, 164.7, 144.1, 139.5, 138.6, 137.3, 136.2, 136.1, 131.9, 131.3, 131.2, 130.7, 128.9, 128.7, 125.1, 124.2, 121.3, 119.3, 79.4, 72.5, 40.6. LC-MS: (ESI) calcd for C₂₃H₁₆N₅O₂S [M+H]⁺ 426.10, found 426.10.

N-(10-(But-2-yn-1-yl)-11-oxo-10,11-dihydrodibenzo[b,f][1,4]thiazepin-7-yl)-2-(7-(dimethylamino)-2-oxo-2H-chromen-4-yl) acetamide. ¹H NMR (400 MHz, Chloroform-d) δ 7.78-7.70 (m, 1H), 7.63 (d, J=8.8 Hz, 1H), 7.55-7.48 (m, 1H), 7.47-7.42 (m, 2H), 7.39 (dd, J=7.5, 1.6 Hz, 1H), 7.29 (dd, J=6.9, 1.9 Hz, 3H), 6.62 (dd, J=9.0, 2.6 Hz, 1H), 6.51 (d, J=2.5 Hz, 1H), 6.12 (s, 1H), 4.90 (dd, J=16.8, 2.7 Hz, 1H), 4.39 (dd, J=16.9, 2.6 Hz, 1H), 3.78 (s, 2H), 3.05 (s, 6H), 1.83 (t, J=2.3 Hz, 3H). LC-MS: (ESI) calcd for C₃₀H₂₆N₃O₄S [M+H]⁺ 524.16, found 524.30.

N-(11-Oxo-10-(prop-2-yn-1-yl)-10,11-dihydrodibenzo[b,f][1,4]thiazepin-7-yl)benzo[d]thiazole-5-carboxamide. ¹H NMR (600 MHz, Chloroform-d) δ 9.11 (s, 1H), 8.59 (d, J 2.4 Hz, 1H), 8.31 (s, 1H), 8.08-8.01 (m, 2H), 7.99 (dd, J=8.4, 1.7 Hz, 1H), 7.80-7.74 (m, 1H), 7.69 (d, J=1.4 Hz, 2H), 7.46-7.40 (m, 1H), 7.35-7.27 (m, 2H), 4.96 (dd, J=17.3, 2.5 Hz, 1H), 4.56 (dd, J=17.3, 2.5 Hz, 1H), 2.37 (t, J=2.5 Hz, 1H). ¹³C NMR (151 MHz, Chloroform-d) δ 168.5, 165.3, 155.9, 153.0, 139.6, 138.6, 137.3, 136.2, 136.1, 132.9, 131.9, 131.3, 131.2, 128.7, 125.1, 124.5, 124.2, 122.6, 122.0, 121.3, 79.4, 72.5, 40.6. LC-MS: (ESI) calcd for C₂₄H₁₆N₃O₂S₂ [M+H]⁺ 442.07, found 442.20.

N-(5-(But-2-yn-1-yl)-6-oxo-6, 11-dihydro-5H-dibenzo[b,e]azepin-2-yl)-4-methoxybenzamide. ¹H NMR (600 MHz, Chloroform-d) δ 7.86-7.80 (m, 4H), 7.75 (d, J=2.5 Hz, 1H), 7.58 (d, J=8.7 Hz, 1H), 7.42-7.32 (m, 2H), 7.25 (d, J=6.2 Hz, 1H), 7.16 (d, J=7.4 Hz, 1H), 6.99-6.93 (m, 2H), 4.89 (dd, J=16.9, 2.4 Hz, 1H), 4.58 (dd, J=17.0, 2.4 Hz, 1H), 4.18 (d, J 13.3 Hz, 1H), 3.87 (s, 3H), 3.54 (d, J=13.3 Hz, 1H), 1.84 (t, J=2.3 Hz, 3H). ¹³C NMR (151 MHz, Chloroform-d) δ 168.2, 165.2, 162.6, 142.1, 138.2, 136.3, 136.0, 132.3, 131.7, 131.1, 128.9, 127.0, 126.8, 126.1, 123.1, 118.8, 118.6, 114.0, 79.8, 75.1, 55.5, 40.1, 38.6, 3.8. LC-MS: (ESI) calcd for C₂₆H₂₃N₂O₃ [M+H]⁺ 411.17, found 411.20.

(E)-10-(Hex-2-en-1-yl)-7-(isoquinolin-3-ylamino)dibenzo[b,f][1,4]thiazepin-11 (10H)-one. ¹H NMR (400 MHz, Chloroform-d): δ 8.95 (s, 1H), 7.82-7.84 (d, J=8.0 Hz, 1H), 7.72-7.73 (d, J=4.0 Hz, 1H), 7.56-7.71 (m, 3H), 7.41-7.43 (m, 1H), 7.26-7.37 (m, 5H), 7.13 (s, 1H), 6.64 (s, 1H), 5.66-5.69 (m, 2H), 4.80-4.84 (m, 1H), 4.48-4.49 (m, 1H), 2.00-2.02 (m, 2H), 1.34-1.40 (m, 2H), 0.83-0.87 (t, J=8.0 Hz, 3H). ¹³C NMR (151 MHz, Chloroform-d) δ 168.8, 152.0, 150.8, 139.1, 138.9, 138.5, 138.4, 137.4, 137.3, 134.3, 131.4, 131.0, 130.8, 130.6, 128.6, 127.8, 126.4, 125.3, 125.2, 124.8, 124.2, 122.4, 119.6, 100.4, 53.2, 34.3, 22.3, 13.6. LC-MS: (ESI) calcd for C₂₈H₂₆N₃OS [M+H]⁺ 452.18, found 452.30.

2-(Benzo[d]oxazol-5-yl)-N-(10-(2-methoxyethyl)-11-oxo-10,11-dihydrodibenzo[b,f][1,4]thiazepin-7-yl) acetamide. ¹H NMR (400 MHz, Chloroform-d): δ 8.26 (s, 1H), 8.15 (s, 1H), 7.95-7.96 (d, J=4.0 Hz, 1H), 7.82-7.89 (m, 1H), 7.61-7.68 (m, 1H), 7.51-7.53 (d, J 8.0 Hz, 1H), 7.40-7.42 (d, J=8.0 Hz, 1H), 7.25-7.32 (m, 2H), 4.51-4.58 (m, 1H), 3.92-3.98 (m, 1H), 3.80-3.85 (m, 1H), 3.63-3.68 (m, 1H), 3.35 (s, 3H). ¹³C NMR (151 MHz, Chloroform-d) δ 169.1, 169.0, 153.3, 149.5, 140.7, 139.9, 138.8, 138.2, 137.0, 135.5, 131.1, 131.0, 130.9, 130.7, 128.6, 127.0, 126.6, 123.6, 121.4, 120.9, 111.6, 70.1, 58.8, 51.5, 44.4. LC-MS: (ESI) calcd for C₂₅H₂₂N₃O₄S [M+H]⁺ 460.14, found 446.10.

N-(10-(2-Methoxyethyl)-11-oxo-10,11-dihydrodibenzo[b,f][1,4]thiazepin-7-yl)-2-oxo-2,3-dihydrobenzo[d]oxazole-5-carboxamide. ¹H NMR (600 MHz, DMSO-d6) δ 10.40 (s, 1H), 8.16 (d, J 2.4 Hz, 1H), 7.80-7.71 (m, 2H), 7.64 (dd, J=5.3, 3.5 Hz, 2H), 7.63-7.57 (m, 1H), 7.56-7.49 (m, 1H), 7.44 (d, J=8.4 Hz, 1H), 7.40 (td, J=6.8, 6.1, 3.4 Hz, 2H), 4.58-4.50 (m, 1H), 3.85 (dt, J=14.0, 5.6 Hz, 1H), 3.58 (dt, J=10.1, 6.0 Hz, 1H), 3.52 (dd, J=10.1, 5.7 Hz, 1H), 3.21 (s, 3H). ¹³C NMR (151 MHz, DMSO-d6) δ 167.1, 164.3, 153.8, 145.2, 137.7, 137.5, 136.4, 135.0, 130.4, 130.3, 130.3, 130.0, 129.5, 128.3, 126.0, 123.0, 121.6, 120.9, 108.6, 108.5, 68.7, 57.5, 49.3. LC-MS: (ESI) calcd for C₂₄H₂₀N₃O₅S [M+H]⁺ 462.11, found 462.10.

N-(10-(2-Methoxyethyl)-11-oxo-10,11-dihydrodibenzo[b,f][1,4]thiazepin-7-yl)-3-methyl-2-oxo-2,3-dihydrobenzo[d]oxazole-5-carboxamide. ¹H NMR (400 MHz, Methanol-d4) δ 8.07 (s, 1H), 7.69-7.78 (m, 3H), 7.59-7.62 (m, 2H), 7.48-7.50 (m, 1H), 7.33-7.37 (m, 3H), 4.55-4.61 (m, 1H), 3.92-3.98 (m, 1H), 3.65-3.75 (m, 1H), 3.51-3.64 (m, 1H), 3.44 (s, 3H), 3.30 (s, 3H). ¹³C NMR (151 MHz, Methanol-d4) δ 169.9, 166.3, 154.9, 145.2, 139.3, 139.0, 138.1, 137.0, 136.8, 132.2, 130.8, 130.8, 130.7, 130.4, 128.5, 126.5, 124.2, 122.5, 121.7, 109.0, 107.9, 69.7, 57.6, 50.9, 27.2. LC-MS: (ESI) calcd for C₂₅H₂₂N₃O₅S [M+H]⁺ 476.13, found 476.10.

2-(2-(7-(Furan-3-carboxamido)-11-oxodibenzo[b,f][1,4]thiazepin-10(11H)-yl) ethoxy) acetic acid. ¹H NMR (400 MHz, Methanol-d4): δ 8.20 (s, 1H), 8.03 (s, 1H), 7.60-7.66 (m, 4H), 7.47-7.49 (d, J=8.0 Hz, 2H), 7.35-7.37 (m, 2H), 6.91 (s, 1H), 4.52 (m, 1H), 3.96-4.08 (m, 4H), 3.82-3.85 (m, 1H). ¹³C NMR (151 MHz, Methanol-d4) δ 172.8, 170.0, 162.1, 145.8, 144.0, 139.2, 138.9, 138.1, 136.7, 136.6, 130.8, 130.7, 130.4, 128.5, 126.6, 124.0, 122.6, 121.6, 108.5, 68.4, 67.8, 51.1. LC-MS: (ESI) calcd for C₂₂H₁₉N₂O₆S [M+H]⁺ 439.10, found 439.10.

6-(7-(Furan-3-carboxamido)-11-oxodibenzo[b,f][1,4]thiazepin-10(11H)-yl) hexanoic acid. ¹H NMR (400 MHz, Chloroform-d): δ 8.49 (s, 1H), 8.08 (s, 1H), 7.78 (d, J=2.4, Hz, 1H), 7.61 (t, J=6.8, Hz, 2H), 7.41 (s, 1H), 7.29 (d, J=1.6, Hz, 1H), 7.20-7.26 (m, 3H), 6.78 (d, J=1.2, Hz, 1H), 4.71-4.74 (m, 1H), 3.50-3.53 (m, 1H), 2.27 (t, J=6.8, Hz, 2H), 1.54-1.65 (m, 4H), 1.36-1.43 (m, 2H). ¹³C NMR (151 MHz, Chloroform-d) δ 178.3, 169.2, 161.3, 145.7, 144.0, 138.9, 138.0, 137.4, 131.2, 131.0, 130.7, 128.6, 126.2, 124.6, 122.6, 121.7, 108.5, 50.5, 33.8, 27.6, 26.2, 24.3. LC-MS: (ESI) calcd for C₂₄H₂₃N₂O₅S [M+H]: 451.13, found 451.10.

(E)-10-(Hex-2-en-1-yl)-7-((4-methoxypyridin-2-yl)amino)dibenzo[b,f][1,4]thiazepin-11 (10H)-one. ¹H NMR (400 MHz, Chloroform-d): δ 8.00 (d, J=6.0 Hz, 1H), 7.71 (t, J=7.6 Hz, 1H), 7.57 (s, 1H), 7.41 (d, J=7.6 Hz, 1H), 7.29-7.35 (m, 3H), 7.19-7.25 (m, 1H), 6.39 (d, J=5.6 Hz, 1H), 6.29 (s, 1H), 5.60-5.71 (m, 2H), 4.76-4.81 (m, 1H), 4.43-4.48 (m, 1H), 3.81 (s, 3H), 1.96-2.06 (m, 2H), 1.33-1.38 (m, 2H), 0.84 (t, J=7.2 Hz, 3H). ¹³C NMR (151 MHz, Chloroform-d) δ 170.0, 168.7, 167.7, 156.5, 148.2, 138.8, 138.4, 138.2, 138.1, 137.1, 134.3, 131.4, 131.0, 130.6, 128.6, 126.3, 125.2, 123.5, 120.7, 103.6, 93.4, 55.3, 53.2, 34.3, 22.3, 13.6. LC-MS: (ESI) calcd for C₂₅H₂₆N₃O₂S [M+H]⁺ 432.18, found 432.10.

(I)-10-(Hex-2-en-1-yl)-7-((4-(trifluoromethyl)pyridin-2-yl)amino)dibenzo[b,f][1,4]thiazepin-11 (10H)-one. ¹H NMR (400 MHz, Chloroform-d): δ 8.14-8.16 (d, J=8.0 Hz, 1H), 7.71-7.73 (d, J=8.0 Hz, 1H), 7.59 (s, 1H), 7.41-7.46 (m, 2H), 7.11 (s, 1H), 6.99-7.00 (m, 1H), 5.30-5.68 (m, 2H), 4.79-4.84 (m, 1H), 4.46-4.82 (m, 1H), 2.00-2.04 (m, 2H), 1.33-1.39 (m, 2H), 0.82-0.85 (t, J=8.0 Hz, 3H). ¹³C NMR (151 MHz, Chloroform-d) δ 168.5, 154.7, 138.3, 138.1, 137.9, 134.7, 131.6, 131.1, 131.0, 128.9, 126.8, 126.3, 125.0, 123.1, 109.8, 106.7, 53.3, 34.3, 22.2, 13.5. LC-MS: (ESI) calcd for C₂₅H₂₃F₃N₃OS [M+H]: 470.15, found 470.20.

(E)-10-(Hex-2-en-1-yl)-7-((4-isopropylpyridin-2-yl)amino)dibenzo[b,f][1,4]thiazepin-11 (10H)-one. ¹H NMR (400 MHz, Chloroform-d): δ 12.03 (s, 1H), 7.77 (d, J=6.4, Hz, 1H), 7.35 (m, 1H), 7.55 (d, J=2.4 Hz, 1H), 7.41-7.46 (m, 2H), 7.31-7.35 (m, 2H), 7.22-7.26 (m, 1H), 6.90 (s, 1H), 6.76-6.77 (m, 1H), 5.64-5.68 (m, 2H), 4.79-4.82 (m, 1H), 4.46-4.51 (m, 1H), 2.85-2.90 (m, 1H), 2.01-2.03 (m, 2H), 1.36-1.43 (m, 2H), 1.21-1.25 (m, 6H), 0.85 (t, J=7.2, Hz, 3H). ¹³C NMR (151 MHz, Chloroform-d) δ 168.4, 167.3, 152.8, 141.7, 138.3, 138.1, 137.8, 137.0, 134.6, 134.4, 131.6, 131.0, 131.0, 128.9, 127.3, 126.8, 125.0, 124.3, 113.0, 107.6, 53.3, 34.7, 34.3, 22.4, 22.3, 22.2, 13.6. LC-MS: (ESI) calcd for C₂₇H₃₀N₃OS [M+H]⁺ 444.21, found 444.40.

(E)-7-((4-Cyclopropylpyridin-2-yl)amino)-10-(hex-2-en-1-yl)dibenzo[b,f][1,4]thiazepin-11(10H)-one. ¹H NMR (400 MHz, Methanol-d4): δ 7.71 (d, J=6.4, Hz, 1H), 7.63-7.67 (m, 3H), 7.49-7.51 (m, 1H), 7.37-7.40 (m, 3H), 6.88 (s, 1H), 6.70-6.72 (m, 1H), 5.71 (s, 1H), 5.62 (s, 1H), 4.91-4.97 (m, 2H), 4.44-4.48 (m, 1H), 1.99-2.04 (m, 3H), 1.30-1.39 (m, 2H), 1.26-1.29 (m, 2H), 0.97-1.00 (m, 2H), 0.84 (t, J=7.2 Hz, 3H). ¹³C NMR (151 MHz, Methanol-d4) δ 169.2, 165.2, 151.0, 141.4, 138.3, 138.0, 137.9, 136.0, 134.6, 134.3, 131.1, 130.9, 130.7, 128.8, 127.6, 127.3, 124.7, 124.5, 111.8, 108.8, 52.6, 34.0, 21.9, 15.7, 12.5, 11.4. LC-MS: (ESI) calcd for C₂₇H₂₈N₃OS [M+H]⁺ 442.20, found 442.30.

(E)-7-((4-Fluoropyridin-2-yl)amino)-10-(hex-2-en-1-yl)dibenzo[b,f][1,4]thiazepin-11(10H)-one. ¹H NMR (400 MHz, Methanol-d4): δ 8.15 (dd, J=14.8, 6.0 Hz, 1H), 7.70-7.72 (m, 1H), 7.58 (d, J=2.4 Hz, 1H), 7.26-7.43 (m, 5H), 6.74 (s, 1H), 6.54 (dd, J=3.6, 2.4 Hz, 1H), 6.45-6.48 (m, 1H), 5.64-5.67 (m, 2H), 4.87 (d, J=14 Hz, 1H), 4.48 (d, J=4.8 Hz, 1H), 1.99-2.01 (m, 2H), 1.32-1.39 (m, 2H), 0.84 (t, J=7.6 Hz, 3H). ¹³C NMR (151 MHz, Chloroform-d) δ 170.9, 169.2, 168.7, 157.5 (d, J=10.4 Hz), 150.6, 138.7, 138.5, 138.4, 137.7, 137.2, 134.4, 131.5, 131.0, 130.7, 128.6, 126.3, 125.1, 123.8, 121.0, 104.3 (d, J=18.0 Hz), 95.5 (d, J=21.2 Hz), 53.3, 34.3, 22.3, 13.6. LC-MS: (ESI) calcd for C₂₄H₂₃FN₃OS [M+H]⁺ 420.16, found 420.30.

3-(1-Cyanoethyl)-N-(11-oxo-10-(2-(prop-2-yn-1-yloxy)ethyl)-10,11-dihydrodibenzo[b,f][1,4]thiazepin-7-yl)benzamide. ¹H NMR (400 MHz, Chloroform-d): δ 8.17 (s, 1H), 7.93 (d, J=2.4 Hz, 1H), 7.84 (s, 1H), 7.78 (d, J=2.4 Hz, 1H), 7.61-7.65 (m, 2H), 7.55 (d, J=2.0 Hz, 1H), 7.47-7.51 (m, 2H), 7.38-7.41 (m, 1H), 7.23-7.30 (m, 2H), 4.53-4.59 (m, 1H), 4.15 (d, J=2.4 Hz, 2H), 3.94-4.00 (m, 3H), 3.79-3.83 (m, 1H), 2.42 (t, J=2.4 Hz, 1H), 1.66 (d, J=7.6 Hz, 3H). ¹³C NMR (151 MHz, Chloroform-d) δ 169.2, 165.1, 140.1, 138.8, 138.2, 138.0, 137.1, 135.7, 135.5, 131.1, 131.0, 130.8, 130.3, 129.7, 128.7, 126.8, 126.7, 125.8, 124.3, 121.4, 121.1, 79.4, 74.7, 67.6, 58.4, 51.5, 31.2, 21.3. LC-MS: (ESI) calcd for C₂₈H₂₄N₃O₃S [M+H]⁺ 482.16, found 482.10.

N-(10-Allyl-11-oxo-10,11-dihydrodibenzo[b,f][1,4]thiazepin-7-yl)benzo[d]thiazole-5-carboxamide. ¹H NMR (400 MHz, Chloroform-d) δ 9.10 (s, 1H), 8.60 (s, 1H), 8.45 (s, 1H), 8.09-7.94 (m, 3H), 7.72-7.63 (m, 2H), 7.42-7.33 (m, 2H), 7.29 (s, 1H), 6.08-5.94 (m, 1H), 5.30 (d, J=17.2 Hz, 1H), 5.20 (d, J=10.3 Hz, 1H), 4.83 (dd, J=15.6, 5.4 Hz, 1H), 4.56 (dd, J=15.6, 5.9 Hz, 1H). ¹³C NMR (151 MHz, Chloroform-d) δ 168.8, 165.4, 156.0, 155.5, 152.8, 139.7, 138.6, 138.1, 137.4, 136.7, 135.9, 133.3, 133.0, 131.4, 131.1, 130.9, 128.7, 126.5, 125.8, 125.8, 124.6, 124.2, 122.5, 121.9, 121.2, 117.5, 54.0. LC-MS: (ESI) calcd for C₂₄H₁₈N₃O₂S₂ [M+H]⁺ 444.09, found 444.00.

N-(10-(But-2-yn-1-yl)-11-oxo-10,11-dihydrodibenzo[b,f][1,4]thiazepin-7-yl) furan-3-carboxamide. ¹H NMR (400 MHz, Chloroform-d) δ 8.07-8.02 (m, 1H), 7.86 (d, J=2.5 Hz, 1H), 7.79-7.73 (m, 1H), 7.69 (d, J=8.7 Hz, 1H), 7.61 (dd, J=8.7, 2.5 Hz, 1H), 7.55 (s, 1H), 7.49 (d, J=1.8 Hz, 1H), 7.40 (td, J=6.3, 5.7, 1.9 Hz, 1H), 7.34-7.28 (m, 2H), 6.71 (d, J=2.0 Hz, 1H), 4.94 (dd, J=17.0, 2.4 Hz, 1H), 4.43 (dd, J=17.0, 2.4 Hz, 1H), 1.85 (t, J=2.4 Hz, 3H). ¹³C NMR (151 MHz, Chloroform-d) δ 168.6, 160.8, 145.5, 144.1, 139.6, 138.6, 137.5, 135.9, 135.7, 131.7, 131.0, 128.6, 125.1, 124.0, 122.7, 121.2, 108.4, 80.2, 74.7, 41.3, 3.8. LC-MS: (ESI) calcd for C₂₂H₁₇N₂O₃S [M+H]⁺ 389.10, found 389.10.

4-Methoxy-N-(6-oxo-5-(2-(prop-2-yn-1-yloxy)ethyl)-6,11-dihydro-5H-dibenzo[b,e]azepin-2-yl)benzamide. ¹H NMR (600 MHz, Chloroform-d) § 7.87 (s, 1H), 7.86-7.79 (m, 2H), 7.76 (dd, J=7.8, 1.4 Hz, 1H), 7.71 (d, J=2.4 Hz, 1H), 7.37-7.27 (m, 3H), 7.23 (t, J=6.9 Hz, 1H), 7.14 (d, J=6.1 Hz, 1H), 6.98-6.93 (m, 2H), 4.77 (ddd, J=13.8, 7.1, 4.3 Hz, 1H), 4.27 (d, J=13.1 Hz, 1H), 4.12 (t, J=2.5 Hz, 2H), 3.96-3.88 (m, 2H), 3.86 (s, 3H), 3.79 (ddd, J=10.0, 7.1, 4.2 Hz, 1H), 3.50 (d, J=13.2 Hz, 1H), 2.39 (t, J=2.4 Hz, 1H). ¹³C NMR (151 MHz, Chloroform-d) δ 168.9, 165.2, 162.6, 142.6, 139.8, 136.0, 132.9, 131.5, 130.6, 128.9, 126.9, 126.8, 126.0, 124.5, 118.8, 118.6, 114.0, 79.4, 74.7, 67.1, 58.0, 55.5, 49.6. LC-MS: (ESI) calcd for C₂₇H₂₅N₂O₄ [M+H]⁺ 441.18, found 441.10.

3-(Fluoromethoxy)-N-(10-(2-methoxyethyl)-11-oxo-10,11-dihydrodibenzo[b,f][1,4]thiazepin-7-yl)benzamide. ¹H NMR (400 MHz, DMSO-d6): δ 7.91 (d, J=8.0 Hz, 1H), 7.86 (s, 1H), 7.67 (d, J=1.6 Hz, 1H), 7.40-7.62 (m, 7H), 7.26-7.30 (m, 3H), 5.82 (s, 1H), 5.69 (s, 1H), 4.51-4.57 (m, 1H), 3.90-3.96 (m, 1H), 3.79-3.84 (m, 1H), 3.62-3.67 (m, 1H), 3.34 (s, 3H). ¹³C NMR (151 MHz, Chloroform-d) δ 169.1, 165.1, 157.0, 140.1, 138.8, 138.2, 137.1, 136.2, 135.7, 131.1, 131.0, 130.7, 130.2, 128.6, 126.8, 124.1, 121.7, 121.3, 120.3, 115.5, 70.1, 58.9, 51.6. LC-MS: (ESI) calcd for C₂₄H₂₂FN₂O₄S [M+H]⁺ 452.50, found 452.12.

2,2-Difluoro-N-(10-(2-methoxyethyl)-11-oxo-10,11-dihydrodibenzo[b,f][1,4]thiazepin-7-yl)benzo[d][1,3]dioxole-5-carboxamide. ¹H NMR (400 MHz, Chloroform-d): δ 7.91 (d, J=7.2 Hz, 2H), 7.64-7.66 (m, 1H), 7.57-7.61 (m, 3H), 7.50 (d, J=8.4 Hz, 1H), 7.39-7.42 (m, 2H), 7.13 (d, J=8.8 Hz, 1H), 4.50-4.56 (m, 1H), 3.91-3.97 (m, 1H), 3.79-3.84 (m, 1H), 3.62-3.67 (m, 1H), 3.45 (s, 3H). ¹³C NMR (151 MHz, Chloroform-d) & 169.2, 164.1, 146.3, 144.1, 140.2, 138.8, 138.2, 137.1, 135.6, 131.7, 131.1, 131.0, 130.8, 128.7, 126.8, 124.2, 123.3, 121.4, 109.4, 109.1, 70.1, 58.9, 51.7. LC-MS: (ESI) calcd for C₂₄H₁₉F₂N₂O₅S [M+H]⁺ 484.47, found 484.09.

(E)-10-(Hex-2-en-1-yl)-7-((3-methylpyrazin-2-yl)amino)dibenzo[b,f][1,4]thiazepin-11 (10H)-one. ¹H NMR (400 MHz, Chloroform-d): δ 7.71-7.76 (m, 2H), 7.54-7.55 (d, J=4.0 Hz, 1H), 7.41-7.46 (m 2H), 7.31-7.37 (m, 2H), 7.20-7.26 (m, 2H), 6.71-6.72 (d, J=4.0 Hz, 1H), 5.64-5.68 (m, 2H), 4.77-4.82 (m, 1H), 4.47-4.52 (m, 1H), 2.38 (s, 3H), 1.99-2.03 (m, 2H), 1.33-1.40 (m, 2H), 0.83-0.86 (t, J=8.0 Hz, 3H). ¹³C NMR (151 MHz, Chloroform-d) δ 168.7, 149.8, 140.9, 139.3, 138.8, 138.5, 138.1, 137.4, 136.7, 134.7, 134.2, 131.4, 131.0, 130.6, 128.6, 125.9, 125.2, 123.3, 120.5, 53.3, 34.3, 22.3, 20.4, 13.6. LC-MS: (ESI) calcd for C₂₄H₂₅N₄OS [M+H]⁺ 417.54, found 417.20.

(E)-10-(Hex-2-en-1-yl)-7-((3-methoxypyrazin-2-yl)amino)dibenzo[b,f][1,4]thiazepin-11 (10H)-one. ¹H NMR (400 MHz, Chloroform-d): δ 7.71-7.76 (m, 2H), 7.54-7.55 (d, J=4.0 Hz, 1H), 7.41-7.46 (m 2H), 7.31-7.37 (m, 2H), 7.20-7.26 (m, 2H), 6.71-6.72 (d, J=4.0 Hz, 1H), 5.64-5.68 (m, 2H), 4.77-4.82 (m, 1H), 4.47-4.52 (m, 1H), 2.38 (s, 3H), 1.99-2.03 (m, 2H), 1.33-1.40 (m, 2H), 0.83-0.86 (t, J=8.0 Hz, 3H). ¹³C NMR (151 MHz, Chloroform-d) δ 168.7, 148.4, 141.7, 138.9, 138.5, 137.7, 137.4, 136.8, 134.2, 133.1, 131.4, 131.0, 130.6, 129.7, 128.5, 126.0, 125.2, 122.3, 119.5, 53.8, 53.3, 34.3, 22.2, 13.6. LC-MS: (ESI) calcd for C₂₄H₂₅N₄O₂S [M+H]⁺ 433.54, found 433.20.

(E)-7-((3,5-Dimethylpyridin-2-yl)amino)-10-(hex-2-en-1-yl)dibenzo[b,f][1,4]thiazepin-11 (10H)-one. ¹H NMR (400 MHz, Chloroform-d): δ10.22 (s, 1H), 7.66-7.75 (m, 3H), 7.26-7.39 (m, 4H), 7.14 (d, J=2.4 Hz, 1H), 7.00-7.02 (m, 1H), 5.62-5.65 (m, 2H), 4.75-4.80 (m, 1H), 4.44-4.50 (m, 1H), 2.34 (s, 3H), 1.97-2.02 (m, 2H), 1.95 (s, 3H), 1.32-1.37 (m, 2H), 1.85 (d, J=7.2 Hz, 3H). ¹³C NMR (151 MHz, Chloroform-d) δ 168.5, 149.3, 147.8, 139.9, 138.3, 138.2, 137.0, 136.9, 135.2, 134.5, 131.5, 131.0, 130.8, 128.8, 127.0, 126.4, 125.1, 124.5, 122.2, 53.2, 34.3, 22.2, 19.2, 17.4, 13.6. LC-MS: (ESI) calcd for C₂₆H₂₈N₃OS [M+H]⁺ 430.59, found 430.20.

10-(But-2-yn-1-yl)-7-((3-ethynylpyridin-2-yl)amino)dibenzo[b,f][1,4]thiazepin-11 (10H)-one. ¹H NMR (400 MHz, Chloroform-d): δ 8.21 (s, 1H), 7.88 (s, 1H), 7.77 (d, J=7.2 Hz, 1H), 7.65-7.72 (m, 3H), 7.43 (d, J=7.2 Hz, 1H), 7.24-7.33 (m, 3H), 6.73-6.76 (m, 1H), 4.94 (d, J=17.2 Hz, 1H), 4.42 (d, J=17.2 Hz, 1H), 3.57 (s, 1H), 1.86 (s, 3H). ¹³C NMR (151 MHz, Chloroform-d) & 168.5, 155.3, 147.9, 140.7, 138.7, 138.0, 137.9, 137.8, 135.6, 131.8, 131.1, 130.9, 128.5, 125.1, 122.9, 120.4, 114.7, 103.7, 85.4, 79.9, 78.6, 75.0, 41.1, 3.9. LC-MS: (ESI) calcd for C₂₄H₁₈N₃OS [M+H]⁺ 496.48, found 396.10.

10-(But-2-yn-1-yl)-7-((3-fluoro-4-methylpyridin-2-yl)amino)dibenzo[b,f][1,4]thiazepin-11 (10H)-one. ¹H NMR (400 MHz, Chloroform-d): δ7.88 (d, J=6.0 Hz, 2H), 7.77 (d, J=7.2 Hz, 1H), 7.63-7.68 (m, 2H), 7.43 (d, J=7.2 Hz, 1H), 7.26-7.33 (m, 2H), 6.58-6.64 (m, 2H), 4.91-4.96 (m, 1H), 4.39-4.43 (m, 1H), 2.28 (s, 3H), 1.86 (s, 3H). ¹³C NMR (151 MHz, Chloroform-d) δ 168.6, 146.7, 145.0, 144.2, 141.7 (d, J=7.5 Hz), 138.8, 138.1, 137.9, 137.5, 135.7, 132.1 (d, J=13.0 Hz), 131.8, 131.1, 130.9, 128.5, 125.1, 122.3, 119.7, 117.9, 79.8, 75.0, 41.1, 13.9, 3.9. LC-MS: (ESI) calcd for C₂₃H₁₉FN₃OS [M+H]⁺ 403.47, found 403.12.

10-(But-2-yn-1-yl)-7-((3-fluoro-5-methylpyridin-2-yl)amino)dibenzo[b,f][1,4]thiazepin-11 (10H)-one. ¹H NMR (400 MHz, Chloroform-d): δ7.86-7.90 (m, 2H), 7.76-7.78 (m, 1H), 7.62-7.67 (m, 1H), 7.59-7.60 (m, 1H), 7.42 (d, J=7.6 Hz, 1H), 7.26-7.33 (m, 2H), 7.14 (d, J=11.6 Hz, 1H), 6.51 (d, J=2.0 Hz, 1H), 4.91-4.96 (m, 1H), 4.39-4.44 (m, 1H), 2.27 (s, 3H), 1.86 (s, 3H). ¹³C NMR (151 MHz, Chloroform-d) & 168.6, 147.7, 146.0, 142.3 (d, J=9.9 Hz), 142.0 (d, J=5.5 Hz), 138.8, 138.3, 137.9, 137.3, 135.7, 131.8, 131.1, 130.9, 128.5, 125.4, 125.1, 122.5, 122.4, 121.8, 119.3, 79.8, 75.0, 41.1, 17.5, 3.9. LC-MS: (ESI) calcd for C₂₃H₁₉FN₃OS [M+H]⁺ 404.47, found 404.20.

10-(But-2-yn-1-yl)-7-((3-fluoro-6-methylpyridin-2-yl)amino)dibenzo[b,f][1,4]thiazepin-11 (10H)-one. ¹H NMR (400 MHz, Chloroform-d): δ 7.86 (d, J=8.0, 1H), 7.73-7.77 (m, 2H), 7.65 (d, J=8.0, 1H), 7.43 (d, J=8.0, 1H), 7.15-7.20 (m, 2H), 6.55-6.60 (m, 2H), 4.91-4.96 (m, 1H), 4.39-4.45 (m, 1H), 2.46 (s, 3H), 1.86 (t, J=2.4 Hz, 3H). ¹³C NMR (151 MHz, Chloroform-d) δ 168.6, 151.6, 146.1, 144.4, 143.3 (d, J=9.5 Hz), 138.8, 138.2, 137.9, 137.3, 135.6, 131.8, 131.1, 130.9, 128.5, 125.1, 121.9, 121.7, 121.5, 119.3, 114.2, 79.9, 75.0, 41.2, 23.7, 3.9. LC-MS: (ESI) calcd for C₂₃H₁₉FN₃OS [M+H]⁺ 404.47, found 404.20.

2-((10-(But-2-yn-1-yl)-11-oxo-10,11-dihydrodibenzo[b,f][1,4]thiazepin-7-yl)amino)-3-methylisonicotinonitrile. ¹H NMR (400 MHz, Chloroform-d): δ 8.50 (s, 1H), 8.18 (d, J=5.2 Hz, 1H), 7.97 (d, J=2.4 Hz, 1H), 7.75 (d, J=2.4 Hz, 1H), 7.61-7.64 (m, 2H), 7.50-7.52 (m, 1H), 7.40-7.43 (m, 2H), 7.16 (d, J=5.2 Hz, 1H), 4.80-4.85 (m, 1H), 4.65-4.70 (m, 1H), 2.44 (s, 3H), 1.78 (s, 3H). LC-MS: (ESI) calcd for C₂₄H₁₉N₄OS [M+H]⁺ 411.49, found 411.20.

10-(But-2-yn-1-yl)-7-(pyridazin-3-ylamino)dibenzo[b,f][1,4]thiazepin-11 (10H)-one. 1H NMR (400 MHz, Chloroform-d): δ 8.73 (d, J=4.4 Hz, 1H), 7.71-7.79 (m, 1H), 7.68-7.70 (m, 2H), 7.40-7.44 (m, 2H), 7.26-7.35 (m, 3H), 7.03-7.06 (m, 1H), 4.93-4.98 (m, 1H), 4.40-4.45 (m, 1H), 1.86 (t, J=4.4 Hz, 3H). ¹³C NMR (151 MHz, Chloroform-d) δ 168.4, 157.4, 145.4, 138.9, 138.5, 137.7, 137.6, 136.2, 131.1, 131.0, 128.7, 127.9, 125.5, 123.7, 121.2, 114.2, 80.1, 74.8, 41.2, 3.9. LC-MS: (ESI) calcd for C₂₁H₁₇N₄OS [M+H]⁺ 373.44, found 373.10.

N-(10-(But-2-yn-1-yl)-11-oxo-10,11-dihydrodibenzo[b,f][1,4]thiazepin-7-yl)-4-(difluoromethoxy)benzamide. ¹H NMR (400 MHz, Chloroform-d): δ9.11 (s, 1H), 8.58 (d, J=1.2 Hz, 1H), 8.21 (s, 1H), 8.05-8.08 (m, 1H), 7.98-8.01 (m, 2H), 7.74-7.79 (m, 2H), 7.68-7.71 (m, 1H), 7.13-7.16 (m, 1H), 6.98-7.03 (m, 1H), 4.90-4.95 (m, 1H), 4.42-4.47 (m, 1H), 1.86 (t, J=2.0 Hz, 3H). ¹³C NMR (151 MHz, Chloroform-d) δ 168.5, 164.7, 153.9, 139.9, 138.5, 137.6, 135.9, 131.8, 131.3, 131.1, 129.1, 128.7, 125.2, 124.1, 121.2, 119.3, 117.1, 115.3, 113.6, 80.2, 74.7, 41.2, 3.8. LC-MS: (ESI) calcd for C₂₅H₁₉F₂N₂O₃S [M+H]⁺ 464.48, found 464.10.

N-(10-(But-2-yn-1-yl)-11-oxo-10,11-dihydrodibenzo[b,f][1,4]thiazepin-7-yl)-4-(fluoromethoxy)benzamide. ¹H NMR (400 MHz, Chloroform-d): δ 7.84-7.86 (m, 4H), 7.62-7.76 (m, 3H), 7.40-7.7.43 (m, 1H), 7.26-7.33 (m, 2H), 7.15 (d, J=8.8 Hz, 1H), 5.84 (s, 1H), 5.70 (s, 1H), 4.91-4.96 (m, 1H), 4.41-4.46 (m, 1H), 1.86 (t, J=4.4 Hz, 3H). ¹³C NMR (151 MHz, Chloroform-d) δ 168.5, 164.9, 159.5, 139.8, 138.5, 137.6, 136.0, 135.8, 131.8, 131.1, 129.3, 129.1, 128.6, 125.1, 124.0, 121.1, 116.4, 100.7, 99.2, 80.2, 74.7, 41.2, 3.8. LC-MS: (ESI) calcd for C₂₅H₂₀FN₂O₃S [M+H]⁺ 447.49, found 447.10.

N-(10-(But-2-yn-1-yl)-11-oxo-10,11-dihydrodibenzo[b,f][1,4]thiazepin-7-yl)-6-methoxynicotinamide. ¹H NMR (400 MHz, Chloroform-d): δ 8.49 (s, 1H), 8.18 (s, 1H), 8.05-8.08 (m, 1H), 7.93 (d, J=2.4 Hz, 1H), 7.68-7.73 (m, 2H), 7.62-7.64 (m, 1H), 7.38-7.40 (m, 1H), 7.28-7.30 (m, 2H), 6.78 (d, J=8.8 Hz, 1H), 4.91-495 (m, 1H), 4.40-4.45 (m, 1H), 3.99 (s, 3H), 1.85 (s, 3H). ¹³C NMR (151 MHz, Chloroform-d) δ 168.5, 166.3, 164.0, 146.7, 139.8, 138.5, 137.8, 137.5, 135.9, 135.8, 131.8, 131.1, 128.6, 125.1, 124.2, 123.5, 121.3, 111.1, 80.2, 74.7, 54.1, 41.2, 3.8. LC-MS: (ESI) calcd for C₂₄H₂₀N₃O₃S [M+H]⁺ 430.49, found 430.10.

(E)-N-(10-(But-2-en-1-yl)-11-oxo-10,11-dihydrodibenzo[b,f][1,4]thiazepin-7-yl)-4-methoxybenzamide. ¹H NMR (400 MHz, Chloroform-d) δ 7.87 (d, J=2.5 Hz, 1H), 7.83-7.77 (m, 2H), 7.73 (d, J=6.1 Hz, 1H), 7.72-7.68 (m, 1H), 7.62 (ddd, J=8.9, 6.8, 2.6 Hz, 1H), 7.44-7.35 (m, 2H), 7.33-7.26 (m, 2H), 7.01-6.93 (m, 2H), 5.77-5.57 (m, 2H), 4.80-4.70 (m, 1H), 4.58-4.43 (m, 1H), 3.87 (s, 3H), 1.76-1.64 (m, 3H). LC-MS: (ESI) calcd for C₂₅H₂₃N₂O₃S [M+H]⁺ 431.14, found 431.20.

N-(5-(But-2-yn-1-yl)-6,11-dioxo-6,11-dihydro-5H-dibenzo[b,e]azepin-2-yl)-4-methoxybenzamide. ¹H NMR (400 MHz, Chloroform-d) δ 8.21 (d, J=7.6 Hz, 1H), 8.09 (dd, J=8.9, 2.7 Hz, 1H), 7.95 (s, 1H), 7.83 (dd, J=8.7, 5.5 Hz, 3H), 7.77-7.48 (m, 4H), 6.97 (d, J=8.7 Hz, 2H), 4.69 (p, J=2.6 Hz, 2H), 3.87 (s, 3H), 1.89 (q, J=4.5, 3.4 Hz, 3H). LC-MS: (ESI) calcd for C₂₆H₂₁N₂O₄ [M+H]⁺ 425.14, found 425.20.

N-(5-(But-2-yn-1-yl)-11-hydroxy-6-oxo-6,11-dihydro-5H-dibenzo[b,e]azepin-2-yl)-4-methoxybenzamide. ¹H NMR (400 MHz, Chloroform-d) δ 7.93-7.74 (m, 5H), 7.61-7.50 (m, 3H), 7.42 (td, J=7.6, 1.4 Hz, 1H), 7.00-6.92 (m, 2H), 5.31 (s, 1H), 4.81 (dt, J=17.0, 2.4 Hz, 1H), 4.52 (dq, J=17.0, 2.3 Hz, 1H), 3.87 (s, 3H), 1.83 (t, J=2.4 Hz, 3H). LC-MS: (ESI) calcd for C₂₆H₂₃N₂O₄ [M+H]⁺ 427.16, found 427.20.

5-(But-2-yn-1-yl)-2-((3-methylpyridin-2-yl)amino)-5H-dibenzo[b,e]azepin-6 (11H)-one. 1H NMR (400 MHz, Chloroform-d): δ 7.91 (d, J=5.6 Hz, 1H), 7.86 (d, J=7.6 Hz, 1H), 7.74 (d, J=7.2 Hz, 1H), 7.59 (d, J=8.8 Hz, 1H), 7.35-7.39 (m, 1H), 7.26-7.31 (m, 1H), 7.15 (d, J=7.2 Hz, 1H), 6.93-7.03 (m, 2H), 6.91 (d, J=2.4 Hz, 1H), 4.88-4.93 (m, 1H), 4.53-4.58 (m, 1H), 4.16-4.20 (m, 1H), 3.48-3.52 (m, 1H), 1.92 (s, 3H), 1.83 (t, J=2.0 Hz, 3H). ¹³C NMR (151 MHz, Chloroform-d) δ 168.1, 151.5, 145.9, 141.7, 138.5, 137.8, 136.0, 135.8, 132.1, 131.9, 131.2, 127.1, 126.1, 125.5, 123.2, 121.4, 121.2, 115.6, 80.0, 74.9, 40.1, 38.6, 19.5, 3.8. LC-MS: (ESI) calcd for C₂₄H₂₂N₃O [M+H]⁺ 368.44, found 368.20.

2-((5-(But-2-yn-1-yl)-6-oxo-6,11-dihydro-5H-dibenzo[b,e]azepin-2-yl)amino) nicotinonitrile. ¹H NMR (400 MHz, Chloroform-d): δ 8.38 (d, J=1.6 Hz, 1H), 7.85 (d, J=6.8 Hz, 1H), 7.78-7.80 (m, 1H), 7.62 (d, J=8.4 Hz, 1H), 7.48-7.51 (m, 2H), 7.33-7.35 (m, 1H), 7.26-7.29 (m, 1H), 7.17-7.18 (m, 1H), 6.95 (s, 1H), 6.80-6.83 (m, 1H), 4.89-4.94 (m, 1H), 4.54-4.59 (m, 1H), 4.20-4.23 (m, 1H), 3.52-3.56 (m, 1H), 1.85 (t, J=2.0 Hz, 3H). ¹³C NMR (151 MHz, Chloroform-d) δ 168.2, 155.8, 152.3, 142.0, 141.7, 138.1, 136.2, 132.4, 131.7, 131.1, 127.0, 126.1, 123.1, 119.6, 119.4, 116.2, 114.4, 93.3, 79.7, 75.1, 40.1, 38.9, 3.8. LC-MS: (ESI) calcd for C₂₄H₁₉N₄O [M+H]⁺ 379.43, found 379.00.

5-(But-2-yn-1-yl)-2-((3-fluoropyridin-2-yl)amino)-5H-dibenzo[b,e]azepin-6 (11H)-one. 1H NMR (400 MHz, Chloroform-d): δ 8.02 (d, J=5.6 Hz, 1H), 7.84 (d, J=2.4 Hz, 1H), 7.51-7.58 (m, 2H), 7.49-7.50 (m, 1H), 7.24-7.35 (m, 3H), 7.16-7.18 (m, 1H), 6.72-6.77 (m, 1H), 6.58 (s, 1H), 4.86-4.91 (m, 1H), 4.56-4.60 (m, 1H), 4.19-4.22 (m, 1H), 3.52-3.55 (m, 1H), 1.84 (t, J=2.0 Hz, 3H). ¹³C NMR (151 MHz, Chloroform-d) δ 168.3, 147.8, 145.0 (d, J=9.1 Hz), 142.6 (d, J=6.1 Hz), 142.2, 138.1, 137.5, 134.8, 132.5, 131.6, 131.1, 126.9, 126.1, 123.1, 121.1 (d, J=15.5 Hz), 117.8, 117.5, 114.7, 79.6, 75.3, 40.1, 38.9, 3.8. LC-MS: (ESI) calcd for C₂₃H₁₉FN₃O [M+H]⁺ 372.41, found 372.00.

6-((10-(But-2-yn-1-yl)-11-oxo-10,11-dihydrodibenzo[b,f][1,4]thiazepin-7-yl)amino)-5-methylnicotinonitrile. ¹H NMR (400 MHz, Chloroform-d): δ 8.40 (d, J=5.2 Hz, 1H), 7.72-7.79 (m, 3H), 7.63-7.66 (m, 1H), 7.55 (d, J=1.2 Hz, 1H), 7.42-7.44 (m, 1H), 7.28-7.35 (m, 2H), 6.46 (s, 1H), 4.94-4.99 (m, 1H), 4.39-4.44 (m, 1H), 2.28 (s, 3H), 1.86 (t, J=4.4 Hz, 3H). ¹³C NMR (151 MHz, Chloroform-d) δ 168.4, 158.9, 155.4, 150.2, 149.9, 143.1, 139.5, 139.2, 138.5, 137.7, 136.8, 135.6, 131.9, 131.0, 128.7, 128.0, 125.1, 124.4, 121.8, 118.0 (d, J=11.3 Hz), 106.5, 100.1, 80.0, 74.8, 41.2, 16.9, 3.9. LC-MS: (ESI) calcd for C₂₄H₁₉N₄OS [M+H]⁺ 411.49, found 411.20.

6-((10-(But-2-yn-1-yl)-11-oxo-10,11-dihydrodibenzo[b,f][1,4]thiazepin-7-yl)amino)-5-methylpicolinonitrile. ¹H NMR (400 MHz, Chloroform-d): δ 7.80-7.85 (m, 2H), 7.77-7.78 (m, 1H), 7.68 (d, J=2.4 Hz, 1H), 7.43-7.46 (m, 2H), 7.26-7.33 (m, 2H), 7.15 (d, J=3.6 Hz, 1H), 6.31 (s, 1H), 4.95-5.00 (m, 1H), 4.39-4.44 (m, 1H), 2.31 (s, 3H), 1.86 (t, J=4.4 Hz, 3H). ¹³C NMR (151 MHz, Chloroform-d) δ 168.5, 153.7, 138.6, 138.5, 138.1, 137.8, 137.4, 135.5, 131.9, 131.0, 130.9, 128.7, 128.6, 125.2, 123.2, 123.0, 120.8, 120.7, 117.8, 80.1, 74.9, 41.2, 17.5, 3.9. LC-MS: (ESI) calcd for C₂₄H₁₉N₄OS [M+H]⁺ 411.49, found 411.20.

10-(But-2-yn-1-yl)-7-((3,5-dimethylpyrazin-2-yl)amino)dibenzo[b,f][1,4]thiazepin-11 (10H)-one. ¹H NMR (400 MHz, Chloroform-d): δ 7.80-7.85 (m, 2H), 7.77-7.78 (m, 1H), 7.68 (d, J=2.4 Hz, 1H), 7.43-7.46 (m, 2H), 7.26-7.33 (m, 2H), 7.15 (d, J=3.6 Hz, 1H), 6.31 (s, 1H), 4.95-5.00 (m, 1H), 4.39-4.44 (m, 1H), 2.31 (s, 3H), 1.86 (t, J=4.4 Hz, 3H). ¹³C NMR (151 MHz, Chloroform-d) & 168.5, 147.5, 143.3, 139.9, 138.7, 138.3, 138.1, 137.8, 137.8, 135.7, 131.8, 131.1, 130.9, 128.6, 125.1, 122.6, 120.1, 79.9, 74.9, 41.2, 20.4, 3.9. LC-MS: (ESI) calcd for C₂₃H₂₁N₄OS [M+H]⁺ 401.50, found 401.20.

10-(But-2-yn-1-yl)-7-((3,6-dimethylpyrazin-2-yl)amino)dibenzo[b,f][1,4]thiazepin-11 (10H)-one. ¹H NMR (400 MHz, Chloroform-d): δ 7.80-7.85 (m, 2H), 7.77-7.78 (m, 1H), 7.68 (d, J=2.4 Hz, 1H), 7.43-7.46 (m, 2H), 7.26-7.33 (m, 2H), 7.15 (d, J=3.6 Hz, 1H), 6.31 (s, 1H), 4.95-5.00 (m, 1H), 4.39-4.44 (m, 1H), 2.31 (s, 3H), 1.86 (t, J=4.4 Hz, 3H). ¹³C NMR (151 MHz, Chloroform-d) δ 168.5, 148.7, 148.5, 138.7, 138.1, 137.8, 137.3, 135.6, 133.9, 131.8, 131.0, 130.9, 128.6, 125.1, 122.8, 120.1, 79.9, 74.9, 41.2, 21.0, 19.9, 3.9. LC-MS: (ESI) calcd for C₂₃H₂₁N₄OS [M+H]⁺ 401.50, found 401.20.

N-(10-(But-2-yn-1-yl)-11-oxo-10,11-dihydrodibenzo[b,f][1,4]thiazepin-7-yl) azetidine-1-carboxamide. ¹H NMR (400 MHz, Chloroform-d): δ 9.11 (s, 1H), 8.58 (d, J=1.2 Hz, 1H), 8.21 (s, 1H), 8.05-8.08 (m, 1H), 7.98-8.01 (m, 2H), 7.74-7.79 (m, 2H), 7.68-7.71 (m, 1H), 7.13-7.16 (m, 1H), 6.98-7.03 (m, 1H), 4.90-4.95 (m, 1H), 4.42-4.47 (m, 1H), 1.86 (t, J=2.0 Hz, 3H). ¹³C NMR (151 MHz, Chloroform-d) δ 168.5, 155.7, 138.7, 138.3, 137.7, 136.9, 135.6, 131.8, 131.1, 130.9, 128.5, 125.1, 122.7, 119.9, 80.0, 74.8, 49.2, 41.1, 15.1, 3.8. LC-MS: (ESI) calcd for C₂₁H₂₀N₃O₂S [M+H]⁺ 378.46, found 378.10.

N-(10-(But-2-yn-1-yl)-11-oxo-10,11-dihydrodibenzo[b,f][1,4]thiazepin-7-yl)-6-(difluoromethoxy) nicotinamide. ¹H NMR (400 MHz, Chloroform-d): δ 8.68 (s, 1H), 8.22 (d, J=2.4 Hz, 1H), 7.99 (s, 1H), 7.92 (d, J=2.4 Hz, 1H), 7.34-7.75 (m, 5H), 7.28-7.31 (m, 2H), 6.99 (d, J=8.8 Hz, 1H), 4.92-4.96 (m, 1H), 4.41-4.46 (m, 1H), 1.85 (t, J=2.0 Hz, 3H). LC-MS: (ESI) calcd for C₂₄H₁₈F₂N₃O₃S [M+H]⁺ 465.47, found 466.10.

N-(5-(But-2-yn-1-yl)-11-hydroxy-11-methyl-6-oxo-6,11-dihydro-5H-dibenzo[b,e]azepin-2-yl)-4-methoxybenzamide. ¹H NMR (400 MHz, Chloroform-d) δ 7.91 (d, J=2.6 Hz, 1H), 7.89-7.85 (m, 1H), 7.85-7.79 (m, 2H), 7.79-7.70 (m, 2H), 7.56 (d, J=8.7 Hz, 1H), 7.48-7.38 (m, 1H), 7.35-7.29 (m, 2H), 6.98 (dd, J=9.3, 2.7 Hz, 2H), 4.98-4.59 (m, 2H), 3.88 (s, 3H), 1.89 (s, 3H), 1.83 (q, J=3.0, 2.3 Hz, 3H). LC-MS: (ESI) calcd for C₂₇H₂₅N₂O₄ [M+H]⁺ 441.17, found 441.30.

(R)—N-(5-(But-2-yn-1-yl)-11-methyl-6-oxo-6,11-dihydro-5H-dibenzo[b,e]azepin-2-yl)-4-methoxybenzamide. ¹H NMR (400 MHz, Chloroform-d): δ 7.78-7.89 (m, 4H), 7.65-7.71 (m, 1H), 7.54-7.58 (m, 1H), 7.34-7.42 (m, 2H), 7.13-7.28 (m, 2H), 6.96 (d, J=8.8 Hz, 2H), 4.87-4.92 (m, 1H), 4.59-4.64 (m, 1H), 4.27 (d, J=7.2 Hz, 0.67H), 3.87-3.92 (m, 0.34H), 3.69 (s, 3H), 1.83-1.84 (m, 3H), 1.78 (d, J=7.2 Hz, 2H), 1.55-1.59 (m, 3H). ¹³C NMR (151 MHz, Chloroform-d) & 168.5, 165.2, 162.6, 146.8, 145.6, 142.0, 141.2, 136.2, 136.1, 136.0, 132.5, 132.0, 131.8, 131.5, 131.2, 130.6, 128.9, 127.1, 126.8, 126.7, 126.6, 123.9, 123.0, 121.2, 119.8, 118.8, 118.3, 114.6, 114.0, 80.1, 79.8, 75.0, 74.6, 55.5, 47.6, 40.2, 40.0, 35.7, 18.8, 13.3, 3.8. LC-MS: (ESI) calcd for C₂₇H₂₅N₂O₃ [M+H]⁺ 425.49, found 425.10.

(S)—N-(5-(But-2-yn-1-yl)-11-methyl-6-oxo-6, 11-dihydro-5H-dibenzo[b,e]azepin-2-yl)-4-methoxybenzamide. ¹H NMR (400 MHz, Chloroform-d): δ 7.78-7.89 (m, 4H), 7.65-7.71 (m, 1H), 7.54-7.58 (m, 1H), 7.34-7.42 (m, 2H), 7.13-7.28 (m, 2H), 6.96 (d, J=8.8 Hz, 2H), 4.87-4.92 (m, 1H), 4.59-4.64 (m, 1H), 4.27 (d, J=7.2 Hz, 0.67H), 3.87-3.92 (m, 0.34H), 3.69 (s, 3H), 1.83-1.84 (m, 3H), 1.78 (d, J=7.2 Hz, 2H), 1.55-1.59 (m, 3H). ¹³C NMR (151 MHz, Chloroform-d) δ 168.5, 165.2, 162.6, 146.8, 145.6, 142.0, 141.2, 136.2, 136.1, 136.0, 132.5, 132.0, 131.8, 131.5, 131.2, 130.6, 128.9, 127.1, 126.8, 126.7, 126.6, 123.9, 123.0, 121.2, 119.8, 118.8, 118.3, 114.6, 114.0, 80.1, 79.8, 75.0, 74.6, 55.5, 47.6, 40.2, 40.0, 35.7, 18.8, 13.3, 3.8. LC-MS: (ESI) calcd for C₂₇H₂₅N₂O₃ [M+H]⁺ 425.49, found 425.10.

N-(5-(But-2-yn-1-yl)-6-oxo-6, 11-dihydro-5H-dibenzo[b,e]azepin-2-yl)benzo[d]oxazole-5-carboxamide. ¹H NMR (400 MHz, Chloroform-d): δ 8.28 (s, 1H), 8.18 (d, J=4.0 Hz, 1H), 8.02 (s, 1H), 7.96-7.99 (m, 1H), 7.81-7.83 (m, 1H), 7.76 (s, 1H), 7.65-7.68 (m, 1H), 7.58-7.61 (m, 1H), 7.38-7.41 (m, 1H), 7.31-7.34 (m, 1H), 7.23-7.26 (m, 1H), 7.14-7.16 (m, 1H), 4.86-4.91 (m, 1H), 4.57-4.62 (m, 1H), 4.16-4.19 (m, 1H), 3.52-3.56 (m, 1H), 1.83 (d, J=2.4 Hz, 1H). ¹³C NMR (151 MHz, Chloroform-d) δ 168.3, 165.2, 153.9, 152.1, 142.1, 140.3, 138.3, 136.7, 135.8, 132.2, 131.9, 131.8, 131.1, 127.0, 126.1, 125.4, 123.1, 119.5, 119.0, 118.8, 111.5, 79.9, 75.0, 40.1, 38.8, 3.8. LC-MS: (ESI) calcd for C₂₆H₂₀N₃O₃ [M+H]⁺ 422.45, found 422.10.

1-(Benzo[d]oxazol-5-yl)-3-(5-(but-2-yn-1-yl)-6-oxo-6,11-dihydro-5H-dibenzo[b,e]azepin-2-yl) urea. ¹H NMR (400 MHz, Chloroform-d): δ 8.09 (s, 1H), 7.82 (d, J=8.8 Hz 1H), 7.76 (d, J=8.8 Hz, 1H), 7.50-7.53 (m, 2H), 7.32-7.42 (m, 3H), 7.22-7.26 (m, 1H), 7.14 (d, J=7.2 Hz, 1H), 7.08-7.10 (m, 1H), 7.04 (s, 1H), 6.97 (s, 1H), 4.86-4.90 (m, 1H), 4.51-4.56 (m, 1H), 4.13 (d, J=13.2 Hz, 1H), 3.48 (d, J=13.2 Hz, 1H), 1.79-1.81 (m, 3H). ¹³C NMR (151 MHz, Chloroform-d) δ 168.9, 153.7, 153.4, 146.6, 142.3, 140.5, 138.0, 136.9, 135.3, 135.1, 132.0, 131.9, 130.8, 127.1, 126.3, 122.9, 119.5, 118.4, 118.3, 112.6, 110.9, 80.2, 74.9, 40.6, 38.6, 3.7. LC-MS: (ESI) calcd for C₂₆H₂₁N₄O₃ [M+H]⁺ 437.46, found 437.00.

3-((5-(But-2-yn-1-yl)-6-oxo-6,11-dihydro-5H-dibenzo[b,e]azepin-2-yl)amino) pyrazine-2-carbonitrile. ¹H NMR (400 MHz, Chloroform-d): δ 8.31 (d, J=2.4 Hz, 1H), 8.09 (d, J=2.0 Hz, 1H), 8.85 (d, J=7.6 Hz, 1H), 7.64-7.67 (m, 1H), 7.46-7.48 (m, 2H), 7.34-7.38 (m, 1H), 7.25-7.29 (m, 1H), 7.14-7.29 (m, 2H), 4.90-4.95 (m, 1H), 4.94-4.59 (m, 1H), 4.22 (d, J=9.6 Hz, 1H), 3.54 (d, J=13.2 Hz, 1H), 1.85 (t, J=2.0 Hz, 3H). ¹³C NMR (151 MHz, Chloroform-d) δ 168.1, 153.3, 145.5, 141.9, 138.2, 137.0, 135.6, 135.1, 132.3, 131.8, 131.2, 127.1, 126.1, 123.2, 119.9, 119.7, 115.7, 114.8, 79.89, 75.1, 40.2, 38.8, 3.8. LC-MS: (ESI) calcd for C₂₃H₁₈N₅O [M+H]⁺ 380.41, found 380.00.

N-(10-(But-2-yn-1-yl)-11-oxo-10,11-dihydrodibenzo[b,f][1,4]thiazepin-7-yl)-6-(fluoromethoxy) nicotinamide. ¹H NMR (400 MHz, Chloroform-d): δ 8.22 (d, J=2.4 Hz, 1H), 7.91 (s, 1H), 7.85 (d, J=2.4 Hz, 1H), 7.71-7.82 (m, 3H), 7.59-7.62 (m, 1H), 7.36-7.39 (m, 1H), 7.24-7.36 (m, 2H), 6.63 (d, J=9.6 Hz, 1H), 6.03 (s, 1H), 5.90 (s, 1H), 4.92-4.97 (m, 1H), 4.40-4.45 (m, 1H), 1.85 (t, J=2.0 Hz, 3H). LC-MS: (ESI) calcd for C₂₄H₁₉FN₃O₃S [M+H]⁺ 448.48, found 448.10.

1-(10-(But-2-yn-1-yl)-11-oxo-10,11-dihydrodibenzo[b,f][1,4]thiazepin-7-yl)-3-(4-(difluoromethoxy)phenyl)thiourea. ¹H NMR (400 MHz, Chloroform-d): δ 7.73-7.78 (m, 3H), 7.51-7.58 (m, 3H), 7.40-7.42 (m, 5H), 7.19 (d, J=8.8 Hz, 1H), 6.34-6.71 (m, 1H), 4.95-5.00 (m, 1H), 4.35-4.40 (m, 1H), 1.85 (t, J=2.4 Hz, 3H). ¹³C NMR (151 MHz, Chloroform-d) δ 180.1, 168.3, 149.7, 142.1, 138.1, 137.5, 135.8, 135.2, 132.0, 131.3, 131.1, 128.9, 128.6, 127.2, 126.1, 125.1, 121.0, 117.2, 115.5, 113.8, 80.4, 74.6, 41.4, 3.8. LC-MS: (ESI) calcd for C₂₅H₂₀F₂N₃O₂S₂ [M+H]⁺ 496.56, found 496.00.

1-(10-(But-2-yn-1-yl)-11-oxo-10,11-dihydrodibenzo[b,f][1,4]thiazepin-7-yl)-3-(6-(difluoromethoxy)pyridin-3-yl)thiourea. ¹H NMR (400 MHz, Chloroform-d): δ 8.12 (d, J=8.8 Hz, 1H), 7.87-7.89 (m, 2H), 7.76-7.83 (m, 2H), 7.70 (s, 1H), 7.57-7.60 (m, 1H), 7.42-7.47 (m, 2H), 7.23-7.35 (m, 3H), 6.93 (d, J=8.8 Hz, 1H), 4.96-5.00 (m, 1H), 4.35-4.40 (m, 1H), 1.85 (t, J=2.4 Hz, 3H). ¹³C NMR (151 MHz, Chloroform-d) δ 180.9, 168.5, 157.1, 143.8, 142.2, 138.3, 138.1, 137.3, 136.1, 134.9, 131.9, 131.5, 131.2, 129.0, 128.7, 126.1, 125.4, 115.7, 114.0, 112.3, 111.6, 80.7, 74.4, 41.6, 29.7, 3.8. LC-MS: (ESI) calcd for C₂₄H₁₉F₂N₄O₂S₂ [M+H]⁺ 497.55, found 496.90.

1-(10-(But-2-yn-1-yl)-11-oxo-10,11-dihydrodibenzo[b,f][1,4]thiazepin-7-yl)-3-(6-methoxypyridin-3-yl)thiourea. ¹H NMR (400 MHz, Chloroform-d): δ 8.09 (s, 1H), 7.73-7.81 (m, 4H), 7.60-7.63 (m, 1H), 7.51-7.57 (m, 2H), 7.39-7.42 (m, 1H), 7.29-7.32 (m, 2H), 6.78 (d, J=8.8, Hz, 1H), 4.93-4.98 (m, 1H), 4.34-4.39 (m, 1H), 3.94 (s, 3H), 2.15 (t, J=2.4 Hz, 3H). ¹³C NMR (151 MHz, Chloroform-d) δ 181.0, 168.3, 163.2, 144.7, 142.0, 138.1, 137.5, 137.4, 135.7, 135.2, 132.0, 131.3, 131.1, 128.8, 128.6, 126.1, 125.2, 111.6, 80.5, 74.6, 53.9, 41.4, 3.8. LC-MS: (ESI) calcd for C₂₄H₂₁N₄O₂S₂ [M+H]⁺ 461.57, found 461.00.

N-(10-(But-2-yn-1-yl)-3-cyano-11-oxo-10,11-dihydrodibenzo[b,f][1,4]thiazepin-7-yl)-6-methoxynicotinamide. ¹H NMR (400 MHz, Chloroform-d): δ 10.4 (s, 1H), 8.77 (d, J=2.0 Hz, 1H), 8.18-8.23 (m, 2H), 8.09 (d, J=1.6 Hz, 1H), 7.86 (d, J=1.6 Hz, 1H), 7.78-7.80 (m, 2H), 7.71 (d, J=8.8 Hz, 1H), 6.96 (d, J=8.4 Hz, 1H), 4.88-4.93 (m, 1H), 4.67-4.72 (m, 1H), 3.94 (s, 3H), 1.77 (t, J=2.4 Hz, 1H). ¹³C NMR (151 MHz, DMSO-d6) δ 165.4, 164.9, 163.3, 147.0, 140.9, 138.8, 138.0, 136.9, 136.6, 134.0, 133.5, 132.1, 131.4, 125.4, 123.1, 121.1, 116.7, 113.1, 109.7, 79.5, 74.0, 53.2, 2.6. LC-MS: (ESI) calcd for C₂₅H₁₉N₄O₃S [M+H]⁺ 455.50, found 455.10.

1-(10-(But-2-yn-1-yl)-11-oxo-10,11-dihydrodibenzo[b,f][1,4]thiazepin-7-yl)-3-(4-(difluoromethoxy)phenyl) urea. ¹H NMR (400 MHz, Chloroform-d): δ 7.73-7.75 (m, 1H), 7.62 (d, J=8.8 Hz, 1H), 7.43-7.47 (m, 3H), 7.27-7.36 (m, 6H), 7.06 (d, J=8.8 Hz, 2H), 6.26-6.63 (m, 1H), 4.91-4.95 (m, 1H), 4.37-4.42 (m, 1H), 1.81 (t, J=2.4 Hz, 3H). LC-MS: (ESI) calcd for C₂₅H₂₀F₂N₃O₃S [M+H]⁺ 480.50, found 480.10.

3-((10-(But-2-yn-1-yl)-11-oxo-10,11-dihydrodibenzo[b,f][1,4]thiazepin-7-yl)amino)pyridazine-4-carbonitrile. ¹H NMR (400 MHz, Chloroform-d): δ 8.92 (d, J=5.2 Hz, 1H), 7.97 (d, J=2.8 Hz, 1H), 7.71-7.79 (m, 3H), 7.52 (d, J=4.8 Hz, 1H), 7.29-7.35 (m, 2H), 6.93 (s, 1H), 4.94-4.99 (m, 1H), 4.42-4.47 (m, 1H), 2.28 (s, 3H), 1.86 (t, J=2.4 Hz, 3H). ¹³C NMR (151 MHz, Chloroform-d) δ 168.3, 154.0, 144.1, 140.1, 138.4, 137.6, 136.0, 135.5, 131.9, 131.1, 129.7, 128.7, 125.2, 124.8, 122.1, 113.5, 99.4, 80.2, 74.7, 41.2, 3.8. LC-MS: (ESI) calcd for C₂₂H₁₆N₅OS [M+H]⁺ 398.45, found 398.00.

4-Methoxy-N-(6-oxo-5-(4,4,4-trifluorobut-2-yn-1-yl)-6, 11-dihydro-5H-dibenzo[b,e]azepin-2-yl)benzamide. ¹H NMR (600 MHz, Chloroform-d) § 7.86-7.80 (m, 4H), 7.75 (d, J=2.5 Hz, 1H), 7.58 (d, J=8.7 Hz, 1H), 7.42-7.32 (m, 2H), 7.25 (d, J=6.2 Hz, 1H), 7.16 (d, J=7.4 Hz, 1H), 6.99-6.93 (m, 2H), δ 4.91 (dd, J=17.2, 2.4 Hz, 1H), 4.71 (dd, J=17.2, 2.5 Hz, 1H), 4.22 (d, J=13.4 Hz, 1H), 3.87 (s, 3H), 3.58 (d, J=13.4 Hz, 1H). LC-MS: (ESI) calcd for C₂₆H₂₀F₃N₂O₃ [M+H]⁺ 465.13, found 465.20.

6-((10-(But-2-yn-1-yl)-11-oxo-10,11-dihydrodibenzo[b,f][1,4]thiazepin-7-yl)amino)pyridazine-3-carbonitrile. ¹H NMR (400 MHz, Chloroform-d): δ 8.09 (s, 1H), 7.78 (d, J=8.4 Hz, 2H), 7.69 (d, J=2.8 Hz, 1H), 7.54 (d, J=9.2 Hz, 1H), 7.41-7.45 (m, 2H), 7.32-7.35 (m, 2H), 7.09 (d, J=9.6 Hz, 1H), 4.95-4.50 (m, 1H), 4.39-4.44 (m, 1H), 1.86 (t, J=2.0 Hz, 3H). 13C NMR (151 MHz, Chloroform-d) δ 168.3, 157.7, 140.8, 138.2, 137.5, 136.5, 135.4, 132.0, 131.8, 131.4, 131.3, 131.2, 128.9, 125.8, 125.6, 122.8, 116.1, 112.2, 80.4, 74.6, 41.3, 3.9. LC-MS: (ESI) calcd for C₂₂H₁₆N₅OS [M+H]⁺ 398.45, found 398.10.

10-(But-2-yn-1-yl)-7-((4-methylpyridazin-3-yl)amino)dibenzo[b,f][1,4]thiazepin-11 (10H)-one. ¹H NMR (400 MHz, Chloroform-d): δ 8.64 (d, J=3.2 Hz, 1H), 7.91 (s, 1H), 7.66-7.76 (m, 3H), 7.40 (d, J=6.8 Hz, 1H), 7.24-7.31 (m, 2H), 7.13 (d, J=3.6 Hz, 1H), 6.30 (s, 1H), 4.91-4.95 (m, 1H), 4.42-4.47 (m, 1H), 2.27 (s, 3H), 1.86 (t, J=2.4 Hz, 3H). ¹³C NMR (151 MHz, Chloroform-d) δ 168.5, 156.3, 145.8, 138.7, 138.2, 137.8, 135.7, 131.8, 131.1, 130.9, 128.5, 128.0, 125.1, 124.8, 123.5, 121.0, 80.0, 74.9, 41.1, 16.7, 3.8. LC-MS: (ESI) calcd for C₂₂H₁₉N₄OS [M+H]⁺ 387.47, found 387.00.

10-(But-2-yn-1-yl)-7-((5-methylpyridazin-3-yl)amino)dibenzo[b,f][1,4]thiazepin-11 (10H)-one. ¹H NMR (400 MHz, Chloroform-d): δ 8.25 (s, 1H), 7.78-7.84 (m, 2H), 7.56 (d, J=2.4 Hz, 1H), 7.42-7.44 (m, 1H), 7.27-7.39 (m, 3H), 7.15 (s, 1H), 4.96-5.01 (m, 1H), 4.38-4.43 (m, 1H), 2.39 (s, 3H), 1.87 (t, J=2.4 Hz, 3H). ¹³C NMR (151 MHz, Chloroform-d) δ 168.1, 154.2, 146.4, 144.7, 142.8, 137.9, 137.4, 137.0, 133.3, 132.1, 131.4, 131.2, 129.0, 128.2, 126.0, 125.1, 117.0, 80.5, 74.6, 41.3, 19.5, 3.9. LC-MS: (ESI) calcd for C₂₂H₁₉N₄OS [M+H]⁺ 387.47, found 387.00.

10-(But-2-yn-1-yl)-7-((6-methylpyridazin-3-yl)amino)dibenzo[b,f][1,4]thiazepin-11 (10H)-one. ¹H NMR (400 MHz, Chloroform-d): δ 12.41 (s, 1H), 7.78-7.81 (m, 2H), 7.60 (s, 1H), 7.42-7.46 (m, 2H), 7.32-7.38 (m, 4H), 4.94-4.99 (m, 1H), 4.40-4.45 (m, 1H), 2.51 (s, 3H), 1.86 (s, 3H). ¹³C NMR (151 MHz, Chloroform-d) δ 168.1, 153.4, 151.5, 137.9, 137.3, 136.8, 135.2, 133.5, 132.1, 131.4, 131.2, 129.0, 127.4, 126.0, 124.5, 80.5, 74.5, 41.2, 20.3, 3.9. LC-MS: (ESI) calcd for C₂₂H₁₉N₄OS [M+H]⁺ 387.47, found 387.00.

6-((10-(But-2-yn-1-yl)-11-oxo-10,11-dihydrodibenzo[b,f][1,4]thiazepin-7-yl)amino)-5-methylpyridazine-3-carbonitrile. ¹H NMR (400 MHz, DMSO-d6): δ 8.89 (s, 1H), 8.07 (d, J=2.4 Hz, 1H), 7.91 (s, 1H), 7.82 (d, J=2.4 Hz, 1H), 7.69 (d, J=2.4 Hz, 1H), 7.51-7.54 (m, 1H), 7.41-7.44 (m, 2H), 4.84-4.89 (m, 1H), 4.67-4.72 (m, 1H), 2.31 (s, 3H), 1.77 (s, 3H). ¹³C NMR (151 MHz, DMSO-d6) δ 166.6, 156.4, 137.3, 136.9, 136.9, 136.8, 134.0, 130.8, 130.7, 130.5, 128.4, 125.4, 124.7, 124.4, 122.4, 116.4, 79.1, 74.4, 41.2, 15.9, 2.6. LC-MS: (ESI) calcd for C₂₃H₁₈N₅OS [M+H]⁺ 412.48, found 412.20.

10-(But-2-yn-1-yl)-7-((3,6-dimethylpyrazin-2-yl)amino)-11-oxo-10,11-dihydrodibenzo[b,f][1,4]thiazepine-3-carbonitrile. ¹H NMR (400 MHz, Chloroform-d): δ 8.44 (s, 1H), 8.04-8.06 (m, 2H), 7.89-7.92 (m, 2H), 7.85-7.87 (m, 1H), 7.68-7.82 (m, 1H), 7.62 (d, J=8.4 Hz, 1H), 4.84-4.89 (m, 1H), 4.65-4.69 (m, 1H), 2.50 (s, 3H), 2.45 (s, 3H), 2.33 (s, 3H). ¹³C NMR (151 MHz, DMSO-d6) δ 165.5, 148.1, 146.6, 141.2, 138.9, 138.9, 138.0, 134.4, 133.9, 133.2, 132.1, 132.0, 131.4, 125.0, 122.3, 120.3, 116.7, 113.0, 79.3, 74.1, 19.9, 19.5, 2.6. LC-MS: (ESI) calcd for C₂₄H₂₀N₅OS [M+H]⁺ 426.51, found 426.20.

2-((10-(But-2-yn-1-yl)-11-oxo-10,11-dihydrodibenzo[b,f][1,4]thiazepin-7-yl)amino)-3-fluoroisonicotinonitrile. ¹H NMR (400 MHz, Chloroform-d): δ 9.55 (s, 1H), 8.19 (d, J=4.8 Hz, 1H), 8.14 (d, J=2.4 Hz, 1H), 7.79 (d, J=2.8 Hz, 1H), 7.62-7.66 (m, 2H), 7.51-7.53 (m, 1H), 7.40-7.43 (m, 2H), 7.23-7.26 (m, 1H), 4.79-4.84 (m, 1H), 4.65-4.69 (m, 1H), 1.77 (d, J=2.4 Hz, 1H). LC-MS: (ESI) calcd for C₂₃H₁₆FN₄OS [M+H]⁺ 415.45, found 414.90.

2-((10-(But-2-yn-1-yl)-11-oxo-10,11-dihydrodibenzo[b,f][1,4]thiazepin-7-yl)amino)pyrimidine-4-carbonitrile. ¹H NMR (400 MHz, Chloroform-d): δ 8.61 (d, J=4.8 Hz, 1H), 7.73-7.83 (m, 2H), 7.64 (d, J=2.8 Hz, 1H), 7.44-7.46 (m, 1H), 7.27-7.35 (m, 3H), 7.06 (d, J=4.8 Hz, 1H), 4.94-4.99 (m, 1H), 4.39-4.44 (m, 1H), 1.87 (t, J=2.4 Hz, 3H). ¹³C NMR (151 MHz, Chloroform-d) δ 168.4, 160.1, 159.7, 141.7, 139.2, 138.5, 137.7, 136.1, 135.8, 131.9, 131.1, 128.7, 125.2, 123.2, 120.5, 115.6, 115.5, 80.1, 74.8, 41.2, 3.9. LC-MS: (ESI) calcd for C₂₂H₁₆N₅OS [M+H]⁺ 398.45, found 397.90.

2-((10-(But-2-yn-1-yl)-11-oxo-10,11-dihydrodibenzo[b,f][1,4]thiazepin-7-yl)amino) isonicotinonitrile. ¹H NMR (400 MHz, Chloroform-d): δ 9.69 (s, 1H), 8.40 (d, J=4.8 Hz, 1H), 8.10 (d, J=1.6 Hz, 1H), 7.62-7.65 (m, 3H), 7.51-7.54 (m, 1H), 7.40-7.43 (m, 2H), 7.13-7.16 (m, 2H), 4.76-4.81 (m, 1H), 4.64-4.69 (m, 1H), 1.77 (s, 3H). LC-MS: (ESI) calcd for C₂₃H₁₇N₄OS [M+H]⁺ 397.46, found 397.00.

6-((10-(But-2-yn-1-yl)-11-oxo-10,11-dihydrodibenzo[b,f][1,4]thiazepin-7-yl)amino)pyrimidine-4-carbonitrile. ¹H NMR (400 MHz, Chloroform-d): δ 8.77 (s, 1H), 8.12 (s, 1H), 7.71 (d, J=8.8 Hz, 1H), 7.63-7.67 (m, 2H), 7.52-7.54 (m, 1H), 7.41-7.43 (m, 2H), 7.27 (s, 1H), 4.79-4.83 (m, 1H), 4.68-4.72 (m, 1H), 1.77 (s, 3H). ¹³C NMR (151 MHz, Chloroform-d) δ 168.4, 160.1, 159.3, 138.5, 137.7, 136.2, 135.8, 131.8, 131.3, 128.8, 125.2, 124.7, 122.0, 116.8, 116.2, 80.2, 74.8, 41.2, 3.8. LC-MS: (ESI) calcd for C₂₂H₁₆N₅OS [M+H]⁺ 398.45, found 397.90.

10-(But-2-yn-1-yl)-7-((4-(pyridin-3-yl)pyrimidin-2-yl)amino)dibenzo[b,f][1,4]thiazepin-11 (10H)-one. ¹H NMR (400 MHz, Chloroform-d): δ 9.43 (s, 1H), 8.82-8.91 (m, 2H), 8.62 (d, J=5.2 Hz, 1H), 8.19 (d, J=2.4 Hz, 1H), 7.88-7.92 (m, 1H), 7.65-7.75 (m, 3H), 7.47-7.54 (m, 2H), 7.37-7.41 (m, 2H), 4.77-4.82 (m, 1H), 4.65-4.70 (m, 1H), 1.81 (d, J=2.4 Hz, 3H). ¹³C NMR (151 MHz, Chloroform-d) δ 168.4, 146.1, 139.2, 138.5, 137.7, 137.5, 136.3, 135.7, 131.9, 131.1, 128.7, 125.2, 123.8, 121.0, 108.1, 80.1, 74.8, 41.2, 3.9. LC-MS: (ESI) calcd for C₂₆H₂₀N₅OS [M+H]⁺ 450.53, found 450.00.

4-((10-(But-2-yn-1-yl)-11-oxo-10,11-dihydrodibenzo[b,f][1,4]thiazepin-7-yl)amino)-1,3,5-triazine-2-carbonitrile. ¹H NMR (400 MHz, Chloroform-d): δ 8.77 (d, J=22.0 Hz, 1H), 8.01 (d, J=46.0 Hz, 1H), 7.71 (s, 2H), 7.65-7.68 (m, 1H), 7.50 (s, 1H), 7.37-7.41 (m, 2H), 4.71-4.72 (m, 1H), 4.67-4.77 (m, 1H), 1.80 (s, 3H). ¹³C NMR (151 MHz, Chloroform-d) δ 168.3, 167.6, 167.0, 163.0, 140.9, 138.2, 137.5, 136.0, 132.0, 131.3, 131.1, 128.8, 125.3, 125.2, 124.9, 124.7, 122.0, 80.6, 74.6, 41.3, 3.8. LC-MS: (ESI) calcd for C₂₁H₁₅N₆OS [M+H]⁺ 399.44, found 398.90.

10-(But-2-yn-1-yl)-7-((4-chloro-1,3,5-triazin-2-yl)amino)dibenzo[b,f][1,4]thiazepin-11 (10H)-one. ¹H NMR (400 MHz, Chloroform-d): δ 8.54 (d, J=18.0 Hz, 1H), 8.02 (d, J=20.0 Hz, 1H), 7.70 (s, 2H), 7.64-7.68 (m, 1H), 7.48-7.51 (m, 1H), 4.76-4.81 (m, 1H), 4.66-4.71 (m, 1H), 1.80 (d, J=2.4 Hz, 3H). ¹³C NMR (151 MHz, Chloroform-d) δ 168.5, 168.1, 142.9, 140.5, 138.3, 137.8, 137.5, 137.2, 136.0, 132.1, 132.0, 131.8, 131.5, 131.3, 131.2, 131.1, 129.0, 128.8, 127.0, 125.3, 121.9, 80.4, 74.5, 41.3, 3.8. LC-MS: (ESI) calcd for C₂₀H₁₅ClN₅OS [M+H]⁺ 408.88, found 408.10.

6-((10-(But-2-yn-1-yl)-11-oxo-10,11-dihydrodibenzo[b,f][1,4]thiazepin-7-yl)amino)-5-methylpyrimidine-4-carbonitrile. ¹H NMR (400 MHz, DMSO-d6): δ 9.16 (s, 1H), 8.56 (s, 1H), 8.01 (d, J=2.4 Hz, 1H), 7.79-7.82 (m, 1H), 7.71 (d, J=9.2 Hz, 1H), 7.63-7.65 (m, 1H), 7.51-7.52 (m, 1H), 7.41-7.44 (m, 2H), 4.83-4.88 (m, 1H), 4.68-4.73 (m, 1H), 2.40 (s, 3H), 1.77 (s, 3H). ¹³C NMR (151 MHz, DMSO-d6) δ 166.6, 158.3, 155.5, 137.5, 137.3, 136.9, 136.8, 136.0, 134.0, 130.7, 130.5, 128.4, 125.2, 124.7, 123.1, 121.0, 115.2, 79.1, 74.4, 13.4, 2.6. LC-MS: (ESI) calcd for C₂₃H₁₈N₅OS [M+H]⁺ 412.48, found 412.00.

10-(But-2-yn-1-yl)-7-((3,5-dimethylpyridin-2-yl)amino)dibenzo[b,f][1,4]thiazepin-11 (10H)-one. ¹H NMR (400 MHz, Chloroform-d): δ 7.76-7.78 (m, 2H), 7.67-7.69 (m, 2H), 7.30-7.40 (m, 3H), 7.16 (d, J=2.4 Hz, 1H), 7.02-7.05 (m, 1H), 4.91-4.95 (m, 1H), 4.41-4.46 (m, 1H), 2.35 (s, 3H), 1.97 (s, 3H), 1.84 (t, J=2.4 Hz, 3H). ¹³C NMR (151 MHz, Chloroform-d) δ 168.3, 149.1, 148.2, 140.2, 138.1, 137.5, 136.9, 136.0, 134.8, 132.0, 131.2, 131.1, 128.8, 127.1, 126.3, 125.6, 124.8, 122.5, 80.3, 74.6, 41.2, 19.3, 17.4, 3.8. LC-MS: (ESI) calcd for C₂₄H₂₂N₃OS [M+H]⁺ 400.51, found 400.10.

2-((5-(But-2-yn-1-yl)-6-oxo-6,11-dihydro-5H-dibenzo[b,e]azepin-2-yl)amino)-3-methylisonicotinonitrile. ¹H NMR (400 MHz, Chloroform-d): δ 8.36 (s, 1H), 8.13 (d, J=4.8 Hz, 1H), 7.64 (d, J=4.4 Hz, 1H), 7.48-7.55 (m, 3H), 7.39-7.43 (m, 1H), 7.30-7.33 (m, 2H), 7.09 (d, J=4.8 Hz, 1H), 4.69-4.81 (m, 2H), 4.05 (d, J=13.2 Hz, 1H), 3.65 (d, J=13.2 Hz, 1H), 2.43 (s, 3H), 1.79 (s, 3H). ¹³C NMR (151 MHz, DMSO-d6) δ 166.9, 154.0, 145.1, 142.3, 138.1, 137.5, 133.6, 132.0, 131.2, 130.0, 126.4, 126.0, 122.3, 120.0, 119.1, 118.9, 116.3, 115.2, 78.7, 78.5, 75.4, 37.4, 15.2, 2.7. LC-MS: (ESI) calcd for C₂₅H₂₁N₄O [M+H]⁺ 393.45, found 393.10.

N-(10-(But-2-yn-1-yl)-11-oxo-10,11-dihydrodibenzo[b,f][1,4]thiazepin-7-yl)-3-methyl-2-oxo-2,3-dihydrobenzo[d]oxazole-5-carboxamide. ¹H NMR (400 MHz, DMSO-d6): δ 10.41 (s, 3H), 8.15 (d, J=2.4 Hz, 1H), 7.80-7.84 (m, 2H), 7.77-7.79 (m, 1H), 7.69-7.71 (m, 1H), 7.62-7.65 (m, 1H), 7.52-7.55 (m, 1H), 7.48-7.50 (m, 1H), 7.40-7.45 (m, 2H), 4.83-4.88 (m, 1H), 4.67-4.72 (m, 1H), 3.41 (s, 3H), 1.78 (t, J=2.4 Hz, 3H). ¹³C NMR (151 MHz, DMSO-d6) δ 166.6, 164.3, 153.4, 143.8, 137.3, 137.2, 136.8, 136.6, 134.3, 131.4, 130.7, 130.5, 129.7, 128.4, 125.0, 123.0, 121.9, 120.8, 108.6, 107.9, 79.1, 74.4, 27.7, 2.6. LC-MS: (ESI) calcd for C₂₆H₂₀N₃O₄S [M+H]⁺ 470.51, found 470.10.

N-(5-(But-2-yn-1-yl)-11-cyano-6-oxo-6,11-dihydro-5H-dibenzo[b,e]azepin-2-yl)-4-methoxybenzamide. ¹H NMR (400 MHz, Chloroform-d): δ 7.95-8.00 (m, 1H), 7.81-7.88 (m, 4H), 7.61-7.65 (m, 2H), 7.48-7.52 (m, 1H), 7.37-7.41 (m, 1H), 6.99 (d, J=8.8 Hz, 2H), 5.43 (s, 1H), 4.76 (s, 2H), 3.88 (s, 3H), 1.83 (s, 3H). ¹³C NMR (151 MHz, Chloroform-d) δ 166.7, 165.3, 162.8, 136.9, 135.9, 134.1, 133.1, 132.4, 131.6, 130.8, 129.3, 129.0, 129.0, 128.8, 126.4, 126.2, 124.4, 124.2, 122.8, 120.6, 120.4, 118.6, 116.7, 115.4, 114.1, 80.7, 74.2, 55.5, 39.9, 38.3, 3.9. LC-MS: (ESI) calcd for C₂₇H₂₂N₃O₃ [M+H]⁺ 436.47, found 436.10.

N-(5-(But-2-yn-1-yl)-11-chloro-6-oxo-6,11-dihydro-5H-dibenzo[b,e]azepin-2-yl)-4-methoxybenzamide. ¹H NMR (600 MHz, Chloroform-d): δ 8.01-7.95 (m, 2H), 7.94 (d, J=2.5 Hz, 1H), 7.87-7.81 (m, 2H), 7.77 (d, J=8.7 Hz, 1H), 7.47-7.43 (m, 1H), 7.42-7.36 (m, 2H), 7.25-7.22 (m, 1H), 7.00-6.94 (m, 2H), 5.77 (s, 1H), 5.14 (dq, J=17.0, 2.3 Hz, 1H), 4.32-4.24 (m, 1H), 3.87 (s, 3H), 1.89 (t, J=2.4 Hz, 3H). LC-MS: (ESI) calcd for C₂₆H₂₂ClN₂O₃ [M+H]⁺ 445.12, found 445.10.

10-(But-2-yn-1-yl)-7-(pyridin-3-ylamino)dibenzo[b,f][1,4]thiazepin-11 (10H)-one. ¹H NMR (400 MHz, Chloroform-d): δ 8.39 (s, 1H), 8.24 (s, 1H), 7.77-7.80 (m, 2H), 7.62 (d, J=8.8 Hz, 1H), 7.40-7.42 (m, 2H), 7.35-7.36 (m, 2H), 7.20-23 (m, 2H), 6.94-6.99 (m, 2H), 5.73 (s, 1H), 4.91-4.96 (m, 1H), 4.38-4.43 (m, 1H), 1.86 (t, J=2.4 Hz, 3H). ¹³C NMR (151 MHz, Chloroform-d) δ 168.5, 143.0, 140.9, 140.6, 138.6, 137.8, 137.2, 136.5, 131.9, 131.1, 131.0, 128.6, 125.8, 124.7, 120.7, 118.2, 80.0, 74.9, 41.2, 3.9. LC-MS: (ESI) calcd for C₂₂H₁₈N₃OS [M+H]⁺ 372.45, found 372.10.

N-(5-(But-2-yn-1-yl)-11-methyl-6-oxo-6,11-dihydro-5H-dibenzo[b,e]azepin-2-yl)-4-methoxybenzamide. ¹H NMR (400 MHz, Chloroform-d): δ 7.78-7.89 (m, 4H), 7.65-7.71 (m, 1H), 7.54-7.58 (m, 1H), 7.34-7.42 (m, 2H), 7.13-7.28 (m, 2H), 6.96 (d, J=8.8 Hz, 2H), 4.87-4.92 (m, 1H), 4.59-4.64 (m, 1H), 4.27 (d, J=7.2 Hz, 0.67H), 3.87-3.92 (m, 0.34H), 3.69 (s, 3H), 1.83-1.84 (m, 3H), 1.78 (d, J=7.2 Hz, 2H), 1.55-1.59 (m, 3H). ¹³C NMR (151 MHz, Chloroform-d) δ 168.5, 165.2, 162.6, 146.8, 145.6, 142.0, 141.2, 136.2, 136.1, 136.0, 132.5, 132.0, 131.8, 131.5, 131.2, 130.6, 128.9, 127.1, 126.8, 126.7, 126.6, 123.9, 123.0, 121.2, 119.8, 118.8, 118.3, 114.6, 114.0, 80.1, 79.8, 75.0, 74.6, 55.5, 47.6, 40.2, 40.0, 35.7, 18.8, 13.3, 3.8. LC-MS: (ESI) calcd for C₂₇H₂₅N₂O₃ [M+H]⁺ 425.49, found 425.10.

(Z)—N-(10-(But-2-en-1-yl)-11-oxo-10,11-dihydrodibenzo[b,f][1,4]thiazepin-7-yl)benzo[d]thiazole-5-carboxamide. ¹H NMR (400 MHz, Chloroform-d) δ 9.10 (s, 1H), 8.60 (s, 1H), 8.45 (s, 1H), 8.09-7.94 (m, 3H), 7.72-7.63 (m, 2H), 7.42-7.33 (m, 2H), 7.29 (s, 1H), 5.77-5.57 (m, 2H), 4.80-4.70 (m, 1H), 4.58-4.43 (m, 1H), 3.87 (s, 3H), 1.76-1.64 (m, 3H). LC-MS: (ESI) calcd for C₂₅H₂₀N₃O₂S₂ [M+H]⁺ 458.09, found 458.20.

Methyl 3-((10-(but-2-yn-1-yl)-11-oxo-10,11-dihydrodibenzo[b,f][1,4]thiazepin-7-yl) carbamoyl) bicyclo[1.1.1]pentane-1-carboxylate. ¹H NMR (400 MHz, Chloroform-d): δ 7.73-7.77 (m, 2H), 7.65-7.67 (m, 1H), 7.55-7.58 (m, 1H), 7.38-7.40 (m, 1H), 7.25-7.31 (m, 3H), 4.89-4.94 (m, 1H), 4.40-4.45 (m, 1H), 3.17 (s, 3H), 2.37 (s, 6H), 1.61 (s, 3H). LC-MS: (ESI) calcd for C₂₅H₂₃N₂O₄S [M+H]⁺ 447.13, found 447.20.

N-(10-(But-2-yn-1-yl)-11-oxo-10,11-dihydrodibenzo[b,f][1,4]thiazepin-7-yl) bicyclo[1.1.1]pentane-1-carboxamide. ¹H NMR (600 MHz, Chloroform-d) δ 7.78 (d, J=2.5 Hz, 1H), 7.77-7.71 (m, 1H), 7.64 (d, J=8.7 Hz, 1H), 7.58 (dd, J=8.8, 2.5 Hz, 1H), 7.40-7.36 (m, 2H), 7.32-7.24 (m, 2H), 4.95-4.88 (m, 1H), 4.45-4.38 (m, 1H), 2.52 (s, 1H), 2.13 (s, 6H), 1.84 (t, J=2.4 Hz, 3H). ¹³C NMR (151 MHz, Chloroform-d) δ 168.4, 168.1, 139.5, 138.5, 137.6, 135.7, 131.8, 131.1, 131.0, 128.6, 125.1, 123.5, 120.6, 80.1, 74.7, 51.5, 51.2, 45.0, 41.1, 26.8, 3.8. LC-MS: (ESI) calcd for C₂₃H₂₁N₂O₂S [M+H]⁺ 389.12, found 389.20.

N-(10-(But-2-yn-1-yl)-11-oxo-10,11-dihydrodibenzo[b,f][1,4]thiazepin-7-yl)-3-fluorobicyclo[1.1.1]pentane-1-carboxamide. ¹H NMR (600 MHz, Chloroform-d) δ 7.75 (d, J=2.5 Hz, 1H), 7.73-7.68 (m, 1H), 7.66 (s, 1H), 7.64 (s, 1H), 7.57 (dd, J=8.8, 2.5 Hz, 1H), 7.40-7.34 (m, 1H), 7.30-7.24 (m, 2H), 4.92 (dq, J=17.0, 2.4 Hz, 1H), 4.39 (dq, J=16.9, 2.3 Hz, 1H), 2.42 (d, J=2.4 Hz, 6H), 1.84 (t, J=2.4 Hz, 3H). ¹³C NMR (151 MHz, Chloroform-d) δ 168.5, 166.4, 166.2, 139.8, 138.5, 137.5, 135.7, 135.5, 131.7, 131.1, 131.1, 128.7, 125.1, 123.7, 120.9, 80.3, 75.7, 74.6, 73.5, 55.2 (d, J=21.8 Hz), 41.2, 3.8. LC-MS: (ESI) calcd for C₂₃H₂₀FN₂O₂S [M+H]⁺ 407.12, found 407.20.

3-((10-(But-2-yn-1-yl)-11-oxo-10,11-dihydrodibenzo[b,f][1,4]thiazepin-7-yl)amino)pyridazine-4-carbonitrile. ¹H NMR (400 MHz, Chloroform-d): δ 8.92 (d, J=5.2 Hz, 1H), 7.97 (d, J=2.8 Hz, 1H), 7.71-7.79 (m, 3H), 7.52 (d, J=4.8 Hz, 1H), 7.29-7.35 (m, 2H), 6.93 (s, 1H), 4.94-4.99 (m, 1H), 4.42-4.47 (m, 1H), 2.28 (s, 3H), 1.86 (t, J=2.4 Hz, 3H). ¹³C NMR (151 MHz, Chloroform-d) δ 168.3, 154.0, 144.1, 140.1, 138.4, 137.6, 136.0, 135.5, 131.9, 131.1, 129.7, 128.7, 125.2, 124.8, 122.1, 113.5, 99.4, 80.2, 74.7, 41.2, 3.8. LC-MS: (ESI) calcd for C₂₂H₁₆N₅OS [M+H]⁺ 398.45, found 398.00.

10-(But-2-yn-1-yl)-7-((1-methyl-1H-pyrazol-4-yl)amino)dibenzo[b,f][1,4]thiazepin-11 (10H)-one. ¹H NMR (400 MHz, MeOH-d4): δ 7.61-7.63 (m, 1H), 7.55 (s, 1H), 7.45 (d, J=8.8 Hz, 2H), 7.35-7.37 (m, 3H), 6.93 (d, J=2.8 Hz, 1H), 4.67-4.73 (m, 1H), 4.57-4.62 (m, 1H), 3.87 (s, 1H), 1.80 (s, 3H). ¹³C NMR (151 MHz, MeOH-d4) δ 169.2, 145.7, 138.8, 137.4, 136.2, 133.3, 132.2, 130.6, 130.5, 130.2, 128.0, 125.3, 124.3, 115.3, 113.5, 79.0, 73.7, 39.8, 37.5, 1.4. LC-MS: (ESI) calcd for C₂₁H₁₉N₄OS [M+H]⁺ 375.46, found 375.10.

10-(But-2-yn-1-yl)-7-(pyridin-4-ylamino)dibenzo[b,f][1,4]thiazepin-11 (10H)-one. ¹H NMR (400 MHz, MeOH-d4): δ 8.19 (d, J=6.0 Hz, 2H), 7.85 (d, J=8.4 Hz, 1H), 7.67 (s, 1H), 7.62 (s, 1H), 7.50 (s, 1H), 7.41 (s, 3H), 7.12 (d, J=6.0 Hz, 2H), 4.68-4.73 (m, 1H), 4.81-4.85 (m, 1H), 1.80 (s, 3H). LC-MS: (ESI) calcd for C₂₂H₁₈N₃OS [M+H]⁺ 372.45, found 372.20.

10-(But-2-yn-1-yl)-7-(pyrimidin-5-ylamino)dibenzo[b,f][1,4]thiazepin-11 (10H)-one. 1H NMR (400 MHz, MeOH-d4): δ 8.65 (s, 1H), 8.56 (s, 2H), 7.64-7.67 (m, 2H), 7.48-7.50 (m, 1H), 7.37-7.40 (m, 3H), 7.19 (d, J=2.4 Hz, 1H), 4.78-4.81 (m, 1H), 4.62-4.68 (m, 1H), 1.80 (s, 3H). ¹³C NMR (151 MHz, MeOH-d4) δ 168.9, 148.7, 144.0, 139.6, 138.3, 137.2, 136.6, 136.5, 130.8, 130.6, 130.5, 128.3, 125.7, 120.9, 118.3, 79.3, 73.5, 62.7, 39.8, 1.4. LC-MS: (ESI) calcd for C₂₁H₁₇N₄OS [M+H]⁺ 373.44, found 373.10.

10-(But-2-yn-1-yl)-7-(pyridazin-4-ylamino)dibenzo[b,f][1,4]thiazepin-11 (10H)-one. 1H NMR (400 MHz, MeOH-d4): δ 8.72-8.78 (m, 1H), 8.64 (d, J=6.0 Hz, 1H), 7.76 (d, J=8.8 Hz, 1H), 7.66-7.68 (m, 1H), 7.56-7.59 (m, 2H), 7.33-7.51 (m, 3H), 7.17-7.19 (m, 1H), 4.77-4.85 (m, 1H), 4.67-4.71 (m, 1H), 1.80 (s, 3H). ¹³C NMR (151 MHz, MeOH-d4) δ 168.7, 148.3, 144.8, 141.5, 139.5, 138.1, 137.0, 136.7, 136.3, 131.0, 130.6, 128.4, 125.8, 124.9, 122.2, 107.3, 79.5, 73.4, 39.8, 1.4. LC-MS: (ESI) calcd for C₂₁H₁₇N₄OS [M+H]⁺ 373.44, found 373.10.

3-((10-(But-2-yn-1-yl)-11-oxo-10,11-dihydrodibenzo[b,f][1,4]thiazepin-7-yl)amino) picolinonitrile. ¹H NMR (400 MHz, MeOH-d4): δ 8.15-8.17 (m, 1H), 7.65-7.70 (m, 3H), 7.44-7.50 (m, 2H), 7.38-7.42 (m, 3H), 7.20-7.23 (m, 1H), 4.76-4.81 (m, 1H), 4.64-4.69 (m, 1H), 1.81 (s, 3H). ¹³C NMR (151 MHz, MeOH-d4) δ 168.8, 144.1, 142.3, 139.1, 138.3, 137.6, 137.1, 136.5, 130.9, 130.6, 130.5, 128.3, 127.5, 125.5, 124.8, 123.0, 121.1, 120.3, 115.2, 79.4, 73.5, 39.9, 1.4. LC-MS: (ESI) calcd for C₂₃H₁₇N₄OS [M+H]⁺ 397.46, found 397.10.

4-((10-(But-2-yn-1-yl)-11-oxo-10,11-dihydrodibenzo[b,f][1,4]thiazepin-7-yl)amino) nicotinonitrile. ¹H NMR (400 MHz, MeOH-d4): δ 8.79 (s, 1H), 8.22 (d, J=7.2 Hz, 1H), 7.88 (d, J=8.4 Hz, 1H), 7.68-7.70 (m, 2H), 7.50-7.52 (m, 1H), 7.40-7.46 (m, 3H), 7.03 (d, J=7.2 Hz, 1H), 4.82-4.87 (m, 1H), 4.68-4.73 (m, 1H), 1.80 (s, 3H). ¹³C NMR (151 MHz, MeOH-d4) δ 168.5, 155.8, 148.0, 143.7, 142.2, 137.8, 136.9, 136.8, 134.3, 131.2, 130.8, 130.7, 128.9, 128.5, 126.2, 125.9, 112.2, 108.4, 79.7, 73.4, 40.0, 1.4. LC-MS: (ESI) calcd for C₂₃H₁₇N₄OS [M+H]⁺ 397.46, found 397.10.

3-((10-(But-2-yn-1-yl)-11-oxo-10,11-dihydrodibenzo[b,f][1,4]thiazepin-7-yl) carbamoyl) bicyclo[1.1.1]pentane-1-carboxylic acid. ¹H NMR (400 MHz, MeOH-d4): δ 7.97 (d, J=2.4 Hz, 1H), 7.61-7.68 (m, 3H), 7.46-7.49 (m, 1H), 7.36-7.40 (m, 2H), 4.64-4.80 (m, 2H), 2.34 (s, 6H), 1.79 (d, J=2.0 Hz, 3H). ¹³C NMR (151 MHz, MeOH-d4) δ 171.7, 169.0, 168.8, 138.5, 138.4, 137.1, 136.3, 135.5, 130.9, 130.6, 130.5, 128.3, 124.7, 123.6, 121.0, 79.4, 73.5, 51.6, 39.8, 39.0, 1.4. LC-MS: (ESI) calcd for C₂₄H₂₁N₂O₄S [M+H]⁺ 433.49, found 433.10.

N-(10-(But-2-yn-1-yl)-2-cyano-11-oxo-10,11-dihydrodibenzo[b,f][1,4]thiazepin-7-yl)-4-(difluoromethoxy)benzamide. ¹H NMR (400 MHz, DMSO-d6): δ 10.46 (s, 1H), 8.22 (d, J=13.6 Hz, 1H), 8.00-8.07 (m, 3H), 7.81-7.84 (m, 1H), 7.87-7.89 (m, 1H), 7.67-7.77 (m, 2H), 7.20-7.57 (m, 3H), 4.88-4.93 (m, 1H), 4.60-4.72 (m, 1H), 1.77 (s, 3H). ¹³C NMR (151 MHz, DMSO-d6) δ 165.0, 164.1, 153.0, 143.2, 137.8, 137.0, 136.6, 134.3, 133.8, 133.1, 131.6, 130.3, 129.3, 125.4, 123.2, 121.3, 117.4, 117.1, 116.9, 115.4, 113.7, 111.3, 79.5, 74.0, 2.6. LC-MS: (ESI) calcd for C₂₆H₁₈F₂N₃O₃S [M+H]⁺ 490.49, found 490.10.

4-(Difluoromethoxy)-N-(10-(4-fluorobut-2-yn-1-yl)-11-oxo-10,11-dihydrodibenzo[b,f][1,4]thiazepin-7-yl)benzamide. ¹H NMR (400 MHz, Chloroform-d): δ 7.98 (s, 1H), 7.93 (s, 1H), 7.87 (d, J=8.8 Hz, 1H), 7.73-7.75 (m, 1H), 7.62 (s, 2H), 7.42-7.44 (m, 1H), 7.19-7.34 (m, 4H), 6.41-6.77 (m, 1H), 5.07 (s, 1H), 4.99-5.05 (m, 1H), 4.95 (s, 1H), 4.58-4.64 (m, 1H). ¹³C NMR (151 MHz, Chloroform-d) δ 168.5, 164.6, 153.9, 139.5, 138.5, 137.2, 136.1, 131.9, 131.3, 131.3, 131.2, 129.1, 128.8, 125.1, 124.2, 121.3, 119.3, 117.1, 113.6, 85.6 (d, J=12.2 Hz), 77.9 (d, J=22.2 Hz), 71.4, 70.3, 40.9. LC-MS: (ESI) calcd for C₂₅H₁₈F₃N₂O₃S [M+H]⁺ 483.47, found 483.10.

6-((5-(But-2-yn-1-yl)-6-oxo-6,11-dihydro-5H-dibenzo[b,e]azepin-2-yl)amino)-5-methylpyrimidine-4-carbonitrile. ¹H NMR (400 MHz, DMSO-d6): δ 9.11 (s, 1H), 8.49 (s, 1H), 7.54-7.66 (m, 4H), 7.41-7.45 (m, 1H), 7.28-7.35 (m, 2H), 4.77 (s, 2H), 4.06-4.09 (m, 1H), 3.62-3.70 (m, 1H), 2.39 (s, 3H), 1.80 (s, 3H). ¹³C NMR (151 MHz, DMSO-d6) δ 166.3, 158.5, 155.5, 141.8, 137.2, 136.6, 135.4, 135.1, 131.4, 131.1, 129.7, 126.2, 125.8, 122.1, 121.2, 121.1, 120.7, 115.3, 78.7, 75.2, 36.7, 13.4, 2.6. LC-MS: (ESI) calcd for C₂₄H₂₀N₅O [M+H]⁺ 394.44, found 394.10.

N-(10-(But-2-yn-1-yl)-3-chloro-11-oxo-10,11-dihydrodibenzo[b,f][1,4]thiazepin-7-yl)-6-methoxynicotinamide. ¹H NMR (400 MHz, DMSO-d6): δ10.42 (s, 1H), 8.77 (s, 1H), 8.16-8.23 (m, 2H), 7.77-7.80 (m, 1H), 7.63-7.71 (m, 3H), 7.49 (d, J=8.0 Hz, 1H), 6.96 (d, J=8.4 Hz, 1H), 4.86 (d, J=17.2 Hz, 1H), 4.68 (d, J=17.2 Hz, 1H), 3.94 (s, 3H), 1.77 (s, 3H). ¹³C NMR (151 MHz, DMSO-d6) δ 165.7, 164.9, 163.3, 147.0, 139.2, 138.0, 137.0, 136.7, 135.5, 134.8, 133.5, 132.4, 129.8, 128.5, 125.1, 123.2, 123.1, 121.0, 109.7, 79.3, 74.2, 53.2, 2.6. LC-MS: (ESI) calcd for C₂₄H₁₉ClN₃O₃S [M+H]⁺ 464.94, found 464.10.

N-(10-(But-2-yn-1-yl)-11-oxo-10,11-dihydrodibenzo[b,f][1,4]thiazepin-7-yl)-3-methylbicyclo[1.1.1]pentane-1-carboxamide. ¹H NMR (400 MHz, Chloroform-d): δ 7.75-7.76 (m, 2H), 7.74-7.75 (m, 1H), 7.55-7.58 (m, 1H), 7.39-7.41 (m, 1H), 7.26-7.32 (m, 3H), 7.10 (s, 1H), 4.89-4.93 (m, 1H), 4.41-4.46 (m, 1H), 1.96 (s, 6H), 1.84 (s, 3H), 1.23 (s, 3H). ¹³C NMR (151 MHz, Chloroform-d) δ 168.5, 168.4, 139.5, 138.5, 137.7, 135.7, 135.7, 131.8, 131.0, 131.0, 128.6, 125.1, 123.4, 120.5, 80.1, 74.7, 52.9, 41.1, 40.1, 35.7, 17.8, 3.8. LC-MS: (ESI) calcd for C₂₄H₂₃N₂O₂S [M+H]⁺ 403.51, found 403.10.

N-(10-(But-2-yn-1-yl)-11-oxo-10,11-dihydrodibenzo[b,f][1,4]thiazepin-7-yl)-3-cyanobicyclo[1.1.1]pentane-1-carboxamide. ¹H NMR (400 MHz, Chloroform-d): δ 7.71-7.74 (m, 2H), 7.65-7.67 (m, 1H), 7.54-7.55 (m, 1H), 7.35-7.39 (m, 2H), 7.26-7.30 (m, 3H), 4.89-4.93 (m, 1H), 4.39-4.44 (m, 1H), 2.57 (s, 6H), 1.84 (s, 3H). ¹³C NMR (151 MHz, Chloroform-d) δ 168.5, 165.5, 140.0, 138.4, 137.5, 135.8, 135.0, 131.8, 131.2, 131.1, 128.7, 125.1, 123.8, 120.9, 116.9, 80.3, 74.6, 54.9, 43.4, 41.2, 3.8. LC-MS: (ESI) calcd for C₂₄H₂₀N₃O₂S [M+H]⁺ 414.49, found 414.20.

N-(10-(But-2-yn-1-yl)-11-oxo-10,11-dihydrodibenzo[b,f][1,4]thiazepin-7-yl)-3-(trifluoromethyl) bicyclo[1.1.1]pentane-1-carboxamide. ¹H NMR (400 MHz, Chloroform-d): δ 7.77-7.78 (m, 2H), 7.60-7.65 (m, 1H), 7.53-7.56 (m, 1H), 7.39-7.41 (m, 1H), 7.28-7.32 (m, 3H), 4.89-4.94 (m, 1H), 4.40-4.45 (m, 1H), 2.31 (s, 6H), 1.84 (t, J=2.4 Hz, 3H). ¹³C NMR (151 MHz, Chloroform-d) & 168.4, 166.4, 140.0, 138.4, 137.6, 135.8, 135.1, 131.8, 131.1, 131.0, 128.7, 125.2, 123.7, 120.8, 80.2, 74.6, 49.7, 41.2, 3.8. LC-MS: (ESI) calcd for C₂₄H₂₀F₃N₂O₂S [M+H]⁺ 457.48, found 457.10.

10-(But-2-yn-1-yl)-7-((4-cyano-3-methylpyridin-2-yl)amino)-11-oxo-10,11-dihydrodibenzo[b,f][1,4]thiazepine-3-carbonitrile. ¹H NMR (400 MHz, DMSO-d6): δ 8.55 (s, 1H), 8.20 (d, J=5.2 Hz, 1H), 8.06 (d, J=1.2 Hz, 1H), 8.01 (d, J=2.4 Hz, 1H), 7.86-7.88 (m, 1H), 7.77-7.80 (m, 2H), 7.64 (m, d, J=8.8 Hz, 1H), 7.18 (d, J=5.2 Hz, 1H), 4.85-4.90 (m, 1H), 4.65-4.70 (m, 1H), 2.45 (s, 3H), 1.77 (s, 3H). ¹³C NMR (151 MHz, DMSO-d6) δ 165.5, 153.2, 144.8, 141.1, 138.9, 138.6, 134.8, 133.9, 133.1, 132.0, 131.4, 125.0, 122.8, 122.0, 121.1, 119.9, 116.7, 116.0, 115.8, 113.0, 79.3, 74.1, 15.1, 2.6. LC-MS: (ESI) calcd for C₂₅H₁₈N₅OS [M+H]⁺ 436.50, found 436.20.

10-(But-2-yn-1-yl)-7-((4-cyanopyridazin-3-yl)amino)-11-oxo-10,11-dihydrodibenzo[b,f][1,4]thiazepine-3-carbonitrile. ¹H NMR (400 MHz, DMSO-d6): δ 9.56 (s, 1H), 8.98 (d, J=5.2 Hz, 1H), 8.05-8.08 (m, 3H), 7.86-7.89 (m, 1H), 7.79 (d, J=8.0 Hz, 1H), 7.69-7.73 (m, 2H), 4.88-4.93 (m, 1H), 4.67-4.72 (m, 1H), 1.77 (s, 3H). ¹³C NMR (151 MHz, DMSO-d6) δ 165.5, 153.6, 144.3, 141.0, 138.8, 137.2, 136.1, 134.0, 133.3, 132.1, 131.6, 131.4, 125.2, 124.0, 122.3, 116.7, 113.6, 113.0, 99.5, 79.4, 74.0, 2.6. LC-MS: (ESI) calcd for C₂₃H₁₅N₅OS [M+H]⁺ 423.46, found 423.10.

10-(But-2-yn-1-yl)-7-((5-cyano-3-methylpyridin-2-yl)amino)-11-oxo-10,11-dihydrodibenzo[b,f][1,4]thiazepine-3-carbonitrile. ¹H NMR (400 MHz, DMSO-d6): δ 8.71 (s, 1H), 8.45 (s, 1H), 8.03-8.06 (m, 2H), 7.77-7.88 (m, 4H), 7.67 (d, J=8.8 Hz, 1H), 4.86-4.90 (m, 1H), 4.66-4.70 (m, 1H), 2.27 (s, 3H), 1.77 (s, 3H). ¹³C NMR (151 MHz, DMSO-d6) δ 165.5, 155.1, 148.9, 141.1, 138.9, 138.8, 137.8, 135.6, 133.9, 133.1, 132.0, 131.4, 124.9, 124.0, 122.2, 119.1, 117.6, 116.7, 113.0, 98.1, 79.3, 74.1, 16.3, 2.6. LC-MS: (ESI) calcd for C₂₅H₁₈N₅OS [M+H]⁺ 436.50, found 436.10.

10-(But-2-yn-1-yl)-11-oxo-7-(pyridazin-3-ylamino)-10,11-dihydrodibenzo[b,f][1,4]thiazepine-3-carbonitrile. ¹H NMR (400 MHz, DMSO-d6): δ 9.65 (s, 1H), 8.76 (d, J=4.0 Hz, 1H), 8.31 (s, 1H), 8.09 (s, 1H), 7.85 (d, J=8.0 Hz, 1H), 7.78 (d, J=8.0 Hz, 1H), 7.67 (s, 2H), 7.55-7.58 (m, 1H), 7.22 (d, J=8.8 Hz, 1H), 4.84-4.88 (m, 1H), 4.65-4.70 (m, 1H), 1.77 (s, 3H). ¹³C NMR (151 MHz, DMSO-d6) δ 165.5, 156.4, 144.2, 141.1, 138.9, 138.3, 134.6, 134.0, 133.7, 132.0, 131.4, 128.3, 125.5, 121.1, 119.6, 116.7, 113.0, 79.4, 74.1, 2.6. LC-MS: (ESI) calcd for C₂₂H₁₆N₅OS [M+H]⁺ 398.45, found 398.10.

N-(10-(But-2-yn-1-yl)-11-oxo-10,11-dihydrodibenzo[b,f][1,4]thiazepin-7-yl)-4-methoxybenzamide. ¹H NMR (400 MHz, Chloroform-d): δ 7.92 (s, 1H), 7.70-7.83 (m, 5H), 7.63 (d, J=8.8 Hz 1H), 7.42 (d, J=6.8 Hz 1H), 7.29-7.34 (m, 2H), 6.98 (d, J=8.8 Hz, 1H), 4.92-4.96 (m, 1H), 4.43-4.47 (m, 1H), 3.88 (s, 3H), 1.86 (s, 3H). ¹³C NMR (151 MHz, Chloroform-d) δ 168.5, 165.2, 162.7, 139.6, 138.6, 137.7, 136.2, 135.8, 131.8, 131.1, 131.0, 129.0, 128.6, 126.5, 125.1, 123.9, 121.0, 114.1, 80.1, 74.7, 55.5, 41.2, 3.9. LC-MS: (ESI) calcd for C₂₅H₂₁N₂O₃S [M+H]⁺ 429.13, found 429.10.

N-(11-(But-2-yn-1-yl)-10-oxo-10,11-dihydrobenzo[f]pyrido[3,2-b][1,4]thiazepin-3-yl)-4-methoxybenzamide. ¹H NMR (400 MHz, Chloroform-d): δ8.57 (d, J=2.4 Hz, 1H), 8.53 (d, J=2.4 Hz, 1H), 8.09 (s, 1H), 7.84 (d, J=8.8 Hz, 2H), 7.67-7.71 (m, 1H), 7.39-7.45 (m, 1H), 7.28-7.33 (m, 2H), 6.96 (d, J=8.4 Hz, 2H), 5.13 (d, J=16.4 Hz, 1H), 4.87 (d, J=16.4 Hz, 1H), 3.87 (s, 3H), 1.76 (s, 3H). ¹³C NMR (151 MHz, Chloroform-d) & 167.8, 165.4, 163.0, 150.7, 139.9, 137.6, 137.1, 132.8, 132.5, 132.0, 131.3, 131.1, 130.3, 129.1, 128.9, 125.8, 114.1, 79.0, 74.6, 55.5, 38.3, 3.8. LC-MS: (ESI) calcd for C₂₄H₂₀N₃O₃S [M+H]⁺ 430.49, found 430.10.

N-(10-(But-2-yn-1-yl)-9-fluoro-11-oxo-10,11-dihydrodibenzo[b,f][1,4]thiazepin-7-yl)-4-methoxybenzamide. ¹H NMR (400 MHz, Chloroform-d): δ 8.05-8.09 (m, 1H), 7.98 (s, 1H), 7.83-7.89 (m, 2H), 7.75 (d, J=7.6 Hz, 1H), 7.49 (d, J=7.6 Hz, 1H), 7.39-7.44 (m, 1H), 7.29-7.32 (m, 1H), 7.00-7.21 (m, 1H), 6.93 (d, J=10.4 Hz, 1H), 4.60-4.65 (m, 1H), 4.60-4.65 (m, 1H), 4.37-4.42 (m, 2H), 3.70 (s, 3H), 1.43 (s, 3H). ¹³C NMR (151 MHz, Chloroform-d) δ 170.0, 165.4, 162.8, 158.2, 156.6, 138.3, 138.0, 135.5, 131.8, 131.5 (d, J=3.2 Hz), 131.0 (d, J=5.2 Hz), 129.4, 129.3, 126.6, 124.6, 124.5, 115.1, 114.9, 114.1, 82.1, 74.0, 55.6, 41.0, 3.4. LC-MS: (ESI) calcd for C₂₅H₂₀FN₂O₃S [M+H]⁺ 447.49, found 447.10.

10-(But-2-yn-1-yl)-7-(pyridin-2-ylamino)dibenzo[b,f][1,4]thiazepin-11 (10H)-one. ¹H NMR (400 MHz, Chloroform-d): δ8.21 (s, 1H), 7.78 (d, J=7.2 Hz, 1H), 7.63-7.68 (m, 2H), 7.51-7.55 (m, 1H), 7.28-7.44 (m, 4H), 6.75-6.82 (m, 3H), 4.94 (d, J=17.2 Hz, 1H), 4.41 (d, J=17.2 Hz, 1H), 1.86 (s, 3H). ¹³C NMR (151 MHz, Chloroform-d) δ 168.5, 154.9, 148.2, 138.8, 138.7, 137.9, 137.8, 137.8, 135.9, 131.9, 131.1, 130.9, 128.6, 125.3, 122.7, 120.2, 115.8, 109.4, 79.9, 74.9, 41.2, 3.9. LC-MS: (ESI) calcd for C₂₂H₁₈N₃OS [M+H]⁺ 372.45, found 372.20.

N-(10-(But-2-yn-1-yl)-11-oxo-10,11-dihydrodibenzo[b,f][1,4]thiazepin-7-yl)-4-methoxybutanamide. ¹H NMR (400 MHz, Chloroform-d): δ 7.96 (s, 1H), 7.80 (s, 1H), 7.75 (d, J=6.8 Hz 1H), 7.65 (d, J=8.4 Hz 1H), 7.47 (d, J=7.6 Hz 1H), 7.41 (d, J=7.2 Hz, 1H), 7.28-7.33 (m, 2H), 4.89-4.94 (m, 1H), 4.39-4.43 (m, 1H), 3.47-3.50 (m, 2H), 3.39 (s, 3H), 2.47-2.50 (m, 2H), 1.96-1.99 (m, 2H), 1.85 (s, 3H). ¹³C NMR (151 MHz, Chloroform-d) δ 171.3, 168.5, 139.3, 138.6, 137.6, 136.2, 135.7, 131.8, 131.1, 131.0, 128.6, 125.1, 123.4, 120.5, 80.1, 74.7, 72.0, 58.7, 41.2, 35.0, 25.2, 3.8. LC-MS: (ESI) calcd for C₂₂H₂₃N₂O₃S [M+H]⁺ 395.49, found 395.10.

N-(11-(But-2-yn-1-yl)-10-oxo-10, 11-dihydrobenzo[f]pyrido[4,3-b][1,4]thiazepin-3-yl)-4-methoxybenzamide. ¹H NMR (400 MHz, Chloroform-d): δ 8.67 (s, 1H), 8.62 (s, 1H), 8.49 (s, 1H), 7.87 (d, J=8.8 Hz, 2H), 7.78-7.81 (m, 1H), 7.45-7.47 (m, 1H), 7.33-7.37 (m, 2H), 6.99 (d, J=8.8 Hz, 1H), 4.90-4.95 (m, 1H), 4.52-4.57 (m, 1H), 3.88 (s, 3H), 1.84 (s, 3H). LC-MS: (ESI) calcd for C₂₄H₂₀N₃O₃S [M+H]⁺ 430.11, found 430.10.

10-(But-2-yn-1-yl)-7-((tetrahydro-2H-pyran-3-yl)amino)dibenzo[b,f][1,4]thiazepin-11 (10H)-one. ¹H NMR (400 MHz, Chloroform-d): δ 7.61-7.63 (m, 1H), 7.44-7.47 (m, 2H), 7.34-7.37 (m, 2H), 6.88 (d, J=2.8 Hz 1H), 6.67-6.70 (m, 2H), 4.68-4.73 (m, 1H), 4.55-4.59 (m, 1H), 3.88-. 391 (m, 1H), 3.76-3.87 (m, 1H), 3.39-3.42 (m, 1H), 3.21-3.32 (m, 1H), 1.95-2.05 (m, 1H), 1.79-1.81 (m, 4H), 1.75-1.78 (m, 1H), 1.54-1.66 (m, 1H), 1.48-1.52 (m, 1H). LC-MS: (ESI) calcd for C₂₂H₂₃N₂O₂S [M+H]⁺ 379.14, found 379.20.

10-(But-2-yn-1-yl)-7-((tetrahydrofuran-3-yl)amino)dibenzo[b,f][1,4]thiazepin-11 (10H)-one. ¹H NMR (400 MHz, Chloroform-d): δ 7.76 (d, J=7.2 Hz, 1H), 7.51 (d, J=8.4 Hz, 1H), 7.41 (d, J=7.2 Hz, 1H), 7.25-7.32 (m, 2H), 6.77 (t, J=2.0 Hz, 1H), 6.52 (dd, J₁₂=2.8 Hz, J₁₃=8.8 Hz, 1H), 4.89 (d, J=16.8 Hz, 1H), 4.36 (d, J=16.8 Hz, 1H), 4.01-4.05 (m, 1H), 3.89-3.97 (m, 2H), 3.80-3.86 (m, 1H), 3.65-3.68 (m, 1H), 2.20-2.31 (m, 1H), 1.85 (s, 3H), 1.78-1.85 (m, 1H). LC-MS: (ESI) calcd for C₂₁H₂₁N₂O₂S [M+H]⁺ 365.12, found 365.10.

2-Methoxyethyl (10-(but-2-yn-1-yl)-11-oxo-10,11-dihydrodibenzo[b,f][1,4]thiazepin-7-yl) carbamate. ¹H NMR (400 MHz, Chloroform-d): δ 7.76 (d, J=6.8 Hz 1H), 7.63-7.67 (m, 2H), 7.41 (d, J=8.0 Hz 1H), 7.28-7.32 (m, 3H), 6.71 (s, 1H), 4.89-4.94 (m, 1H), 4.39-4.44 (m, 1H), 4.31-4.33 (m, 2H), 3.62-3.64 (m, 2H), 3.41 (s, 3H), 1.84 (s, 3H). LC-MS: (ESI) calcd for C₂₁H₂₁N₂O₄S [M+H]⁺ 397.11, found 397.10.

N-(5-(But-2-yn-1-yl)-11-methyl-6-oxo-6,11-dihydro-5H-dibenzo[b,e]azepin-2-yl)-3-methyl-2-oxo-2,3-dihydrobenzo[d]oxazole-5-carboxamide. ¹H NMR (400 MHz, Chloroform-d): δ7.88-7.91 (m, 1.3H), 7.78-7.81 (m, 0.7H), 7.56-7.68 (m, 4H), 7.33-7.46 (m, 2H), 7.14-7.24 (m, 2H), 4.86-4.94 (m, 1H), 4.58-4.66 (m, 1H), 4.26-4.32 (m, 0.7H), 3.90-3.96 (m, 0.3H), 3.45 (s, 3H), 1.84 (s, 3H), 1.80 (d, J=7.2 Hz, 2H), 1.58 (d, J=7.2 Hz, 1H). LC-MS: (ESI) calcd for C₂₈H₂₄N₃O₄ [M+H]⁺ 466.17, found 466.10.

N-(10-(But-2-yn-1-yl)-11-oxo-10,11-dihydrodibenzo[b,f][1,4]thiazepin-7-yl)-3-cyanopropanamide. ¹H NMR (400 MHz, Chloroform-d): δ 7.80-7.81 (m, 1H), 7.74-7.76 (m, 1H), 7.67 (d, J=8.4 Hz 1H), 7.47-7.48 (m, 2H), 7.40-7.42 (m, 1H), 7.29-7.32 (m, 2H), 4.89-4.94 (m, 1H), 4.40-4.45 (m, 1H), 2.71-2.77 (m, 4H), 1.84 (s, 3H). LC-MS: (ESI) calcd for C₂₁H₁₈N₃O₂S [M+H]⁺ 376.10, found 376.00.

11-(But-2-yn-1-yl)-3-(cyclopentylamino)benzo[f]pyrido[3,2-b][1,4]thiazepin-10(11H)-one. ¹H NMR (400 MHz, Chloroform-d): δ 7.80 (d, J=2.9 Hz, 1H), 7.74-7.67 (m, 1H), 7.46-7.38 (m, 1H), 7.37-7.29 (m, 2H), 7.09 (d, J=2.8 Hz, 1H), 5.01 (dd, J=16.6, 2.5 Hz, 1H), 4.77 (dd, J=16.8, 2.6 Hz, 1H), 4.34 (t, J=3.1 Hz, 1H), 3.92 (tt, J=6.2, 4.6 Hz, 1H), 3.79-3.68 (m, 1H). 1.86 (t, J=2.4 Hz, 3H), 1.80-1.56 (m, 4H), 1.45-1.40 (m, 2H). LC-MS: (ESI) calcd for C₂₁H₂₂N₃OS [M+H]⁺ 364.14, found 364.20.

11-(But-2-yn-1-yl)-3-((tetrahydrofuran-3-yl)amino)benzo[f]pyrido[3,2-b][1,4]thiazepin-10(11H)-one. ¹H NMR (400 MHz, Chloroform-d): δ 7.81 (d, J=2.8 Hz, 1H), 7.72 (d, J=7.6 Hz, 1H), 7.40-7.42 (m, 1H), 7.28-7.35 (m, 2H), 7.09 (s, 1H), 5.06 (d, J=16.4 Hz, 1H), 4.79 (d, J=16.4 Hz, 1H), 4.01-4.09 (m, 1H), 3.90-3.99 (m, 2H), 3.82-3.88 (m, 1H), 3.69-3.71 (m, 1H), 2.23-2.34 (m, 1H), 1.80-1.90 (m, 1H), 1.77 (s, 3H). LC-MS: (ESI) calcd for C₂₀H₂₀N₃O₂S [M+H]: 366.12, found 366.10.

1-(10-(But-2-yn-1-yl)-11-oxo-10, 11-dihydrodibenzo[b,f][1,4]thiazepin-7-yl)-3-(2-methoxyethyl) urea. ¹H NMR (400 MHz, Chloroform-d): δ 7.75 (d, J=6.8 Hz 1H), 7.60-7.62 (m, 2H), 7.40 (d, J=8.0 Hz 1H), 7.27-7.30 (m, 3H), 7.05 (s, 1H), 5.17 (s, 1H), 4.88-4.92 (m, 1H), 4.39-4.43 (m, 1H), 3.50-3.52 (m, 2H), 3.42-3.43 (m, 2H), 3.86 (s, 3H), 1.84 (s, 3H). LC-MS: (ESI) calcd for C₂₁H₂₂N₃O₃S [M+H]⁺ 396.13, found 396.10.

N-(10-(But-2-yn-1-yl)-11-oxo-10, 11-dihydrodibenzo[b,f][1,4]thiazepin-7-yl)-4-hydroxybutanamide. ¹H NMR (400 MHz, Chloroform-d): δ 7.74-7.79 (m, 3H), 7.65 (d, J=8.8 Hz 1H), 7.48-7.51 (m, 1H), 7.40-7.42 (m, 1H), 7.28-7.31 (m, 2H), 4.89-4.94 (m, 1H), 4.39-4.44 (m, 1H), 3.75-3.77 (m, 2H), 2.51-2.54 (m, 2H), 2.18 (s, 1H), 1.93-2.00 (m, 2H), 1.84 (s, 3H). LC-MS: (ESI) calcd for C₂₁H₂₁N₂O₃S [M+H]⁺ 381.12, found 381.10.

11-(But-2-yn-1-yl)-3-((tetrahydrofuran-3-yl)amino)benzo[f]pyrido[3,2-b][1,4]thiazepin-10(11H)-one. ¹H NMR (400 MHz, Chloroform-d): δ 7.81 (d, J=2.8 Hz, 1H), 7.72 (d, J=7.6 Hz, 1H), 7.40-7.42 (m, 1H), 7.28-7.35 (m, 2H), 7.09 (s, 1H), 5.06 (d, J=16.4 Hz, 1H), 4.79 (d, J=16.4 Hz, 1H), 4.01-4.09 (m, 1H), 3.90-3.99 (m, 2H), 3.82-3.88 (m, 1H), 3.69-3.71 (m, 1H), 2.23-2.34 (m, 1H), 1.80-1.90 (m, 1H), 1.77 (s, 3H). LC-MS: (ESI) calcd for C₂₀H₂₀N₃O₂S [M+H]⁺ 366.12, found 366.10.

10-(But-2-yn-1-yl)-7-((2-methyltetrahydrofuran-3-yl)amino)dibenzo[b,f][1,4]thiazepin-11 (10H)-one. ¹H NMR (400 MHz, Chloroform-d): δ 7.76 (d, J=7.6 Hz, 1H), 7.48-7.52 (m, 1H), 7.41 (d, J=7.2 Hz, 1H), 7.26-7.33 (m, 2H), 6.77-6.80 (m, 1H), 6.50-6.56 (m, 1H), 4.89 (d, J=16.4 Hz, 1H), 4.36 (d, J=16.8 Hz, 1H), 3.85-4.03 (m, 2.5H), 3.74-3.81 (m, 1H), 3.55-3.59 (m, 0.5H), 2.26-2.41 (m, 1H), 1.85 (s, 3H), 1.72-1.81 (m, 1H), 1.28-1.31 (m, 1.5H), 1.19-1.22 (m, 1.5H). LC-MS: (ESI) calcd for C₂₂H₂₃N₂O₂S [M+H]⁺ 379.14, found 379.20.

(E)-N-(10-(But-2-yn-1-yl)-11-oxo-10,11-dihydrodibenzo[b,f][1,4]thiazepin-7-yl)-4-methoxybut-2-enamide. ¹H NMR (400 MHz, Chloroform-d): δ 7.76 (d, J=2.0 Hz 1H), 7.64-7.75 (m, 3H), 7.50-7.53 (m, 1H), 7.40-7.42 (m, 1H), 7.28-7.33 (m, 2H), 6.18 (d, J=6.0 Hz 1H), 4.88-4.93 (m, 1H), 4.63-4.65 (m, 1H), 4.40-4.45 (m, 1H), 3.71 (s, 3H), 3.13 (d, J=7.6 Hz 1H), 1.84 (s, 3H). LC-MS: (ESI) calcd for C₂₂H₂₁N₂O₃S [M+H]⁺ 393.12, found 393.10.

N-(10-(But-2-yn-1-yl)-11-oxo-10,11-dihydrodibenzo[b,f][1,4]thiazepin-7-yl)-3-(methoxymethyl)cyclobutane-1-carboxamide. ¹H NMR (400 MHz, Chloroform-d): δ 7.74-7.82 (m, 5H), 7.64 (d, J=8.8 Hz 1H), 7.49-7.52 (m, 2H), 7.40 (d, J=7.6 Hz 2H), 7.28-7.32 (m, 4H), 7.18 (s, 1H), 4.88-4.93 (m, 1H), 4.41-4.46 (m, 1H), 3.37-3.44 (m, 10H), 3.02-3.06 (m, 2H), 2.45-2.46 (m, 2H), 2.36-2.39 (m, 4H), 2.16-2.21 (m, 4H), 1.84 (s, 6H). LC-MS: (ESI) calcd for C₂₄H₂₅N₂O₃S [M+H]⁺ 421.15, found 421.20.

N-(10-(But-2-yn-1-yl)-11-oxo-10,11-dihydrodibenzo[b,f][1,4]thiazepin-7-yl)-3-methoxybicyclo[1.1.1]pentane-1-carboxamide. ¹H NMR (400 MHz, Chloroform-d): δ 7.74-7.77 (m, 1H), 7.65 (d, J=8.8 Hz 1H), 7.54-7.57 (m, 1H), 7.38-7.41 (m, 1H), 7.24-7.33 (m, 3H), 4.88-4.93 (m, 1H), 4.41-4.46 (m, 1H), 3.33 (s, 3H), 2.23 (s, 6H), 1.84 (s, 3H). LC-MS: (ESI) calcd for C₂₄H₂₃N₂O₃S [M+H]⁺ 419.14, found 419.20.

10-(But-2-yn-1-yl)-7-(cyclopentylamino)dibenzo[b,f][1,4]thiazepin-11 (10H)-one. ¹H NMR (400 MHz, Chloroform-d): δ 7.80-7.73 (m, 1H), 7.48 (d, J=8.7 Hz, 1H), 7.40 (dd, J=7.4, 1.5 Hz, 1H), 7.34-7.22 (m, 2H), 6.76 (d, J=2.7 Hz, 1H), 6.50 (dd, J=8.7, 2.8 Hz, 1H), 4.89 (dq, J=16.8, 2.4 Hz, 1H), 4.35 (dq, J=16.8, 2.4 Hz, 1H), 3.72 (s, 2H), 2.01 (td, J=12.7, 11.0, 4.7 Hz, 2H), 1.86 (t, J=2.4 Hz, 3H), 1.80-1.56 (m, 4H), 1.45-1.40 (m, 2H). LC-MS: (ESI) calcd for C₂₂H₂₃N₂OS [M+H]⁺ 363.15, found 363.10.

10-(But-2-yn-1-yl)-7-(oxetan-3-ylamino)dibenzo[b,f][1,4]thiazepin-11 (10H)-one. ¹H NMR (400 MHz, Chloroform-d): δ 7.76 (d, J=6.8 Hz, 1H), 7.52 (d, J=8.4 Hz, 1H), 7.41 (d, J=7.6 Hz, 1H), 7.26-7.33 (m, 2H), 6.67 (d, J=2.4 Hz, 1H), 6.44 (dd, J₁₂=2.8 Hz, J₁₃=8.8 Hz, 1H), 4.96-5.00 (m, 2H), 4.86-4.91 (m, 1H), 4.55-4.61 (m, 1H), 4.46-4.51 (m, 2H), 4.33-4.38 (m, 1H), 1.85 (s, 3H). LC-MS: (ESI) calcd for C₂₀H₁₉N₂O₂S [M+H]⁺ 351.11, found 351.10.

N-(10-(But-2-yn-1-yl)-11-oxo-10,11-dihydrodibenzo[b,f][1,4]thiazepin-7-yl)-2-(oxetan-3-yl) acetamide. ¹H NMR (400 MHz, Chloroform-d): δ 7.91 (d, J=2.4 Hz 1H), 7.63-7.66 (m, 2H), 7.46-7.53 (m, 2H), 7.36-7.39 (m, 2H), 4.84-4.87 (m, 2H), 4.64-4.78 (m, 2H), 4.46-4.49 (m, 2H), 3.39-3.43 (m, 1H), 2.79 (d, J=7.6 Hz 1H), 3.41 (s, 3H), 1.78 (s, 3H). LC-MS: (ESI) calcd for C₂₂H₂₁N₂O₃S [M+H]⁺ 393.12, found 393.10.

10-(But-2-yn-1-yl)-7-((4-methyltetrahydrofuran-3-yl)amino)dibenzo[b,f][1,4]thiazepin-11 (10H)-one. ¹H NMR (400 MHz, Chloroform-d): δ 7.76 (d, J=7.6 Hz, 1H), 7.48-7.51 (m, 1H), 7.39-7.41 (m, 1H), 7.25-7.32 (m, 2H), 6.74-6.81 (m, 1H), 6.49-6.56 (m, 1H), 4.85-4.91 (m, 1H), 4.34-4.40 (m, 1H), 3.97-4.09 (m, 2.6H), 3.49-3.64 (m, 2H), 3.40-3.43 (m, 0.4H), 2.50-2.60 (m, 0.6H), 2.13-2.22 (m, 0.4H), 1.85 (s, 3H), 1.13-1.16 (m, 1H), 0.97-1.00 (m, 2H). LC-MS: (ESI) calcd for C₂₂H₂₃N₂O₂S [M+H]⁺ 379.14, found 379.20.

4-((10-(But-2-yn-1-yl)-11-oxo-10,11-dihydrodibenzo[b,f][1,4]thiazepin-7-yl)amino)tetrahydrofuran-3-carbonitrile. ¹H NMR (400 MHz, Chloroform-d): δ 7.76 (d, J=7.2 Hz 1H), 7.55 (d, J=8.4 Hz 1H), 7.41 (d, J=7.2 Hz 1H), 7.29-7.34 (m, 2H), 6.88 (s, 1H), 6.63 (d, J=7.6 Hz 1H), 4.88-4.942 (m, 1H), 4.31-4.41 (m, 2H), 4.13-4.20 (m, 3H), 3.63-3.67 (m, 1H), 3.46-3.51 (m, 1H), 1.84 (s, 3H). LC-MS: (ESI) calcd for C₂₂H₂₀N₃O₂S [M+H]⁺ 390.12, found 390.10.

(1s,3s)-N-(10-(But-2-yn-1-yl)-11-oxo-10,11-dihydrodibenzo[b,f][1,4]thiazepin-7-yl)-3-methoxycyclobutane-1-carboxamide. ¹H NMR (400 MHz, Chloroform-d): δ 7.81 (s, 1H), 7.75 (d, J=6.8 Hz 1H), 7.65 (d, J=8.8 Hz 1H), 7.31-7.52 (m, 2H), 7.28-7.30 (m, 2H), 7.11 (s, 1H), 4.83-4.93 (m, 1H), 4.37-4.45 (m, 1H), 4.09-4.18 (m, 1H), 3.26 (s, 3H), 3.00-3.09 (m, 2H), 2.55-2.60 (m, 2H), 2.16-2.29 (m, 2H), 1.84 (s, 3H). LC-MS: (ESI) calcd for C₂₃H₂₃N₂O₃S [M+H]⁺ 407.14, found 407.10.

(1r,3r)-N-(10-(But-2-yn-1-yl)-11-oxo-10,11-dihydrodibenzo[b,f][1,4]thiazepin-7-yl)-3-methoxycyclobutane-1-carboxamide. ¹H NMR (400 MHz, Chloroform-d): δ 7.86 (s, 1H), 7.74-7.76 (m, 1H), 7.65 (d, J=8.8 Hz 1H), 7.49-7.52 (m, 1H), 7.40-7.42 (m, 1H), 7.28-7.33 (m, 3H), 4.88-4.93 (m, 1H), 4.41-4.45 (m, 1H), 3.83-3.87 (m, 1H), 3.27 (s, 3H), 2.61-2.67 (m, 1H), 2.52-2.59 (m, 2H), 2.22-2.29 (m, 2H), 1.84 (s, 3H). LC-MS: (ESI) calcd for C₂₃H₂₃N₂O₃S [M+H]: 407.14, found 407.10.

N-(10-(But-2-yn-1-yl)-11-oxo-10,11-dihydrodibenzo[b,f][1,4]thiazepin-7-yl)tetrahydrofuran-3-carboxamide. ¹H NMR (400 MHz, Chloroform-d): δ 7.81 (s, 1H), 7.75 (d, J=7.2 Hz 1H), 7.50-7.59 (m, 2H), 7.41 (d, J=7.6 Hz 1H), 7.26-7.30 (m, 2H), 4.89-4.93 (m, 1H), 4.40-4.44 (m, 1H), 3.99-4.06 (m, 2H), 3.83-3.90 (m, 2H), 3.02-3.05 (m, 1H), 2.22-2.67 (m, 2H), 1.84 (s, 3H). LC-MS: (ESI) calcd for C₂₂H₂₁N₂O₃S [M+H]⁺ 393.12, found 393.10.

N-(10-(But-2-yn-1-yl)-11-oxo-10,11-dihydrodibenzo[b,f][1,4]thiazepin-7-yl)tetrahydrofuran-2-carboxamide. ¹H NMR (400 MHz, Chloroform-d): δ 8.47 (s, 1H), 7.88-7.89 (m, 1H), 7.81-7.82 (m, 1H), 7.76-7.77 (m, 1H), 7.62-7.75 (m, 2H), 7.52-7.55 (m, 1H), 7.30-7.42 (m, 1H), 7.26-7.28 (m, 2H), 4.89-4.93 (m, 1H), 4.42-4.46 (m, 2H), 3.92-4.03 (m, 2H), 2.33-2.38 (m, 2H), 2.12-2.17 (m, 2H), 1.87-1.99 (m, 2H), 1.84 (s, 3H). LC-MS: (ESI) calcd for C₂₂H₂₁N₂O₃S [M+H]⁺ 393.12, found 393.10.

10-(But-2-yn-1-yl)-7-((tetrahydro-2H-pyran-2-yl)amino)dibenzo[b,f][1,4]thiazepin-11 (10H)-one. ¹H NMR (400 MHz, Chloroform-d): δ 7.72-7.76 (m, 1H), 7.52 (t, J=8.0 Hz, 1H), 7.38-7.41 (m, 1H), 7.24-7.31 (m, 2H), 6.89-6.92 (m, 1H), 6.63-6.68 (m, 1H), 4.84-4.91 (m, 1H), 4.59-4.68 (m, 1H), 4.33-4.40 (m, 2H), 3.94-3.99 (m, 1H), 3.52-3.61 (m, 1H), 1.85-1.93 (m, 2H), 1.84 (s, 3H), 1.48-1.69 (m, 4H). LC-MS: (ESI) calcd for C₂₂H₂₃N₂O₂S [M+H]⁺ 379.14, found 379.20.

N-(10-(3-Chloroprop-2-yn-1-yl)-11-oxo-10,11-dihydrodibenzo[b,f][1,4]thiazepin-7-yl)benzo[d]thiazole-5-carboxamide. ¹H NMR (400 MHz, Chloroform-d) δ 9.11 (s, 1H), 8.58 (d, J=1.7 Hz, 1H), 8.14 (s, 1H), 8.08 (d, J=8.4 Hz, 1H), 8.04-7.96 (m, 2H), 7.81-7.72 (m, 1H), 7.69 (dd, J=8.7, 2.5 Hz, 1H), 7.62 (d, J=8.7 Hz, 1H), 7.47-7.39 (m, 1H), 7.37-7.26 (m, 2H), 4.96 (d, J=17.2 Hz, 1H), 4.56 (d, J=17.2 Hz, 1H). LC-MS: (ESI) calcd for C₂₄H₁₅ClN₃O₂S₂ [M+H]⁺ 476.02, found 476.00.

10-(But-2-yn-1-yl)-7-((4-methoxytetrahydrofuran-3-yl)amino)dibenzo[b,f][1,4]thiazepin-11 (10H)-one. ¹H NMR (400 MHz, Chloroform-d): δ 7.77 (dd, J₁₂=1.6 Hz, J₁₃=7.2 Hz, 1H), 7.50 (d, J=8.4 Hz, 1H), 7.41 (d, J=7.2 Hz, 1H), 7.26-7.33 (m, 2H), 6.80 (d, J=2.4 Hz, 1H), 6.52-6.56 (m, 1H), 4.89 (d, J=16.8 Hz, 1H), 4.37 (d, J=16.8 Hz, 1H), 4.08-4.13 (m, 1H), 3.89-4.00 (m, 4H), 3.52-3.56 (m, 1H), 3.38 (s, 3H), 1.85 (s, 3H). LC-MS: (ESI) calcd for C₂₂H₂₃N₂O₃S [M+H]⁺ 395.14, found 395.10.

10-(But-2-yn-1-yl)-7-((3-methoxycyclobutyl)amino)dibenzo[b,f][1,4]thiazepin-11 (10H)-one. ¹H NMR (400 MHz, Chloroform-d): δ 7.77 (dd, J=8.0 Hz 1H), 7.48-7.75 (m, 1H), 7.32-7.41 (m, 1H), 7.28-7.31 (m, 2H), 6.70-6.72 (m, 1H), 6.45-6.49 (m, 1H), 4.86-4.91 (m, 1H), 4.33-4.38 (m, 1H), 3.91-4.09 (m, 1H), 3.69-3.72 (m, 0.63H), 3.47-3.50 (m, 0.65H), 3.25-3.26 (m, 3H), 2.80-2.84 (m, 1H), 2.38-2.41 (m, 1H), 2.09-2.13 (m, 1H), 1.84 (s, 3H), 1.72-1.76 (m, 1H). LC-MS: (ESI) calcd for C₂₂H₂₃N₂O₂S [M+H]⁺ 379.14, found 379.20.

10-(But-2-yn-1-yl)-7-((3-methoxycyclobutyl)amino)dibenzo[b,f][1,4]thiazepin10-(but-2-yn-1-yl)-7-((3-(methoxymethyl)cyclobutyl)amino)dibenzo[b,f][1,4]thiazepin-11 (10H)-one. 1H NMR (400 MHz, Chloroform-d): δ 7.75-7.77 (m, 1H), 7.47-7.49 (m, 2H), 7.41 (d, J=8.0 Hz 1H), 7.23-7.32 (m, 2H), 6.67-6.72 (m, 1H), 6.43-6.48 (m, 1H), 4.86-4.90 (m, 1H), 4.33-4.38 (m, 1H), 3.93-3.99 (m, 0.54H), 3.75-3.79 (m, 0.71H), 3.45 (d, J=6.8 Hz 1H), 3.38 (s, 1H), 3.34 (s, 3H), 2.54-2.56 (m, 1.6H), 2.27-2.29 (m, 1.5H), 1.84-1.85 (m, 1H), 1.84 (s, 3H), 1.58-1.63 (s, 3H). LC-MS: (ESI) calcd for C₂₃H₂₅N₂O₂S [M+H]⁺ 393.16, found 393.20.

7-(Bicyclo[2.2.1]heptan-2-ylamino)-10-(but-2-yn-1-yl)dibenzo[b,f][1,4]thiazepin-11 (10H)-one. ¹H NMR (400 MHz, Chloroform-d): δ 7.77 (d, J=6.8 Hz 1H), 7.47 (d, J=8.8 Hz 1H), 7.41 (d, J=7.6 Hz 1H), 7.25-7.32 (m, 2H), 6.75-6.77 (m, 1H), 6.49-6.52 (m, 1H), 4.86-4.91 (m, 1H), 4.33-4.38 (m, 1H), 3.60-3.62 (m, 1H), 2.48 (s, 1H), 2.25 (s, 1H), 2.14-2.18 (m, 1H), 1.84 (s, 3H), 1.53-1.60 (m, 2H), 1.46-1.48 (m, 1H), 1.37-1.39 (m, 1H), 1.23-1.25 (m, 1H), 0.72-0.75 (m, 1H). LC-MS: (ESI) calcd for C₂₄H₂₅N₂OS [M+H]⁺ 389.16, found 389.20.

N-(10-(But-2-yn-1-yl)-11-oxo-10,11-dihydrodibenzo[b,f][1,4]thiazepin-7-yl)-6-oxospiro[3.3]heptane-2-carboxamide. ¹H NMR (400 MHz, Chloroform-d): δ 7.83 (s, 1H), 7.74-7.76 (m, 1H), 7.50 (d, J=8.8 Hz 1H), 7.40-7.42 (m, 1H), 7.28-7.33 (m, 1H), 7.25-7.26 (m, 1H), 4.89-4.94 (m, 1H), 4.40-4.45 (m, 1H), 3.07-3.36 (m, 5H), 2.64-2.69 (m, 2H), 2.43-2.48 (m, 2H), 1.85 (s, 3H). LC-MS: (ESI) calcd for C₂₅H₂₃N₂O₃S [M+H]⁺ 431.14, found 431.10.

10-(But-2-yn-1-yl)-7-((oxetan-3-ylmethyl)amino)dibenzo[b,f][1,4]thiazepin-11 (10H)-one. ¹H NMR (400 MHz, Chloroform-d): δ 7.77 (d, J=2.0 Hz 1H), 7.75 (d, J=1.6 Hz 1H), 7.40-7.42 (m, 1H), 7.27-7.31 (m, 2H), 6.80 (s, 1H), 6.53-6.56 (m, 1H), 4.84-4.91 (m, 3H), 4.35-4.44 (m, 3H), 3.79 (s, 1H), 3.43 (m, 2H), 3.20-3.25 (m, 2H), 3.41 (s, 3H), 1.85 (s, 3H). LC-MS: (ESI) calcd for C₂₁H₂₁N₂O₂S [M+H]⁺ 365.12, found 365.10.

10-(But-2-yn-1-yl)-7-(((3-methyloxetan-3-yl)methyl)amino)dibenzo[b,f][1,4]thiazepin-11 (10H)-one. ¹H NMR (400 MHz, Chloroform-d): δ 7.77 (d, J=2.0 Hz 1H), 7.51 (d, J=8.4 Hz 1H), 7.40 (d, J=1.2 Hz 1H), 7.27-7.33 (m, 2H), 6.84 (s, 1H), 6.58-6.61 (m, 1H), 4.86-4.91 (m, 1H), 4.45-4.51 (m, 4H), 4.36-4.41 (m, 1H), 3.98 (s, 1H), 3.24-3.30 (m, 2H), 1.86 (s, 3H), 1.36 (s, 3H). LC-MS: (ESI) calcd for C₂₂H₂₃N₂O₂S [M+H]⁺ 379.14, found 379.20.

N-(10-(But-2-yn-1-yl)-11-oxo-10,11-dihydrodibenzo[b,f][1,4]thiazepin-7-yl)-5-oxopyrrolidine-3-carboxamide. ¹H NMR (400 MHz, Chloroform-d): δ 10.23 (s, 1H), 8.02-8.04 (m, 1H), 7.59-7.65 (m, 3H), 7.49-7.54 (m, 2H), 7.39-7.43 (m, 2H), 4.81 (d, J=17.2 Hz, 1H), 4.66 (d, J=17.2 Hz, 1H), 3.45-3.50 (m, 1H), 3.28-3.33 (m, 2H), 2.37 (d, J=8.0 Hz, 2H), 1.76 (s, 3H). LC-MS: (ESI) calcd for C₂₂H₂₀N₃O₃S [M+H]⁺ 406.11, found 406.10.

N-(5-(But-2-yn-1-yl)-6-oxo-5,6-dihydrobenzo[f]pyrido[2,3-b][1,4]thiazepin-2-yl)-4-methoxybenzamide. ¹H NMR (400 MHz, Chloroform-d): δ 8.54 (s, 1H), 8.39 (d, J=8.8 Hz 1H), 8.06 (d, J=8.8 Hz 1H), 7.81-7.87 (m, 3H), 7.50-7.53 (m, 1H), 7.35-7.38 (m, 2H), 6.98 (d, J=8.8 Hz 1H), 4.88-4.93 (m, 1H), 4.49-4.50 (m, 1H), 3.88 (s, 3H), 1.85 (s, 3H). LC-MS: (ESI) calcd for C₂₄H₂₀N₃O₃S [M+H]⁺ 430.11, found 430.10.

N-(10-(But-2-yn-1-yl)-11-oxo-10,11-dihydrodibenzo[b,f][1,4]thiazepin-7-yl)-2-oxaspiro[3.3]heptane-6-carboxamide. ¹H NMR (400 MHz, Chloroform-d): δ 7.83 (s, 1H), 7.74-7.76 (m, 1H), 7.50 (d, J=8.8 Hz 1H), 7.40-7.42 (m, 1H), 7.28-7.33 (m, 1H), 7.25-7.26 (m, 1H), 4.89-4.94 (m, 1H), 4.40-4.45 (m, 1H), 3.07-3.36 (m, 5H), 2.64-2.69 (m, 2H), 2.43-2.48 (m, 2H), 1.85 (s, 3H). LC-MS: (ESI) calcd for C₂₄H₂₃N₂O₃S [M+H]⁺ 419.14, found 419.10.

7-((2-Oxaspiro[3.3]heptan-6-yl)amino)-10-(but-2-yn-1-yl)dibenzo[b,f][1,4]thiazepin-11 (10H)-one. ¹H NMR (400 MHz, Chloroform-d): δ 7.77 (d, J=1.6 Hz 1H), 7.49 (d, J=8.8 Hz 1H), 7.39-7.42 (m, 1H), 7.25-7.33 (m, 2H), 6.67 (d, J=2.4 Hz 1H), 6.42-6.45 (m, 1H), 4.86-4.91 (m, 1H), 4.76 (s, 1H), 4.63 (s, 1H), 4.33-4.37 (m, 4H), 3.82 (s, 1H), 3.66-3.70 (m, 1H), 2.73-2.78 (m, 2H), 1.97-2.04 (m, 2H), 1.86 (s, 3H). LC-MS: (ESI) calcd for C₂₃H₂₃N₂O₂S [M+H]⁺ 391.14, found 391.20.

N-(10-(But-2-yn-1-yl)-11-oxo-10,11-dihydrodibenzo[b,f][1,4]thiazepin-7-yl) oxetane-3-carboxamide. ¹H NMR (400 MHz, Chloroform-d): δ 7.86 (s, 1H), 7.76-7.78 (m, 1H), 7.69 (d, J=8.8 Hz 1H), 7.56 (d, J=8.8 Hz 1H), 7.43-7.45 (m, 2H), 7.29-7.35 (m, 2H), 4.85-4.96 (m, 5H), 4.44-4.49 (m, 1H), 3.84-3.88 (m, 1H), 1.86 (s, 3H). LC-MS: (ESI) calcd for C₂₁H₁₉N₂O₃S [M+H]⁺ 379.10, found 379.10.

(E)-N-(11-(But-2-yn-1-yl)-10-oxo-10,11-dihydrobenzo[f]pyrido[3,2-b][1,4]thiazepin-3-yl)-4-methoxybut-2-enamide. ¹H NMR (400 MHz, Chloroform-d): δ 8.43 (d, J=2.4 Hz, 1H), 8.34 (d, J=2.4 Hz, 1H), 7.87 (br, 1H), 7.70-7.73 (m, 1H), 7.41-7.44 (m, 1H), 7.29-7.35 (m, 2H), 6.20 (d, J=6.0 Hz, 1H), 5.10-5.14 (m, 1H), 4.82-4.87 (m, 1H), 4.62-4.68 (m, 1H), 3.72 (s, 3H), 3.15 (d, J=8.0 Hz, 2H), 1.76 (s, 3H). LC-MS: (ESI) calcd for C₂₁H₂₀N₃O₃S [M+H]⁺ 394.11, found 394.10.

N-(11-(But-2-yn-1-yl)-10-oxo-10, 11-dihydrobenzo[f]pyrido[3,2-b][1,4]thiazepin-3-yl)-3-methoxycyclobutane-1-carboxamide. ¹H NMR (400 MHz, Chloroform-d): δ 8.50 (s, 1H), 8.37 (s, 1H), 7.69-7.72 (m, 1H), 7.60 (br, 1H), 7.41-7.43 (m, 1H), 7.31-7.35 (m, 2H), 5.09-5.14 (m, 1H), 4.82-4.87 (m, 1H), 3.83-3.90 (m, 1H), 3.27 (s, 3H), 2.65-2.75 (m, 1H), 2.54-2.60 (m, 2H), 2.22-2.29 (m, 2H), 1.75 (s, 3H). LC-MS: (ESI) calcd for C₂₃H₂₃N₂O₃S [M+H]⁺ 408.13, found 408.20.

N-(11-(But-2-yn-1-yl)-10-oxo-10,11-dihydrobenzo[f]pyrido[3,2-b][1,4]thiazepin-3-yl)tetrahydrofuran-2-carboxamide. ¹H NMR (400 MHz, Chloroform-d): δ 8.56 (s, 1H), 8.44-8.51 (m, 2H), 7.70-7.72 (m, 1H), 7.40-7.43 (m, 1H), 7.29-7.35 (m, 2H), 5.10-5.15 (m, 1H), 4.82-4.87 (m, 1H), 4.44-4.48 (m, 1H), 3.92-4.08 (m, 2H), 2.33-2.42 (m, 1H), 2.08-2.20 (m, 1H), 1.88-2.01 (m, 2H), 1.75 (s, 3H). LC-MS: (ESI) calcd for C₂₁H₂₀N₃O₃S [M+H]⁺ 394.11, found 394.10.

N-(11-(But-2-yn-1-yl)-10-oxo-10, 11-dihydrobenzo[f]pyrido[3,2-b][1,4]thiazepin-3-yl)-3-methoxybicyclo[1.1.1]pentane-1-carboxamide. ¹H NMR (400 MHz, Chloroform-d): δ 8.42-8.44 (m, 2H), 7.69-7.71 (m, 1H), 7.37-7.42 (m, 2H), 7.29-7.34 (m, 2H), 5.09-5.14 (m, 1H), 4.82-4.87 (m, 1H), 3.33 (s, 3H), 2.25 (s, 6H), 1.75 (s, 3H). LC-MS: (ESI) calcd for C₂₃H₂₂N₃O₃S [M+H]⁺ 420.13, found 420.20.

4-((10-(But-2-yn-1-yl)-11-oxo-10,11-dihydrodibenzo[b,f][1,4]thiazepin-7-yl)amino)-4-oxobutanoic acid. ¹H NMR (400 MHz, Methonal-d4): δ 7.92 (d, J=2.4 Hz, 1H), 7.63-7.65 (m, 2H), 7.47-7.55 (m, 2H), 7.36-7.40 (m, 2H), 4.63-4.86 (m, 2H), 2.65 (s, 3H), 1.79 (s, 3H). LC-MS: (ESI) calcd for C₂₁H₁₉N₂O₄S [M+H]⁺ 395.10, found 395.10.

10-(But-2-yn-1-yl)-7-((3-(hydroxymethyl)cyclobutyl)amino)dibenzo[b,f][1,4]thiazepin-11 (10H)-one. ¹H NMR (400 MHz, Chloroform-d): LC-MS: (ESI) calcd for C₂₃H₂₅N₂O₂S [M+H]⁺ 393.16, found 393.20.

N-(10-(But-2-yn-1-yl)-11-oxo-10,11-dihydrodibenzo[b,f][1,4]thiazepin-7-yl)-6-hydroxyspiro[3.3]heptane-2-carboxamide. ¹H NMR (400 MHz, Chloroform-d): δ 7.82 (s, 1H), 7.74-7.76 (m, 1H), 7.64 (d, J=8.8 Hz, 1H), 7.41-7.49 (m, 1H), 7.39 (d, J=2.0 Hz, 1H), 7.26-7.32 (m, 2H), 7.05 (s, 1H), 4.89-4.94 (m, 1H), 4.39-4.44 (m, 1H), 4.12-4.26 (m, 1H), 2.99-3.03 (m, 1H), 2.49-2.52 (m, 1H), 2.36-2.41 (m, 3H), 2.20-2.22 (m, 2H), 1.90-1.99 (m, 2H), 1.87 (s, 3H), 1.64-1.65 (m, 1H). LC-MS: (ESI) calcd for C₂₅H₂₅N₂O₃S [M+H]⁺ 433.15, found 433.20.

N-(10-(But-2-yn-1-yl)-11-oxo-10,11-dihydrodibenzo[b,f][1,4]thiazepin-7-yl) pyrrolidine-3-carboxamide. ¹H NMR (400 MHz, MeOH-d4): δ 8.55 (br, 1H), 7.99 (s, 1H), 7.63-7.68 (m, 2H), 7.54-7.57 (m, 1H), 7.45-7.49 (m, 1H), 7.36-7.39 (m, 2H), 4.65-4.88 (m, 2H), 3.52-3.56 (m, 1H), 3.35-3.42 (m, 2H), 3.31-3.34 (m, 1H), 3.26-3.31 (m, 1H), 2.29-2.39 (m, 1H), 2.16-2.24 (m, 1H), 1.19 (s, 3H). LC-MS: (ESI) calcd for C₂₂H₂₂N₃O₂S [M+H]⁺ 392.14, found 392.20.

N-(10-(But-2-yn-1-yl)-11-oxo-10,11-dihydrodibenzo[b,f][1,4]thiazepin-7-yl)-2-oxopyrrolidine-3-carboxamide. ¹H NMR (400 MHz, Chloroform-d): δ 9.67 (s, 1H), 7.88 (d, J=7.6 Hz 1H), 7.60 (d, J=7.2 Hz 1H), 7.65 (d, J=8.8 Hz 1H), 7.53 (d, J=8.8 Hz 1H), 7.41 (d, J=6.8 Hz 1H), 7.26-7.32 (m, 2H), 5.74 (s, 1H), 4.89-4.93 (m, 1H), 4.41-4.45 (m, 1H), 3.35-3.45 (m, 3H), 2.51-2.67 (m, 2H), 1.85 (s, 3H). LC-MS: (ESI) calcd for C₂₂H₂₀N₃O₃S [M+H]⁺ 406.11, found 406.10.

N-(10-(But-2-yn-1-yl)-11-oxo-10,11-dihydrodibenzo[b,f][1,4]thiazepin-7-yl)-1-methyl-2-oxopyrrolidine-3-carboxamide. ¹H NMR (400 MHz, Chloroform-d): δ 9.86 (s, 1H), 7.88-7.91 (m, 1H), 7.77 (d, J=6.8 Hz 1H), 7.64-7.67 (m, 1H), 7.56-7.59 (m, 1H), 7.42-7.44 (m, 1H), 7.29-7.33 (m, 2H), 4.90-4.95 (m, 1H), 4.44-4.49 (m, 1H), 3.41-3.45 (m, 3H), 2.94 (s, 3H), 2.53-2.58 (m, 1H), 2.41-2.45 (m, 1H), 1.86 (s, 3H). LC-MS: (ESI) calcd for C₂₃H₂₂N₃O₃S [M+H]⁺ 420.13, found 420.20.

11-(But-2-yn-1-yl)-3-((4-methyltetrahydrofuran-3-yl)amino)benzo[f]pyrido[3,2-b][1,4]thiazepin-10(11H)-one. ¹H NMR (400 MHz, Chloroform-d): δ7.84 (d, J=2.8 Hz, 1H), 7.72-7.74 (m, 1H), 7.40-7.42 (m, 1H), 7.28-7.35 (m, 2H), 7.12 (s, 1H), 5.04-5.08 (m, 1H), 4.76-4.80 (m, 1H), 4.04-4.09 (m, 2H), 3.97-4.02 (m, 1H), 3.60-3.65 (m, 1H), 3.50-3.54 (m, 1H), 2.54-2.61 (m, 1H), 1.77 (s, 3H), 0.98-1.01 (m, 3H). LC-MS: (ESI) calcd for C₂₁H₂₂N₃O₂S [M+H]⁺ 380.14, found 380.20.

(E)-4-((10-(But-2-yn-1-yl)-11-oxo-10,11-dihydrodibenzo[b,f][1,4]thiazepin-7-yl)amino)-4-oxobut-2-enoic acid. ¹H NMR (400 MHz, MeOH-d4): δ 7.99 (m, 1H), 7.59-7.70 (m, 3H), 7.48-7.50 (m, 1H), 7.37-7.39 (m, 2H), 6.52 (d, J=12.4 Hz, 1H), 6.30 (d, J=12.4 Hz, 1H), 4.75-4.87 (m, 1H), 4.65-4.70 (m, 1H), 1.80 (s, 3H). LC-MS: (ESI) calcd for C₂₁H₂₂N₃O₃S [M+H]⁺ 393.08, found 393.10.

N-(10-(But-2-yn-1-yl)-11-oxo-10, 11-dihydrodibenzo[b,f][1,4]thiazepin-7-yl) but-2-ynamide. ¹H NMR (400 MHz, Chloroform-d): δ 7.76-7.86 (m, 2H), 7.61-7.69 (m, 1H), 7.00-7.49 (m, 5H), 4.90-4.98 (m, 1H), 4.33-4.46 (m, 1H), 2.16 (s, 3H), 1.38 (s, 3H). LC-MS: (ESI) calcd for C₂₁H₁₇N₂O₂S [M+H]⁺ 361.09, found 361.10.

N-(10-(But-2-yn-1-yl)-11-oxo-10,11-dihydrodibenzo[b,f][1,4]thiazepin-7-yl)-4-methoxybut-2-ynamide. ¹H NMR (400 MHz, Chloroform-d): δ 7.75-7.80 (m, 2H), 7.67-7.70 (m, 1H), 7.61 (br, 1H), 7.48-7.51 (m, 1H), 7.41-7.43 (m, 1H), 7.29-7.34 (m, 2H), 4.90-4.96 (m, 1H), 4.37-4.43 (m, 1H), 4.24 (s, 2H), 3.43 (s, 3H), 1.84 (s, 3H). LC-MS: (ESI) calcd for C₂₂H₁₉N₂O₃S [M+H]⁺ 391.10, found 391.10.

(1s,3s)-N-(10-(But-2-yn-1-yl)-11-oxo-10,11-dihydrodibenzo[b,f][1,4]thiazepin-7-yl)-3-hydroxycyclobutane-1-carboxamide. ¹H NMR (400 MHz, Methonal-d4): δ 7.94 (s, 1H), 7.63-7.65 (m, 2H), 7.53-7.56 (m, 1H), 7.47-7.49 (m, 1H), 7.36-7.40 (m, 2H), 4.64-4.86 (m, 2H), 4.10-4.14 (m, 1H), 2.63-2.68 (m, 1H), 2.46-2.52 (m, 2H), 2.13-2.20 (m, 2H), 1.78 (s, 3H). LC-MS: (ESI) calcd for C₂₂H₂₁N₂O₃S [M+H]⁺ 393.12, found 393.20.

(1r,3r)-N-(10-(But-2-yn-1-yl)-11-oxo-10,11-dihydrodibenzo[b,f][1,4]thiazepin-7-yl)-3-hydroxycyclobutane-1-carboxamide. ¹H NMR (400 MHz, Methonal-d4): δ 7.94 (s, 1H), 7.63-7.65 (m, 2H), 7.53-7.55 (m, 1H), 7.47-7.49 (m, 1H), 7.36-7.39 (m, 2H), 4.64-4.78 (m, 2H), 4.45-4.48 (m, 1H), 3.08-3.12 (m, 1H), 2.50-2.56 (m, 2H), 2.17-2.24 (m, 2H), 1.78 (s, 3H). LC-MS: (ESI) calcd for C₂₂H₂₁N₂O₃S [M+H]⁺ 393.12, found 393.20.

Methyl (1S,2S)-2-((10-(but-2-yn-1-yl)-11-oxo-10,11-dihydrodibenzo[b,f][1,4]thiazepin-7-yl) carbamoyl)cyclopropane-1-carboxylate. ¹H NMR (400 MHz, Chloroform-d): δ 7.81-7.83 (m, 1H), 7.75-7.77 (m, 1H), 7.66 (d, J=8.8 Hz 1H), 7.59 (s, 1H), 7.40-7.47 (m, 2H), 7.26-7.33 (m, 2H), 4.90-4.95 (m, 1H), 4.39-4.44 (m, 1H), 3.72 (s, 3H), 2.26-2.27 (m, 1H), 2.06-2.08 (m, 1H), 1.85 (s, 3H), 1.54-1.58 (m, 1H), 1.43-1.46 (m, 1H). LC-MS: (ESI) calcd for C₂₃H₂₁N₂O₄S [M+H]⁺ 421.11, found 421.10.

Methyl (1R,2S)-2-((10-(but-2-yn-1-yl)-11-oxo-10,11-dihydrodibenzo[b,f][1,4]thiazepin-7-yl) carbamoyl)cyclopropane-1-carboxylate. ¹H NMR (400 MHz, Chloroform-d): δ 8.56 (s, 1H), 7.80 (s, 1H), 7.72-7.75 (m, 1H), 7.63 (d, J=8.8 Hz, 1H), 7.48-7.52 (m, 1H), 7.39-7.41 (m, 1H), 7.26-7.32 (m, 2H), 4.88-4.93 (m, 1H), 4.38-4.43 (m, 1H), 3.73 (s, 3H), 2.07-2.19 (m, 2H), 1.83 (s, 3H), 1.66-1.71 (m, 1H), 1.38-1.44 (m, 1H). LC-MS: (ESI) calcd for C₂₃H₂₁N₂O₄S [M+H]⁺ 421.11, found 421.10.

(1S,2S)—N-(10-(But-2-yn-1-yl)-11-oxo-10,11-dihydrodibenzo[b,f][1,4]thiazepin-7-yl)-2-(hydroxymethyl)cyclopropane-1-carboxamide. ¹H NMR (400 MHz, Chloroform-d): δ 7.82 (d, J=5.6 Hz 1H), 7.74-7.76 (m, 1H), 7.64 (d, J=8.8 Hz 1H), 7.59 (s, 1H), 7.39-7.47 (m, 2H), 7.26-7.33 (m, 2H), 4.89-4.94 (m, 1H), 4.39-4.44 (m, 1H), 3.77-3.80 (m, 1H), 3.44-3.46 (m, 1H), 1.86 (s, 3H), 1.77-1.78 (m, 1H), 1.49-1.50 (m, 1H), 1.29-1.34 (m, 1H), 0.86-0.90 (m, 3H). LC-MS: (ESI) calcd for C₂₂H₂₁N₂O₃S [M+H]⁺ 393.12, found 393.20.

(1S,2R)—N-(10-(But-2-yn-1-yl)-11-oxo-10,11-dihydrodibenzo[b,f][1,4]thiazepin-7-yl)-2-(hydroxymethyl)cyclopropane-1-carboxamide. ¹H NMR (400 MHz, Chloroform-d): δ 8.01 (s, 1H), 7.82 (s, 1H), 7.73-7.75 (m, 1H), 7.63-7.65 (m, 1H), 7.45-7.48 (m, 1H), 7.38-7.42 (m, 1H), 7.26-7.32 (m, 2H), 4.88-4.93 (m, 1H), 4.38-4.42 (m, 1H), 4.00-4.04 (m, 1H), 3.71-7.37 (m, 1H), 2.72 (br, 1H), 1.83 (s, 3H), 1.55-1.64 (m, 2H), 1.23-1.28 (m, 1H), 1.08-1.13 (m, 1H). LC-MS: (ESI) calcd for C₂₂H₂₁N₂O₃S [M+H]⁺ 393.12, found 393.20.

11-(But-2-yn-1-yl)-3-((3-(methoxymethyl)cyclobutyl)amino)benzo[f]pyrido[3,2-b][1,4]thiazepin-10(11H)-one. ¹H NMR (400 MHz, Chloroform-d): δ 7.70-7.75 (m, 2H), 7.39-7.41 (m, 1H), 7.28-7.33 (m, 2H), 6.96-7.01 (m, 1H), 5.03-5.08 (m, 1H), 4.75-4.79 (m, 1H), 3.89-3.97 (m, 0.5H), 3.72-3.80 (m, 0.5H), 3.44-3.46 (m, 1H), 3.33-3.38 (m, 4H), 2.52-2.62 (m, 1.5H), 2.27-2.39 (m, 1.5H), 1.93-2.01 (m, 1H), 1.76 (s, 3H), 1.59-1.68 (m, 1H). LC-MS: (ESI) calcd for C₂₂H₂₄N₃O₂S [M+H]⁺ 394.15, found 394.20.

N-(11-(But-2-yn-1-yl)-10-oxo-10,11-dihydrobenzo[f]pyrido[3,2-b][1,4]thiazepin-3-yl)-3-(methoxymethyl)cyclobutane-1-carboxamide. ¹H NMR (400 MHz, Chloroform-d): δ 8.05 (d, J=2.4 Hz, 1H), 8.37-8.40 (m, 1H), 8.27 (br, 0.6H), 7.67-7.70 (m, 1H), 7.62 (br, 0.4H), 7.40-7.42 (m, 1H), 7.28-7.33 (m, 2H), 5.08-5.13 (m, 1H), 4.82-4.87 (m, 1H), 3.36-3.42 (m, 5H), 3.04-3.16 (m, 1H), 2.54-2.67 (m, 1H), 2.34-2.49 (m, 2H), 2.14-2.22 (m, 1H), 2.03-2.10 (m, 1H), 1.74 (s, 3H). LC-MS: (ESI) calcd for C₂₃H₂₄N₃O₃S [M+H]⁺ 422.15, found 422.20.

### Generation of NVL Mutant Cell Lines

HCT116 cells were transfected with TransIT-LT (Mirus #MIR2304) according to manufacturer's protocol with a guide RNA (either mock or NVL targeting) in the px330 vector (Addgene, #42230), and 10 nM single stranded deoxyoligonucleotide encoding a specific NVL mutation (see below for specific sequences). 72 hours after transfection, cells were selected by MM017 treatment. The resulting cells were evaluated by cresyl violet staining or collected and the relevant NVL mutations were analyzed by either amplicon or sanger sequencing.

### Generation of Knock-In Cell Lines

Guide RNA targeting genomic region surrounding the stop codon of the targeted gene was cloned into pX330. The repair template was constructed in a pGEM-T Easy Vector to include 1 kilobase homology arms matching upstream and downstream sequences of the genomic locus. The repair template sequence contains the knock-in construct, as well as an IRES Neo cassette flanked by two LoxP sites. Plasmid was sequence verified. Cells were transfected with the guide RNA and the repair template using TransIT LT. Cells were selected with G418 (1 mg/mL) for 2 weeks. Single clones were verified by Western blotting.

### Genome-Wide CRISPR/Cas9 Knockout Screen

HCT116 cells stably expressing Cas9 were generated by infecting cells with lentiCas9-Blast (Addgene #52962) and selection with 10 μg/mL blasticidin. A Cas9-expressing clone was isolated and expanded to confirm Cas9 protein expression by Western blotting. Lentivirus carrying the human Brunello CRISPR knockout pooled sgRNA library was prepared and provided by Anderson Frank in the McFadden lab. 300 million cells were infected in 8 μg/mL polybrene (Sigma) at a multiplicity of infection of approximately 30% and selected with puromycin (2 μg/mL) for 7 days. Approximately 150 million cells were maintained at each passage for each condition: vehicle (DMSO) or 1 μM MM017. Cells were treated for 24 hours with vehicle or MM017 and allowed to recover in fresh media without compound until confluency. Viable cells were counted at each passage and collected after 21 days. Genomic DNA was extracted using Blood & Cell Culture DNA Maxi kit (QIAGEN #13362), and concentrations were determined using Quant-iT PicoGreen dsDNA assay kits (Invitrogen #P7589). sgRNAs were PCR-amplified using 28 cycles from approximately 650 μg genomic DNA (10 μg DNA/100 μL reaction) using Ex Taq DNA Polymerase (TaKaRa #RR001B), 8 staggered P5 primers, and 1 unique P7 index primer per sample as described in the “Protocol PCR of sgRNAs from gDNA for Illumina sequencing” published by the Broad Institute (51). PCR products were purified using SPRIselect bead-based reagent (Beckman #B23318) before sequencing on NextSeq 2000 P2 flowcell (100 cycles, 400M reads) at UTSW McDermott Center Next Generation Sequencing Core. Burrows-Wheeler Aligner (BWA, v0.7.17) was used to map the sequencing reads to the sgRNA sequences concatenated with 5′ and 3′ flanking sequences. The mapping reads were counted for each sgRNA using SAMtools (v1.9) with the options “-F 2308-q 1”. MAGeCK was used to determine the relative depletion and enrichment of genes in test samples compared to the control sample. The size factor was estimated using control sgRNAs in normalization.

### Pharmacokinetic Studies.

Pharmacokinetic studies were performed by dosing 6-7 week old CD-1 female mice with analog MM927 formulated as 10% DMSO/10% Kolliphor EL/10% Solutal/70% D5W via the intraperitoneal route. Animals were euthanized in groups of three, blood was obtained by cheek bleeds at each time point (0, 10, 30, 90, 180, 360, 960 and 1440 minutes post dose) using the anticoagulant ACD (acidified citrate dextrose) and plasma isolated by centrifugation. For the dose escalation study, mice were dosed with 20 mg/kg, 35 mg/kg, or 60 mg/kg and blood was collected at 10, 180, and 720 minutes post dose. For the efficacy study, blood and tumor tissue was collected from tumor bearing NOD.CB17-Prkdc^{scid} Il2rg^{tmlWjl}/SzJ (NSG) mice (Jackson Laboratories, Bar Harbor, ME) 3 hours after the final dose of MM927 or vehicle (from 10 mice per group). Aliquots of plasma or tumor tissue homogenate were mixed with a two-fold volume of methanol containing 0.15% formic acid and 37.5 ng/mL N-benzylbenzamide internal standard. The samples were vortexed 15 sec, incubated at room temp for 10′ and spun twice at 16,100×g 4° C. in a refrigerated microcentrifuge. The resulting supernatants were evaluated by LC-MS/MS using a Sciex (Framingham, MA) 4500 Triple Quad coupled to a Shimadzu (Columbia, MD) Prominence LC. Briefly, MM927 was detected in multiple reaction monitoring mode (MRM) using the (m+1) transition 488.059/176.1. N-benzylbenzamide (transition 212.12/91.2) was used as an internal standard. Samples were chromatographed prior to introduction into the mass spectrometer using a C18 column (Agilent XDB column, 5 micron packing, 50×4.6 mm) with the following conditions. Buffer A (water+0.1% formic acid) and Buffer B (MeOH+0.1% formic acid) were used to create a gradient over the following times: 0-1.5 min 3% B, 1.5-2.0 min gradient to 100% B, 2.0-3.5 min 100% B, 3.5-3.6 min gradient to 3% B, 3.6-4.5 min 3% B. Standard curves were generated using naïve tumor tissue or blank plasma (Bioreclamation, Westbury, NY) spiked with known concentrations of analog MM927 and processed as described above. The concentrations of drug in each time-point sample were quantified using Analyst software (Sciex). A value of 3-fold above the signal obtained from blank plasma was designated the limit of detection (LOD). The limit of quantitation (LOQ) was defined as the lowest concentration at which back calculation yielded a concentration within 20% of theoretical and which was above the LOD. Cmax and AUC values were calculated using the noncompartmental analysis tool of Phoenix WinNonLin (Certara/Pharsight, Sunnyvale, CA).

### Pharmacodynamic and Efficacy Studies

Pharmacodynamic study: Five million parental HCT116 cells or HCT116 NVL^{R403W} in a volume of 0.1 mL were implanted using a subcutaneous injection in the left and right side of four-to-eight-week-old male and female NSG mice, respectively. Tumor volumes were measured every 2 to 4 days with calipers. When the tumor diameter reached 0.5 cm in diameter, mice were dosed with 35 mg/kg MM927 or vehicle formulated as 10% DMSO/10% Kolliphor EL/10% Solutal/70% D5W and administered via the intraperitoneal route. At each time point (0, 3, 6, 12, 24 hours post dose), tumors were harvested and flash frozen for Western blot analysis for p53 and p21 as described.

Efficacy study: Five million parental HCT116 cells or HCT116 NVL^{R403W} in a volume of 0.1 mL were implanted in the right flank. Tumor volumes were measured every 2 to 4 days with calipers. Volume is calculated as [(L*W²)*3.14]/6. When volumes reached ˜110 mm³, treatment was started with 35 mg/kg MM927 or vehicle control (0.2 mL/mouse) twice a day for 21 days. Mice were euthanized at the end of the 21-day treatment period or when any tumor reached 2 cm in its largest diameter, in agreement with the approved animal protocol. At the end of treatment, blood and tumor tissue were collected for pharmacokinetic analysis, measurement of complete blood counts, and blood chemistry measurements as described. Serum alanine transaminase and blood urea nitrogen were analyzed using VITROS MicroSlide technology.

### Cell Based Cytotoxicity Assay in HCT116 Human Colorectal Cancer Cells.

Cell culture: The HCT116 cell line was obtained from ATCC and cultured in Dulbecco's Modified Eagle's Medium (DMEM)-high glucose (Sigma Aldrich #D5796) supplemented with 10% fetal bovine serum (FBS) (Sigma-Aldrich #F2442) and 2 mM L-Glutamine (Sigma Aldrich #G7513). Cells were cultured at 37° C. in 5% CO₂. The identity of all cell lines was confirmed by short tandem repeat (STR) analysis and confirmed to be mycoplasma-free using a PCR based assay (Genatlantis).

Cell viability assay: In 96-well assay plates (Corning #2903), 2,000 cells were plated per well in 100 μL of medium and allowed to adhere overnight. The next day, compounds suspended in DMSO were dispensed to the assayed wells using a D300e Digital Dispenser (Tecan). All wells were normalized to 0.5% DMSO. 72 hours after incubation at 37° C. in 5% CO₂, cell viability was assayed using an ATP-based luminescent viability assay, CellTiter-Glo (Promega #G8462). Substrate was mixed 1:1 with phosphate-buffered saline (PBS) supplemented with 1% Triton X-100, and 40 μL of this mixture was added to the assay wells. Plates were shaken on a microplate shaker for 10 minutes at room temperature. Luminescence (CellTiter-Glo) was recorded using a Synergy 2 microplate reader (BioTek). In Prism 9 software (GraphPad), ten-point dose response curves were plotted with baseline-correction to the vehicle control well, and IC50 values were calculated by curve fitting using asymmetric (five parameter), least squares fit.

The analogs in Tables 1-10 using the above cell viability assay are listed as highly active (+++; IC50<500 nM), intermediate active (++; IC50 between 500-1,000 nM), or moderately active (+; IC50 between 1,000 and 5,000 nM).

TABLE 1

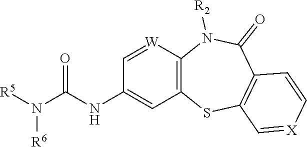

Examples:

| R² | —NR⁵R⁶ | X | W | Activity |
|---|---|---|---|---|
| —CH₂CCMe | azetidin-1-yl | CH | CH | +++ |
| —CH₂CCMe | pyrrolidine-1-yl | CH | CH | +++ |
| —CH₂CCMe | piperidin-1-yl | CH | CH | +++ |
| —CH₂CCMe | azetidin-1-yl | CH | N | +++ |
| —CH₂CCMe | pyrrolidine-1-yl | CH | N | +++ |
| —CH₂CCMe | piperidin-1-yl | CH | N | +++ |
| —(CH₂)₂OCH₂OCCH | azetidin-1-yl | CH | CH | +++ |
| —CH₂CCMe | azetidin-1-yl | CCl | CH | +++ |
| —CH₂CCMe | pyrrolidine-1-yl | CCl | CH | +++ |
| —CH₂CCMe | piperidin-1-yl | CCl | CH | +++ |

TABLE 2

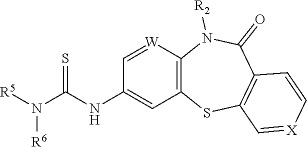

Examples:

| R² | —NR⁵R⁶ | X | W | Activity |
|---|---|---|---|---|
| —CH₂CCMe | azetidin-1-yl | CH | CH | +++ |
| —CH₂CCMe | pyrrolidine-1-yl | CH | CH | +++ |
| —CH₂CCMe | piperidin-1-yl | CH | CH | +++ |
| —CH₂CCMe | azetidin-1-yl | CH | N | +++ |
| —CH₂CCMe | pyrrolidine-1-yl | CH | N | +++ |
| —CH₂CCMe | piperidin-1-yl | CH | N | +++ |
| —CH₂CCMe | azetidin-1-yl | CCl | CH | +++ |
| —CH₂CCMe | pyrrolidine-1-yl | CCl | CH | +++ |
| —CH₂CCMe | piperidin-1-yl | CCI | CH | +++ |

TABLE 3

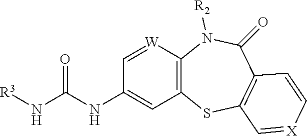

Examples:

| R² | R³ | X/W | Activity |
|---|---|---|---|
| —(CH₂)₂OCH₂CCH | Ph | CH/CH | +++ |
| —(CH₂)₂OCH₂CCH | cHex | CH/CH | +++ |
| —CH₂CCMe | 4-MeOPh | CH/CH | +++ |
| —CH₂CCMe | 3-MeOPh | CH/CH | +++ |
| —CH₂CCMe | 6-MeO-pyridin-3-yl | CH/CH | +++ |
| —CH₂CCMe | 3-FH₂COPh | CH/CH | +++ |
| —CH₂CCMe | 4-F₂HCOPh | CH/CH | +++ |
| —CH₂CCMe | 4-FH₂COPh | CH/CH | +++ |
| —CH₂CCMe | 2-Me-benzo[d]oxazol-5-yl | CH/CH | +++ |
| —CH₂CCMe | benzo[d]thiazol-5-yl | CH/CH | +++ |
| —CH₂CCMe | 3-methyl-2-oxo-2,3-dihydrobenzo[d]oxazol-5-yl | CH/CH | +++ |
| —CH₂CCMe | 3-MeO-bicyclo[1.1.1]pentan-1-yl | CH/CH | +++ |
| —CH₂CCMe | 3-MeOCH₂-cBu | CH/CH | +++ |
| —CH₂CCMe | cis-3-MeOcBu | CH/CH | +++ |
| —CH₂CCMe | trans-3-MeOcBu | CH/CH | +++ |
| —CH₂CCMe | oxetan-3-yl | CH/CH | +++ |
| —CH₂CCMe | cis-3-OH-cBu | CH/CH | +++ |
| —CH₂CCMe | trans-3-OH-cBu | CH/CH | +++ |
| —CH₂CCMe | 4-MeOPh | CH/N | +++ |
| —CH₂CCMe | benzo[d]thiazol-5-yl | CH/N | +++ |
| —CH₂CCMe | 3-methyl-2-oxo-2,3-dihydrobenzo[d]oxazol-5-yl | CH/N | +++ |
| —CH₂CCMe | 3-MeO-bicyclo[1.1.1]pentan-1-yl | CH/N | +++ |
| —CH₂CCMe | 3-MeOCH₂-cBu | CH/N | +++ |
| —CH₂CCMe | cis-3-MeOcBu | CH/N | +++ |
| —CH₂CCMe | trans-3-MeOcBu | CH/N | +++ |
| —CH₂CCMe | oxetan-3-yl | CH/N | +++ |
| —CH₂CCMe | cis-3-OH-cBu | CH/N | +++ |
| —CH₂CCMe | trans-3-OH-cBu | CH/N | +++ |

TABLE 4

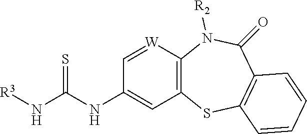

Examples:

| R² | —NR⁵R⁶ | W | Activity |
|---|---|---|---|
| —CH₂CCMe | 4-MeOPh | CH | +++ |
| —CH₂CCMe | 3-MeOPh | CH | +++ |
| —CH₂CCMe | 6-MeO-pyridin-3-yl | CH | +++ |
| —CH₂CCMe | 3-FH₂COPh | CH | +++ |
| —CH₂CCMe | 4-F₂HCOPh | CH | +++ |
| —CH₂CCMe | 4-FH₂COPh | CH | +++ |
| —CH₂CCMe | benzo[d]thiazol-5-yl | CH | +++ |
| —CH₂CCMe | 3-methyl-2-oxo-2,3-dihydrobenzo[d]oxazol-5-yl | CH | +++ |
| —CH₂CCMe | 3-MeO-bicyclo[1.1.1]]pentan-1-yl | CH | +++ |
| —CH₂CCMe | 3-MeOCH₂-cBu | CH | +++ |
| —CH₂CCMe | cis-3-MeOcBu | CH | +++ |
| —CH₂CCMe | trans-3-MeOcBu | CH | +++ |
| —CH₂CCMe | oxetan-3-yl | CH | +++ |
| —CH₂CCMe | cis-3-OH-cBu | CH | +++ |
| —CH₂CCMe | trans-3-OH-cBu | CH | +++ |
| —CH₂CCMe | 4-MeOPh | N | +++ |
| —CH₂CCMe | 3-MeOPh | N | +++ |
| —CH₂CCMe | 6-MeO-pyridin-3-yl | N | +++ |
| —CH₂CCMe | 3-FH₂COPh | N | +++ |
| —CH₂CCMe | 4-F₂HCOPh | N | +++ |
| —CH₂CCMe | 4-FH₂COPh | N | +++ |
| —CH₂CCMe | benzo[d]thiazol-5-yl | N | +++ |
| —CH₂CCMe | 3-methyl-2-oxo-2,3-dihydrobenzo[d]oxazol-5-yl | N | +++ |
| —CH₂CCMe | 3-MeO-bicyclo[1.1.1]]pentan-1-yl | N | +++ |
| —CH₂CCMe | 3-MeOCH₂-cBu | N | +++ |
| —CH₂CCMe | cis-3-MeOcBu | N | +++ |
| —CH₂CCMe | trans-3-MeOcBu | N | +++ |
| —CH₂CCMe | oxetan-3-yl | N | +++ |
| —CH₂CCMe | cis-3-OH-cBu | N | +++ |
| —CH₂CCMe | trans-3-OH-cBu | N | +++ |

TABLE 5

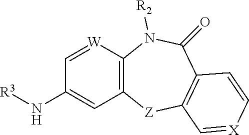

Examples:

| R² | R³ | X/W | Z | Activity |
|---|---|---|---|---|
| —CH₂CH═CHPr | pyridine-2-yl | CH/CH | S | ++ |
| —CH₂CH═CHPr | isoquinolin-1-yl | CH/CH | S | +++ |
| —CH₂CH═CHPr | 4-Me-pyridine-2-yl | CH/CH | S | ++ |
| —CH₂CH═CHPr | 4-CN-pyridine-2-yl | CH/CH | S | ++ |
| —CH₂CH═CHPr | pyrazin-2-yl | CH/CH | S | +++ |
| —CH₂CH═CHPr | pyrimidin-2-yl | CH/CH | S | + |
| —CH₂CH═CHPr | 3-Me-pyridine-2-yl | CH/CH | S | +++ |
| —CH₂CH═CHPr | 3-CN-pyridine-2-yl | CH/CH | S | +++ |
| —CH₂CH═CHPr | 3-MeO-pyridine-2-yl | CH/CH | S | ++ |
| —CH₂CH═CHPr | 3-CF3-pyridine-2-yl | CH/CH | S | ++ |
| —CH₂CH═CHPr | 3-F-pyridine-2-yl | CH/CH | S | +++ |
| —CH₂CH═CHPr | 3-CN-pyrazin-2-yl | CH/CH | S | ++ |
| —CH₂CH═CHPr | isoquinolin-3-yl | CH/CH | S | +++ |
| —CH₂CH═CHPr | 4-MeO-pyridine-2-yl | CH/CH | S | +++ |
| —CH₂CH═CHPr | 4-CF3-pyridine-2-yl | CH/CH | S | + |
| —CH₂CH═CHPr | 4-iPr-pyridine-2-yl | CH/CH | S | +++ |
| —CH₂CH═CHPr | 4-cPr-pyridine-2-yl | CH/CH | S | ++ |
| —CH₂CH═CHPr | 4-F-pyridine-2-yl | CH/CH | S | +++ |
| —CH₂CH═CHPr | 3-Me-pyrazin-2-yl | CH/CH | S | +++ |
| —CH₂CH═CHPr | 3-MeO-pyrazin-2-yl | CH/CH | S | ++ |
| —CH₂CH═CHPr | 3,5-Me₂-pyridine-2-yl | CH/CH | S | +++ |
| —CH₂CH═CHPr | 3-Me-pyridine-2-yl | CH/N | S | +++ |
| —CH₂CH═CHPr | 3-CN-pyridine-2-yl | CH/N | S | +++ |
| —CH₂CH═CHPr | 3-F-pyridine-2-yl | CH/N | S | +++ |
| —CH₂CH═CHPr | 3,5-Me₂-pyridine-2-yl | CH/N | S | +++ |
| —CH₂CH═CHPr | 3-Me-pyrazin-2-yl | CH/N | S | +++ |
| —CH₂CH═CHMe | 3-Me-pyridine-2-yl | CH/CH | S | +++ |
| —CH₂CH═CHMe | 3-CN-pyridine-2-yl | CH/CH | S | +++ |
| —CH₂CH═CHMe | 3-F-pyridine-2-yl | CH/CH | S | +++ |
| —CH₂CH═CHMe | 3,5-Me₂-pyridine-2-yl | CH/CH | S | +++ |
| —CH₂CH═CHMe | 3-Me-pyrazin-2-yl | CH/CH | S | +++ |
| —CH₂CH═CHMe | 3-Me-pyridine-2-yl | CH/N | S | +++ |
| —CH₂CH═CHMe | 3-CN-pyridine-2-yl | CH/N | S | +++ |
| —CH₂CH═CHMe | 3-F-pyridine-2-yl | CH/N | S | +++ |
| —CH₂CH═CHMe | 3,5-Me₂-pyridine-2-yl | CH/N | S | +++ |
| —CH₂CH═CHMe | 3-Me-pyrazin-2-yl | CH/N | S | +++ |
| —CH₂CCMe | 3-Me-pyrazin-2-yl | CF/CH | S | +++ |
| —CH₂CCMe | 3-MeO-pyrazin-2-yl | CF/CH | S | ++ |
| —CH₂CCMe | 3-Me-pyridin-2-yl | CF/CH | S | +++ |
| —CH₂CCMe | 3-CN-pyridin-2-yl | CF/CH | S | +++ |
| —CH₂CCMe | 3-F-pyridin-2-yl | CF/CH | S | +++ |
| —CH₂CCMe | 3-MeO-pyridin-2-yl | CF/CH | S | +++ |
| —CH₂CCMe | 3-cPr-pyridin-2-yl | CF/CH | S | +++ |
| —CH₂CCMe | 3-MeO₂C-pyridin-2-yl | CH/CH | S | + |
| —CH₂CCMe | 3-CF₃-pyridin-2-yl | CH/CH | S | +++ |
| —CH₂CCMe | 3-HCC-pyridin-2-yl | CH/CH | S | +++ |
| —CH₂CCMe | 3-F,4-Me-pyridin-2-yl | CH/CH | S | +++ |
| —CH₂CCMe | 3-F,5-Me-pyridin-2-yl | CH/CH | S | +++ |
| —CH₂CCMe | 3-F,6-Me-pyridin-2-yl | CH/CH | S | +++ |
| —CH₂CCMe | 3-Me,4-CN-pyridin-2-yl | CH/CH | S | +++ |
| —CH₂CCMe | pyridazine-3-yl | CH/CH | S | +++ |
| —CH₂CCMe | 3-Me,5-CN-pyridin-2-yl | CH/CH | S | +++ |
| —CH₂CCMe | 3-Me,6-CN-pyridin-2-yl | CH/CH | S | +++ |
| —CH₂CCMe | 3,5-Me₂-pyrazin-2-yl | CH/CH | S | +++ |
| —CH₂CCMe | 3,6-Me₂-pyrazin-2-yl | CH/CH | S | +++ |
| —CH₂CCMe | 4-CN-pyridazin-3-yl | CH/CH | S | +++ |
| —CH₂CCMe | 6-CN-pyridazin-3-yl | CH/CH | S | +++ |
| —CH₂CCMe | 4-Me-pyridazin-3-yl | CH/CH | S | +++ |
| —CH₂CCMe | 5-Me-pyridazin-3-yl | CH/CH | S | +++ |
| —CH₂CCMe | 6-Me-pyridazin-3-yl | CH/CH | S | +++ |
| —CH₂CCMe | 6-CN,4-Me-pyridazin-3-yl | CH/CH | S | +++ |
| —CH₂CCMe | 3-F,4-CN-pyridin-2-yl | CH/CH | S | +++ |
| —CH₂CCMe | 6-CN-pyrimidin-2-yl | CH/CH | S | +++ |
| —CH₂CCMe | 4-CN-pyridin-2-yl | CH/CH | S | +++ |
| —CH₂CCMe | 6-CN-pyrimidin-4-yl | CH/CH | S | +++ |
| —CH₂CCMe | 4-(3′-pyridyl)pyrimidin-2-yl | CH/CH | S | +++ |
| —CH₂CCMe | 4-CN-1,3,5-triazin-2-yl | CH/CH | S | + |
| —CH₂CCMe | 4-Cl-1,3,5-triazin-2-yl | CH/CH | S | + |
| —CH₂CCMe | 5-Me,6-CN-pyrimidin-4-yl | CH/CH | S | +++ |
| —CH₂CCMe | 3,5-Me₂-pyridin-2-yl | CH/CH | S | +++ |
| —CH₂CCMe | pyridine-3-yl | CH/CH | S | +++ |
| —CH₂CCMe | 4-CN-pyridazin-3-yl | CH/CH | S | +++ |
| —CH₂CCMe | 1-Me-1H-pyrazol-4-yl | CH/CH | S | +++ |
| —CH₂CCMe | pyridine-4-yl | CH/CH | S | +++ |
| —CH₂CCMe | pyrimidin-5-yl | CH/CH | S | +++ |
| —CH₂CCMe | pyridazine-4-yl | CH/CH | S | +++ |
| —CH₂CCMe | 2-CN-pyridin-3-yl | CH/CH | S | +++ |
| —CH₂CCMe | 3-CN-pyridin-4-yl | CH/CH | S | +++ |
| —CH₂CCMe | pyridin-2-yl | CH/CH | S | +++ |
| —CH₂CCMe | 3,6-Me₂-pyrazin-2-yl | CCN/CH | S | +++ |
| —CH₂CCMe | 3-Me,4-CN-pyridin-2-yl | CCN/CH | S | +++ |
| —CH₂CCMe | 4-CN-pyridazin-3-yl | CCN/CH | S | +++ |
| —CH₂CCMe | 3-Me,5-CN-pyridin-2-yl | CCN/CH | S | +++ |
| —CH₂CCMe | pyridazine-3-yl | CCN/CH | S | + |
| —CH₂CCMe | 3-Me-pyridin-2-yl | CH/CH | CH₂ | +++ |
| —CH₂CCMe | 3-CN-pyridin-2-yl | CH/CH | CH₂ | +++ |
| —CH₂CCMe | 3-F-pyridin-2-yl | CH/CH | CH₂ | +++ |
| —CH₂CCMe | 3-CN-pyrazin-2-yl | CH/CH | CH₂ | +++ |
| —CH₂CCMe | 3-Me,4-CN-pyridin-2-yl | CH/CH | CH₂ | +++ |
| —CH₂CCMe | 5-Me,6-CN-pyrimidin-4-yl | CH/CH | CH₂ | +++ |
| —CH₂CCMe | 3-Me-pyridin-2-yl | CH/N | S | +++ |
| —CH₂CCMe | 3-CN-pyridin-2-yl | CH/N | S | +++ |
| —CH₂CCMe | 3-F-pyridin-2-yl | CH/N | S | +++ |
| —CH₂CCMe | 3-MeO-pyridin-2-yl | CH/N | S | +++ |
| —CH₂CCMe | 3-F,4-Me-pyridin-2-yl | CH/N | S | +++ |
| —CH₂CCMe | 3-F,5-Me-pyridin-2-yl | CH/N | S | +++ |
| —CH₂CCMe | 3-F,6-Me-pyridin-2-yl | CH/N | S | +++ |
| —CH₂CCMe | 3-Me,4-CN-pyridin-2-yl | CH/N | S | +++ |
| —CH₂CCMe | pyridazine-3-yl | CH/N | S | +++ |
| —CH₂CCMe | 3-Me,5-CN-pyridin-2-yl | CH/N | S | +++ |
| —CH₂CCMe | 3-Me, 6-CN-pyridin-2-yl | CH/N | S | +++ |
| —CH₂CCMe | 3,5-Me₂-pyrazin-2-yl | CH/N | S | +++ |
| —CH₂CCMe | 3,6-Me₂-pyrazin-2-yl | CH/N | S | +++ |
| —CH₂CCMe | 4-CN-pyridazin-3-yl | CH/N | S | +++ |
| —CH₂CCMe | 4-Me-pyridazin-3-yl | CH/N | S | +++ |
| —CH₂CCMe | 5-Me-pyridazin-3-yl | CH/N | S | +++ |
| —CH₂CCMe | 6-Me-pyridazin-3-yl | CH/N | S | +++ |
| —CH₂CCMe | 3-F,4-CN-pyridin-2-yl | CH/N | S | +++ |
| —CH₂CCMe | 5-Me,6-CN-pyrimidin-4-yl | CH/N | S | +++ |
| —CH₂CCMe | 3,5-Me₂-pyridin-2-yl | CH/N | S | +++ |
| —CH₂CCMe | 4-CN-pyridazin-3-yl | CH/N | S | +++ |
| —CH₂CCCl | 3-F,6-Me-pyridin-2-yl | CH/CH | S | +++ |
| —CH₂CCCl | pyridazine-3-yl | CH/CH | S | +++ |
| —CH₂CCCl | 3,5-Me₂-pyrazin-2-yl | CH/CH | S | +++ |
| —CH₂CCCl | 3,6-Me₂-pyrazin-2-yl | CH/CH | S | +++ |
| —CH₂CCCl | 4-Me-pyridazin-3-yl | CH/CH | S | +++ |
| —CH₂CCCl | 3,5-Me₂-pyridin-2-yl | CH/CH | S | +++ |
| —CH₂CCCl | 3-F,6-Me-pyridin-2-yl | CH/N | S | +++ |
| —CH₂CCCl | pyridazine-3-yl | CH/N | S | +++ |
| —CH₂CCCl | 3,5-Me₂-pyrazin-2-yl | CH/N | S | +++ |
| —CH₂CCCl | 3,6-Me₂-pyrazin-2-yl | CH/N | S | +++ |
| —CH₂CCCl | 4-Me-pyridazin-3-yl | CH/N | S | +++ |
| —CH₂CCCl | 3,5-Me₂-pyridin-2-yl | CH/N | S | +++ |
| —CH₂CCH | 3-F,6-Me-pyridin-2-yl | CH/CH | S | +++ |
| —CH₂CCH | pyridazine-3-yl | CH/CH | S | +++ |
| —CH₂CCH | 3,5-Me₂-pyrazin-2-yl | CH/CH | S | +++ |
| —CH₂CCH | 3,6-Me₂-pyrazin-2-yl | CH/CH | S | +++ |
| —CH₂CCH | 4-Me-pyridazin-3-yl | CH/CH | S | +++ |
| —CH₂CCH | 3,5-Me₂-pyridin-2-yl | CH/CH | S | +++ |
| —CH₂CCH | 3-F,6-Me-pyridin-2-yl | CH/N | S | +++ |
| —CH₂CCH | pyridazine-3-yl | CH/N | S | +++ |
| —CH₂CCH | 3,5-Me₂-pyrazin-2-yl | CH/N | S | +++ |
| —CH₂CCH | 3,6-Me₂-pyrazin-2-yl | CH/N | S | +++ |
| —CH₂CCH | 4-Me-pyridazin-3-yl | CH/N | S | +++ |
| —CH₂CCH | 3,5-Me₂-pyridin-2-yl | CH/N | S | +++ |

TABLE 6

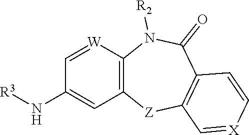

Examples:

| R² | R³ | X/W | Z | Activity |
|---|---|---|---|---|
| —CH₂CCMe | oxetan-3-yl | CH/CH | S | +++ |
| —CH₂CCMe | (oxetan-3-yl)CH₂ | CH/CH | S | + |
| —CH₂CCMe | (3-Me-oxetan-3-yl)CH₂ | CH/CH | S | +++ |
| —CH₂CCMe | 3-MeOcBu | CH/CH | S | +++ |
| —CH₂CCMe | 3-HOcBu | CH/CH | S | +++ |
| —CH₂CCMe | 3-MeOCH₂-cBu | CH/CH | S | +++ |
| —CH₂CCMe | 3-HOCH₂-cBu | CH/CH | S | +++ |
| —CH₂CCMe | 2-oxaspiro[3.3]heptan-6-yl | CH/CH | S | +++ |
| —CH₂CCMe | tetrahydrofuran-3-yl | CH/CH | S | +++ |
| —CH₂CCMe | 4-Me-tetrahydrofuran-3-yl | CH/CH | S | +++ |
| —CH₂CCMe | 4-MeO-tetrahydrofuran-3-yl | CH/CH | S | +++ |
| —CH₂CCMe | 4-CN-tetrahydrofuran-3-yl | CH/CH | S | + |
| —CH₂CCMe | 2-Me-tetrahydrofuran-3-yl | CH/CH | S | +++ |
| —CH₂CCMe | cPent | CH/CH | S | +++ |
| —CH₂CCMe | tetrahydropyran-3-yl | CH/CH | S | +++ |
| —CH₂CCMe | bicyclo[2.2.1]]heptane-2-yl | CH/CH | S | +++ |
| —CH₂CCMe | bicyclo[1.1.1]pentan-1-yl | CH/CH | S | +++ |
| —CH₂CCMe | 3-MeO-bicyclo[1.1.1]]pentan-1-yl | CH/CH | S | +++ |
| —CH₂CCMe | 6-oxospiro[3.3]heptan-2-yl | CH/CH | S | +++ |
| —CH₂CCMe | 6-HO-spiro[3.3]]heptan-2-yl | CH/CH | S | +++ |
| —CH₂CCMe | (CH₂)₃OMe | CH/CH | S | +++ |
| —CH₂CCMe | (CH₂)₃OH | CH/CH | S | +++ |
| —CH₂CCMe | oxetan-3-yl | CH/N | S | +++ |
| —CH₂CCMe | (3-Me-oxetan-3-yl)CH₂ | CH/N | S | +++ |
| —CH₂CCMe | 3-MeOcBu | CH/N | S | +++ |
| —CH₂CCMe | 3-HOcBu | CH/N | S | +++ |
| —CH₂CCMe | 3-MeOCH₂-cBu | CH/N | S | +++ |
| —CH₂CCMe | 3-HOCH₂-cBu | CH/N | S | +++ |
| —CH₂CCMe | 2-oxaspiro[3.3]heptan-6-yl | CH/N | S | +++ |
| —CH₂CCMe | tetrahydrofuran-3-yl | CH/N | S | +++ |
| —CH₂CCMe | 4-Me-tetrahydrofuran-3-yl | CH/N | S | +++ |
| —CH₂CCMe | 4-MeO-tetrahydrofuran-3-yl | CH/N | S | +++ |
| —CH₂CCMe | 2-Me-tetrahydrofuran-3-yl | CH/N | S | +++ |
| —CH₂CCMe | cPent | CH/N | S | +++ |
| —CH₂CCMe | tetrahydropyran-3-yl | CH/N | S | +++ |
| —CH₂CCMe | bicyclo[1.1.1]pentan-1-yl | CH/N | S | +++ |
| —CH₂CCMe | 3-MeO-bicyclo[1.1.1]]pentan-1-yl | CH/N | S | +++ |
| —CH₂CCMe | 6-oxospiro[3.3]]heptan-2-yl | CH/N | S | +++ |
| —CH₂CCMe | 6-HO-spiro[3.3]]heptan-2-yl | CH/N | S | +++ |
| —CH₂CCMe | (CH₂)₃OMe | CH/N | S | +++ |
| —CH₂CCMe | (CH₂)₃OH | CH/N | S | +++ |
| —CH₂CCCl | oxetan-3-yl | CH/CH | S | +++ |
| —CH₂CCCl | 3-MeOcBu | CH/CH | S | +++ |
| —CH₂CCCl | 3-HOcBu | CH/CH | S | +++ |
| —CH₂CCCl | 3-MeOCH₂-cBu | CH/CH | S | +++ |
| —CH₂CCCl | 2-oxaspiro[3.3]]heptan-6-yl | CH/CH | S | +++ |
| —CH₂CCCl | oxetan-3-yl | CH/N | S | +++ |
| —CH₂CCCl | 3-MeOcBu | CH/N | S | +++ |
| —CH₂CCCl | 3-HOcBu | CH/N | S | +++ |
| —CH₂CCCl | 3-MeOCH₂-cBu | CH/N | S | +++ |
| —CH₂CCCl | 2-oxaspiro[3.3]heptan-6-yl | CH/N | S | +++ |
| —CH₂CCH | oxetan-3-yl | CH/CH | S | +++ |
| —CH₂CCH | 3-MeOcBu | CH/CH | S | +++ |
| —CH₂CCH | 3-HOcBu | CH/CH | S | +++ |
| —CH₂CCH | 3-MeOCH₂-cBu | CH/CH | S | +++ |
| —CH₂CCH | 2-oxaspiro[3.3]heptan-6-yl | CH/CH | S | +++ |
| —CH₂CCH | oxetan-3-yl | CH/N | S | +++ |
| —CH₂CCH | 3-MeOcBu | CH/N | S | +++ |
| —CH₂CCH | 3-HOcBu | CH/N | S | +++ |
| —CH₂CCH | 3-MeOCH₂-cBu | CH/N | S | +++ |
| —CH₂CCH | 2-oxaspiro[3.3]heptan-6-yl | CH/N | S | +++ |
| —CH₂CCMe | oxetan-3-yl | CH/CH | CH₂ | +++ |
| —CH₂CCMe | (3-Me-oxetan-3-yl)CH₂ | CH/CH | CH₂ | +++ |
| —CH₂CCMe | 3-MeOcBu | CH/CH | CH₂ | +++ |
| —CH₂CCMe | 3-HOcBu | CH/CH | CH₂ | +++ |
| —CH₂CCMe | 3-MeOCH₂-cBu | CH/CH | CH₂ | +++ |
| —CH₂CCMe | 3-HOCH₂-cBu | CH/CH | CH₂ | +++ |
| —CH₂CCMe | 2-oxaspiro[3.3]heptan-6-yl | CH/CH | CH₂ | +++ |
| —CH₂CCMe | tetrahydropyran-3-yl | CH/CH | CH₂ | +++ |
| —CH₂CCMe | 3-MeO-bicyclo[1.1.1]pentan-1-yl | CH/CH | CH₂ | +++ |
| —CH₂CCMe | 6-oxospiro[3.3]]heptan-2-yl | CH/CH | CH₂ | +++ |
| —CH₂CCMe | 6-HO-spiro[3.3]]heptan-2-yl | CH/CH | CH₂ | +++ |
| —CH₂CCMe | (CH₂)3OMe | CH/CH | CH₂ | +++ |
| —CH₂CCMe | (CH₂)3 OH | CH/CH | CH₂ | +++ |

TABLE 7

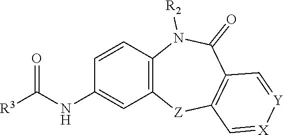

Examples:

| R² | R³ | X/Y | Z | Activity |
|---|---|---|---|---|
| —(CH₂)2OMe | Me | CH/CH | S | + |
| —(CH₂)2OMe | 4-FPh | CH/CH | S | + |
| —(CH₂)2OMe | Ph | CH/CH | S | ++ |
| —(CH₂)2OMe | 4-CF₃Ph | CH/CH | S | + |
| —(CH₂)2OMe | cHex | CH/CH | S | + |
| —(CH₂)2OMe | pyrid-3-yl | CH/CH | S | + |
| —(CH₂)2OMe | pyrid-4-yl | CH/CH | S | + |
| —(CH₂)2OMe | 4-MePh | CH/CH | S | + |
| —(CH₂)2OMe | 4-N₃Ph | CH/CH | S | + |
| —(CH₂)2OMe | oxazol-4-yl | CH/CH | S | + |
| —(CH₂)2OMe | 3-N3Ph | CH/CH | S | ++ |
| —(CH₂)2OMe | cBu | CH/CH | S | ++ |
| —(CH₂)2OMe | tetrahydropyran-4-yl | CH/CH | S | + |
| —(CH₂)2OMe | cHeptyl | CH/CH | S | + |
| —(CH₂)2OMe | 4-ClPh | CH/CH | S | + |
| —(CH₂)2OMe | 3-ClPh | CH/CH | S | + |
| —(CH₂)2OMe | 4-MeOPh | CH/CH | S | +++ |
| —(CH₂)2OMe | 4-CNPh | CH/CH | S | + |
| —(CH₂)2OMe | 4-EtPh | CH/CH | S | + |
| —(CH₂)2OMe | 4-HCCPh | CH/CH | S | ++ |
| —(CH₂)2OMe | 2-naphtyl | CH/CH | S | + |
| —(CH₂)2OMe | cPr | CH/CH | S | + |
| —(CH₂)2OMe | 3-oxo-cBu | CH/CH | S | + |
| —(CH₂)2OMe | 4-CF₃OPh | CH/CH | S | + |
| —(CH₂)2OMe | 4-FH₂COPh | CH/CH | S | +++ |
| —(CH₂)2OMe | 3,4-Cl₂Ph | CH/CH | S | + |
| —(CH₂)2OMe | spiro[2.2]pentanyl | CH/CH | S | + |
| —(CH₂)2OMe | spiro[3.3]]heptan-2-yl | CH/CH | S | ++ |
| —(CH₂)2OMe | 2-MeOPh | CH/CH | S | +++ |
| —(CH₂)2OMe | 3-MeOPh | CH/CH | S | +++ |
| —(CH₂)2OMe | 3-Br,4-MeOPh | CH/CH | S | + |
| —(CH₂)2OMe | 3-F,4MeOPh | CH/CH | S | ++ |
| —(CH₂)2OMe | 2-Me-furan-3-yl | CH/CH | S | ++ |
| —(CH₂)2OMe | 2,5-Me₂-furan-3-yl | CH/CH | S | + |
| —(CH₂)2OMe | benzo[d]oxazol-5-yl | CH/CH | S | +++ |
| —(CH₂)2OMe | 4-PrOPh | CH/CH | S | + |
| —(CH₂)2OMe | benzo[d][1,3]dioxol-5-yl | CH/CH | S | +++ |
| —(CH₂)2OMe | 3,4-F₂Ph | CH/CH | S | + |
| —(CH₂)2OMe | 3-Cl,4-FPh | CH/CH | S | + |
| —(CH₂)2OMe | 3-MeO,4-ClPh | CH/CH | S | ++ |
| —(CH₂)2OMe | 3,5-F₂,4-MeOPh | CH/CH | S | ++ |
| —(CH₂)2OMe | thiazol-4-yl | CH/CH | S | + |
| —(CH₂)2OMe | benzo[d]thiazol-5-yl | CH/CH | S | +++ |
| —(CH₂)2OMe | imidazo[1,2-a]pyridin-6-yl | CH/CH | S | + |
| —(CH₂)2OMe | imidazo[1,2-a]pyridin-7-yl | CH/CH | S | + |
| —(CH₂)2OMe | pyrimidin-2-yl | CH/CH | S | + |
| —(CH₂)2OMe | pyrimidin-4-yl | CH/CH | S | + |
| —(CH₂)2OMe | pyrazin-2-yl | CH/CH | S | + |
| —(CH₂)2OMe | 3-F,4-HC(O)NHPh | CH/CH | S | + |
| —(CH₂)2OMe | 3-F,4-F₃COPh | CH/CH | S | + |
| —(CH₂)2OMe | isoxazol-5-yl | CH/CH | S | + |
| —(CH₂)2OMe | 3-Cl,4-HC(O)NHPh | CH/CH | S | + |
| —(CH₂)2OMe | 3-F,4-F₂HCOPh | CH/CH | S | + |
| —(CH₂)2OMe | 2-Me,4-MeOPh | CH/CH | S | + |
| —(CH₂)2OMe | 2-F,4-MeOPh | CH/CH | S | ++ |
| —(CH₂)2OMe | 3-H2NC(O)Ph | CH/CH | S | + |
| —(CH₂)2OMe | 3-MeNHC(O)Ph | CH/CH | S | + |
| —(CH₂)2OMe | 3-(2′-CNEt)Ph | CH/CH | S | +++ |
| —(CH₂)2OMe | 3-(CNCH₂)Ph | CH/CH | S | ++ |
| —(CH₂)2OMe | 4-(NH2CH₂)Ph | CH/CH | S | + |
| —(CH₂)2OMe | 3-Me₂NC(O)Ph | CH/CH | S | ++ |
| —(CH₂)2OMe | 4-F₂HCOPh | CH/CH | S | ++ |
| —(CH₂)2OMe | 6-(HCC)pyrid-3-yl | CH/CH | S | + |
| —(CH₂)2OMe | 2-(HCC)pyrid-4-yl | CH/CH | S | + |
| —(CH₂)2OMe | 6-MeO-pyrid-3-yl | CH/CH | S | +++ |
| —(CH₂)2OMe | 2-MeO-pyrid-4-yl | CH/CH | S | ++ |
| —(CH₂)2OMe | 3-MeO,4-FPh | CH/CH | S | ++ |
| —(CH₂)2OMe | benzo[d]oxazol-6-yl | CH/CH | S | + |
| —(CH₂)2OMe | 2-Me-benzo[d]oxazol-5-yl | CH/CH | S | +++ |
| —(CH₂)2OMe | 3-CNPh | CH/CH | S | + |
| —(CH₂)2OMe | 3-FPh | CH/CH | S | + |
| —(CH₂)2OMe | 3-CF₃Ph | CH/CH | S | + |
| —(CH₂)2OMe | 3-F₂HCOPh | CH/CH | S | ++ |
| —(CH₂)2OMe | 2-oxo-2,3-dihydrobenzo[d]oxazol-5-yl | CH/CH | S | + |
| —(CH₂)2OMe | 3-Me-2-oxo-2,3-dihydrobenzo[d]oxazol-5-yl | CH/CH | S | ++ |
| —(CH₂)2OMe | 3-FH₂COPh | CH/CH | S | +++ |
| —(CH₂)2OMe | 2,2-F2-benzo[d][1,3]dioxol-5-yl | CH/CH | S | + |
| —(CH₂)2OMe | furan-3-yl | CH/CF | S | +++ |
| —(CH₂)2OMe | furan-3-yl | CF/CH | S | +++ |
| —(CH₂)2OMe | furan-3-yl | CH/CH | CH₂ | + |
| —(CH₂)2OH | furan-3-yl | CH/CH | S | + |
| —CH₂CHMeOMe | furan-3-yl | CH/CH | S | + |
| —(CH₂)2OPr | 4-MeOPh | CH/CH | S | +++ |
| -nHexyl | furan-3-yl | CH/CH | S | +++ |
| —CH₂CH═CHPr | furan-3-yl | CH/CH | S | +++ |
| —CH₂CH═CH₂ | furan-3-yl | CH/CH | S | +++ |
| —CH₂CH═CH₂ | benzo[d]thiazol-5-yl | CH/CH | S | +++ |
| —CH₂CH═CH₂ | benzo[d]thiazol-5-yl | CF/CH | S | +++ |
| —CH₂CH═CHMe | benzo[d]thiazol-5-yl | CH/CH | S | +++ |
| —CH₂CH═CHMe | 4-MeOPh | CH/CH | S | +++ |
| —(CH₂)5CONHMe | furan-3-yl | CH/CH | S | + |
| —(CH₂)2O(CH₂)3CH═CH₂ | furan-3-yl | CH/CH | S | +++ |
| —(CH₂)₂OCH₂CCH | furan-3-yl | CH/CH | S | +++ |
| —(CH₂)₂OCH₂CCH | 4-MeOPh | CH/CH | S | +++ |
| —(CH₂)₂OCH₂CCH | 4-MeOPh | CH/CH | CH₂ | +++ |
| —(CH₂)₂OCH₂CCH | 4-N₃Ph | CH/CH | S | +++ |
| —(CH₂)₂OCH₂CCH | 3,4-Cl₂Ph | CH/CH | S | ++ |
| —(CH₂)₂OCH₂CCH | 3-F,4-MeOPh | CH/CH | S | + |
| —(CH₂)₂OCH₂CCH | benzo[d]thiazol-5-yl | CH/CH | S | +++ |
| —(CH₂)₂OCH₂CCH | benzo[d]thiazol-5-yl | CF/CH | S | +++ |
| —(CH₂)₂OCH₂CCH | 3-(2′-CNEt)Ph | CH/CH | S | +++ |

TABLE 8

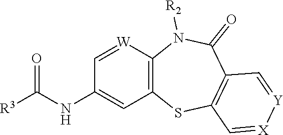

Examples:

| R² | R³ | X/Y | W | Activity |
|---|---|---|---|---|
| —CH₂CCMe | furan-3-yl | CH/CH | CH | +++ |
| —CH₂CCMe | cPent | CH/CH | CH | +++ |
| —CH₂CCMe | cHex | CH/CH | CH | +++ |
| —CH₂CCMe | cBu | CH/CH | CH | +++ |
| —CH₂CCMe | 4-MeOPh | CH/CH | CH | +++ |
| —CH₂CCMe | cPr | CH/CH | CH | +++ |
| —CH₂CCMe | 4-CF₃OPh | CH/CH | CH | +++ |
| —CH₂CCMe | spiro[3.3]heptan-2-yl | CH/CH | CH | +++ |
| —CH₂CCMe | 3-MeOPh | CH/CH | CH | +++ |
| —CH₂CCMe | benzo[d]oxazol-5-yl | CH/CH | CH | +++ |
| —CH₂CCMe | benzo[d]thiazol-5-yl | CH/CH | CH | +++ |
| —CH₂CCMe | 3-(2′-CNEt)Ph | CH/CH | CH | +++ |
| —CH₂CCMe | 3-Me₂NC(O)Ph | CH/CH | CH | +++ |
| —CH₂CCMe | 4-F₂HCOPh | CH/CH | CH | +++ |
| —CH₂CCMe | 4-FH₂COPh | CH/CH | CH | +++ |
| —CH₂CCMe | 6-(HCC)pyrid-3-yl | CH/CH | CH | +++ |
| —CH₂CCMe | 6-MeO-pyrid-3-yl | CH/CH | CH | +++ |
| —CH₂CCMe | 3-Me-2-oxo-2,3-dihydrobenzo[d]oxazol-5-yl | CH/CH | CH | +++ |
| —CH₂CCMe | 3-MeO₂C-bicyclo [1.1.1]pentan-1-yl | CH/CH | CH | + |
| —CH₂CCMe | bicyclo[1.1.1]]pentan-1-yl | CH/CH | CH | +++ |
| —CH₂CCMe | 3-F-bicyclo[1.1.1]pentan-1-yl | CH/CH | CH | +++ |
| —CH₂CCMe | 3-Me-bicyclo[1.1.1]pentan-1-yl | CH/CH | CH | +++ |
| —CH₂CCMe | 3-CN-bicyclo[1.1.1]]pentan-1-yl | CH/CH | CH | +++ |
| —CH₂CCMe | 3-CF₃-bicyclo[1.1.1]pentan-1-yl | CH/CH | CH | ++ |
| —CH₂CCMe | 3-MeO-bicyclo[1.1.1]pentan-1-yl | CH/CH | CH | +++ |
| —CH₂CCMe | 3-HO-bicyclo[1.1.1]pentan-1-yl | CH/CH | CH | +++ |
| —CH₂CCMe | (CH₂)₃OMe | CH/CH | CH | +++ |
| —CH₂CCMe | MeO(CH₂)₂CHMe- | CH/CH | CH | +++ |
| —CH₂CCMe | O(CH₂)₂OMe | CH/CH | CH | +++ |
| —CH₂CCMe | (CH₂)₂CN | CH/CH | CH | +++ |
| —CH₂CCMe | NH(CH₂)₂OMe | CH/CH | CH | +++ |
| —CH₂CCMe | (CH₂)₃OH | CH/CH | CH | +++ |
| —CH₂CCMe | CH═CHCH₂OMe | CH/CH | CH | +++ |
| —CH₂CCMe | CH—CHCH₂OH | CH/CH | CH | +++ |
| —CH₂CCMe | 3-MeOCH₂-cBu | CH/CH | CH | +++ |
| —CH₂CCMe | (oxetan-3-yl)CH₂ | CH/CH | CH | +++ |
| —CH₂CCMe | cis-3-MeOcBu | CH/CH | CH | +++ |
| —CH₂CCMe | trans-3-MeOcBu | CH/CH | CH | +++ |
| —CH₂CCMe | tetrahydrofuran-3-yl | CH/CH | CH | +++ |
| —CH₂CCMe | tetrahydrofuran-2-yl | CH/CH | CH | +++ |
| —CH₂CCMe | 6-oxospiro[3.3]heptan-2-yl | CH/CH | CH | +++ |
| —CH₂CCMe | 6-(hydroxyimino)-spiro[3.3]]heptane-2-yl | CH/CH | CH | +++ |
| —CH₂CCMe | 6-(methoxyimino)-spiro[3.3]]heptane-2-yl | CH/CH | CH | + |
| —CH₂CCMe | 5-oxopyrrolidin-3-yl | CH/CH | CH | +++ |
| —CH₂CCMe | 2-oxaspiro[3.3]heptan-6-yl | CH/CH | CH | +++ |
| —CH₂CCMe | oxetan-3-yl | CH/CH | CH | +++ |
| —CH₂CCMe | 6-HO-spiro[3.3]]heptan-2-yl | CH/CH | CH | +++ |
| —CH₂CCMe | pyrrolidin-3-yl | CH/CH | CH | + |
| —CH₂CCMe | 1-Me-pyrrolidin-3-yl | CH/CH | CH | + |
| —CH₂CCMe | 1-Me-pyrrolidin-2-yl | CH/CH | CH | +++ |
| —CH₂CCMe | 2-oxo-pyrrolidin-3-yl | CH/CH | CH | ++ |
| —CH₂CCMe | N-Me-2-oxo-pyrrolidin-3-yl | CH/CH | CH | ++ |
| —CH₂CCMe | 2-oxo-pyrrolidin-4-yl | CH/CH | CH | + |
| —CH₂CCMe | CCMe | CH/CH | CH | +++ |
| —CH₂CCMe | CCCH₂OMe | CH/CH | CH | ++ |
| —CH₂CCMe | cis-3-OH-cBu | CH/CH | CH | +++ |
| —CH₂CCMe | trans-3-OH-cBu | CH/CH | CH | +++ |
| —CH₂CCMe | trans-2-MeO2C-cPr | CH/CH | CH | +++ |
| —CH₂CCMe | trans-2-HOCH₂-cPr | CH/CH | CH | +++ |
| —CH₂CCMe | trans-2-MeOCH₂-cPr | CH/CH | CH | +++ |
| —CH₂CCMe | tetrahydropyran-4-ethylidenyl | CH/CH | CH | +++ |
| —CH₂CCMe | 4-MeOPh | CF/CH | CH | +++ |
| —CH₂CCMe | 3-MeOPh | CF/CH | CH | +++ |
| —CH₂CCMe | benzo[d]thiazol-5-yl | CF/CH | CH | +++ |
| —CH₂CCMe | 3-(2′-CNEt)Ph | CF/CH | CH | +++ |
| —CH₂CCMe | 4-F₂HCOPh | CF/CH | CH | +++ |
| —CH₂CCMe | 4-FH₂COPh | CF/CH | CH | +++ |
| —CH₂CCMe | 6-MeO-pyrid-3-yl | CF/CH | CH | +++ |
| —CH₂CCMe | 3-Me-2-oxo-2,3-dihydrobenzo[d]oxazol-5-yl | CF/CH | CH | +++ |
| —CH₂CCMe | 3-MeO-bicyclo[1.1.1]pentan-1-yl | CF/CH | CH | +++ |
| —CH₂CCMe | 3-MeOCH₂-cBu | CF/CH | CH | +++ |
| —CH₂CCMe | (oxetan-3-yl)CH₂ | CF/CH | CH | +++ |
| —CH₂CCMe | cis-3-MeOcBu | CF/CH | CH | +++ |
| —CH₂CCMe | trans-3-MeOcBu | CF/CH | CH | +++ |
| —CH₂CCMe | 6-(hydroxyimino)-spiro[3.3]heptane-2-yl | CF/CH | CH | +++ |
| —CH₂CCMe | 6-(methoxyimino)-spiro[3.3]]heptane-2-yl | CF/CH | CH | +++ |
| —CH₂CCMe | 2-oxaspiro[3.3]heptan-6-yl | CF/CH | CH | +++ |
| —CH₂CCMe | 6-HO-spiro[3.3]]heptan-2-yl | CF/CH | CH | +++ |
| —CH₂CCMe | 4-MeOPh | CH/CF | CH | +++ |
| —CH₂CCMe | 3-MeOPh | CH/CF | CH | +++ |
| —CH₂CCMe | 3-Me-2-oxo-2,3-dihydrobenzo[d]oxazol-5-yl | CH/CF | CH | +++ |
| —CH₂CCMe | 3-MeO-bicyclo[1.1.1]]pentan-1-yl | CH/CF | CH | +++ |
| —CH₂CCMe | 3-MeOCH₂-cBu | CH/CF | CH | +++ |
| —CH₂CCMe | (oxetan-3-yl)CH₂ | CH/CF | CH | +++ |
| —CH₂CCMe | 6-(hydroxyimino)-spiro[3.3]heptane-2-yl | CH/CF | CH | +++ |
| —CH₂CCMe | 6-(methoxyimino)-spiro[3.3]heptane-2-yl | CH/CF | CH | +++ |
| —CH₂CCMe | 2-oxaspiro[3.3]heptan-6-yl | CH/CF | CH | +++ |
| —CH₂CCMe | 6-HO-spiro[3.3]]heptan-2-yl | CH/CF | CH | +++ |
| —CH₂CCMe | 3-HO-bicyclo[1.1.1]]pentan-1-yl | CH/CF | CH | +++ |
| —CH₂CCMe | (CH₂)₃OMe | CH/CF | CH | +++ |
| —CH₂CCMe | O(CH₂)₂OMe | CH/CF | CH | +++ |
| —CH₂CCMe | NH(CH₂)₂OMe | CH/CF | CH | +++ |
| —CH₂CCMe | MeO(CH₂)₂CHMe- | CH/CF | CH | +++ |
| —CH₂CCMe | (CH₂)₃OH | CH/CF | CH | +++ |
| —CH₂CCMe | CH—═CHCH₂OMe | CH/CF | CH | +++ |
| —CH₂CCMe | CH═CHCH₂OH | CH/CF | CH | +++ |
| —CH₂CCMe | 3-MeOCH₂-cBu | CH/CF | CH | +++ |
| —CH₂CCMe | cis-3-MeOcBu | CH/CF | CH | +++ |
| —CH₂CCMe | trans-3-MeOcBu | CH/CF | CH | +++ |
| —CH₂CCMe | tetrahydrofuran-3-yl | CH/CF | CH | +++ |
| —CH₂CCMe | tetrahydrofuran-2-yl | CH/CF | CH | +++ |
| —CH₂CCMe | 6-oxospiro[3.3]heptan-2-yl | CH/CF | CH | +++ |
| —CH₂CCMe | oxetan-3-yl | CH/CF | CH | +++ |
| —CH₂CCMe | cis-3-OH-cBu | CH/CF | CH | +++ |
| —CH₂CCMe | trans-3-OH-cBu | CH/CF | CH | +++ |
| —CH₂CCMe | trans-2-MeO₂C-cPr | CH/CF | CH | +++ |
| —CH₂CCMe | trans-2-HOCH₂-cPr | CH/CF | CH | +++ |
| —CH₂CCMe | trans-2-MeOCH₂-cPr | CH/CF | CH | +++ |
| —CH₂CCMe | furan-3-yl | CH/CH | N | +++ |
| —CH₂CCMe | cPent | CH/CH | N | +++ |
| —CH₂CCMe | cHex | CH/CH | N | +++ |
| —CH₂CCMe | cBu | CH/CH | N | +++ |
| —CH₂CCMe | 4-MeOPh | CH/CH | N | +++ |
| —CH₂CCMe | cPr | CH/CH | N | +++ |
| —CH₂CCMe | benzo[d]thiazol-5-yl | CH/CH | N | +++ |
| —CH₂CCMe | 6-MeO-pyrid-3-yl | CH/CH | N | +++ |
| —CH₂CCMe | 3-Me-2-oxo-2,3-dihydrobenzo[d]oxazol-5-yl | CH/CH | N | +++ |
| —CH₂CCMe | bicyclo[1.1.1]]pentan-1-yl | CH/CH | N | +++ |
| —CH₂CCMe | 3-F-bicyclo[1.1.1]pentan-1-yl | CH/CH | N | +++ |
| —CH₂CCMe | 3-Me-bicyclo[1.1.1]pentan-1-yl | CH/CH | N | +++ |
| —CH₂CCMe | 3-CN-bicyclo[1.1.1]]pentan-1-yl | CH/CH | N | +++ |
| —CH₂CCMe | 3-MeO-bicyclo[1.1.1]pentan-1-yl | CH/CH | N | +++ |
| —CH₂CCMe | 3-HO-bicyclo[1.1.1]]pentan-1-yl | CH/CH | N | +++ |
| —CH₂CCMe | (CH₂)₃OMe | CH/CH | N | +++ |
| —CH₂CCMe | O(CH₂)₂OMe | CH/CH | N | +++ |
| —CH₂CCMe | NH(CH₂)₂OMe | CH/CH | N | +++ |
| —CH₂CCMe | MeO(CH₂)₂CHMe- | CH/CH | N | +++ |
| —CH₂CCMe | (CH₂)3 OH | CH/CH | N | +++ |
| —CH₂CCMe | CH—CHCH₂OMe | CH/CH | N | +++ |
| —CH₂CCMe | CH—CHCH₂OH | CH/CH | N | +++ |
| —CH₂CCMe | 3-MeOCH₂-cBu | CH/CH | N | +++ |
| —CH₂CCMe | (oxetan-3-yl)CH₂ | CH/CH | N | +++ |
| —CH₂CCMe | cis-3-MeOcBu | CH/CH | N | +++ |
| —CH₂CCMe | trans-3-MeOcBu | CH/CH | N | +++ |
| —CH₂CCMe | tetrahydrofuran-3-yl | CH/CH | N | +++ |
| —CH₂CCMe | tetrahydrofuran-2-yl | CH/CH | N | +++ |
| —CH₂CCMe | 6-oxospiro[3.3]heptan-2-yl | CH/CH | N | +++ |
| —CH₂CCMe | 6-(hydroxyimino)-spiro[3.3]]heptane-2-yl | CH/CH | N | +++ |
| —CH₂CCMe | 6-(methoxyimino)-spiro[3.3]]heptane-2-yl | CH/CH | N | +++ |
| —CH₂CCMe | 2-oxaspiro[3.3]]heptan-6-yl | CH/CH | N | +++ |
| —CH₂CCMe | oxetan-3-yl | CH/CH | N | +++ |
| —CH₂CCMe | 6-HO-spiro[3.3]]heptan-2-yl | CH/CH | N | +++ |
| —CH₂CCMe | cis-3-OH-cBu | CH/CH | N | +++ |
| —CH₂CCMe | trans-3-OH-cBu | CH/CH | N | +++ |
| —CH₂CCMe | trans-2-MeO₂C-cPr | CH/CH | N | +++ |
| —CH₂CCMe | trans-2-HOCH₂-cPr | CH/CH | N | +++ |
| —CH₂CCMe | trans-2-MeOCH₂-cPr | CH/CH | N | +++ |
| —CH₂CCMe | 3-MeO-bicyclo[1.1.1]pentan-1-yl | CF/CH | N | +++ |
| —CH₂CCMe | 3-MeOCH₂-cBu | CF/CH | N | +++ |
| —CH₂CCMe | cis-3-MeOcBu | CF/CH | N | +++ |
| —CH₂CCMe | trans-3-MeOcBu | CF/CH | N | +++ |
| —CH₂CCMe | tetrahydrofuran-3-yl | CF/CH | N | +++ |
| —CH₂CCMe | tetrahydrofuran-2-yl | CF/CH | N | +++ |
| —CH₂CCMe | 6-(hydroxyimino)-spiro[3.3]heptane-2-yl | CF/CH | N | +++ |
| —CH₂CCMe | 6-(methoxyimino)-spiro[3.3]heptane-2-yl | CF/CH | N | +++ |
| —CH₂CCMe | 6-HO-spiro[3.3]heptan-2-yl | CF/CH | N | +++ |
| —CH₂CCMe | trans-2-HOCH₂-cPr | CF/CH | N | +++ |
| —CH₂CCMe | trans-2-MeOCH₂-cPr | CF/CH | N | +++ |
| —CH₂CCMe | 3-MeO-bicyclo[1.1.1]pentan-1-yl | CH/CF | N | +++ |
| —CH₂CCMe | 3-HO-bicyclo[1.1.1]pentan-1-yl | CH/CF | N | +++ |
| —CH₂CCMe | CH—═CHCH₂OMe | CH/CF | N | +++ |
| —CH₂CCMe | CH═CHCH₂OH | CH/CF | N | +++ |
| —CH₂CCMe | 3-MeOCH₂-cBu | CH/CF | N | +++ |
| —CH₂CCMe | cis-3-MeOcBu | CH/CF | N | +++ |
| —CH₂CCMe | trans-3-MeOcBu | CH/CF | N | +++ |
| —CH₂CCMe | tetrahydrofuran-3-yl | CH/CF | N | +++ |
| —CH₂CCMe | tetrahydrofuran-2-yl | CH/CF | N | +++ |
| —CH₂CCMe | 6-(hydroxyimino)-spiro[3.3]heptane-2-yl | CH/CF | N | +++ |
| —CH₂CCMe | 6-(methoxyimino)-spiro[3.3]heptane-2-yl | CH/CF | N | +++ |
| —CH₂CCMe | 6-HO-spiro[3.3]]heptan-2-yl | CH/CF | N | +++ |
| —CH₂CCMe | trans-2-HOCH₂-cPr | CH/CF | N | +++ |
| —CH₂CCMe | trans-2-MeOCH₂-cPr | CH/CF | N | +++ |
| —CH₂CC^{c}Pr | (oxetan-3-yl)CH₂ | CH/CH | CH | +++ |
| —CH₂CC^{c}Pr | 2-oxaspiro[3.3]]heptan-6-yl | CH/CH | CH | +++ |
| —CH₂CC^{c}Pr | 6-HO-spiro[3.3]]heptan-2-yl | CH/CH | CH | +++ |
| —CH₂CC^{c}Pr | (oxetan-3-yl)CH₂ | CH/CF | CH | +++ |
| —CH₂CC^{c}Pr | 6-(hydroxyimino)-spiro[3.3]heptane-2-yl | CH/CF | CH | +++ |
| —CH₂CC^{c}Pr | 6-(methoxyimino)-spiro[3.3]heptane-2-yl | CH/CF | CH | +++ |
| —CH₂CC^{c}Pr | 2-oxaspiro[3.3]heptan-6-yl | CH/CF | CH | +++ |
| —CH₂CC^{c}Pr | 6-HO-spiro[3.3]]heptan-2-yl | CH/CF | CH | +++ |
| —CH₂CC^{c}Pr | bicyclo[1.1.1]pentan-1-yl | CH/CH | N | +++ |
| —CH₂CC^{c}Pr | 3-F-bicyclo[1.1.1]pentan-1-yl | CH/CH | N | +++ |
| —CH₂CC^{c}Pr | 3-Me-bicyclo[1.1.1]pentan-1-yl | CH/CH | N | +++ |
| —CH₂CC^{c}Pr | 3-CN-bicyclo[1.1.1]]pentan-1-yl | CH/CH | N | +++ |
| —CH₂CC^{c}Pr | 3-MeO-bicyclo[1.1.1]]pentan-1-yl | CH/CH | N | +++ |
| —CH₂CC^{c}Pr | 3-HO-bicyclo[1.1.1]]pentan-1-yl | CH/CH | N | +++ |
| —CH₂CC^{c}Pr | (CH₂)₃OMe | CH/CH | N | +++ |
| —CH₂CC^{c}Pr | O(CH₂)₂OMe | CH/CH | N | +++ |
| —CH₂CC^{c}Pr | NH(CH₂)₂OMe | CH/CH | N | +++ |
| —CH₂CC^{c}Pr | MeO(CH₂)₂CHMe- | CH/CH | N | +++ |
| —CH₂CC^{c}Pr | (CH₂)3OH | CH/CH | N | +++ |
| —CH₂CC^{c}Pr | CH═CHCH₂OMe | CH/CH | N | +++ |
| —CH₂CC^{c}Pr | CH═CHCH₂OH | CH/CH | N | +++ |
| —CH₂CC^{c}Pr | 3-MeOCH₂-cBu | CH/CH | N | +++ |
| —CH₂CC^{c}Pr | (oxetan-3-yl)CH₂ | CH/CH | N | +++ |
| —CH₂CC^{c}Pr | cis-3-MeOcBu | CH/CH | N | +++ |
| —CH₂CC^{c}Pr | trans-3-MeOcBu | CH/CH | N | +++ |
| —CH₂CC^{c}Pr | tetrahydrofuran-3-yl | CH/CH | N | +++ |
| —CH₂CC^{c}Pr | tetrahydrofuran-2-yl | CH/CH | N | +++ |
| —CH₂CC^{c}Pr | 6-oxospiro[3.3]heptan-2-yl | CH/CH | N | +++ |
| —CH₂CC^{c}Pr | 6-(hydroxyimino)-spiro[3.3]]heptane-2-yl | CH/CH | N | +++ |
| —CH₂CC^{c}Pr | 6-(methoxyimino)-spiro[3.3]heptane-2-yl | CH/CH | N | +++ |
| —CH₂CC^{c}Pr | 2-oxaspiro[3.3]]heptan-6-yl | CH/CH | N | +++ |
| —CH₂CC^{c}Pr | oxetan-3-yl | CH/CH | N | +++ |
| —CH₂CC^{c}Pr | 6-HO-spiro[3.3]]heptan-2-yl | CH/CH | N | +++ |
| —CH₂CC^{c}Pr | cis-3-OH-cBu | CH/CH | N | +++ |
| —CH₂CC^{c}Pr | trans-3-OH-cBu | CH/CH | N | +++ |
| —CH₂CC^{c}Pr | trans-2-MeO₂C-cPr | CH/CH | N | +++ |
| —CH₂CC^{c}Pr | trans-2-HOCH₂-cPr | CH/CH | N | +++ |
| —CH₂CC^{c}Pr | trans-2-MeOCH₂-cPr | CH/CH | N | +++ |
| —CH₂CC^{c}Pr | 3-MeO-bicyclo[1.1.1]]pentan-1-yl | CH/CF | N | +++ |
| —CH₂CC^{c}Pr | 3-HO-bicyclo[1.1.1]pentan-1-yl | CH/CF | N | +++ |
| —CH₂CC^{c}Pr | (oxetan-3-yl)CH₂ | CH/CF | N | +++ |
| —CH₂CC^{c}Pr | 6-(hydroxyimino)-spiro[3.3]heptane-2-yl | CH/CF | N | +++ |
| —CH₂CC^{c}Pr | 6-(methoxyimino)-spiro[3.3]heptane-2-yl | CH/CF | N | +++ |
| —CH₂CC^{c}Pr | 2-oxaspiro[3.3]]heptan-6-yl | CH/CF | N | +++ |
| —CH₂CC^{c}Pr | 6-HO-spiro[3.3]]heptan-2-yl | CH/CF | N | +++ |
| —CH₂CCMe | 3-MeO-bicyclo[1.1.1]]pentan-1-yl | CCl/CH | CH | +++ |
| —CH₂CCMe | 3-HO-bicyclo[1.1.1]pentan-1-yl | CCl/CH | CH | +++ |
| —CH₂CCMe | (CH₂)₃OMe | CCl/CH | CH | +++ |
| —CH₂CCMe | O(CH₂)₂OMe | CCl/CH | CH | +++ |
| —CH₂CCMe | NH(CH₂)₂OMe | CCl/CH | CH | +++ |
| —CH₂CCMe | MeO(CH₂)₂CHMe- | CCI/CH | CH | +++ |
| —CH₂CCMe | (CH₂)₃OH | CCl/CH | CH | +++ |
| —CH₂CCMe | CH═CHCH₂OMe | CCl/CH | CH | +++ |
| —CH₂CCMe | CH═CHCH₂OH | CCI/CH | CH | +++ |
| —CH₂CCMe | 3-MeOCH₂-cBu | CCl/CH | CH | +++ |
| —CH₂CCMe | (oxetan-3-yl)CH₂ | CCl/CH | CH | +++ |
| —CH₂CCMe | cis-3-MeOcBu | CCI/CH | CH | +++ |
| —CH₂CCMe | trans-3-MeOcBu | CCl/CH | CH | +++ |
| —CH₂CCMe | tetrahydrofuran-3-yl | CCI/CH | CH | +++ |
| —CH₂CCMe | tetrahydrofuran-2-yl | CCI/CH | CH | +++ |
| —CH₂CCMe | 6-oxospiro[3.3]heptan-2-yl | CCl/CH | CH | +++ |
| —CH₂CCMe | 6-(hydroxyimino)-spiro[3.3]]heptane-2-yl | CCl/CH | CH | +++ |
| —CH₂CCMe | 6-(methoxyimino)-spiro[3.3]heptane-2-yl | CCl/CH | CH | +++ |
| —CH₂CCMe | 2-oxaspiro[3.3]heptan-6-yl | CCl/CH | CH | +++ |
| —CH₂CCMe | oxetan-3-yl | CCl/CH | CH | +++ |
| —CH₂CCMe | 6-HO-spiro[3.3]heptan-2-yl | CCl/CH | CH | +++ |
| —CH₂CCMe | cis-3-OH-cBu | CCI/CH | CH | +++ |
| —CH₂CCMe | trans-3-OH-cBu | CCI/CH | CH | +++ |
| —CH₂CCMe | trans-2-MeO₂C-cPr | CCl/CH | CH | +++ |
| —CH₂CCMe | trans-2-HOCH₂-cPr | CCl/CH | CH | +++ |
| —CH₂CCMe | trans-2-MeOCH₂-cPr | CCl/CH | CH | +++ |
| —CH₂CCMe | 3-MeO-bicyclo[1.1.1]pentan-1-yl | CCI/CH | N | +++ |
| —CH₂CCMe | 3-HO-bicyclo[1.1.1]pentan-1-yl | CCl/CH | N | +++ |
| —CH₂CCMe | (CH₂)₃OMe | CCl/CH | N | +++ |
| —CH₂CCMe | O(CH₂)₂OMe | CCI/CH | N | +++ |
| —CH₂CCMe | NH(CH₂)₂OMe | CCl/CH | N | +++ |
| —CH₂CCMe | MeO(CH₂)₂CHMe- | CCl/CH | N | +++ |
| —CH₂CCMe | (CH₂)3OH | CCI/CH | N | +++ |
| —CH₂CCMe | CH—═CHCH₂OMe | CCl/CH | N | +++ |
| —CH₂CCMe | CH—═CHCH₂OH | CCI/CH | N | +++ |
| —CH₂CCMe | 3-MeOCH₂-cBu | CCl/CH | N | +++ |
| —CH₂CCMe | (oxetan-3-yl)CH₂ | CCl/CH | N | +++ |
| —CH₂CCMe | cis-3-MeOcBu | CCl/CH | N | +++ |
| —CH₂CCMe | trans-3-MeOcBu | CCl/CH | N | +++ |
| —CH₂CCMe | tetrahydrofuran-3-yl | CCI/CH | N | +++ |
| —CH₂CCMe | tetrahydrofuran-2-yl | CCl/CH | N | +++ |
| —CH₂CCMe | 6-oxospiro[3.3]heptan-2-yl | CCI/CH | N | +++ |
| —CH₂CCMe | 6-(hydroxyimino)-spiro[3.3]heptane-2-yl | CCl/CH | N | +++ |
| —CH₂CCMe | 6-(methoxyimino)-spiro[3.3]]heptane-2-yl | CCl/CH | N | +++ |
| —CH₂CCMe | 2-oxaspiro[3.3]heptan-6-yl | CCI/CH | N | +++ |
| —CH₂CCMe | oxetan-3-yl | CCl/CH | N | +++ |
| —CH₂CCMe | 6-HO-spiro[3.3]]heptan-2-yl | CCl/CH | N | +++ |
| —CH₂CCMe | cis-3-OH-cBu | CCI/CH | N | +++ |
| —CH₂CCMe | trans-3-OH-cBu | CCl/CH | N | +++ |
| —CH₂CCMe | trans-2-MeO2C-cPr | CCl/CH | N | +++ |
| —CH₂CCMe | trans-2-HOCH₂-cPr | CCI/CH | N | +++ |
| —CH₂CCMe | trans-2-MeOCH₂-cPr | CCl/CH | N | +++ |
| —CH₂CCMe | CH—═CHCH₂OMe | CCN/CH | CH | +++ |
| —CH₂CCMe | trans-2-MeOCH₂-cPr | CCN/CH | CH | +++ |
| —CH₂CCMe | CH═CHCH₂OMe | CCN/CH | N | +++ |
| —CH₂CCMe | trans-2-MeOCH₂-cPr | CCN/CH | N | +++ |
| —CH₂CCCH₂F | 3-MeO-bicyclo[1.1.1]]pentan-1-yl | CH/CF | CH | +++ |
| —CH₂CCCH₂F | 3-MeOCH₂-cBu | CH/CF | CH | +++ |
| —CH₂CCCH₂F | cis-3-MeOcBu | CH/CF | CH | +++ |
| —CH₂CCCH₂F | trans-3-MeOcBu | CH/CF | CH | +++ |
| —CH₂CCCH₂F | 3-MeO-bicyclo[1.1.1]pentan-1-yl | CH/CH | N | +++ |
| —CH₂CCCH₂F | 3-MeOCH₂-cBu | CH/CH | N | +++ |
| —CH₂CCCH₂F | cis-3-MeOcBu | CH/CH | N | +++ |
| —CH₂CCCH₂F | trans-3-MeOcBu | CH/CH | N | +++ |
| —CH₂CCCl | 3-MeOCH₂-cBu | CH/CH | CH | +++ |
| —CH₂CCCl | cis-3-MeOcBu | CH/CH | CH | +++ |
| —CH₂CCCl | trans-3-MeOcBu | CH/CH | CH | +++ |
| —CH₂CCCl | 6-(hydroxyimino)-spiro[3.3]heptane-2-yl | CH/CH | CH | +++ |
| —CH₂CCCl | 6-(methoxyimino)-spiro[3.3]heptane-2-yl | CH/CH | CH | +++ |
| —CH₂CCCl | 3-MeOCH₂-cBu | CH/CF | CH | +++ |
| —CH₂CCCl | cis-3-MeOcBu | CH/CF | CH | +++ |
| —CH₂CCCl | trans-3-MeOcBu | CH/CF | CH | +++ |
| —CH₂CCCl | 6-(hydroxyimino)-spiro[3.3]heptane-2-yl | CH/CF | CH | +++ |
| —CH₂CCCl | 6-(methoxyimino)-spiro[3.3]]heptane-2-yl | CH/CF | CH | +++ |
| —CH₂CCCl | 3-MeOCH₂-cBu | CH/CH | N | +++ |
| —CH₂CCCl | cis-3-MeOcBu | CH/CH | N | +++ |
| —CH₂CCCl | trans-3-MeOcBu | CH/CH | N | +++ |
| —CH₂CCCl | 6-(hydroxyimino)-spiro[3.3]]heptane-2-yl | CH/CH | N | +++ |
| —CH₂CCCl | 6-(methoxyimino)-spiro[3.3]heptane-2-yl | CH/CH | N | +++ |
| —CH₂CCCl | 3-MeOCH₂-cBu | CCl/CH | CH | +++ |
| —CH₂CCCl | cis-3-MeOcBu | CCl/CH | CH | +++ |
| —CH₂CCCl | trans-3-MeOcBu | CCI/CH | CH | +++ |
| —CH₂CCCl | 6-(hydroxyimino)-spiro[3.3]heptane-2-yl | CCI/CH | CH | +++ |
| —CH₂CCCl | 6-(methoxyimino)-spiro[3.3]]heptane-2-yl | CCI/CH | CH | +++ |
| —CH₂CCCl | trans-2-HOCH₂-cPr | CCl/CH | CH | +++ |
| —CH₂CCCl | trans-2-MeOCH₂-cPr | CCl/CH | CH | +++ |
| —CH₂CCCl | 3-MeOCH₂-cBu | CCI/CH | N | +++ |
| —CH₂CCCl | cis-3-MeOcBu | CCl/CH | N | +++ |
| —CH₂CCCl | trans-3-MeOcBu | CCI/CH | N | +++ |
| —CH₂CCCl | 6-(hydroxyimino)-spiro[3.3]heptane-2-yl | CCl/CH | N | +++ |
| —CH₂CCCl | 6-(methoxyimino)-spiro[3.3]heptane-2-yl | CCl/CH | N | +++ |
| —CH₂CCCl | trans-2-HOCH₂-cPr | CCl/CH | N | +++ |
| —CH₂CCCl | trans-2-MeOCH₂-cPr | CCI/CH | N | +++ |
| —CH₂CCH | 3-MeOCH₂-cBu | CH/CH | CH | +++ |
| —CH₂CCH | cis-3-MeOcBu | CH/CH | CH | +++ |
| —CH₂CCH | trans-3-MeOcBu | CH/CH | CH | +++ |
| —CH₂CCH | 6-(hydroxyimino)-spiro[3.3]heptane-2-yl | CH/CH | CH | +++ |
| —CH₂CCH | 6-(methoxyimino)-spiro[3.3]]heptane-2-yl | CH/CH | CH | +++ |
| —CH₂CCH | 3-MeOCH₂-cBu | CH/CF | CH | +++ |
| —CH₂CCH | cis-3-MeOcBu | CH/CF | CH | +++ |
| —CH₂CCH | trans-3-MeOcBu | CH/CF | CH | +++ |
| —CH₂CCH | 6-(hydroxyimino)-spiro[3.3]]heptane-2-yl | CH/CF | CH | +++ |
| —CH₂CCH | 6-(methoxyimino)-spiro[3.3]]heptane-2-yl | CH/CF | CH | +++ |
| —CH₂CCH | 3-MeOCH₂-cBu | CH/CH | N | +++ |
| —CH₂CCH | cis-3-MeOcBu | CH/CH | N | +++ |
| —CH₂CCH | trans-3-MeOcBu | CH/CH | N | +++ |
| —CH₂CCH | 6-(hydroxyimino)-spiro[3.3]]heptane-2-yl | CH/CH | N | +++ |
| —CH₂CCH | 6-(methoxyimino)-spiro[3.3]heptane-2-yl | CH/CH | N | +++ |
| —CH₂CCH | 3-MeOCH₂-cBu | CCl/CH | CH | +++ |
| —CH₂CCH | cis-3-MeOcBu | CCl/CH | CH | +++ |
| —CH₂CCH | trans-3-MeOcBu | CCl/CH | CH | +++ |
| —CH₂CCH | 6-(hydroxyimino)-spiro[3.3]heptane-2-yl | CCl/CH | CH | +++ |
| —CH₂CCH | 6-(methoxyimino)-spiro[3.3]]heptane-2-yl | CCl/CH | CH | +++ |
| —CH₂CCH | trans-2-HOCH₂-cPr | CCI/CH | CH | +++ |
| —CH₂CCH | trans-2-MeOCH₂-cPr | CCl/CH | CH | +++ |
| —CH₂CCH | 6-(hydroxyimino)-spiro[3.3]heptane-2-yl | CCl/CH | N | +++ |
| —CH₂CCH | 6-(methoxyimino)-spiro[3.3]heptane-2-yl | CCI/CH | N | +++ |
| —CH₂CCH | trans-2-HOCH₂-cPr | CCl/CH | N | +++ |
| —CH₂CCH | trans-2-MeOCH₂-cPr | CCl/CH | N | +++ |
| —CH₂CCCF3 | 4-MeOPh | CH/CH | CH | +++ |
| —CH₂CCCF3 | 3-MeO-bicyclo[1.1.1]pentan-1-yl | CH/CH | CH | +++ |
| —CH₂CCCF3 | CH═CHCH₂OMe | CH/CH | CH | +++ |
| —CH₂CCCF3 | 3-MeOCH₂-cBu | CH/CH | CH | +++ |
| —CH₂CCCF3 | cis-3-MeOcBu | CH/CH | CH | +++ |
| —CH₂CCCF3 | trans-3-MeOcBu | CH/CH | CH | +++ |
| —CH₂CCCF3 | 3-MeO-bicyclo[1.1.1]pentan-1-yl | CH/CH | N | +++ |
| —CH₂CCCF3 | CH═CHCH₂OMe | CH/CH | N | +++ |
| —CH₂CCCF3 | CH—═CHCH₂OH | CH/CH | N | +++ |
| —CH₂CCCF3 | 3-MeOCH₂-cBu | CH/CH | N | +++ |
| —CH₂CCCF3 | cis-3-MeOcBu | CH/CH | N | +++ |
| —CH₂CCCF3 | trans-3-MeOcBu | CH/CH | N | +++ |
| —CH₂CCCF3 | 6-(hydroxyimino)-spiro[3.3]]heptane-2-yl | CH/CH | N | +++ |

TABLE 9

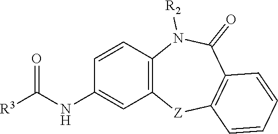

Examples:

| R² | R³ | Z | Activity |
|---|---|---|---|
| —CH₂CCMe | 4-MeOPh | CHMe | +++ |
| —CH₂CCMe | 3-Me-2-oxo-2,3-dihydrobenzo[d]oxazol-5-yl | CHMe | +++ |
| —CH₂CCMe | 3-MeO-bicyclo[1.1.1]pentan-1-yl | CHMe | +++ |
| —CH₂CCMe | 3-HO-bicyclo[1.1.1]]pentan-1-yl | CHMe | +++ |
| —CH₂CCMe | (CH₂)₃OMe | CHMe | +++ |
| —CH₂CCMe | O(CH₂)₂OMe | CHMe | +++ |
| —CH₂CCMe | NH(CH₂)₂OMe | CHMe | +++ |
| —CH₂CCMe | MeO(CH₂)₂CHMe- | CHMe | +++ |
| —CH₂CCMe | (CH₂)₃OH | CHMe | +++ |
| —CH₂CCMe | CH═CHCH₂OMe | CHMe | +++ |
| —CH₂CCMe | CH═CHCH₂OH | CHMe | +++ |
| —CH₂CCMe | 3-MeOCH₂-cBu | CHMe | +++ |
| —CH₂CCMe | (oxetan-3-yl)CH₂ | CHMe | +++ |
| —CH₂CCMe | cis-3-MeOcBu | CHMe | +++ |
| —CH₂CCMe | trans-3-MeOcBu | CHMe | +++ |
| —CH₂CCMe | tetrahydrofuran-3-yl | CHMe | +++ |
| —CH₂CCMe | tetrahydrofuran-2-yl | CHMe | +++ |
| —CH₂CCMe | 6-oxospiro[3.3]heptan-2-yl | CHMe | +++ |
| —CH₂CCMe | 6-(hydroxyimino)-spiro[3.3]]heptane-2-yl | CHMe | +++ |
| —CH₂CCMe | 2-oxaspiro[3.3]heptan-6-yl | CHMe | +++ |
| —CH₂CCMe | oxetan-3-yl | CHMe | +++ |
| —CH₂CCMe | 6-HO-spiro[3.3]heptan-2-yl | CHMe | +++ |
| —CH₂CCMe | cis-3-OH-cBu | CHMe | +++ |
| —CH₂CCMe | trans-3-OH-cBu | CHMe | +++ |
| —CH₂CCMe | trans-2-MeO₂C-cPr | CHMe | +++ |
| —CH₂CCMe | trans-2-HOCH₂-cPr | CHMe | +++ |
| —CH₂CCMe | trans-2-MeOCH₂-cPr | CHMe | +++ |
| —CH₂CC^{c}Pr | 3-MeO-bicyclo[1.1.1]pentan-1-yl | CHMe | +++ |
| —CH₂CC^{c}Pr | (CH₂)₃OMe | CHMe | +++ |
| —CH₂CC^{c}Pr | O(CH₂)2OMe | CHMe | +++ |
| —CH₂CC^{c}Pr | NH(CH₂)₂OMe | CHMe | +++ |
| —CH₂CC^{c}Pr | MeO(CH₂)₂CHMe- | CHMe | +++ |
| —CH₂CC^{c}Pr | (CH₂)3OH | CHMe | +++ |
| —CH₂CC^{c}Pr | CH═CHCH₂OMe | CHMe | +++ |
| —CH₂CC^{c}Pr | CH═CHCH₂OH | CHMe | +++ |
| —CH₂CC^{c}Pr | 3-MeOCH₂-cBu | CHMe | +++ |
| —CH₂CC^{c}Pr | (oxetan-3-yl)CH₂ | CHMe | +++ |
| —CH₂CC^{c}Pr | cis-3-MeOcBu | CHMe | +++ |
| —CH₂CC^{c}Pr | trans-3-MeOcBu | CHMe | +++ |
| —CH₂CC^{c}Pr | tetrahydrofuran-3-yl | CHMe | +++ |
| —CH₂CC^{c}Pr | tetrahydrofuran-2-yl | CHMe | +++ |
| —CH₂CC^{c}Pr | 6-oxospiro[3.3]]heptan-2-yl | CHMe | +++ |
| —CH₂CC^{c}Pr | 6-(hydroxyimino)-spiro[3.3]heptane-2-yl | CHMe | +++ |
| —CH₂CC^{c}Pr | 2-oxaspiro[3.3]]heptan-6-yl | CHMe | +++ |
| —CH₂CC^{c}Pr | oxetan-3-yl | CHMe | +++ |
| —CH₂CC^{c}Pr | 6-HO-spiro[3.3]heptan-2-yl | CHMe | +++ |
| —CH₂CC^{c}Pr | cis-3-OH-cBu | CHMe | +++ |
| —CH₂CC^{c}Pr | trans-3-OH-cBu | CHMe | +++ |
| —CH₂CC^{c}Pr | trans-2-MeO₂C-cPr | CHMe | +++ |
| —CH₂CC^{c}Pr | trans-2-HOCH₂-cPr | CHMe | +++ |
| —CH₂CC^{c}Pr | trans-2-MeOCH₂-cPr | CHMe | +++ |
| —CH₂CCMe | 3-MeO-bicyclo[1.1.1]pentan-1-yl | CH₂ | +++ |
| —CH₂CCMe | 3-HO-bicyclo[1.1.1]]pentan-1-yl | CH₂ | +++ |
| —CH₂CCMe | (oxetan-3-yl)CH₂ | CH₂ | +++ |
| —CH₂CCMe | cis-3-MeOcBu | CH₂ | +++ |
| —CH₂CCMe | trans-3-MeOcBu | CH₂ | +++ |
| —CH₂CCMe | 6-(hydroxyimino)-spiro[3.3]]heptane-2-yl | CH₂ | +++ |
| —CH₂CCMe | 6-HO-spiro[3.3]heptan-2-yl | CH₂ | +++ |
| —CH₂CCCPr | 3-MeOCH₂-cBu | CH₂ | +++ |
| —CH₂CCcPr | (oxetan-3-yl)CH₂ | CH₂ | +++ |
| —CH₂CCCPr | cis-3-MeOcBu | CH₂ | +++ |
| —CH₂CCCPr | trans-3-MeOcBu | CH₂ | +++ |
| —CH₂CCCPr | 6-(hydroxyimino)-spiro[3.3]heptane-2-yl | CH₂ | +++ |
| —CH₂CC Pr | 2-oxaspiro[3.3]heptan-6-yl | CH₂ | +++ |
| —CH₂CCcPr | 6-HO-spiro[3.3]]heptan-2-yl | CH₂ | +++ |
| —CH₂CCMe | 4-MeOPh | CHCN | +++ |
| —CH₂CCMe | 3-MeO-bicyclo[1.1.1]pentan-1-yl | CHCN | +++ |
| —CH₂CCMe | 3-MeOCH₂-cBu | CHCN | +++ |
| —CH₂CCMe | cis-3-MeOcBu | CHCN | +++ |
| —CH₂CCMe | trans-3-MeOcBu | CHCN | +++ |
| —CH₂CCMe | 6-(hydroxyimino)-spiro[3.3]]heptane-2-yl | CHCN | +++ |
| —CH₂CCcPr | 4-MeOPh | CHCN | +++ |
| —CH₂CCcPr | 3-MeO-bicyclo[1.1.1]pentan-1-yl | CHCN | +++ |
| —CH₂CCcPr | 3-MeOCH₂-cBu | CHCN | +++ |
| —CH₂CCºPr | cis-3-MeOcBu | CHCN | +++ |
| —CH₂CC°Pr | trans-3-MeOcBu | CHCN | +++ |
| —CH₂CCcPr | trans-2-HOCH₂-cPr | CHCN | +++ |
| —CH₂CCcPr | trans-2-MeOCH₂-cPr | CHCN | +++ |
| —CH₂CCMe | 4-MeOPh | CMeOH | +++ |
| —CH₂CCMe | 6-MeO-pyrid-3-yl | CMeOH | +++ |
| —CH₂CCMe | 3-MeO-bicyclo[1.1.1]pentan-1-yl | CMeOH | +++ |
| —CH₂CCMe | 3-MeOCH₂-cBu | CMeOH | +++ |
| —CH₂CCMe | cis-3-MeOcBu | CMeOH | +++ |
| —CH₂CCMe | trans-3-MeOcBu | CMeOH | +++ |
| —CH₂CCMe | 6-(hydroxyimino)-spiro[3.3]heptane-2-yl | CMeOH | +++ |
| —CH₂CC Pr | 3-MeO-bicyclo[1.1.1]pentan-1-yl | CMeOH | +++ |
| —CH₂CCcPr | 3-MeOCH₂-cBu | CMeOH | +++ |
| —CH₂CCcPr | cis-3-MeOcBu | CMeOH | +++ |
| —CH₂CCºPr | trans-3-MeOcBu | CMeOH | +++ |
| —CH₂CCcPr | trans-2-HOCH₂-cPr | CMeOH | +++ |
| —CH₂CCCPr | trans-2-MeOCH₂-cPr | CMeOH | +++ |
| —CH₂CCCH₂F | 3-MeO-bicyclo[1.1.1]pentan-1-yl | CHCN | +++ |
| —CH₂CCCH₂F | 3-MeOCH₂-cBu | CHCN | +++ |
| —CH₂CCCH₂F | cis-3-MeOcBu | CHCN | +++ |
| —CH₂CCCH₂F | trans-3-MeOcBu | CHCN | +++ |
| —CH₂CCCl | 4-MeOPh | CMeOH | +++ |
| —CH₂CCCl | 6-MeO-pyrid-3-yl | CMeOH | +++ |
| —CH₂CCCl | 3-MeOCH₂-cBu | CMeOH | +++ |
| —CH₂CCCl | cis-3-MeOcBu | CMeOH | +++ |
| —CH₂CCCl | trans-3-MeOcBu | CMeOH | +++ |
| —CH₂CCH | 3-MeOCH₂-cBu | CHCN | +++ |
| —CH₂CCH | cis-3-MeOcBu | CHCN | +++ |
| —CH₂CCH | trans-3-MeOcBu | CHCN | +++ |
| —CH₂CCCF3 | 4-MeOPh | CH₂ | +++ |

TABLE 10

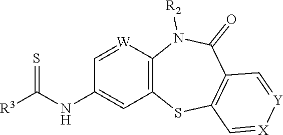

Examples:

| R² | R³ | X/Y | W | Activity |
|---|---|---|---|---|
| —CH₂CCMe | 3-MeO-bicyclo[1.1.1]]pentan-1-yl | CH/CH | CH | +++ |
| —CH₂CCMe | 6-HO-spiro[3.3]]heptan-2-yl | CH/CH | CH | +++ |
| —CH₂CCMe | 3-MeOCH₂-cBu | CH/CH | CH | +++ |
| —CH₂CCMe | (CH₂)₃OMe | CH/CH | CH | +++ |
| —CH₂CCMe | cis-3-MeOcBu | CH/CH | CH | +++ |
| —CH₂CCMe | trans-3-MeOcBu | CH/CH | CH | +++ |
| —CH₂CCMe | 3-MeO-bicyclo[1.1.1]pentan-1-yl | CF/CH | CH | +++ |
| —CH₂CCMe | 6-HO-spiro[3.3]]heptan-2-yl | CF/CH | CH | +++ |
| —CH₂CCMe | 3-MeOCH₂-cBu | CF/CH | CH | +++ |
| —CH₂CCMe | (CH₂)₃OMe | CF/CH | CH | +++ |
| —CH₂CCMe | cis-3-MeOcBu | CF/CH | CH | +++ |
| —CH₂CCMe | trans-3-MeOcBu | CF/CH | CH | +++ |
| —CH₂CCMe | 3-MeO-bicyclo[1.1.1]]pentan-1-yl | CF/CH | N | +++ |
| —CH₂CCMe | 6-HO-spiro[3.3]heptan-2-yl | CF/CH | N | +++ |
| —CH₂CCMe | 3-MeOCH₂-cBu | CF/CH | N | +++ |
| —CH₂CCMe | (CH₂)₃OMe | CF/CH | N | +++ |
| —CH₂CCMe | cis-3-MeOcBu | CF/CH | N | +++ |
| —CH₂CCMe | trans-3-MeOcBu | CF/CH | N | +++ |
| —CH₂CCMe | 3-MeO-bicyclo[1.1.1]pentan-1-yl | CCI/CH | CH | +++ |
| —CH₂CCMe | 6-HO-spiro[3.3]]heptan-2-yl | CCl/CH | CH | +++ |
| —CH₂CCMe | 3-MeOCH₂-cBu | CCl/CH | CH | +++ |
| —CH₂CCMe | (CH₂)₃OMe | CCl/CH | CH | +++ |
| —CH₂CCMe | cis-3-MeOcBu | CCl/CH | CH | +++ |
| —CH₂CCMe | trans-3-MeOcBu | CCl/CH | CH | +++ |
| —CH₂CCMe | 3-MeO-bicyclo[1.1.1]]pentan-1-yl | CCl/CH | N | +++ |
| —CH₂CCMe | 6-HO-spiro[3.3]]heptan-2-yl | CCl/CH | N | +++ |
| —CH₂CCMe | 3-MeOCH₂-cBu | CCI/CH | N | +++ |
| —CH₂CCMe | (CH₂)₃OMe | CCl/CH | N | +++ |
| —CH₂CCMe | cis-3-MeOcBu | CCI/CH | N | +++ |
| —CH₂CCMe | trans-3-MeOcBu | CCl/CH | N | +++ |
| —CH₂CCMe | (CH₂)₃OMe | CH/CH | N | +++ |
| —CH₂CCMe | 3-MeOCH₂-cBu | CH/CH | N | +++ |
| —CH₂CCMe | cis-3-MeOcBu | CH/CH | N | +++ |
| —CH₂CCMe | trans-3-MeOcBu | CH/CH | N | +++ |
| —CH₂CCMe | 6-HO-spiro[3.3]]heptan-2-yl | CH/CH | N | +++ |
| —CH₂CCºPr | 3-MeO-bicyclo[1.1.1]]pentan-1-yl | CH/CH | N | +++ |
| —CH₂CCcPr | 3-MeOCH₂-cBu | CH/CH | N | +++ |
| —CH₂CCºPr | cis-3-MeOcBu | CH/CH | N | +++ |
| —CH₂CCcPr | trans-3-MeOcBu | CH/CH | N | +++ |
| —CH₂CCºPr | 6-HO-spiro[3.3]]heptan-2-yl | CH/CH | N | +++ |
| —CH₂CCCH₂F | 3-MeO-bicyclo[1.1.1]]pentan-1-yl | CH/CH | N | +++ |
| —CH₂CCCH₂F | 3-MeOCH₂-cBu | CH/CH | N | +++ |
| —CH₂CCCH₂F | cis-3-MeOcBu | CH/CH | N | +++ |
| —CH₂CCCH₂F | trans-3-MeOcBu | CH/CH | N | +++ |
| —CH₂CCCl | 3-MeOCH₂-cBu | CH/CH | CH | +++ |
| —CH₂CCCl | cis-3-MeOcBu | CH/CH | CH | +++ |
| —CH₂CCCl | trans-3-MeOcBu | CH/CH | CH | +++ |
| —CH₂CCCl | 3-MeOCH₂-cBu | CH/CH | N | +++ |
| —CH₂CCCl | cis-3-MeOcBu | CH/CH | N | +++ |
| —CH₂CCCl | trans-3-MeOcBu | CH/CH | N | +++ |
| —CH₂CCH | 3-MeOCH₂-cBu | CH/CH | CH | +++ |
| —CH₂CCH | cis-3-MeOcBu | CH/CH | CH | +++ |
| —CH₂CCH | trans-3-MeOcBu | CH/CH | CH | +++ |
| —CH₂CCH | 3-MeOCH₂-cBu | CH/CH | N | +++ |
| —CH₂CCH | cis-3-MeOcBu | CH/CH | N | +++ |
| —CH₂CCH | trans-3-MeOcBu | CH/CH | N | +++ |

### REFERENCES

ADDIN EN.REFLIST 1. J. de la Cruz, K. Karbstein, J. L. Woolford, Jr., Functions of ribosomal proteins in assembly of eukaryotic ribosomes in vivo. Annu Rev Biochem 84, 93-129 (2015).
2. K. Dorner, C. Ruggeri, I. Zemp, U. Kutay, Ribosome biogenesis factors—from names to functions. EMBO J 42, e112699 (2023).
3. S. Klinge, J. L. Woolford, Ribosome assembly coming into focus. Nature Reviews Molecular Cell Biology, 1-16 (2018).
4. S. Bursac, Y. Prodan, N. Pullen, J. Bartek, S. Volarevic, Dysregulated Ribosome Biogenesis Reveals Therapeutic Liabilities in Cancer. Trends Cancer 7, 57-76 (2021).
5. C. Deisenroth, D. A. Franklin, Y. Zhang, The Evolution of the Ribosomal Protein-MDM2-p53 Pathway. Cold Spring Harb Perspect Med 6, (2016).
6. B. Lau et al., Mechanism of 5S RNP recruitment and helicase-surveilled rRNA maturation during pre-60S biogenesis. EMBO Rep 24, e56910 (2023).
7. M. S. Lindstrom, J. Bartek, A. Maya-Mendoza, p53 at the crossroad of DNA replication and ribosome biogenesis stress pathways. Cell Death Differ 29, 972-982 (2022).
8. N. M. Castillo Duque de Estrada et al., Structure of nascent 5S RNPs at the crossroad between ribosome assembly and MDM2-p53 pathways. Nat Struct Mol Biol 30, 1119-1131 (2023).
9. J. Zheng et al., Structure of human MDM2 complexed with RPL11 reveals the molecular basis of p53 activation. Genes Dev 29, 1524-1534 (2015).
10. A. Hafner, M. L. Bulyk, A. Jambhekar, G. Lahav, The multiple mechanisms that regulate p53 activity and cell fate. Nat Rev Mol Cell Biol 20, 199-210 (2019).
11. N. Hein, K. M. Hannan, A. J. George, E. Sanij, R. D. Hannan, The nucleolus: an emerging target for cancer therapy. Trends Mol Med 19, 643-654 (2013).
12. J. Pelletier, G. Thomas, S. Volarevic, Ribosome biogenesis in cancer: new players and therapeutic avenues. Nat Rev Cancer 18, 51-63 (2018).
13. J. E. Quin et al., Targeting the nucleolus for cancer intervention. Biochim Biophys Acta 1842, 802-816 (2014).
14. K. Burger et al., Chemotherapeutic drugs inhibit ribosome biogenesis at various levels. J Biol Chem 285, 12416-12425 (2010).
15. J. M. Woynarowski et al., Oxaliplatin-induced damage of cellular DNA. Mol Pharmacol 58, 920-927 (2000).
16. E. C. Sutton et al., Nucleolar Stress Induction by Oxaliplatin and Derivatives. J Am Chem Soc 141, 18411-18415 (2019).
17. E. C. Sutton, V. J. DeRose, Early nucleolar responses differentiate mechanisms of cell death induced by oxaliplatin and cisplatin. J Biol Chem 296, 100633 (2021).
18. H. B. Schmidt et al., Oxaliplatin disrupts nucleolar function through biophysical disintegration. Cell Rep 41, 111629 (2022).
19. P. M. Bruno et al., A subset of platinum-containing chemotherapeutic agents kills cells by inducing ribosome biogenesis stress. Nat Med 23, 461-471 (2017).
20. P. M. Bruno et al., The primary mechanism of cytotoxicity of the chemotherapeutic agent CX-5461 is topoisomerase II poisoning. Proc Natl Acad Sci USA 117, 4053-4060 (2020).
21. S. S. Negi, P. Brown, rRNA synthesis inhibitor, CX-5461, activates ATM/ATR pathway in acute lymphoblastic leukemia, arrests cells in G2 phase and induces apoptosis. Oncotarget 6, 18094-18104 (2015).
22. E. Sanij et al., CX-5461 activates the DNA damage response and demonstrates therapeutic efficacy in high-grade serous ovarian cancer. Nat Commun 11, 2641 (2020).
23. G. C. C. Koh et al., The chemotherapeutic drug CX-5461 is a potent mutagen in cultured human cells. Nat Genet 56, 23-26 (2024).
24. C. Ni et al., Labeling of heterochronic ribosomes reveals C1ORF109 and SPATA5 control a late step in human ribosome assembly. Cell Rep 38, 110597 (2022).
25. K. Galani, T. A. Nissan, E. Petfalski, D. Tollervey, E. Hurt, Real, a dynein-related nuclear AAA-ATPase, is involved in late rRNA processing and nuclear export of 60 S subunits. J Biol Chem 279, 55411-55418 (2004).
26. D. Kressler, D. Roser, B. Pertschy, E. Hurt, The AAA ATPase Rix7 powers progression of ribosome biogenesis by stripping Nsa1 from pre-60S particles. The Journal of Cell Biology 181, 935-944 (2008).
27. I. Cooney et al., Structure of the Cdc48 segregase in the act of unfolding an authentic substrate. Science 365, 502-505 (2019).
28. M. Prattes, Y. H. Lo, H. Bergler, R. E. Stanley, Shaping the Nascent Ribosome: AAA-ATPases in Eukaryotic Ribosome Biogenesis. Biomolecules 9, (2019).
29. M. Loibl et al., The Drug Diazaborine Blocks Ribosome Biogenesis by Inhibiting the AAA-ATPase Drg1*. J Biol Chem 289, 3913-3922 (2014).
30. M. Prattes et al., Structural basis for inhibition of the AAA-ATPase Drg1 by diazaborine. Nat Commun 12, 3483 (2021).
31. S. A. Kawashima et al., Potent, Reversible, and Specific Chemical Inhibitors of Eukaryotic Ribosome Biogenesis. Cell 167, 512-518.e513 (2016).
32. T. P. Nguyen et al., Inducible mismatch repair streamlines forward genetic approaches to target identification of cytotoxic small molecules. Cell Chem Biol, (2023).
33. Y.-H. Lo et al., Cryo-EM structure of the essential ribosome assembly AAA-ATPase Rix7. Nature Communications, 1-12 (2019).
34. S. Kocaman et al., Communication network within the essential AAA-ATPase Rix7 drives ribosome assembly. PNAS Nexus 1, pgac118 (2022).
35. M. Prattes et al., Visualizing maturation factor extraction from the nascent ribosome by the AAA-ATPase Drg1. Nat Struct Mol Biol 29, 942-953 (2022).
36. M. O. Rotenberg, M. Moritz, J. L. Woolford, Jr., Depletion of Saccharomyces cerevisiae ribosomal protein L16 causes a decrease in 60S ribosomal subunits and formation of half-mer polyribosomes. Genes Dev 2, 160-172 (1988).
37. O. Gadal et al., A nuclear AAA-type ATPase (Rix7p) is required for biogenesis and nuclear export of 60S ribosomal subunits. EMBO J 20, 3695-3704 (2001).
38. Y. Yoshikatsu et al., NVL2, a nucleolar AAA-ATPase, is associated with the nuclear exosome and is involved in pre-rRNA processing. Biochem Biophys Res Commun 464, 780-786 (2015).
39. A. Vanden Broeck, S. Klinge, Principles of human pre-60S biogenesis. Science 381, eadh3892 (2023).
40. M. Pan et al., Mechanistic insight into substrate processing and allosteric inhibition of human p97. Nat Struct Mol Biol 28, 614-625 (2021).
41. Y. Shiloh, ATM and ATR: networking cellular responses to DNA damage. Curr Opin Genet Dev 11, 71-77 (2001).
42. E. B. Maldonado, S. Parsons, E. Y. Chen, A. Haslam, V. Prasad, Estimation of US patients with cancer who may respond to cytotoxic chemotherapy. Future Sci OA 6, Fso600 (2020).
43. M. Nechay, D. Wang, R. E. Kleiner, Inhibition of nucleolar transcription by oxaliplatin involves ATM/ATR kinase signaling. Cell Chem Biol 30, 906-919 e904 (2023).
44. S. Nishikori, M. Esaki, K. Yamanaka, S. Sugimoto, T. Ogura, Positive cooperativity of the p97 AAA ATPase is critical for essential functions. J Biol Chem 286, 15815-15820 (2011).
45. I. Cooney et al., Visualization of the Cdc48 AAA+ ATPase protein unfolding pathway. bioRxiv, 2023.2005.2013.540638 (2023).
46. S. Banerjee et al., 2.3 Å resolution cryo-EM structure of human p97 and mechanism of allosteric inhibition. Science 351, 871-875 (2016).
47. J. van Riggelen, A. Yetil, D. W. Felsher, MYC as a regulator of ribosome biogenesis and protein synthesis. Nat Rev Cancer 10, 301-309 (2010).
48. E. Feldman et al., Homoharringtonine is safe and effective for patients with acute myelogenous leukemia. Leukemia 6, 1185-1188 (1992).
49. E. W. Mills, R. Green, Ribosomopathies: There's strength in numbers. Science 358, (2017).
50. K. I. Farley-Barnes, L. M. Ogawa, S. J. Baserga, Ribosomopathies: Old Concepts, New Controversies. Trends Genet 35, 754-767 (2019).
51. https://portals.broadinstitute.org/gpp/public/dir/download?dirpath=protocols/production&filename=sgRNA_PCR_for_Illumina_sept2015.pdf.
52. S. Frey, D. Gorlich, A new set of highly efficient, tag-cleaving proteases for purifying recombinant proteins. J Chromatogr A 1337, 95-105 (2014).
53. D. N. Mastronarde, Automated electron microscope tomography using robust prediction of specimen movements. Journal of Structural Biology 152, 36-51 (2005).
54. S. H. W. Scheres, RELION: Implementation of a Bayesian approach to cryo-EM structure determination. Journal of Structural Biology 180, 519-530 (2012).
55. D. Kimanius, L. Dong, G. Sharov, T. Nakane, S. H. W. Scheres, New tools for automated cryo-EM single-particle analysis in RELION-4.0. Biochem J 478, 4169-4185 (2021).
56. S. Q. Zheng et al., MotionCor2: anisotropic correction of beam-induced motion for improved cryo-electron microscopy. 1-2 (2017).
57. A. Kucukelbir, F. J. Sigworth, H. D. Tagare, Quantifying the local resolution of cryo-EM density maps. Nature Methods 11, 63-65 (2013).
58. J. Jumper et al., Highly accurate protein structure prediction with AlphaFold. Nature 596, 583-589 (2021).
59. P. Emsley, K. Cowtan, Coot: model-building tools for molecular graphics. Acta Cryst (2004). D60, 2126-2132 [doi: 10.1107 S0907444904019158], 1-7 (2004).
60. P. D. Adams et al., PHENIX: a comprehensive Python-based system for macromolecular structure solution. Acta Crystallogr D Biol Crystallogr 66, 213-221 (2010).
61. E. F. Pettersen et al., UCSF ChimeraX: Structure visualization for researchers, educators, and developers. Protein Science 30, 70-82 (2021).

1. A compound of formula (I): 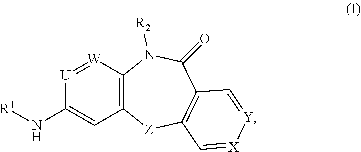
wherein:
R1 is selected from: R3; —C(O)R3; —C(S)R3; —C(O)NHR3; —C(S)NHR3; —C(O)NR5R6; —C(S)NR5R6;
R2 is selected from: C1-C8alkyl; C2-C8alkenyl; C2-C8alkynyl including all isomers thereof and all optionally substituted with one or more of: D, F, OH, C(O)NHR4 and all optionally having one or more methylene units replaced with O, S, NH, NR4, C(O);
R3 is selected from: C1-C7alkyl, C3-C7cycloalkyl optionally bridged with CH₂ or CH₂CH₂, CH₂-linked or CHMe-linked C3-C7cycloalkyl optionally bridged with CH₂ or CH₂CH₂, C2-C7alkenyl, C5-C7cycloalkenyl optionally bridged with CH₂ or CH₂CH₂, CH₂-linked or CHMe-linked C5-C7cycloalkenyl optionally bridged with CH₂ or CH₂CH₂, C2-C7alkynyl, bridged or fused C5-C9cycloalkyl, CH₂-linked or CHMe-linked bridged or fused C5-C9cycloalkyl C5-C10spiroalkanes, CH₂-linked or CHMe-linked C5-C10spiroalkanes, all including all possible isomers thereof and all optionally substituted with one or more of: D, F, CN, R4, OR4, OH, NHC(O)H, NHC(O)R4, NHSO₂R4, CO₂H, CO₂R4, C(O)NH₂, C(O)NHR4, C(O)NR4R4 and all optionally having one or more methylene units replaced with O, S, SO₂, S(O), NH, NR4, NC(O)H, NC(O)R4, NSO₂R4, C(O), C═NOH, C═NOR4 or C═NR4, phenyl or naphtyl (optionally substituted at one or more of each position with R7), 5- and 6-membered heteroaryls and fused heteroaryls including but not limited to oxazoles, isoxazoles, thiazoles, isothiazoles, furans, thiophenes, pyrazoles, imidazoles, triazoles, tetrazoles, pyridines, pyrimidines, pyrazines, pyridazines, quinolines, isoquinolines, quinazolines, quinoxalines, cinnolines, phtalazines, naphthyridines, pyridopyrazines, pyrazolopyridines, pyridopyrimidines, pyridopyridazines, benzoxazoles, benzoisoxazoles, benzothiazoles, benzoisothiazoles, indoles, indazoles, benzimidazoles, dioxolopyridines, dioxolopyridazines, dioxolopyrimidines, dioxolopyrazines, benzofurans, benzothiophenes, imidazopyridines, benzodioxoles, benzoxazolones, benzoisoxazolones, benzothiazolones, benzoisothiazolones, 1,3-dihydro-2H-benzimidazoles, benzo[d]imidazol-2-ones, benzodioxolones, dioxolopyridines, dioxolopyridazines, dioxolopyrimidines, dioxolopyrazines, coumarins, isocoumarins, and including all possible isomers thereof, and all optionally substituted at one or more of each position with R7;
R4 is selected from: Me; Et; Pr; iPr; cPr; cBu optionally substituted with one or more of: D, F, CN, OH, OCD₃, OCHₙF₍₃₋ₙ₎ (n=0-3);
NR5R6 completes a 4- to 7-membered azacycle or a bridged or fused 6- to 9-membered azacycle or 5- to 9-membered azaspiroalkane including all possible isomers thereof and all optionally substituted with one or more of: D, F, CN, R4, OR4, OH, NHC(O)H, NHC(O)R4, NHSO₂R4, CO₂H, CO₂R4, C(O)NH₂, C(O)NHR4, C(O)NR4R4 and all optionally having one or more methylene units replaced with O, S, SO₂, NH, NR4, NC(O)H, NC(O)R4, NSO₂R4, C(O), C═NOH, C═NR4;
R7 is selected from: D, F, Cl, Br, CN, N₃, OH, OR4, R4, oxetanyl, NHC(O)H, NHC(O)R4, CO₂H, CO₂R4, C(O)NH₂, C(O)NHR4, C(O)NR4R4, SO₂NH₂, SO₂NHR4, SO₂NR4R4, C2-C6alkenyl or C2-C6alkynyl including all isomers thereof and all optionally substituted with one or more of: D, F, CN, OH and all optionally having one or more methylene units replaced with O, S, S(O), SO₂, NH, NR4, NC(O)H, NC(O)R4, NSO₂R4, C(O), C═NOH, C═NR4; aryl or 5- or 6-membered heteroaryl or heterocyclyl all optionally substituted with one or more of: D, F, Cl, CN, R4, OH, OR4);
Z is selected from: S, CH₂, CD₂, CHR4, CDR4, CR4R4, CHOH, CDOH, CHOR4, CDOR4, CR4OH, CR4OR4, CHCN, CDCN, CHF, CDF, CF₂, NH, NR4, NC(O)H, NC(O)R4, NSO₂R4;
X is selected from: CH, CD, CF, CCl, CCN;
Y is selected from: CH, CD, CF, CCl;
U and W are independently selected from: CH, CD, N;
excluding R1=C(O) furan-3-yl, X, Y, U, W=CH, Z=S, R2=(CH₂)₂OMe (MM017), or a salt, hydrate or stereoisomer thereof.

2. The compound of claim 1, further of formula II, III, IV, V, VI, VII or VIII:

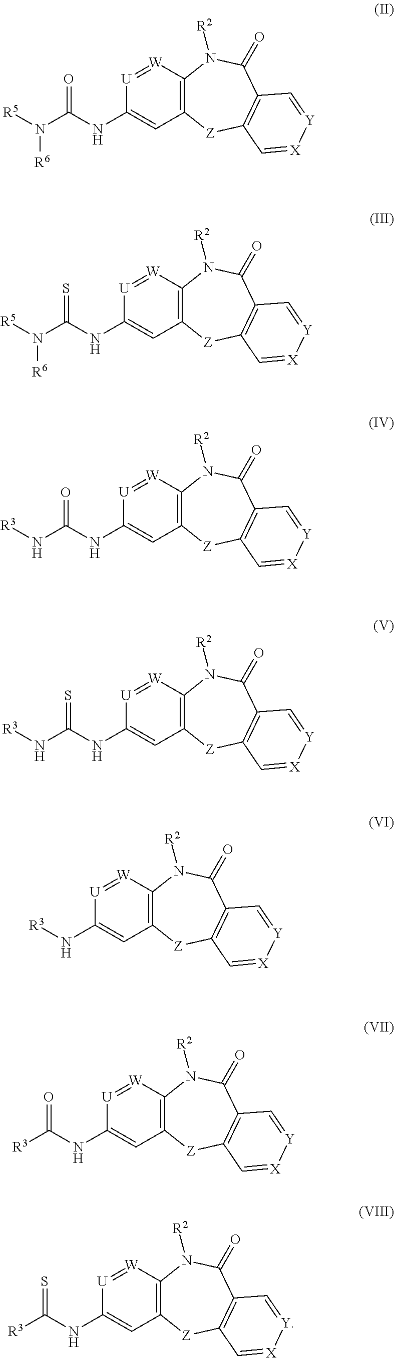

3. A compound of claim 1, wherein:
Z=S, CH₂, CD₂, CHMe, CHCD₃, CDMe, CDCD₃, CHCN, CDCN, CMeOH, CCD₃OH, preferably S, CH₂, CHMe, CHCD₃;
X=CH, CD, CF, CCl, CCN, preferably CH, CF, or CCl;
Y=CH, CD, CF, CCl, preferably CH, or CF;
W=CH, CD, N;
U=CH, CD, N, preferably CH;
R2=C2-C7alkyl; C3-C7alkenyl; C3-C7alkynyl including all isomers thereof and all optionally substituted with one or more of: D, F, Cl and all optionally having one or more methylene units replaced with O;
R2=CH₂C≡CH, CH₂C≡CR4, CH₂C≡CCl, CH₂CH═CH₂, CH₂CH═CHR4, (CH₂)₂OR4, (CH₂)₂OCH₂C≡CH, where R2 can contain one or more deuterium; or
R2=CH₂C≡CH, CH₂C≡CMe, CH₂C≡CCH₂F, CH₂C≡C^{c}Pr, CH₂C≡CCl, CH₂CH═CHMe, CH₂C≡CCF₃ where R2 can contain one or more deuterium.

4. A compound of claim 1,
(a) of formula II or III, wherein:
R2=CH₂C≡CH, CH₂C≡CMe, CH₂C≡CCH₂F, CH₂C≡CCl, CH₂C≡C^{c}Pr, CH₂CH═CHMe, where R2 can contain one or more deuterium;
NR5R6 completes a 4- to 7-membered azacycle or a bridged or fused 6- to 9-membered azacycle or 5- to 9-membered azaspiroalkane including all possible isomers thereof and all optionally substituted with one or more of: D, F, CN, R4, OR4, OH, NHC(O)H, NHC(O)R4, NHSO₂R4, CO₂H, CO₂R4, C(O)NH₂, C(O)NHR4, C(O)NR4R4 and all optionally having one or more methylene units replaced with O, S, SO₂, NH, NR4, NC(O)H, NC(O)R4, NSO₂R4, C(O), C═NOH, C═NR4;
R4=Me; Et; Pr; iPr; cPr; cBu optionally substituted with one or more of: D, F, CN, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CD, CF, CCl;
Y=CH, CD, CF;
W=CH, N; and
U=CH, CD, N preferably CH; or
(b) of formula II or III, wherein:
R2=CH₂C≡CH, CH₂C≡CMe, CH₂C≡CCH₂F, CH₂C≡CCl, CH₂C≡C^{c}Pr preferably CH₂C═CMe, CH₂C≡C^{c}Pr;
NR5R6 completes a 4- to 7-membered azacycle or a 6- to 8-membered azabicycle or a 5- to 7-membered azaspiroalkane, all optionally substituted with one or more of: D, F, CN, R4, OR4, OH and all optionally having one or more methylene units replaced with O, S, SO₂, NH, NR4, C(O), C═NOH, C═NR4;
R4=Me; Et; Pr; iPr; cPr; cBu optionally substituted with one or more of: D, F, CN, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

5. A compound of claim 1,
(a) of formula IV, V or VIII, wherein:
R2=CH₂C≡CH, CH₂C≡CMe, CH₂C≡CCH₂F, CH₂C≡CCl, CH₂C≡C^{c} Pr, CH₂CH═CHMe, where R2 can contain one or more deuterium;
R3=phenyl (optionally substituted at one or more of each individual position with R7); 5- and 6-membered heteroaryls and fused heteroaryls including but not limited to oxazoles, isoxazoles, thiazoles, isothiazoles, furans, thiophenes, pyrazoles, imidazoles, triazoles, tetrazoles, pyridines, pyrimidines, pyrazines, pyridazines, quinolines, isoquinolines, quinazolines, quinoxalines, cinnolines, phtalazines, naphthyridines, pyridopyrazines, pyrazolopyridines, pyridopyrimidines, pyridopyridazines, benzoxazoles, benzoisoxazoles, benzothiazoles, benzoisothiazoles, indoles, indazoles, benzimidazoles, dioxolopyridines, dioxolopyridazines, dioxolopyrimidines, dioxolopyrazines, benzofurans, benzothiophenes, imidazopyridines, benzodioxoles, benzoxazolones, benzoisoxazolones, benzothiazolones, benzoisothiazolones, 1,3-dihydro-2H-benzimidazoles, benzo[d]imidazol-2-ones, benzodioxolones, dioxolopyridines, dioxolopyridazines, dioxolopyrimidines, dioxolopyrazines, coumarins, isocoumarins, and including all possible isomers thereof, and all optionally substituted at one or more of each individual position with R7;
R4=Me, Et, Pr, iPr, cPr, cBu optionally substituted with one or more of: D, F, CN, OH, OCD₃, OCHₙF₍₃₋ₙ₎ (n=0-3);
R7=D, F, Cl, Br, CN, N₃, OH, R4, OR4, C≡CH, NHC(O)H, C(O)NH₂, C(O)NHR4, C(O)NR4R4;
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH; or
(b) of formula IV, V or VIII, wherein:
R2=CH₂C≡CH, CH₂C≡CMe, CH₂C≡CCH₂F, CH₂C≡CCl, CH₂C≡C^{c}Pr preferably CH₂C≡CMe, CH₂C≡C^{c}Pr;
R3=a mono-or-disubstituted phenyl ring with substituents at the ortho-position selected from H, D or F and substituents at the meta and para-position selected from H, D, F, Cl, R4, OR4, N₃, C≡CH; a heteroaromatic ring selected from furan, pyridine, pyrimidine, pyrazine, pyridazine, benzo[d]oxazol-5-yl, benzo[d]thiazol-5-yl, 2-oxo-2,3-dihydrobenzo[d]oxazol-5-yl, benzo[d][1,3]dioxol-5-yl and all optionally substituted at one or more of each individual position with H, D, F, Cl, CCH, R4, OR4;
R4=Me; Et; Pr; iPr; cPr; cBu optionally substituted with one or more of: D, F;
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH; or
(c) of formula IV, V or VIII, wherein:
R2=CH₂C≡CH, CH₂C≡CMe, CH₂C≡CCH₂F, CH₂C≡CCl, CH₂C≡C^{c}Pr, CH₂CH═CHMe, where R2 can contain one or more deuterium;
R3=C1-C7alkyl, C3-C7cycloalkyl optionally bridged with CH₂ or CH₂CH₂, CH₂-linked or CHMe-linked C3-C6cycloalkyl optionally bridged with CH₂ or CH₂CH₂, C2-C7alkenyl, C5-C6cycloalkenyl optionally bridged with CH₂ or CH₂CH₂, CH₂-linked or CHMe-linked C5-C6cycloalkenyl optionally bridged with CH₂ or CH₂CH₂, C2-C7alkynyl, bridged or fused C5-C9cycloalkyl, CH₂-linked or CHMe-linked bridged or fused C5-C9cycloalkyl, C5-C10spiroalkanes, CH₂-linked or CHMe-linked C5-C10spiroalkanes, all including all possible isomers thereof and all optionally substituted with one or more of: D, F, CN, R4, OR4, OH, NHC(O)H, NHC(O)R4, NHSO₂R4, CO₂H, CO₂R4, C(O)NH₂, C(O)NHR4, C(O)NR4R4 and all optionally having one or more methylene units replaced with O, S, SO₂, S(O), NH, NR4, NC(O)H, NC(O)R4, NSO₂R4, C(O), C═NOH, C═NOR4 or C═NR4;
R4=Me, Et, Pr, iPr, cPr, cBu optionally substituted with one or more of: D, F, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

6. A compound of claim 1,
(a) of formula VI, wherein:
R2=CH₂C≡CH, CH₂C≡CMe, CH₂C≡CCH₂F, CH₂C≡CCl, CH₂C≡C^{c}Pr, CH₂CH═CHMe, CH₂═CHEt, CH₂CH═CHPr, (CH₂)₂OCH₂CCH where R2 can contain one or more deuterium;
R3=phenyl (optionally substituted at one or more of each individual position with R7); 5- and 6-membered heteroaryls and fused heteroaryls including but not limited to oxazoles, isoxazoles, thiazoles, isothiazoles, furans, thiophenes, pyrazoles, imidazoles, triazoles, tetrazoles, pyridines, pyrimidines, pyrazines, pyridazines, quinolines, isoquinolines, quinazolines, quinoxalines, cinnolines, phtalazines, naphthyridines, pyridopyrazines, pyrazolopyridines, pyridopyrimidines, pyridopyridazines, benzoxazoles, benzoisoxazoles, benzothiazoles, benzoisothiazoles, indoles, indazoles, benzimidazoles, dioxolopyridines, dioxolopyridazines, dioxolopyrimidines, dioxolopyrazines, benzofurans, benzothiophenes, imidazopyridines, benzodioxoles, benzoxazolones, benzoisoxazolones, benzothiazolones, benzoisothiazolones, 1,3-dihydro-2H-benzimidazoles, benzo[d]imidazol-2-ones, benzodioxolones, dioxolopyridines, dioxolopyridazines, dioxolopyrimidines, dioxolopyrazines, coumarins, isocoumarins, and including all possible isomers thereof and all optionally substituted at one or more of each individual position with R7;
R7=D, F, Cl, Br, CN, N₃, OH, R4, OR4, C≡CH, NHC(O)H, C(O)NH₂, C(O)NHR4, C(O)NR4R4;
R4=Me, Et, Pr, iPr, cPr, cBu optionally substituted with one or more of: D, F, CN, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH; or
(b) of formula VI, wherein:
R2=CH₂C≡CH, CH₂C≡CMe, CH₂C≡CCH₂F, CH₂C≡CCl, CH₂C≡C^{c}Pr, CH₂CH═CHMe, CH₂=CHEt, CH₂CH═CHPr;
R3=5- and 6-membered heteroaryls including but not limited to pyrazoles, imidazoles, pyridines, pyrimidines, pyrazines, pyridazines, quinolines, isoquinolines, quinazolines, quinoxalines, cinnolines, phtalazines, and including all possible isomers thereof, and all optionally substituted at one or more of each individual position with D, F, Cl, CN, Me, cPr, CD₃, OCD₃, OH, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH; or
(c) of formula VI, wherein:
R2=CH₂C≡CH, CH₂C≡CMe, CH₂C≡CCH₂F, CH₂C≡CCl, CH₂C≡C^{c}Pr preferably CH₂C≡CMe, CH₂C≡C^{c}Pr;
R3=pyrazoles, pyridines, pyrimidines, pyrazines, pyridazines all optionally substituted at one or more of each individual position with D, F, Cl, CN, Me, cPr, CD₃, OCD₃, OH, OCHₙF₍₃₋ₙ₎ (n=0-3);
Z=S, CH₂, CHMe, CHCN, CMeOH preferably S, CH₂, CHMe;
X=CH, CF, CCl;
Y=CH, CF;
W=CH, N; and
U=CH, N preferably CH.

7. A compound of claim 1, of formula VI, wherein:
R2=CH₂C≡CH, CH₂C≡CMe, CH₂C≡CCH₂F, CH₂C≡CCl, CH₂C≡C^{c}Pr, where R2 can contain one or more deuterium;
R3=C2-C4alkyl, C2-C4alkenyl, C3-C6cycloalkyl optionally bridged with CH₂ or CH₂CH₂, CH₂-linked or CHMe-linked C3-C6cycloalkyl optionally bridged with CH₂ or CH₂CH₂, C5-C6cycloalkenyl optionally bridged with CH₂ or CH₂CH₂, CH₂-linked or CHMe-linked C5-C6cycloalkenyl optionally bridged with CH₂ or CH₂CH₂, C5-C8spiroalkanes, CH₂-linked or CHMe-linked C5-C8spiroalkanes, and all optionally substituted with one or more of: D, F, CN, OH, R4, OCD₃, OCHₙF₍₃₋ₙ₎ (n=0-3) and all optionally having one or more methylene units replaced with O, S, SO₂, S(O), NH, NR4, NC(O)H, NC(O)R4, NSO₂R4, C(O), C≡NOH, C≡NOR4 or C═NR4;
R4=Me, Et, Pr, iPr, cPr, cBu optionally substituted with one or more of: D, F, OH, OCD₃, OCHₙF₍₃₋ₙ₎ (n=0-3);
Z=S, CH₂, CHMe, CHCD₃, CHCN, CMeOH, CCD₃OH, preferably S, CH₂, CHMe, CHCD₃;
X=CH, CF, CCl;
Y=CH, CF;
W=CH, N; and
U=CH, N preferably CH.

8. A compound of claim 1, of formula II or III, wherein:
R2=CH₂C≡CMe, CH₂C≡CCD₃, CH₂C≡CCH₂F, CH₂C≡C^{c} Pr, CH₂C≡CCl, CH₂C≡CH preferably CH₂C≡CMe, CH₂C≡C^{c} Pr;
NR5R6=N-linked azetidine, pyrrolidine, piperidine, azepine optionally substituted with one or more of: D, F, CN, R4, OR4, OH and optionally bridged with CH₂ or CH₂CH₂ and all optionally having one or more methylene units replaced with O, NR4, N═OH, N═OMe, N═OCD₃;
R4=Me, cPr optionally substituted with one or more of: D, F, CN, OH, OCD₃, OCHₙF₍₃₋ₙ₎ (n=0-3);
Z=S, CH₂, CHMe preferably S;
X=CH, CF, CCl;
Y=CH, F preferably CH;
W=CH, N; and
U=CH.

9. A compound claim 1, of formula VI, wherein:
R2=CH₂C≡CMe, CH₂C≡CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=pyridines, pyrimidines, pyrazines, pyridazines all optionally substituted at one or more of each individual position with D, F, Cl, CN, Me, Et, iPr, cPr, OH, OCD₃, OCHₙF₍₃₋ₙ₎ (n=0-3);
Z=S, CH₂, CHMe preferably S;
X=CH, CF, CCl;
Y=CH, CF preferably CH;
W=CH, N; and
U=CH.

10. A compound claim 1, of formula VI, wherein:
R2=CH₂C≡CMe, CH₂C≡CCD₃, CH₂C≡CPr, CH₂C≡CCH₂F, CH₂C≡CCl, CH₂C≡CH preferably CH₂C≡CMe, CH₂C≡C^{c}Pr;
R3=C2-C4alkyl, C2-C4alkenyl, C3-C6cycloalkyl optionally bridged with CH₂ or CH₂CH₂, CH₂-linked or CHMe-linked C3-C6cycloalkyl optionally bridged with CH₂ or CH₂CH₂, C5-C6cycloalkenyl optionally bridged with CH₂ or CH₂CH₂, CH₂-linked or CHMe-linked C5-C6cycloalkenyl optionally bridged with CH₂ or CH₂CH₂, C5-C8spiroalkanes, CH₂-linked or CHMe-linked C5-C8spiroalkanes, all including all possible isomers thereof and all optionally substituted with one or more of: D, F, Me, CD₃, cPr, OH, OCD₃, OCHₙF₍₃₋ₙ₎ (n=0-3), CH₂OH, CHMeOH, CH₂OCD₃, CH₂OCHₙF₍₃₋ₙ₎ (n=0-3), CHMeOCD₃, CHMeOCHₙF₍₃₋ₙ₎ (n=0-3), and all optionally having one or more methylene units replaced with O, S, SO₂NH, NMe, NCD₃, CO, N═OH, N═OMe, N═OCD₃;
Z=S, CH₂, CHMe preferably S;
X=CH, CF, CCl;
Y=CH, CF preferably CH;
W=CH, N; and
U=CH.

11. A compound of claim 1, of formula IV, V or VIII, wherein:
R2=CH₂C≡CMe, CH₂C≡CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=a mono-or-disubstituted phenyl ring with substituents at the ortho-position selected from H, D or F and substituents at the meta and para-position selected from H, D, F, Cl, R4, OR4, N₃, C≡CH; a heteroaromatic ring selected from furan, pyridine, pyrimidine, pyrazine, pyridazine, benzo[d]oxazol-5-yl, benzo[d]thiazol-5-yl, 2-oxo-2,3-dihydrobenzo[d]oxazol-5-yl, benzo[d][1,3]dioxol-5-yl and all optionally substituted at one or more of each individual position with H, D, F, Cl, Me, CD₃, cPr, CCH, OCD₃, OCHₙF₍₃₋ₙ₎ (n=0-3);
Z=S, CH₂, CHMe preferably S;
X=CH, CF, CCl;
Y=CH, CF preferably CH;
W=CH, N; and
U=CH.

12. A compound of claim 1, of formula IV, V or VIII, wherein:
R2=CH₂C≡CMe, CH₂C≡CCD₃, CH₂C≡C^{c} Pr, CH₂C≡CCH₂F, CH₂C≡CCl, CH₂C≡CH preferably CH₂C≡CMe, CH₂C≡C^{c}Pr;
R3=C2-C4alkyl, C2-C4alkenyl, C2-C3alkynyl, C3-C6cycloalkyl optionally bridged with CH₂ or CH₂CH₂, CH₂-linked or CHMe-linked C3-C6cycloalkyl optionally bridged with CH₂ or CH₂CH₂, C5-C6cycloalkenyl optionally bridged with CH₂ or CH₂CH₂, CH₂-linked or CHMe-linked C5-C6cycloalkenyl optionally bridged with CH₂ or CH₂CH₂, C5-C8spiroalkanes, CH₂-linked or CHMe-linked C5-C8spiroalkanes, and all including all possible isomers thereof and all optionally substituted with one or more of: D, F, CN, Me, CD₃, cPr, OH, OCD₃, OCHₙF₍₃₋ₙ₎ (n=0-3), CH₂OH, CHMeOH, CH₂OCD₃, CH₂OCHₙF₍₃₋ₙ₎ (n=0-3), CHMeOCD₃, CHMeOCHₙF₍₃₋ₙ₎ (n=0-3), CO₂Me, CO₂CD₃, and all optionally having one or more methylene units replaced with O, S, SO₂, NH, NMe, NCD₃, C(O), N═OH, N═OMe, N═OCD₃;
Z=S, CH₂, CHMe preferably S;
X=CH, CF, CCl;
Y=CH, CF preferably CH;
W=CH, N; and
U=CH.

13. A compound of claim 1, of VII, wherein:
R2=CH₂C≡CMe, CH₂C≡CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=a mono-or-disubstituted phenyl ring with substituents at the ortho-position selected from H, D or F and substituents at the meta and para-position selected from H, D, F, Cl, R4, OR4, N₃, C≡CH; a heteroaromatic ring selected from furan, pyridine, pyrimidine, pyrazine, pyridazine, benzo[d]oxazol-5-yl, benzo[d]thiazol-5-yl, 2-oxo-2,3-dihydrobenzo[d]oxazol-5-yl, benzo[d][1,3]dioxol-5-yl and all optionally substituted at one or more of each individual position with H, D, F, Cl, Me, CD₃, cPr, CCH, OCD₃, OCHₙF₍₃₋ₙ₎ (n=0-3);
Z=S, CH₂, CHMe preferably S;
X=CH, CF, CCl;
Y=CH, CF preferably CH;
W=CH, N; and
U=CH.

14. A compound of claim 1, of VII, wherein:
R2=CH₂C≡CMe, CH₂C≡CCD₃, CH₂C≡C^{c}Pr, CH₂C≡CCH₂F, CH₂C≡CCl, CH₂C≡CH preferably CH₂C≡CMe, CH₂C≡C^{c}Pr;
R3=C2-C4alkyl, C2-C4alkenyl, C2-C3alkynyl, C3-C6cycloalkyl optionally bridged with CH₂ or CH₂CH₂, CH₂-linked or CHMe-linked C3-C6cycloalkyl optionally bridged with CH₂ or CH₂CH₂, C5-C6cycloalkenyl optionally bridged with CH₂ or CH₂CH₂, CH₂-linked or CHMe-linked C5-C6cycloalkenyl optionally bridged with CH₂ or CH₂CH₂, C5-C8spiroalkanes, CH₂-linked or CHMe-linked C5-C8spiroalkanes, and all including all possible isomers thereof and all optionally substituted with one or more of: D, F, CN, Me, CD₃, cPr, OH, OCD₃, OCHₙF₍₃₋ₙ₎ (n=0-3), CH₂OH, CHMeOH, CH₂OCD₃, CH₂OCHₙF₍₃₋ₙ₎ (n=0-3), CHMeOCD₃, CHMeOCHₙF₍₃₋ₙ₎ (n=0-3), CO₂Me, CO₂CD₃, and all optionally having one or more methylene units replaced with O, S, SO₂, NH, NMe, NCD₃, C(O), N═OH, N═OMe, N═OCD₃;
Z=S, CH₂, CHMe preferably S;
X=CH, CF, CCl;
Y=CH, CF preferably CH;
W=CH, N; and
U=CH.

15. A compound of claim 1, selected from any of Tables 1-10.

16. A pharmaceutical composition comprising a compound of a claim 1, or a pharmaceutically acceptable salt, a hydrate or a stereoisomer thereof, and a pharmaceutically acceptable carrier or excipient, preferably in a pharmaceutically acceptable unit dosage.

17. A method to inhibit ribosome biogenesis, induce p53 or inhibit cancer cells, comprising administering to a person in need thereof a compound of claim 1.

18. A method to inhibit cancer cells, or to treat a disease or condition comprising a cancer, tumor or neoplasia, comprising comprising administering to a person in need thereof a compound of a claim 1, such as wherein the cancer, tumor or neoplasia comprises breast cancer, lung cancer, colorectal cancer, ovarian cancer, bladder cancer, kidney cancer, esophageal cancer, stomach cancer, cervical cancer, head and neck cancers, liver cancer, prostate cancer, pancreatic cancer, sarcoma, melanoma, leukemia, lymphoma, brain cancer, skin cancer (melanoma), thyroid cancer, testicular cancer, and multiple myeloma, optionally further comprising the antecedent step of detecting or diagnosing a disease or condition indicating the need thereof, and/or the subsequent step of detecting a resultant improvement or delay of progression of the disease or condition.

19. A method of screening for candidate therapeutics for treating cancer, comprising assaying for inhibitors or NVL2.

20. A cell line for evaluating cancer therapeutics comprising and expressing a mutant NVL gene sufficient for MM017 resistance, preferably comprising one or more mutations within the D1 AAA+ ATPase domain, such as NVL^{P30T}, NVL^{R403W} or NVL^{H304R}.
